(19) United States
(12) Patent Application Publication
(10) Pub. No.: US 2022/0323410 A1
(43) Pub. Date: Oct. 13, 2022

(54) OXYTOCIN ANTAGONIST DOSING REGIMENS FOR PROMOTING EMBRYO IMPLANTATION AND PREVENTING MISCARRIAGE

(71) Applicant: ObsEva S.A., Plan-les-Ouates (CH)

(72) Inventors: Ernest LOUMAYE, Cologny (CH); Oliver POHL, Plan-Les-Ouates (CH); Jean-Pierre GOTTELAND, Geneva (CH)

(21) Appl. No.: 17/640,238

(22) PCT Filed: Aug. 31, 2020
(86) PCT No.: PCT/EP2020/074245

§ 371 Date: Mar. 3, 2022

Related U.S. Application Data
(60) Provisional application No. 63/046,131, filed on Jun. 30, 2020.
(60) Provisional application No. 62/895,262, filed on Sep. 3, 2019.

(51) Int. Cl. A61K 31/40; A61K 38/24; A61P 15/06

(52) U.S. Cl. CPC A61K 31/40; A61K 38/24; A61P 15/06; A61K 9/0053

(57) ABSTRACT

The disclosure provides compositions and methods for the use of oxytocin antagonists, such as substituted pyrrolidin-3-one oxime derivatives of formula (I), among other compounds, in the treatment of subjects undergoing embryo transfer therapy. The compositions and methods of the disclosure can be used to dose subjects with oxytocin antagonists, including (3Z,5S)-5-(hydroxymethyl)-1-[(2′-methyl-1,1′-biphenyl-4-yl)carbonyl]pyrrolidin-3-one O-methyloxime, among others, so as to improve endometrial receptivity and reduce the likelihood of embryo implantation failure and miscarriage following, for example, in vitro fertilization (IVF) and intra cytoplasmic sperm injection (ICSI) embryo transfer procedures.

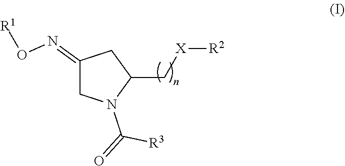

### FIELD OF THE INVENTION

The disclosure relates to composition and methods for dosing subjects with oxytocin receptor antagonists to enhance endometrial receptivity and reduce the likelihood of embryo implantation failure in subjects undergoing embryo transfer therapy.

### BACKGROUND OF THE INVENTION

Despite recent progress in assisted reproductive technology, the overall effectiveness of even advanced treatments, such as in vitro fertilization (IVF) followed by embryo transfer (IVF/ET) remains relatively low, resulting in an average of about 30% live births per treatment cycle (Andersen et al., Human Reproduction 24:1267-1287 (2009)). Moreover, the embryo implantation success rate tends to decrease with age. Many current treatment strategies to promote successful embryo implantation in a subject undergoing embryo transfer therapy have focused on the inhibition of uterine contractions prior to embryo transfer. Such treatment modalities include the administration of A-adrenergic receptor agonists and non-steroidal anti-inflammatory drugs (NSAIDS), which have not been shown to provide sufficient clinical benefit (Bemabeu et al., Human Reproduction 21:364-368 (2006); Moon et al., Fertility and Sterility 82:816-820 (2004); and Tsirigotis et al., Human Reproduction 15:10 (2000)). There remains a need for improved treatment procedures and dosing regimens that can be used to promote successful embryo implantation, for instance, by enhancing endometrial receptivity upon embryo transfer in patients undergoing assisted reproductive technology procedures.

### SUMMARY OF THE INVENTION

The present disclosure provides methods of dosing a subject undergoing embryo transfer therapy with an oxytocin receptor antagonist, for example, to enhance endometrial receptivity upon embryo implantation, thereby reducing the likelihood of embryo implantation failure and of miscarriage. Oxytocin receptor antagonists that can be used in conjunction with the compositions and methods described herein include substituted pyrrolidin-3-one oxime compounds, such as (3Z,5S)-5-(hydroxymethyl)-1-[(2′-methyl-1,1′-biphenyl-4-yl)carbonyl]pyrrolidin-3-one O-methyloxime. Additional examples of oxytocin receptor antagonists that may be used in conjunction with the compositions and methods described herein include epelsiban, retosiban, barusiban, and atosiban, as well as derivatives and variants thereof. Using the compositions and methods described herein, oxytocin antagonists such as the foregoing can be administered to a subject prior to, concurrently with, or after embryo transfer so as to improve endometrial receptivity and reduce the likelihood of embryo implantation failure and miscarriage. The oxytocin antagonist can be administered to the subject in a single dose or in multiple doses, such as doses of varying strength or repeat doses of the same strength. For instance, the oxytocin antagonist may be administered to the subject undergoing embryo transfer in a single high dose or in multiple, lower-strength doses so as to achieve a maximal plasma concentration of the oxytocin antagonist (for instance, of from about 1 μM to about 20 μM, such as from about 1 μM to about 20 μM of a compound represented by formula (I) or (II) as described herein). According to the methods of the disclosure, oxytocin receptor antagonists such as those described herein can be administered to a subject prior to, concurrently with, or after intrauterine transfer of one or more embryos produced ex vivo, for instance, by in vitro fertilization (IVF) or intracytoplasmic sperm injection (ICSI) procedures. The one or more embryos may, for example, be produced by fertilization of an ovum derived from the subject that is undergoing the embryo transfer procedure, or may be derived from a donor that is not undergoing the embryo transfer procedure.

The present disclosure is based, in part, on the discovery of doses of oxytocin receptor antagonists, such as compounds of formula (I) (e.g., (3Z,5S)-5-(hydroxymethyl)-1-[(2′-methyl-1,1′-biphenyl-4-yl)carbonyl]pyrrolidin-3-one O-methyloxime), that are particularly effective at enhancing endometrial receptivity by multiple modes of action. Particularly, doses of compounds of formula (I), such as (3Z,5S)-5-(hydroxymethyl)-1-[(2′-methyl-1,1′-biphenyl-4-yl)carbonyl]pyrrolidin-3-one O-methyloxime, have presently been discovered to reduce uterine contractility and augment the flow of blood to the endometrium. These effects provide important therapeutic benefits to patients undergoing embryo transfer therapy. Together, the reduced uterine contractile activity and enhanced endometrial perfusion engendered by compounds of formula (I), such as (3Z,5S)-5-(hydroxymethyl)-1-[(2′-methyl-1,1′-biphenyl-4-yl)carbonyl]pyrrolidin-3-one O-methyloxime, create an environment in the uterus that is conducive to successful embryo implantation. Without being limited by mechanism, these biological activities represent ways in which compounds of the disclosure may reduce the likelihood of embryo implantation failure and of miscarriage.

In a first aspect, the disclosure provides a method of treating a subject undergoing embryo transfer therapy by administering to the subject a therapeutically effective amount of an oxytocin antagonist, such as a compound represented by formula (I)

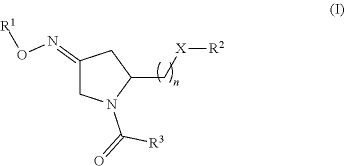

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in an amount of from about 1,500 mg to about 2,700 mg per dose. For example, the compound may be administered to the subject in an amount of from about 1,500 mg to about 2,100 mg per dose, from about 1,550 mg to about 2,050 mg per dose, from about 1,600 mg to about 2,000 mg per dose, from about 1,650 mg to about 1,950 mg per dose, from about 1,700 mg to about 1,900 mg per dose, from about 1,750 mg to about 1,850 mg per dose, from about 1,760 mg to about 1,840 mg per dose, from about 1,770 mg to about 1,830 mg per dose, from about 1,780 mg to about 1,820 mg per dose, from about 1,790 mg to about 1,810 mg per dose, from about 1,791 mg to about 1,809 mg per dose, from about 1,792 mg to about 1,808 mg per dose, from about 1,793 mg to about 1,807 mg per dose, from about 1,794 mg to about 1,806 mg per dose, from about 1,795 mg to about 1,805 mg per dose, from about 1,796 mg to about 1,804 mg per dose, from about 1,797 mg to about 1,803 mg per dose, from about 1,798 mg to about 1,802 mg per dose, or from about 1,799 mg to about 1,801 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of from about 2,100 mg to about 2,700 mg per dose, from about 2,150 mg to about 2,650 mg per dose, from about 2,200 mg to about 2,600 mg per dose, from about 2,250 mg to about 2,550 mg per dose, from about 2,300 mg to about 2,500 mg per dose, from about 2,350 mg to about 2,450 mg per dose, from about 2,360 mg to about 2,440 mg per dose, from about 2,370 mg to about 2,430 mg per dose, from about 2,380 mg to about 2,420 mg per dose, from about 2,390 mg to about 2,410 mg per dose, from about 2,391 mg to about 2,409 mg per dose, from about 2,392 mg to about 2,408 mg per dose, from about 2,393 mg to about 2,407 mg per dose, from about 2,394 mg to about 2,406 mg per dose, from about 2,395 mg to about 2,405 mg per dose, from about 2,396 mg to about 2,404 mg per dose, from about 2,397 mg to about 2,403 mg per dose, from about 2,398 mg to about 2,402 mg per dose, or from about 2,399 mg to about 2,401 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg per dose, such as in an amount of about 1,800 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg per dose, such as in an amount of about 2,400 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of from about 1,600 mg to about 2,600 mg per dose, such as in an amount of from about 1,610 mg to about 2,590 mg per dose, from about 1,620 mg to about 2,580 mg per dose, from about 1,630 mg to about 2,570 mg per dose, from about 1,640 mg to about 2,560 mg per dose, from about 1,650 mg to about 2,550 mg per dose, from about 1,660 mg to about 2,540 mg per dose, from about 1,670 mg to about 2,530 mg per dose, from about 1,680 mg to about 2,520 mg per dose, from about 1,690 mg to about 2,510 mg per dose, from about 1,700 mg to about 2,500 mg per dose, from about 1,710 mg to about 2,490 mg per dose, from about 1,720 mg to about 2,480 mg per dose, from about 1,730 mg to about 2,470 mg per dose, from about 1,740 mg to about 2,460 mg per dose, from about 1,750 mg to about 2,450 mg per dose, from about 1,760 mg to about 2,440 mg per dose, from about 1,770 mg to about 2,430 mg per dose, from about 1,780 mg to about 2,420 mg per dose, from about 1,790 mg to about 2,410 mg per dose, from about 1,800 mg to about 2,400 mg per dose, from about 1,810 mg to about 2,390 mg per dose, from about 1,820 mg to about 2,380 mg per dose, from about 1,830 mg to about 2,370 mg per dose, from about 1,840 mg to about 2,360 mg per dose, from about 1,850 mg to about 2,350 mg per dose, from about 1,860 mg to about 2,340 mg per dose, from about 1,870 mg to about 2,330 mg per dose, from about 1,880 mg to about 2,320 mg per dose, from about 1,890 mg to about 2,310 mg per dose, from about 1,900 mg to about 2,300 mg per dose, from about 1,910 mg to about 2,290 mg per dose, from about 1,920 mg to about 2,280 mg per dose, from about 1,930 mg to about 2,270 mg per dose, from about 1,940 mg to about 2,260 mg per dose, from about 1,950 mg to about 2,250 mg per dose, from about 1,960 mg to about 2,240 mg per dose, from about 1,970 mg to about 2,230 mg per dose, from about 1,980 mg to about 2,220 mg per dose, from about 1,990 mg to about 2,210 mg per dose, from about 2,000 mg to about 2,200 mg per dose, from about 2,010 mg to about 2,190 mg per dose, from about 2,020 mg to about 2,180 mg per dose, from about 2,030 mg to about 2,170 mg per dose, from about 2,040 mg to about 2,160 mg per dose, from about 2,050 mg to about 2,150 mg per dose, from about 2,060 mg to about 2,140 mg per dose, from about 2,070 mg to about 2,130 mg per dose, from about 2,080 mg to about 2,120 mg per dose, or from about 2,090 mg to about 2,110 mg per dose, such as about 2,100 mg per dose e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In another aspect, the disclosure provides a method of treating a subject undergoing embryo transfer therapy by administering to the subject a therapeutically effective amount of an oxytocin antagonist, such as a compound represented by formula (I)

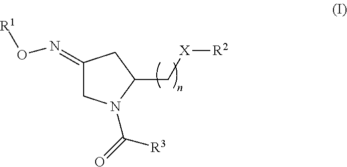

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in one or more doses (e.g., in 1, 2, 3, 4, 5, 6, 7, 8, 9, 10, or more doses) totaling from about 1,500 mg to about 2,700 mg. For example, the compound may be administered to the subject in one or more doses (e.g., in 1, 2, 3, 4, 5, 6, 7, 8, 9, 10, or more doses) totaling from about 1,500 mg to about 2,100 mg, from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg, such as in an amount of about 1,800 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in an amount of about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling from about 1,600 mg to about 2,600 mg, such as in one or more doses (e.g., in 1, 2, 3, 4, 5, 6, 7, 8, 9, 10, or more doses) totaling from about 1,610 mg to about 2,590 mg, from about 1,620 mg to about 2,580 mg, from about 1,630 mg to about 2,570 mg, from about 1,640 mg to about 2,560 mg, from about 1,650 mg to about 2,550 mg, from about 1,660 mg to about 2,540 mg, from about 1,670 mg to about 2,530 mg, from about 1,680 mg to about 2,520 mg, from about 1,690 mg to about 2,510 mg, from about 1,700 mg to about 2,500 mg, from about 1,710 mg to about 2,490 mg, from about 1,720 mg to about 2,480 mg, from about 1,730 mg to about 2,470 mg, from about 1,740 mg to about 2,460 mg, from about 1,750 mg to about 2,450 mg, from about 1,760 mg to about 2,440 mg, from about 1,770 mg to about 2,430 mg, from about 1,780 mg to about 2,420 mg, from about 1,790 mg to about 2,410 mg, from about 1,800 mg to about 2,400 mg, from about 1,810 mg to about 2,390 mg, from about 1,820 mg to about 2,380 mg, from about 1,830 mg to about 2,370 mg, from about 1,840 mg to about 2,360 mg, from about 1,850 mg to about 2,350 mg, from about 1,860 mg to about 2,340 mg, from about 1,870 mg to about 2,330 mg, from about 1,880 mg to about 2,320 mg, from about 1,890 mg to about 2,310 mg, from about 1,900 mg to about 2,300 mg, from about 1,910 mg to about 2,290 mg, from about 1,920 mg to about 2,280 mg, from about 1,930 mg to about 2,270 mg, from about 1,940 mg to about 2,260 mg, from about 1,950 mg to about 2,250 mg, from about 1,960 mg to about 2,240 mg, from about 1,970 mg to about 2,230 mg, from about 1,980 mg to about 2,220 mg, from about 1,990 mg to about 2,210 mg, from about 2,000 mg to about 2,200 mg, from about 2,010 mg to about 2,190 mg, from about 2,020 mg to about 2,180 mg, from about 2,030 mg to about 2,170 mg, from about 2,040 mg to about 2,160 mg, from about 2,050 mg to about 2,150 mg, from about 2,060 mg to about 2,140 mg, from about 2,070 mg to about 2,130 mg, from about 2,080 mg to about 2,120 mg, or from about 2,090 mg to about 2,110 mg, such as about 2,100 mg e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses, such as in a single dose) totaling about 1,800 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses, such as in a single dose) totaling about 2,100 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses, such as in a single dose) totaling about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In a further aspect, the disclosure provides a method of treating a subject undergoing embryo transfer therapy by administering to the subject a therapeutically effective amount of an oxytocin antagonist, such as a compound represented by formula (I)

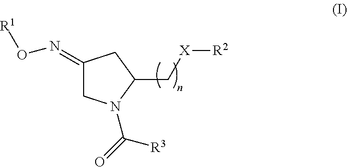

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in a single dose (e.g., on the day of the embryo transfer therapy) of from about 1,500 mg to about 2,700 mg. For example, the compound may be administered to the subject in a single dose (e.g., on the day of the embryo transfer therapy) of from about 1,500 mg to about 2,100 mg, from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (I)).

In some embodiments, the compound is administered to the subject in a single dose (e.g., on the day of the embryo transfer therapy) of from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg, such as in an amount of about 1,800 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in an amount of about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of from about 1,600 mg to about 2,600 mg, such as in a single dose (e.g., on the day of the embryo transfer therapy) of from about 1,610 mg to about 2,590 mg, from about 1,620 mg to about 2,580 mg, from about 1,630 mg to about 2,570 mg, from about 1,640 mg to about 2,560 mg, from about 1,650 mg to about 2,550 mg, from about 1,660 mg to about 2,540 mg, from about 1,670 mg to about 2,530 mg, from about 1,680 mg to about 2,520 mg, from about 1,690 mg to about 2,510 mg, from about 1,700 mg to about 2,500 mg, from about 1,710 mg to about 2,490 mg, from about 1,720 mg to about 2,480 mg, from about 1,730 mg to about 2,470 mg, from about 1,740 mg to about 2,460 mg, from about 1,750 mg to about 2,450 mg, from about 1,760 mg to about 2,440 mg, from about 1,770 mg to about 2,430 mg, from about 1,780 mg to about 2,420 mg, from about 1,790 mg to about 2,410 mg, from about 1,800 mg to about 2,400 mg, from about 1,810 mg to about 2,390 mg, from about 1,820 mg to about 2,380 mg, from about 1,830 mg to about 2,370 mg, from about 1,840 mg to about 2,360 mg, from about 1,850 mg to about 2,350 mg, from about 1,880 mg to about 2,340 mg, from about 1,870 mg to about 2,330 mg, from about 1,880 mg to about 2,320 mg, from about 1,890 mg to about 2,310 mg, from about 1,900 mg to about 2,300 mg, from about 1,910 mg to about 2,290 mg, from about 1,920 mg to about 2,280 mg, from about 1,930 mg to about 2,270 mg, from about 1,940 mg to about 2,260 mg, from about 1,950 mg to about 2,250 mg, from about 1,960 mg to about 2,240 mg, from about 1,970 mg to about 2,230 mg, from about 1,980 mg to about 2,220 mg, from about 1,990 mg to about 2,210 mg, from about 2,000 mg to about 2,200 mg, from about 2,010 mg to about 2,190 mg, from about 2,020 mg to about 2,180 mg, from about 2,030 mg to about 2,170 mg, from about 2,040 mg to about 2,160 mg, from about 2,050 mg to about 2,150 mg, from about 2,060 mg to about 2,140 mg, from about 2,070 mg to about 2,130 mg, from about 2,080 mg to about 2,120 mg, or from about 2,090 mg to about 2,110 mg, such as about 2,100 mg e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of about 1,800 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (I)).

In some embodiments, the compound is administered to the subject in a single dose (e.g., on the day of the embryo transfer therapy) of about 2,100 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In another aspect, the disclosure features an oxytocin antagonist, such as a compound represented by formula (I), for use in the method described in any of the preceding aspects of the disclosure.

In some embodiments of any of the above aspects of the disclosure, the oxytocin antagonist is administered to the subject prior to transfer of one or more embryos (e.g., one, two, three, or more embryos) to the uterus of the subject. In some embodiments, the administering reduces the likelihood of embryo implantation failure and/or miscarriage.

In another aspect, the disclosure provides a method of treating a subject undergoing embryo transfer therapy by transferring one or more embryos (e.g., one, two, three, or more embryos) to the uterus of the subject, wherein the subject has been previously administered a therapeutically effective amount of an oxytocin antagonist, such as a compound represented by formula (I)

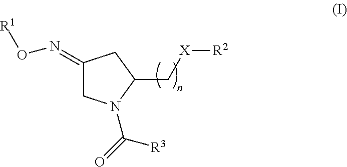

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in an amount of from about 1,500 mg to about 2,700 mg per dose. For example, the compound may be administered to the subject in an amount of from about 1,500 mg to about 2,100 mg per dose, 1,550 mg to about 2,050 mg per dose, from about 1,600 mg to about 2,000 mg per dose, from about 1,650 mg to about 1,950 mg per dose, from about 1,700 mg to about 1,900 mg per dose, from about 1,750 mg to about 1,850 mg per dose, from about 1,760 mg to about 1,840 mg per dose, from about 1,770 mg to about 1,830 mg per dose, from about 1,780 mg to about 1,820 mg per dose, from about 1,790 mg to about 1,810 mg per dose, from about 1,791 mg to about 1,809 mg per dose, from about 1,792 mg to about 1,808 mg per dose, from about 1,793 mg to about 1,807 mg per dose, from about 1,794 mg to about 1,806 mg per dose, from about 1,795 mg to about 1,805 mg per dose, from about 1,796 mg to about 1,804 mg per dose, from about 1,797 mg to about 1,803 mg per dose, from about 1,798 mg to about 1,802 mg per dose, or from about 1,799 mg to about 1,801 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of from about 2,100 mg to about 2,700 mg per dose, from about 2,150 mg to about 2,650 mg per dose, from about 2,200 mg to about 2,600 mg per dose, from about 2,250 mg to about 2,550 mg per dose, from about 2,300 mg to about 2,500 mg per dose, from about 2,350 mg to about 2,450 mg per dose, from about 2,360 mg to about 2,440 mg per dose, from about 2,370 mg to about 2,430 mg per dose, from about 2,380 mg to about 2,420 mg per dose, from about 2,390 mg to about 2,410 mg per dose, from about 2,391 mg to about 2,409 mg per dose, from about 2,392 mg to about 2,408 mg per dose, from about 2,393 mg to about 2,407 mg per dose, from about 2,394 mg to about 2,406 mg per dose, from about 2,395 mg to about 2,405 mg per dose, from about 2,396 mg to about 2,404 mg per dose, from about 2,397 mg to about 2,403 mg per dose, from about 2,398 mg to about 2,402 mg per dose, or from about 2,399 mg to about 2,401 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg per dose, such as in an amount of about 1,800 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg per dose, such as in an amount of about 2,400 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of from about 1,600 mg to about 2,600 mg per dose, such as in an amount of from about 1,610 mg to about 2,590 mg per dose, from about 1,620 mg to about 2,580 mg per dose, from about 1,630 mg to about 2,570 mg per dose, from about 1,640 mg to about 2,560 mg per dose, from about 1,650 mg to about 2,550 mg per dose, from about 1,660 mg to about 2,540 mg per dose, from about 1,670 mg to about 2,530 mg per dose, from about 1,680 mg to about 2,520 mg per dose, from about 1,690 mg to about 2,510 mg per dose, from about 1,700 mg to about 2,500 mg per dose, from about 1,710 mg to about 2,490 mg per dose, from about 1,720 mg to about 2,480 mg per dose, from about 1,730 mg to about 2,470 mg per dose, from about 1,740 mg to about 2,460 mg per dose, from about 1,750 mg to about 2,450 mg per dose, from about 1,760 mg to about 2,440 mg per dose, from about 1,770 mg to about 2,430 mg per dose, from about 1,780 mg to about 2,420 mg per dose, from about 1,790 mg to about 2,410 mg per dose, from about 1,800 mg to about 2,400 mg per dose, from about 1,810 mg to about 2,390 mg per dose, from about 1,820 mg to about 2,380 mg per dose, from about 1,830 mg to about 2,370 mg per dose, from about 1,840 mg to about 2,360 mg per dose, from about 1,850 mg to about 2,350 mg per dose, from about 1,860 mg to about 2,340 mg per dose, from about 1,870 mg to about 2,330 mg per dose, from about 1,880 mg to about 2,320 mg per dose, from about 1,890 mg to about 2,310 mg per dose, from about 1,900 mg to about 2,300 mg per dose, from about 1,910 mg to about 2,290 mg per dose, from about 1,920 mg to about 2,280 mg per dose, from about 1,930 mg to about 2,270 mg per dose, from about 1,940 mg to about 2,260 mg per dose, from about 1,950 mg to about 2,250 mg per dose, from about 1,960 mg to about 2,240 mg per dose, from about 1,970 mg to about 2,230 mg per dose, from about 1,980 mg to about 2,220 mg per dose, from about 1,990 mg to about 2,210 mg per dose, from about 2,000 mg to about 2,200 mg per dose, from about 2,010 mg to about 2,190 mg per dose, from about 2,020 mg to about 2,180 mg per dose, from about 2,030 mg to about 2,170 mg per dose, from about 2,040 mg to about 2,160 mg per dose, from about 2,050 mg to about 2,150 mg per dose, from about 2,060 mg to about 2,140 mg per dose, from about 2,070 mg to about 2,130 mg per dose, from about 2,080 mg to about 2,120 mg per dose, from about 2,090 mg to about 2,110 mg per dose, such as about 2,100 mg per dose e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In another aspect, the disclosure provides a method of treating a subject undergoing embryo transfer therapy by transferring one or more embryos (e.g., one, two, three, or more embryos) to the uterus of the subject, wherein the subject has been previously administered a therapeutically effective amount of an oxytocin antagonist, such as a compound represented by formula (I)

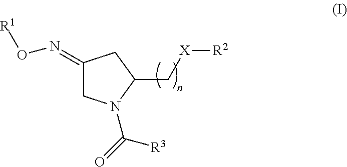

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in one or more doses (e.g., in 1, 2, 3, 4, 5, 6, 7, 8, 9, 10, or more doses) totaling from about 1,500 mg to about 2,700 mg. For example, the compound may be administered to the subject in one or more doses (e.g., in 1, 2, 3, 4, 5, 6, 7, 8, 9, 10, or more doses) totaling from about 1,500 mg to about 2,100 mg, from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg, such as in an amount of about 1,800 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in an amount of about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling from about 1,600 mg to about 2,600 mg, such as in one or more doses (e.g., in 1, 2, 3, 4, 5, 6, 7, 8, 9, 10, or more doses) totaling from about 1,610 mg to about 2,590 mg, from about 1,620 mg to about 2,580 mg, from about 1,630 mg to about 2,570 mg, from about 1,640 mg to about 2,560 mg, from about 1,650 mg to about 2,550 mg, from about 1,660 mg to about 2,540 mg, from about 1,670 mg to about 2,530 mg, from about 1,680 mg to about 2,520 mg, from about 1,690 mg to about 2,510 mg, from about 1,700 mg to about 2,500 mg, from about 1,710 mg to about 2,490 mg, from about 1,720 mg to about 2,480 mg, from about 1,730 mg to about 2,470 mg, from about 1,740 mg to about 2,460 mg, from about 1,750 mg to about 2,450 mg, from about 1,760 mg to about 2,440 mg, from about 1,770 mg to about 2,430 mg, from about 1,780 mg to about 2,420 mg, from about 1,790 mg to about 2,410 mg, from about 1,800 mg to about 2,400 mg, from about 1,810 mg to about 2,390 mg, from about 1,820 mg to about 2,380 mg, from about 1,830 mg to about 2,370 mg, from about 1,840 mg to about 2,360 mg, from about 1,850 mg to about 2,350 mg, from about 1,860 mg to about 2,340 mg, from about 1,870 mg to about 2,330 mg, from about 1,880 mg to about 2,320 mg, from about 1,890 mg to about 2,310 mg, from about 1,900 mg to about 2,300 mg, from about 1,910 mg to about 2,290 mg, from about 1,920 mg to about 2,280 mg, from about 1,930 mg to about 2,270 mg, from about 1,940 mg to about 2,260 mg, from about 1,950 mg to about 2,250 mg, from about 1,960 mg to about 2,240 mg, from about 1,970 mg to about 2,230 mg, from about 1,980 mg to about 2,220 mg, from about 1,990 mg to about 2,210 mg, from about 2,000 mg to about 2,200 mg, from about 2,010 mg to about 2,190 mg, from about 2,020 mg to about 2,180 mg, from about 2,030 mg to about 2,170 mg, from about 2,040 mg to about 2,160 mg, from about 2,050 mg to about 2,150 mg, from about 2,060 mg to about 2,140 mg, from about 2,070 mg to about 2,130 mg, from about 2,080 mg to about 2,120 mg, or from about 2,090 mg to about 2,110 mg, such as about 2,100 mg e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling about 1,800 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling about 2,100 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In a further aspect, the disclosure provides a method of treating a subject undergoing embryo transfer therapy by transferring one or more embryos (e.g., one, two, three, or more embryos) to the uterus of the subject, wherein the subject has been previously administered a therapeutically effective amount of an oxytocin antagonist, such as a compound represented by formula (I)

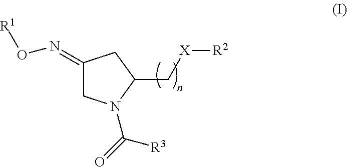

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in a single dose (e.g., on the day of the embryo transfer therapy) of from about 1,500 mg to about 2,700 mg. For example, the compound may be administered to the subject in a single dose (e.g., on the day of the embryo transfer therapy) of from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg, such as in an amount of about 1,800 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in an amount of about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of from about 1,600 mg to about 2,600 mg, such as in a single dose (e.g., on the day of the embryo transfer therapy) of from about 1,610 mg to about 2,590 mg, from about 1,620 mg to about 2,580 mg, from about 1,630 mg to about 2,570 mg, from about 1,640 mg to about 2,560 mg, from about 1,650 mg to about 2,550 mg, from about 1,660 mg to about 2,540 mg, from about 1,670 mg to about 2,530 mg, from about 1,680 mg to about 2,520 mg, from about 1,690 mg to about 2,510 mg, from about 1,700 mg to about 2,500 mg, from about 1,710 mg to about 2,490 mg, from about 1,720 mg to about 2,480 mg, from about 1,730 mg to about 2,470 mg, from about 1,740 mg to about 2,460 mg, from about 1,750 mg to about 2,450 mg, from about 1,760 mg to about 2,440 mg, from about 1,770 mg to about 2,430 mg, from about 1,780 mg to about 2,420 mg, from about 1,790 mg to about 2,410 mg, from about 1,800 mg to about 2,400 mg, from about 1,810 mg to about 2,390 mg, from about 1,820 mg to about 2,380 mg, from about 1,830 mg to about 2,370 mg, from about 1,840 mg to about 2,360 mg, from about 1,850 mg to about 2,350 mg, from about 1,860 mg to about 2,340 mg, from about 1,870 mg to about 2,330 mg, from about 1,880 mg to about 2,320 mg, from about 1,890 mg to about 2,310 mg, from about 1,900 mg to about 2,300 mg, from about 1,910 mg to about 2,290 mg, from about 1,920 mg to about 2,280 mg, from about 1,930 mg to about 2,270 mg, from about 1,940 mg to about 2,260 mg, from about 1,950 mg to about 2,250 mg, from about 1,960 mg to about 2,240 mg, from about 1,970 mg to about 2,230 mg, from about 1,980 mg to about 2,220 mg, from about 1,990 mg to about 2,210 mg, from about 2,000 mg to about 2,200 mg, from about 2,010 mg to about 2,190 mg, from about 2,020 mg to about 2,180 mg, from about 2,030 mg to about 2,170 mg, from about 2,040 mg to about 2,160 mg, from about 2,050 mg to about 2,150 mg, from about 2,060 mg to about 2,140 mg, from about 2,070 mg to about 2,130 mg, from about 2,080 mg to about 2,120 mg, or from about 2,090 mg to about 2,110 mg, such as about 2,100 mg e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of about 1,800 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (I)).

In some embodiments, the compound is administered to the subject in a single dose (e.g., on the day of the embryo transfer therapy) of about 2,100 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (I)).

In some embodiments, the compound is administered to the subject in a single dose (e.g., on the day of the embryo transfer therapy) of about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (I)).

In another aspect, the disclosure features an oxytocin antagonist, such as a compound represented by formula (I), for use in the method described in any of the preceding aspects of the disclosure.

In some embodiments of any of the above aspects of the disclosure, administration of the oxytocin antagonist reduces the likelihood of embryo implantation failure and/or miscarriage.

In an additional aspect, the disclosure provides a method of treating a subject undergoing embryo transfer therapy by:

a. administering to the subject a therapeutically effective amount of an oxytocin antagonist, such as a compound represented by formula (I)

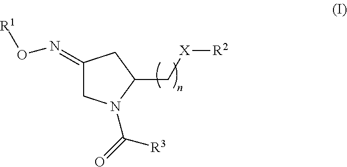

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in an amount of from about 1,500 mg to about 2,700 mg per dose (e.g., in an amount of from about 1,500 mg to about 2,100 mg per dose, from about 1,550 mg to about 2,050 mg per dose, from about 1,600 mg to about 2,000 mg per dose, from about 1,650 mg to about 1,950 mg per dose, from about 1,700 mg to about 1,900 mg per dose, from about 1,750 mg to about 1,850 mg per dose, from about 1,760 mg to about 1,840 mg per dose, from about 1,770 mg to about 1,830 mg per dose, from about 1,780 mg to about 1,820 mg per dose, from about 1,790 mg to about 1,810 mg per dose, from about 1,791 mg to about 1,809 mg per dose, from about 1,792 mg to about 1,808 mg per dose, from about 1,793 mg to about 1,807 mg per dose, from about 1,794 mg to about 1,806 mg per dose, from about 1,795 mg to about 1,805 mg per dose, from about 1,796 mg to about 1,804 mg per dose, from about 1,797 mg to about 1,803 mg per dose, from about 1,798 mg to about 1,802 mg per dose, or from about 1,799 mg to about 1,801 mg per dose, such as an amount of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg per dose, such as in an amount of about 1,800 mg per dose, or in an amount of from about 2,100 mg to about 2,700 mg per dose, from about 2,150 mg to about 2,650 mg per dose, from about 2,200 mg to about 2,600 mg per dose, from about 2,250 mg to about 2,550 mg per dose, from about 2,300 mg to about 2,500 mg per dose, from about 2,350 mg to about 2,450 mg per dose, from about 2,360 mg to about 2,440 mg per dose, from about 2,370 mg to about 2,430 mg per dose, from about 2,380 mg to about 2,420 mg per dose, from about 2,390 mg to about 2,410 mg per dose, from about 2,391 mg to about 2,409 mg per dose, from about 2,392 mg to about 2,408 mg per dose, from about 2,393 mg to about 2,407 mg per dose, from about 2,394 mg to about 2,406 mg per dose, from about 2,395 mg to about 2,405 mg per dose, from about 2,396 mg to about 2,404 mg per dose, from about 2,397 mg to about 2,403 mg per dose, from about 2,398 mg to about 2,402 mg per dose, or from about 2,399 mg to about 2,401 mg per dose, such as in an amount of about 2,400 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II))); and

b. transferring one or more embryos (e.g., one, two, three, or more embryos) to the uterus of the subject following administration of the oxytocin antagonist.

In another aspect, the disclosure provides a method of treating a subject undergoing embryo transfer therapy by:

a. administering to the subject a therapeutically effective amount of an oxytocin antagonist, such as a compound represented by formula (I)

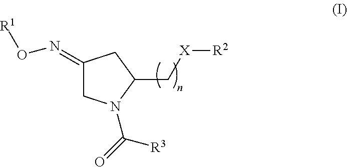

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in one or more doses (e.g., in 1, 2, 3, 4, 5, 6, 7, 8, 9, 10, or more doses) totaling from about 1,500 mg to about 2,700 mg (e.g., in one or more doses (e.g., in 1, 2, 3, 4, 5, 6, 7, 8, 9, 10, or more doses) totaling from about 1,500 mg to about 2,100 mg, from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg, such as in one or more doses (e.g., in 1, 2, 3, 4, 5, 6, 7, 8, 9, 10, or more doses) totaling 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg, such as in one or more doses (e.g., in 1, 2, 3, 4, 5, 6, 7, 8, 9, 10, or more doses) totaling about 1,800 mg, or in one or more doses (e.g., in 1, 2, 3, 4, 5, 6, 7, 8, 9, 10, or more doses) totaling from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg, such as in one or more doses (e.g., in 1, 2, 3, 4, 5, 6, 7, 8, 9, 10, or more doses) totaling about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in one or more doses (e.g., in 1, 2, 3, 4, 5, 6, 7, 8, 9, 10, or more doses) totaling about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II))); and

b. transferring one or more embryos (e.g., one, two, three, or more embryos) to the uterus of the subject following administration of the oxytocin antagonist.

In a further aspect, the disclosure provides a method of treating a subject undergoing embryo transfer therapy by:

a. administering to the subject a therapeutically effective amount of an oxytocin antagonist, such as a compound represented by formula (I)

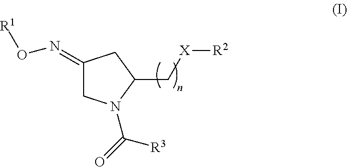

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in a single dose (e.g., on the day of the embryo transfer therapy) of from about 1,500 mg to about 2,700 mg (e.g., in a single dose (e.g., on the day of the embryo transfer therapy) of from about 1,500 mg to about 2,100 mg, from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg, such as in a single dose (e.g., on the day of the embryo transfer therapy) of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg, such as in a single dose (e.g., on the day of the embryo transfer therapy) of about 1,800 mg, or in a single dose (e.g., on the day of the embryo transfer therapy) of from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg, such as in a single dose (e.g., on the day of the embryo transfer therapy) of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in an amount of about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II))); and

b. transferring one or more embryos (e.g., one, two, three, or more embryos) to the uterus of the subject following administration of the oxytocin antagonist.

In another aspect, the disclosure features an oxytocin antagonist, such as a compound represented by formula (I), for use in the method described in any of the preceding aspects of the disclosure.

In some embodiments of any of the above aspects of the disclosure, the administering reduces the likelihood of embryo implantation failure and/or miscarriage.

In some embodiments of any of the above aspects of the disclosure, the oxytocin antagonist is administered to the subject from about 1 hour to about 24 hours prior to the transfer of the one or more embryos to the subject. In some embodiments, the oxytocin antagonist is administered to the subject from about 1 hour to about 12 hours prior the transfer of the one or more embryos to the subject. In some embodiments, the oxytocin antagonist is administered to the subject from about 12 hours to about 24 hours prior the transfer of the one or more embryos to the subject.

In some embodiments of any of the above aspects of the disclosure, the oxytocin antagonist is administered to the subject from about 1 hour to about 10 hours prior the transfer of the one or more embryos to the subject. In some embodiments, the oxytocin antagonist is administered to the subject from about 1 hour to about 9 hours prior the transfer of the one or more embryos to the subject. In some embodiments, the oxytocin antagonist is administered to the subject from about 1 hour to about 8 hours prior the transfer of the one or more embryos to the subject. In some embodiments, the oxytocin antagonist is administered to the subject from about 1 hour to about 7 hours prior the transfer of the one or more embryos to the subject. In some embodiments, the oxytocin antagonist is administered to the subject from about 1 hour to about 6 hours prior the transfer of the one or more embryos to the subject. In some embodiments, the oxytocin antagonist is administered to the subject from about 1 hour to about 5 hours prior the transfer of the one or more embryos to the subject. In some embodiments, the oxytocin antagonist is administered to the subject from about 1 hour to about 4 hours prior the transfer of the one or more embryos to the subject.

In some embodiments of any of the above aspects of the disclosure, the oxytocin antagonist is administered to the subject from about 2 hours to about 6 hours prior the transfer of the one or more embryos to the subject. In some embodiments, the oxytocin antagonist is administered to the subject from about 3 hours to about 5 hours prior the transfer of the one or more embryos to the subject. In some embodiments, the oxytocin antagonist is administered to the subject about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours, or more prior to the transfer of the one or more embryos to the subject.

In some embodiments of any of the above aspects of the disclosure, the oxytocin antagonist is administered to the subject about 4 hours prior to the transfer of the one or more embryos to the subject.

In some embodiments of any of the above aspects of the disclosure, the oxytocin antagonist is administered to the subject prior to embryo transfer in a single dose.

In some embodiments of any of the above aspects of the disclosure, the oxytocin antagonist is administered to the subject prior to embryo transfer (i.e., prior to the transfer of the one or more embryos to the uterus of the subject) in multiple doses (for instance, in multiple periodic doses), such as from 1 to 20 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, prior to embryo transfer. In some embodiments, the oxytocin antagonist is administered to the subject prior to embryo transfer in from 1 to 20 doses per 24 hours, such as 1 dose per 24 hours, 2 doses per 24 hours, 3 doses per 24 hours, 4 doses per 24 hours, 5 doses per 24 hours, 6 doses per 24 hours, 7 doses per 24 hours, 8 doses per 24 hours, 9 doses per 24 hours, 10 doses per 24 hours, 11 doses per 24 hours, 12 doses per 24 hours, 13 doses per 24 hours, 14 doses per 24 hours, 15 doses per 24 hours, 16 doses per 24 hours, 17 doses per 24 hours, 18 doses per 24 hours, 19 doses per 24 hours, 20 doses per 24 hours. In some embodiments, the oxytocin antagonist is administered to the subject prior to embryo transfer in more than 20 doses per 24 hours.

For instance, in some embodiments, the oxytocin antagonist is administered to the subject in from 1 to 10 doses, for example, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, prior to embryo transfer. In some embodiments, the oxytocin antagonist is administered to the subject prior to embryo transfer in from 1 to 10 doses per 24 hours, such as 1 dose per 24 hours, 2 doses per 24 hours, 3 doses per 24 hours, 4 doses per 24 hours, 5 doses per 24 hours, 6 doses per 24 hours, 7 doses per 24 hours, 8 doses per 24 hours, 9 doses per 24 hours, 10 doses per 24 hours.

In some embodiments, the oxytocin antagonist is administered to the subject in from 1 to 5 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, prior to embryo transfer. In some embodiments, the oxytocin antagonist is administered to the subject in from 10 to 20 doses, for instance, per 12 hours, per 24 hours, per 36 hours, per 48 hours, per 60 hours, per 72 hours, per 84 hours, per 96 hours, per 108 hours, per 120 hours, per 132 hours, per 144 hours, per 156 hours, per 168 hours, or longer, prior to embryo transfer. In some embodiments, the oxytocin antagonist is administered to the subject in from 10 to 15 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, prior to embryo transfer.

In some embodiments, the oxytocin antagonist is administered to the subject in 1, 2, 3, 4, 5, 6, 7, 8, 9, 10, 11, 12, 13, 14, 15, 16, 17, 18, 19, 20, or more, doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, prior to embryo transfer.

In some embodiments, the oxytocin antagonist is administered to the subject prior to embryo transfer in up to 7 doses (e.g., 1, 2, 3, 4, 5, 6, or 7 doses) per 24 hours. In some embodiments, the oxytocin antagonist is administered to the subject prior to embryo transfer in 1 dose per 24 hours, such as 1 dose per 24 hours of compound (II), below. In some embodiments, the oxytocin antagonist is administered to the subject prior to embryo transfer in 2 doses per 24 hours, such as 2 doses per 24 hours of compound (II), below. In some embodiments, the oxytocin antagonist is administered to the subject prior to embryo transfer in 3 doses per 24 hours, such as 3 doses per 24 hours of compound (II), below. In some embodiments, the oxytocin antagonist is administered to the subject prior to embryo transfer in 4 doses per 24 hours, such as 4 doses per 24 hours of compound (II), below. In some embodiments, the oxytocin antagonist is administered to the subject prior to embryo transfer in 5 doses per 24 hours, such as 5 doses per 24 hours of compound (II), below. In some embodiments, the oxytocin antagonist is administered to the subject prior to embryo transfer in 6 doses per 24 hours, such as 6 doses per 24 hours of compound (II), below. In some embodiments, the oxytocin antagonist is administered to the subject prior to embryo transfer in 7 doses per 24 hours, such as 7 doses per 24 hours of compound (II), below.

The multiple doses may be administered, for example, starting at from about 1 hour to about 14 days, or more, prior to embryo transfer. In some embodiments, the multiple doses are administered starting at from about 1 hour to about 7 days, or more, prior to embryo transfer. In some embodiments, the multiple doses may be administered starting at from about 1 day to about 14 days prior to embryo transfer. In some embodiments, the multiple doses may be administered starting at from about 3 days to about 11 days prior to embryo transfer. In some embodiments, the multiple doses may be administered starting at from about 1 day to about 7 days prior to embryo transfer. In some embodiments, the multiple doses may be administered starting at from about 2 days to about 5 days prior to embryo transfer. In some embodiments, the multiple doses may be administered starting at from about 3 days to about 4 days prior to embryo transfer. For instance, the multiple doses may be administered starting at 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, 96 hours, 108 hours, 120 hours, 132 hours, 144 hours, 156 hours, 168 hours, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more, prior to embryo transfer to the subject.

In some embodiments, the multiple doses are administered starting at about 2 days prior to embryo transfer.

In some embodiments, the multiple doses are administered starting at about 3 days prior to embryo transfer.

In some embodiments, the multiple doses are administered starting at about 4 days prior to embryo transfer.

In some embodiments, the multiple doses are administered starting at about 5 days prior to embryo transfer.

In some embodiments, the multiple doses are administered starting at about 6 days prior to embryo transfer.

In some embodiments, the multiple doses are administered starting at about 7 days prior to embryo transfer.

In some embodiments, the multiple doses terminate on the day of embryo transfer to the subject. In some embodiments, the multiple doses terminate with a final dose of the oxytocin antagonist that is administered concurrently with (e.g., within 60 minutes of) transfer of the one or more embryos to the subject.

In some embodiments of any of the above aspects of the disclosure, the multiple doses continue following embryo transfer. For instance, the oxytocin antagonist may be administered to the subject in one or more additional doses concurrently with embryo transfer. In some embodiments, the oxytocin antagonist is administered to the subject in one or more additional doses following embryo transfer (for instance, in multiple periodic doses), such as in one or more additional doses administered within about 1 hour to about 1 week, or longer (e.g., within about 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, 96 hours, 108 hours, 120 hours, 132 hours, 144 hours, 156 hours, 168 hours, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more) following the transfer of the one or more embryos to the subject.

For example, in some embodiments, the oxytocin antagonist is administered to the subject in one or more additional doses within about 1 hour to about 24 hours following the transfer of the one or more embryos to the subject. In some embodiments, the oxytocin antagonist is administered to the subject in one or more additional doses within about 1 hour to about 12 hours following the transfer of the one or more embryos to the subject. In some embodiments, the oxytocin antagonist is administered to the subject in one or more additional doses within from about 12 hours to about 24 hours following the transfer of the one or more embryos to the subject. In some embodiments, the oxytocin antagonist is administered to the subject in one or more additional doses within from about 1 hour to about 10 hours following the transfer of the one or more embryos to the subject. In some embodiments, the oxytocin antagonist is administered to the subject in one or more additional doses within from about 1 hour to about 9 hours following the transfer of the one or more embryos to the subject. In some embodiments, the oxytocin antagonist is administered to the subject in one or more additional doses within from about 1 hour to about 8 hours following the transfer of the one or more embryos to the subject. In some embodiments, the oxytocin antagonist is administered to the subject in one or more additional doses within from about 1 hour to about 7 hours following the transfer of the one or more embryos to the subject. In some embodiments, the oxytocin antagonist is administered to the subject in one or more additional doses within from about 1 hour to about 6 hours following the transfer of the one or more embryos to the subject. In some embodiments, the oxytocin antagonist is administered to the subject in one or more additional doses within from about 1 hour to about 5 hours following the transfer of the one or more embryos to the subject. In some embodiments, the oxytocin antagonist is administered to the subject in one or more additional doses within from about 1 hour to about 4 hours following the transfer of the one or more embryos to the subject. In some embodiments, the oxytocin antagonist is administered to the subject in one or more additional doses within from about 2 hours to about 6 hours following the transfer of the one or more embryos to the subject. In some embodiments, the oxytocin antagonist is administered to the subject in one or more additional doses within from about 3 hours to about 5 hours following the transfer of the one or more embryos to the subject.

In some embodiments, the oxytocin antagonist is administered to the subject in one or more additional doses starting at about 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, 96 hours, 108 hours, 120 hours, 132 hours, 144 hours, 156 hours, 168 hours, or more, following the transfer of the one or more embryos to the subject.

In some embodiments, the oxytocin antagonist is administered to the subject in multiple additional doses following embryo transfer, such as in from 1 to 20 additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is additionally administered to the subject following embryo transfer in from 1 to 20 doses per 24 hours, such as 1 dose per 24 hours, 2 doses per 24 hours, 3 doses per 24 hours, 4 doses per 24 hours, 5 doses per 24 hours, 6 doses per 24 hours, 7 doses per 24 hours, 8 doses per 24 hours, 9 doses per 24 hours, 10 doses per 24 hours, 11 doses per 24 hours, 12 doses per 24 hours, 13 doses per 24 hours, 14 doses per 24 hours, 15 doses per 24 hours, 16 doses per 24 hours, 17 doses per 24 hours, 18 doses per 24 hours, 19 doses per 24 hours, 20 doses per 24 hours. In some embodiments, the oxytocin antagonist is additionally administered to the subject following embryo transfer in more than 20 doses per 24 hours.

For instance, in some embodiments, the oxytocin antagonist is administered to the subject in from 1 to 10 additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is additionally administered to the subject following embryo transfer in from 1 to 10 doses per 24 hours, such as 1 dose per 24 hours, 2 doses per 24 hours, 3 doses per 24 hours, 4 doses per 24 hours, 5 doses per 24 hours, 6 doses per 24 hours, 7 doses per 24 hours, 8 doses per 24 hours, 9 doses per 24 hours, 10 doses per 24 hours.

In some embodiments, the oxytocin antagonist is administered to the subject in from 1 to 5 additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is administered to the subject in from 10 to 20 additional doses, for instance, per 12 hours, per 24 hours, per 36 hours, per 48 hours, per 60 hours, per 72 hours, per 84 hours, per 96 hours, per 108 hours, per 120 hours, per 132 hours, per 144 hours, per 156 hours, per 168 hours, or longer, following embryo transfer. In some embodiments, the oxytocin antagonist is administered to the subject in from 10 to 15 additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer.

In some embodiments, the oxytocin antagonist is administered to the subject in 1, 2, 3, 4, 5, 6, 7, 8, 9, 10, 11, 12, 13, 14, 15, 16, 17, 18, 19, 20, or more, additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer.

In some embodiments, the oxytocin antagonist is administered to the subject following embryo transfer in up to 7 additional doses (e.g., 1, 2, 3, 4, 5, 6, or 7 doses) per 24 hours. In some embodiments, the oxytocin antagonist is additionally administered to the subject following embryo transfer in 1 dose per 24 hours, such as 1 additional dose per 24 hours of compound (II), below. In some embodiments, the oxytocin antagonist is additionally administered to the subject following embryo transfer in 2 doses per 24 hours, such as 2 additional doses per 24 hours of compound (II), below. In some embodiments, the oxytocin antagonist is additionally administered to the subject following embryo transfer in 3 doses per 24 hours, such as 3 additional doses per 24 hours of compound (II), below. In some embodiments, the oxytocin antagonist is additionally administered to the subject following embryo transfer in 4 doses per 24 hours, such as 4 additional doses per 24 hours of compound (II), below. In some embodiments, the oxytocin antagonist is additionally administered to the subject following embryo transfer in 5 doses per 24 hours, such as 5 additional doses per 24 hours of compound (II), below. In some embodiments, the oxytocin antagonist is additionally administered to the subject following embryo transfer in 6 doses per 24 hours, such as 6 additional doses per 24 hours of compound (II), below. In some embodiments, the oxytocin antagonist is additionally administered to the subject following embryo transfer in 7 doses per 24 hours, such as 7 additional doses per 24 hours of compound (II), below.

When one or more additional doses of the oxytocin antagonist are administered to the subject following embryo transfer, administration of the oxytocin antagonist may terminate, for instance, within from about 1 hour to about 14 days, or more, following embryo transfer. For instance, administration of the oxytocin antagonist may terminate within about 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, 96 hours, 108 hours, 120 hours, 132 hours, 144 hours, 156 hours, 168 hours, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more, following embryo transfer.

Thus, in some embodiments, the oxytocin antagonist is administered to the subject in additional daily doses following embryo transfer for about 1 day to about 14 days following embryo transfer. In some embodiments, the additional daily doses are administered to the subject for about 3 days to about 11 days following embryo transfer. In some embodiments, the additional daily doses are administered to the subject for 7 days following embryo transfer.

In another aspect, the disclosure provides a method of treating a subject undergoing embryo transfer therapy by administering to the subject a therapeutically effective amount of an oxytocin antagonist, such as a compound represented by formula (I)

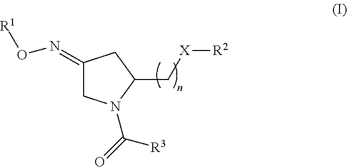

or a geometric isomer, enantiomer, diastereomer, racemate, or sat thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in an amount of from about 1,500 mg to about 2,700 mg per dose (e.g., in an amount of from about 1,500 mg to about 2,100 mg per dose, from about 1,550 mg to about 2,050 mg per dose, from about 1,600 mg to about 2,000 mg per dose, from about 1,650 mg to about 1,950 mg per dose, from about 1,700 mg to about 1,900 mg per dose, from about 1,750 mg to about 1,850 mg per dose, from about 1,760 mg to about 1,840 mg per dose, from about 1,770 mg to about 1,830 mg per dose, from about 1,780 mg to about 1,820 mg per dose, from about 1,790 mg to about 1,810 mg per dose, from about 1,791 mg to about 1,809 mg per dose, from about 1,792 mg to about 1,808 mg per dose, from about 1,793 mg to about 1,807 mg per dose, from about 1,794 mg to about 1,806 mg per dose, from about 1,795 mg to about 1,805 mg per dose, from about 1,796 mg to about 1,804 mg per dose, from about 1,797 mg to about 1,803 mg per dose, from about 1,798 mg to about 1,802 mg per dose, or from about 1,799 mg to about 1,801 mg per dose, such as an amount of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg per dose, such as in an amount of about 1,800 mg per dose, or in an amount of from about 2,100 mg to about 2,700 mg per dose, from about 2,150 mg to about 2,650 mg per dose, from about 2,200 mg to about 2,600 mg per dose, from about 2,250 mg to about 2,550 mg per dose, from about 2,300 mg to about 2,500 mg per dose, from about 2,350 mg to about 2,450 mg per dose, from about 2,360 mg to about 2,440 mg per dose, from about 2,370 mg to about 2,430 mg per dose, from about 2,380 mg to about 2,420 mg per dose, from about 2,390 mg to about 2,410 mg per dose, from about 2,391 mg to about 2,409 mg per dose, from about 2,392 mg to about 2,408 mg per dose, from about 2,393 mg to about 2,407 mg per dose, from about 2,394 mg to about 2,406 mg per dose, from about 2,395 mg to about 2,405 mg per dose, from about 2,396 mg to about 2,404 mg per dose, from about 2,397 mg to about 2,403 mg per dose, from about 2,398 mg to about 2,402 mg per dose, or from about 2,399 mg to about 2,401 mg per dose, such as in an amount of about 2,400 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II))), and

wherein the oxytocin antagonist is administered concurrently with transfer of one or more embryos (e.g., one, two, three, or more embryos) to the uterus of the subject.

In another aspect, the disclosure provides a method of treating a subject undergoing embryo transfer therapy by administering to the subject a therapeutically effective amount of an oxytocin antagonist, such as a compound represented by formula (I)

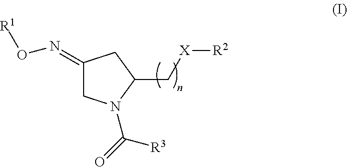

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in one or more doses (e.g., in 1, 2, 3, 4, 5, 6, 7, 8, 9, 10, or more doses) totaling from about 1,500 mg to about 2,700 mg (e.g., in one or more doses (e.g., in 1, 2, 3, 4, 5, 6, 7, 8, 9, 10, or more doses) totaling from about 1,500 mg to about 2,100 mg, from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg, such as in one or more doses (e.g., in 1, 2, 3, 4, 5, 6, 7, 8, 9, 10, or more doses) totaling 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg, such as in one or more doses (e.g., in 1, 2, 3, 4, 5, 6, 7, 8, 9, 10, or more doses) totaling about 1,800 mg, or in one or more doses (e.g., in 1, 2, 3, 4, 5, 6, 7, 8, 9, 10, or more doses) totaling from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg, such as in one or more doses (e.g., in 1, 2, 3, 4, 5, 6, 7, 8, 9, 10, or more doses) totaling about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in one or more doses (e.g., in 1, 2, 3, 4, 5, 6, 7, 8, 9, 10, or more doses) totaling about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II))), and

wherein the oxytocin antagonist is administered concurrently with transfer of one or more embryos (e.g., one, two, three, or more embryos) to the uterus of the subject.

In a further aspect, the disclosure provides a method of treating a subject undergoing embryo transfer therapy by administering to the subject a therapeutically effective amount of an oxytocin antagonist, such as a compound represented by formula (I)

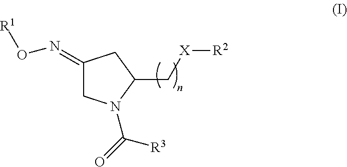

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in a single dose (e.g., on the day of the embryo transfer therapy) of from about 1,500 mg to about 2,700 mg (e.g., in a single dose (e.g., on the day of the embryo transfer therapy) of from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg, such as in a single dose (e.g., on the day of the embryo transfer therapy) of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg, such as in a single dose (e.g., on the day of the embryo transfer therapy) of about 1,800 mg, or in a single dose (e.g., on the day of the embryo transfer therapy) of from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg, such as in a single dose (e.g., on the day of the embryo transfer therapy) of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in a single dose (e.g., on the day of the embryo transfer therapy) of about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II))), and

wherein the oxytocin antagonist is administered concurrently with transfer of one or more embryos (e.g., one, two, three, or more embryos) to the uterus of the subject.

In another aspect, the disclosure features an oxytocin antagonist, such as a compound represented by formula (I), for use in the method described in any of the preceding aspects of the disclosure.

In some embodiments of any of the above aspects of the disclosure, the administering reduces the likelihood of embryo implantation failure and/or miscarriage.

In another aspect, the disclosure provides a method of treating a subject undergoing embryo transfer therapy by transferring one or more embryos (e.g., one, two, three, or more embryos) to the uterus of the subject, wherein the subject is concurrently administered a therapeutically effective amount of an oxytocin antagonist, such as a compound represented by formula (I)

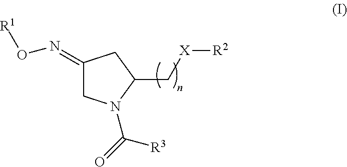

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in an amount of from about 1,500 mg to about 2,700 mg per dose. For example, the compound may be administered to the subject in an amount of from about 1,500 mg to about 2,100 mg per dose, from about 1,550 mg to about 2,050 mg per dose, from about 1,600 mg to about 2,000 mg per dose, from about 1,650 mg to about 1,950 mg per dose, from about 1,700 mg to about 1,900 mg per dose, from about 1,750 mg to about 1,850 mg per dose, from about 1,760 mg to about 1,840 mg per dose, from about 1,770 mg to about 1,830 mg per dose, from about 1,780 mg to about 1,820 mg per dose, from about 1,790 mg to about 1,810 mg per dose, from about 1,791 mg to about 1,809 mg per dose, from about 1,792 mg to about 1,808 mg per dose, from about 1,793 mg to about 1,807 mg per dose, from about 1,794 mg to about 1,806 mg per dose, from about 1,795 mg to about 1,805 mg per dose, from about 1,796 mg to about 1,804 mg per dose, from about 1,797 mg to about 1,803 mg per dose, from about 1,798 mg to about 1,802 mg per dose, or from about 1,799 mg to about 1,801 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of from about 2,100 mg to about 2,700 mg per dose, from about 2,150 mg to about 2,650 mg per dose, from about 2,200 mg to about 2,600 mg per dose, from about 2,250 mg to about 2,550 mg per dose, from about 2,300 mg to about 2,500 mg per dose, from about 2,350 mg to about 2,450 mg per dose, from about 2,360 mg to about 2,440 mg per dose, from about 2,370 mg to about 2,430 mg per dose, from about 2,380 mg to about 2,420 mg per dose, from about 2,390 mg to about 2,410 mg per dose, from about 2,391 mg to about 2,409 mg per dose, from about 2,392 mg to about 2,408 mg per dose, from about 2,393 mg to about 2,407 mg per dose, from about 2,394 mg to about 2,406 mg per dose, from about 2,395 mg to about 2,405 mg per dose, from about 2,396 mg to about 2,404 mg per dose, from about 2,397 mg to about 2,403 mg per dose, from about 2,398 mg to about 2,402 mg per dose, or from about 2,399 mg to about 2,401 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg per dose, such as in an amount of about 1,800 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg per dose, such as in an amount of about 2,400 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of from about 1,600 mg to about 2,600 mg per dose, such as in an amount of from about 1,610 mg to about 2,590 mg per dose, from about 1,620 mg to about 2,580 mg per dose, from about 1,630 mg to about 2,570 mg per dose, from about 1,640 mg to about 2,560 mg per dose, from about 1,650 mg to about 2,550 mg per dose, from about 1,660 mg to about 2,540 mg per dose, from about 1,670 mg to about 2,530 mg per dose, from about 1,680 mg to about 2,520 mg per dose, from about 1,690 mg to about 2,510 mg per dose, from about 1,700 mg to about 2,500 mg per dose, from about 1,710 mg to about 2,490 mg per dose, from about 1,720 mg to about 2,480 mg per dose, from about 1,730 mg to about 2,470 mg per dose, from about 1,740 mg to about 2,460 mg per dose, from about 1,750 mg to about 2,450 mg per dose, from about 1,760 mg to about 2,440 mg per dose, from about 1,770 mg to about 2,430 mg per dose, from about 1,780 mg to about 2,420 mg per dose, from about 1,790 mg to about 2,410 mg per dose, from about 1,800 mg to about 2,400 mg per dose, from about 1,810 mg to about 2,390 mg per dose, from about 1,820 mg to about 2,380 mg per dose, from about 1,830 mg to about 2,370 mg per dose, from about 1,840 mg to about 2,360 mg per dose, from about 1,850 mg to about 2,350 mg per dose, from about 1,860 mg to about 2,340 mg per dose, from about 1,870 mg to about 2,330 mg per dose, from about 1,880 mg to about 2,320 mg per dose, from about 1,890 mg to about 2,310 mg per dose, from about 1,900 mg to about 2,300 mg per dose, from about 1,910 mg to about 2,290 mg per dose, from about 1,920 mg to about 2,280 mg per dose, from about 1,930 mg to about 2,270 mg per dose, from about 1,940 mg to about 2,260 mg per dose, from about 1,950 mg to about 2,250 mg per dose, from about 1,960 mg to about 2,240 mg per dose, from about 1,970 mg to about 2,230 mg per dose, from about 1,980 mg to about 2,220 mg per dose, from about 1,990 mg to about 2,210 mg per dose, from about 2,000 mg to about 2,200 mg per dose, from about 2,010 mg to about 2,190 mg per dose, from about 2,020 mg to about 2,180 mg per dose, from about 2,030 mg to about 2,170 mg per dose, from about 2,040 mg to about 2,160 mg per dose, from about 2,050 mg to about 2,150 mg per dose, from about 2,060 mg to about 2,140 mg per dose, from about 2,070 mg to about 2,130 mg per dose, from about 2,080 mg to about 2,120 mg per dose, or from about 2,090 mg to about 2,110 mg per dose, such as about 2,100 mg per dose e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In another aspect, the disclosure provides a method of treating a subject undergoing embryo transfer therapy by transferring one or more embryos (e.g., one, two, three, or more embryos) to the uterus of the subject, wherein the subject is concurrently administered a therapeutically effective amount of an oxytocin antagonist, such as a compound represented by formula (I)

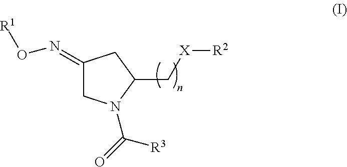

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in one or more doses (e.g., in 1, 2, 3, 4, 5, 6, 7, 8, 9, 10, or more doses) totaling from about 1,500 mg to about 2,700 mg. For example, the compound may be administered to the subject in one or more doses (e.g., in 1, 2, 3, 4, 5, 6, 7, 8, 9, 10, or more doses) totaling from about 1,500 mg to about 2,100 mg, from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg, such as in an amount of about 1,800 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in an amount of about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling from about 1,600 mg to about 2,600 mg, such as in one or more doses (e.g., in 1, 2, 3, 4, 5, 6, 7, 8, 9, 10, or more doses) totaling from about 1,610 mg to about 2,590 mg, from about 1,620 mg to about 2,580 mg, from about 1,630 mg to about 2,570 mg, from about 1,640 mg to about 2,560 mg, from about 1,650 mg to about 2,550 mg, from about 1,660 mg to about 2,540 mg, from about 1,670 mg to about 2,530 mg, from about 1,680 mg to about 2,520 mg, from about 1,690 mg to about 2,510 mg, from about 1,700 mg to about 2,500 mg, from about 1,710 mg to about 2,490 mg, from about 1,720 mg to about 2,480 mg, from about 1,730 mg to about 2,470 mg, from about 1,740 mg to about 2,460 mg, from about 1,750 mg to about 2,450 mg, from about 1,760 mg to about 2,440 mg, from about 1,770 mg to about 2,430 mg, from about 1,780 mg to about 2,420 mg, from about 1,790 mg to about 2,410 mg, from about 1,800 mg to about 2,400 mg, from about 1,810 mg to about 2,390 mg, from about 1,820 mg to about 2,380 mg, from about 1,830 mg to about 2,370 mg, from about 1,840 mg to about 2,360 mg, from about 1,850 mg to about 2,350 mg, from about 1,860 mg to about 2,340 mg, from about 1,870 mg to about 2,330 mg, from about 1,880 mg to about 2,320 mg, from about 1,890 mg to about 2,310 mg, from about 1,900 mg to about 2,300 mg, from about 1,910 mg to about 2,290 mg, from about 1,920 mg to about 2,280 mg, from about 1,930 mg to about 2,270 mg, from about 1,940 mg to about 2,260 mg, from about 1,950 mg to about 2,250 mg, from about 1,960 mg to about 2,240 mg, from about 1,970 mg to about 2,230 mg, from about 1,980 mg to about 2,220 mg, from about 1,990 mg to about 2,210 mg, from about 2,000 mg to about 2,200 mg, from about 2,010 mg to about 2,190 mg, from about 2,020 mg to about 2,180 mg, from about 2,030 mg to about 2,170 mg, from about 2,040 mg to about 2,160 mg, from about 2,050 mg to about 2,150 mg, from about 2,060 mg to about 2,140 mg, from about 2,070 mg to about 2,130 mg, from about 2,080 mg to about 2,120 mg, or from about 2,090 mg to about 2,110 mg, such as about 2,100 mg e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses, such as in a single dose) totaling about 1,800 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses, such as in a single dose) totaling about 2,100 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses, such as in a single dose) totaling about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In a further aspect, the disclosure provides a method of treating a subject undergoing embryo transfer therapy by transferring one or more embryos (e.g., one, two, three, or more embryos) to the uterus of the subject, wherein the subject is concurrently administered a therapeutically effective amount of an oxytocin antagonist, such as a compound represented by formula (I)

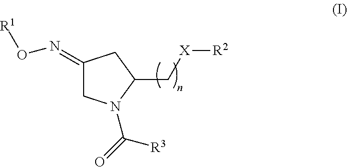

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in a single dose (e.g., on the day of the embryo transfer therapy) of from about 1,500 mg to about 2,700 mg. For example, the compound may be administered to the subject in a single dose (e.g., on the day of the embryo transfer therapy) of from about 1,500 mg to about 2,100 mg, from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg, such as in an amount of about 1,800 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in an amount of about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of from about 1,600 mg to about 2,600 mg, such as in a single dose (e.g., on the day of the embryo transfer therapy) of from about 1,610 mg to about 2,590 mg, from about 1,620 mg to about 2,580 mg, from about 1,630 mg to about 2,570 mg, from about 1,640 mg to about 2,560 mg, from about 1,650 mg to about 2,550 mg, from about 1,660 mg to about 2,540 mg, from about 1,670 mg to about 2,530 mg, from about 1,680 mg to about 2,520 mg, from about 1,690 mg to about 2,510 mg, from about 1,700 mg to about 2,500 mg, from about 1,710 mg to about 2,490 mg, from about 1,720 mg to about 2,480 mg, from about 1,730 mg to about 2,470 mg, from about 1,740 mg to about 2,460 mg, from about 1,750 mg to about 2,450 mg, from about 1,760 mg to about 2,440 mg, from about 1,770 mg to about 2,430 mg, from about 1,780 mg to about 2,420 mg, from about 1,790 mg to about 2,410 mg, from about 1,800 mg to about 2,400 mg, from about 1,810 mg to about 2,390 mg, from about 1,820 mg to about 2,380 mg, from about 1,830 mg to about 2,370 mg, from about 1,840 mg to about 2,360 mg, from about 1,850 mg to about 2,350 mg, from about 1,860 mg to about 2,340 mg, from about 1,870 mg to about 2,330 mg, from about 1,880 mg to about 2,320 mg, from about 1,890 mg to about 2,310 mg, from about 1,900 mg to about 2,300 mg, from about 1,910 mg to about 2,290 mg, from about 1,920 mg to about 2,280 mg, from about 1,930 mg to about 2,270 mg, from about 1,940 mg to about 2,260 mg, from about 1,950 mg to about 2,250 mg, from about 1,960 mg to about 2,240 mg, from about 1,970 mg to about 2,230 mg, from about 1,980 mg to about 2,220 mg, from about 1,990 mg to about 2,210 mg, from about 2,000 mg to about 2,200 mg, from about 2,010 mg to about 2,190 mg, from about 2,020 mg to about 2,180 mg, from about 2,030 mg to about 2,170 mg, from about 2,040 mg to about 2,160 mg, from about 2,050 mg to about 2,150 mg, from about 2,060 mg to about 2,140 mg, from about 2,070 mg to about 2,130 mg, from about 2,080 mg to about 2,120 mg, or from about 2,090 mg to about 2,110 mg, such as about 2,100 mg e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of about 1,800 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (I)).

In some embodiments, the compound is administered to the subject in a single dose (e.g., on the day of the embryo transfer therapy) of about 2,100 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (I)).

In some embodiments, the compound is administered to the subject in a single dose (e.g., on the day of the embryo transfer therapy) of about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (I)).

In another aspect, the disclosure features an oxytocin antagonist, such as a compound represented by formula (I), for use in the method described in any of the preceding aspects of the disclosure.

In some embodiments of any of the above aspects of the disclosure, administration of the oxytocin antagonist reduces the likelihood of embryo implantation failure and/or miscarriage.

In another aspect, the disclosure provides a method of treating a subject undergoing embryo transfer therapy by:

a. administering to the subject a therapeutically effective amount of an oxytocin antagonist, such as a compound represented by formula (I)

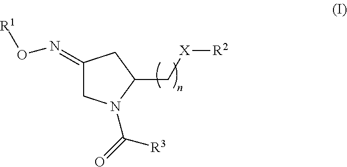

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in an amount of from about 1,500 mg to about 2,700 mg per dose (e.g., in an amount of from about 1,500 mg to about 2,100 mg per dose, from about 1,550 mg to about 2,050 mg per dose, from about 1,600 mg to about 2,000 mg per dose, from about 1,650 mg to about 1,950 mg per dose, from about 1,700 mg to about 1,900 mg per dose, from about 1,750 mg to about 1,850 mg per dose, from about 1,760 mg to about 1,840 mg per dose, from about 1,770 mg to about 1,830 mg per dose, from about 1,780 mg to about 1,820 mg per dose, from about 1,790 mg to about 1,810 mg per dose, from about 1,791 mg to about 1,809 mg per dose, from about 1,792 mg to about 1,808 mg per dose, from about 1,793 mg to about 1,807 mg per dose, from about 1,794 mg to about 1,806 mg per dose, from about 1,795 mg to about 1,805 mg per dose, from about 1,796 mg to about 1,804 mg per dose, from about 1,797 mg to about 1,803 mg per dose, from about 1,798 mg to about 1,802 mg per dose, or from about 1,799 mg to about 1,801 mg per dose, such as an amount of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg per dose, such as in an amount of about 1,800 mg per dose, or in an amount of from about 2,100 mg to about 2,700 mg per dose, from about 2,150 mg to about 2,650 mg per dose, from about 2,200 mg to about 2,600 mg per dose, from about 2,250 mg to about 2,550 mg per dose, from about 2,300 mg to about 2,500 mg per dose, from about 2,350 mg to about 2,450 mg per dose, from about 2,360 mg to about 2,440 mg per dose, from about 2,370 mg to about 2,430 mg per dose, from about 2,380 mg to about 2,420 mg per dose, from about 2,390 mg to about 2,410 mg per dose, from about 2,391 mg to about 2,409 mg per dose, from about 2,392 mg to about 2,408 mg per dose, from about 2,393 mg to about 2,407 mg per dose, from about 2,394 mg to about 2,406 mg per dose, from about 2,395 mg to about 2,405 mg per dose, from about 2,396 mg to about 2,404 mg per dose, from about 2,397 mg to about 2,403 mg per dose, from about 2,398 mg to about 2,402 mg per dose, or from about 2,399 mg to about 2,401 mg per dose, such as in an amount of about 2,400 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II))); and

b. transferring one or more embryos (e.g., one, two, three, or more embryos) to the uterus of the subject concurrently with administration of the oxytocin antagonist.

In another aspect, the disclosure provides a method of treating a subject undergoing embryo transfer therapy by:

a. administering to the subject a therapeutically effective amount of an oxytocin antagonist, such as a compound represented by formula (I)

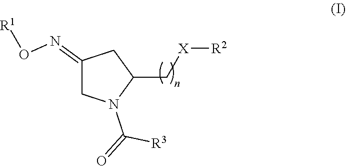

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in one or more doses (e.g., in 1, 2, 3, 4, 5, 6, 7, 8, 9, 10, or more doses) totaling from about 1,500 mg to about 2,700 mg (e.g., in one or more doses (e.g., in 1, 2, 3, 4, 5, 6, 7, 8, 9, 10, or more doses) totaling from about 1,500 mg to about 2,100 mg, from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg, such as in one or more doses (e.g., in 1, 2, 3, 4, 5, 6, 7, 8, 9, 10, or more doses) totaling 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg, such as in one or more doses (e.g., in 1, 2, 3, 4, 5, 6, 7, 8, 9, 10, or more doses) totaling about 1,800 mg, or in one or more doses (e.g., in 1, 2, 3, 4, 5, 6, 7, 8, 9, 10, or more doses) totaling from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg, such as in one or more doses (e.g., in 1, 2, 3, 4, 5, 6, 7, 8, 9, 10, or more doses) totaling about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in one or more doses (e.g., in 1, 2, 3, 4, 5, 6, 7, 8, 9, 10, or more doses) totaling about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II))); and

b. transferring one or more embryos (e.g., one, two, three, or more embryos) to the uterus of the subject concurrently with administration of the oxytocin antagonist.

In a further aspect, the disclosure provides a method of treating a subject undergoing embryo transfer therapy by:

a. administering to the subject a therapeutically effective amount of an oxytocin antagonist, such as a compound represented by formula (I)

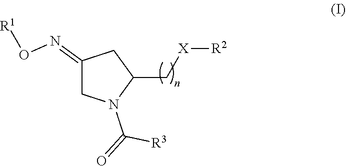

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in a single dose (e.g., on the day of the embryo transfer therapy) of from about 1,500 mg to about 2,700 mg (e.g., in a single dose (e.g., on the day of the embryo transfer therapy) of from about 1,500 mg to about 2,100 mg, from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg, such as in a single dose (e.g., on the day of the embryo transfer therapy) of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg, such as in a single dose (e.g., on the day of the embryo transfer therapy) of about 1,800 mg, or in a single dose (e.g., on the day of the embryo transfer therapy) of from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg, such as in a single dose (e.g., on the day of the embryo transfer therapy) of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in an amount of about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II))); and

b. transferring one or more embryos (e.g., one, two, three, or more embryos) to the uterus of the subject concurrently with administration of the oxytocin antagonist.

In another aspect, the disclosure features an oxytocin antagonist, such as a compound represented by formula (I), for use in the method described in any of the preceding aspects of the disclosure.

In some embodiments of any of the above aspects of the disclosure, the administering reduces the likelihood of embryo implantation failure and/or miscarriage.

In some embodiments, the oxytocin antagonist is administered to the subject concurrently with embryo transfer in a single dose.

In some embodiments, the oxytocin antagonist is administered to the subject in multiple doses beginning during embryo transfer (for instance, in multiple periodic doses) and continuing after embryo transfer, such as from 1 to 20 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, beginning during embryo transfer and continuing following embryo transfer. For instance, in some embodiments, the oxytocin antagonist is administered to the subject in from 1 to 10 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, beginning during embryo transfer and continuing following embryo transfer. In some embodiments, the oxytocin antagonist is administered to the subject in from 1 to 5 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, beginning during embryo transfer and continuing following embryo transfer. In some embodiments, the oxytocin antagonist is administered to the subject in from 10 to 20 doses, for instance, per 12 hours, per 24 hours, per 36 hours, per 48 hours, per 60 hours, per 72 hours, per 84 hours, per 96 hours, per 108 hours, per 120 hours, per 132 hours, per 144 hours, per 156 hours, per 168 hours, or longer, beginning during embryo transfer and continuing following embryo transfer. In some embodiments, the oxytocin antagonist is administered to the subject in from 10 to 15 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, beginning during embryo transfer and continuing following embryo transfer. In some embodiments, the oxytocin antagonist is administered to the subject in 1, 2, 3, 4, 5, 6, 7, 8, 9, 10, 11, 12, 13, 14, 15, 16, 17, 18, 19, 20, or more, doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is administered to the subject beginning during embryo transfer and continuing following embryo transfer in up to 7 doses (e.g., 1, 2, 3, 4, 5, 6, or 7 doses) per 24 hours.

For example, in some embodiments, the oxytocin antagonist is first administered to the subject concurrently with the transfer of the one or more embryos to the uterus of the subject, and the oxytocin antagonist is subsequently administered to the subject in one or more additional doses within about 1 hour to about 24 hours following the transfer of the one or more embryos to the subject. For instance, in some embodiments, the oxytocin antagonist is administered to the subject in one or more additional doses within about 1 hour to about 12 hours following the transfer of the one or more embryos to the subject. In some embodiments, the oxytocin antagonist is administered to the subject in one or more additional doses within from about 12 hours to about 24 hours following the transfer of the one or more embryos to the subject. In some embodiments, the oxytocin antagonist is administered to the subject in one or more additional doses within from about 1 hour to about 10 hours following the transfer of the one or more embryos to the subject. In some embodiments, the oxytocin antagonist is administered to the subject in one or more additional doses within from about 1 hour to about 9 hours following the transfer of the one or more embryos to the subject. In some embodiments, the oxytocin antagonist is administered to the subject in one or more additional doses within from about 1 hour to about 8 hours following the transfer of the one or more embryos to the subject. In some embodiments, the oxytocin antagonist is administered to the subject in one or more additional doses within from about 1 hour to about 7 hours following the transfer of the one or more embryos to the subject. In some embodiments, the oxytocin antagonist is administered to the subject in one or more additional doses within from about 1 hour to about 6 hours following the transfer of the one or more embryos to the subject. In some embodiments, the oxytocin antagonist is administered to the subject in one or more additional doses within from about 1 hour to about 5 hours following the transfer of the one or more embryos to the subject. In some embodiments, the oxytocin antagonist is administered to the subject in one or more additional doses within from about 1 hour to about 4 hours following the transfer of the one or more embryos to the subject. In some embodiments, the oxytocin antagonist is administered to the subject in one or more additional doses within from about 2 hours to about 6 hours following the transfer of the one or more embryos to the subject. In some embodiments, the oxytocin antagonist is administered to the subject in one or more additional doses within from about 3 hours to about 5 hours following the transfer of the one or more embryos to the subject.

In some embodiments, the oxytocin antagonist is first administered to the subject concurrently with the transfer of the one or more embryos to the uterus of the subject, and the oxytocin antagonist is subsequently administered to the subject in one or more additional doses starting at about 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, 96 hours, 108 hours, 120 hours, 132 hours, 144 hours, 156 hours, 168 hours, or more, following the transfer of the one or more embryos to the subject.

In some embodiments, the oxytocin antagonist is first administered to the subject concurrently with the transfer of the one or more embryos to the uterus of the subject, and the oxytocin antagonist is subsequently administered to the subject in multiple additional doses following embryo transfer, such as in from 1 to 20 additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is additionally administered to the subject following embryo transfer in from 1 to 20 doses per 24 hours, such as 1 dose per 24 hours, 2 doses per 24 hours, 3 doses per 24 hours, 4 doses per 24 hours, 5 doses per 24 hours, 6 doses per 24 hours, 7 doses per 24 hours, 8 doses per 24 hours, 9 doses per 24 hours, 10 doses per 24 hours, 11 doses per 24 hours, 12 doses per 24 hours, 13 doses per 24 hours, 14 doses per 24 hours, 15 doses per 24 hours, 16 doses per 24 hours, 17 doses per 24 hours, 18 doses per 24 hours, 19 doses per 24 hours, 20 doses per 24 hours. In some embodiments, the oxytocin antagonist is additionally administered to the subject following embryo transfer in more than 20 doses per 24 hours.

For instance, in some embodiments, the oxytocin antagonist is first administered to the subject concurrently with the transfer of the one or more embryos to the uterus of the subject, and the compound is subsequently administered to the subject in from 1 to 10 additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is additionally administered to the subject following embryo transfer in from 1 to 10 doses per 24 hours, such as 1 dose per 24 hours, 2 doses per 24 hours, 3 doses per 24 hours, 4 doses per 24 hours, 5 doses per 24 hours, 6 doses per 24 hours, 7 doses per 24 hours, 8 doses per 24 hours, 9 doses per 24 hours, 10 doses per 24 hours.

In some embodiments, the oxytocin antagonist is first administered to the subject concurrently with the transfer of the one or more embryos to the uterus of the subject, and the oxytocin antagonist is subsequently administered to the subject in from 1 to 5 additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is administered to the subject in from 10 to 20 additional doses, for instance, per 12 hours, per 24 hours, per 36 hours, per 48 hours, per 60 hours, per 72 hours, per 84 hours, per 96 hours, per 108 hours, per 120 hours, per 132 hours, per 144 hours, per 156 hours, per 168 hours, or longer, following embryo transfer. In some embodiments, the oxytocin antagonist is administered to the subject in from 10 to 15 additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer.

In some embodiments, the oxytocin antagonist is first administered to the subject concurrently with the transfer of the one or more embryos to the uterus of the subject, and the compound is subsequently administered to the subject in 1, 2, 3, 4, 5, 6, 7, 8, 9, 10, 11, 12, 13, 14, 15, 16, 17, 18, 19, 20, or more, additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer.

In some embodiments, the oxytocin antagonist is first administered to the subject concurrently with the transfer of the one or more embryos to the uterus of the subject, and the oxytocin antagonist is subsequently administered to the subject following embryo transfer in up to 7 additional doses (e.g., 1, 2, 3, 4, 5, 6, or 7 doses) per 24 hours. In some embodiments, the oxytocin antagonist is additionally administered to the subject following embryo transfer in 1 dose per 24 hours, such as 1 additional dose per 24 hours of compound (II), below. In some embodiments, the oxytocin antagonist is additionally administered to the subject following embryo transfer in 2 doses per 24 hours, such as 2 additional doses per 24 hours of compound (II), below. In some embodiments, the oxytocin antagonist is additionally administered to the subject following embryo transfer in 3 doses per 24 hours, such as 3 additional doses per 24 hours of compound (II), below. In some embodiments, the oxytocin antagonist is additionally administered to the subject following embryo transfer in 4 doses per 24 hours, such as 4 additional doses per 24 hours of compound (II), below. In some embodiments, the oxytocin antagonist is additionally administered to the subject following embryo transfer in 5 doses per 24 hours, such as 5 additional doses per 24 hours of compound (II), below. In some embodiments, the oxytocin antagonist is additionally administered to the subject following embryo transfer in 6 doses per 24 hours, such as 6 additional doses per 24 hours of compound (II), below. In some embodiments, the oxytocin antagonist is additionally administered to the subject following embryo transfer in 7 doses per 24 hours, such as 7 additional doses per 24 hours of compound (II), below.

When one or more additional doses of the oxytocin antagonist are administered to the subject following embryo transfer, administration of the oxytocin antagonist may terminate, for instance, within from about 1 hour to about 14 days, or more, following embryo transfer. For instance, administration of the oxytocin antagonist may terminate within about 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, 96 hours, 108 hours, 120 hours, 132 hours, 144 hours, 156 hours, 168 hours, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more, following embryo transfer.

Thus, in some embodiments, the oxytocin antagonist is first administered to the subject concurrently with the transfer of the one or more embryos to the uterus of the subject, and the oxytocin antagonist is subsequently administered to the subject in additional daily doses following embryo transfer for about 1 day to about 14 days following embryo transfer. In some embodiments, the additional daily doses are administered to the subject for about 3 days to about 11 days following embryo transfer. In some embodiments, the additional daily doses are administered to the subject for 7 days following embryo transfer.

In another aspect, the disclosure provides a method of treating a subject undergoing embryo transfer therapy by administering to the subject a therapeutically effective amount of an oxytocin antagonist, such as a compound represented by formula (I)

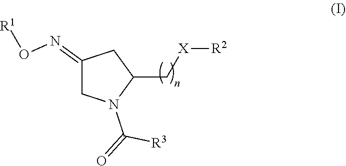

or a geometric isomer, enantiomer, diastereomer, racemate, or sat thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in an amount of from about 1,500 mg to about 2,700 mg per dose (e.g., in an amount of from about 1,500 mg to about 2,100 mg per dose, from about 1,550 mg to about 2,050 mg per dose, from about 1,600 mg to about 2,000 mg per dose, from about 1,650 mg to about 1,950 mg per dose, from about 1,700 mg to about 1,900 mg per dose, from about 1,750 mg to about 1,850 mg per dose, from about 1,760 mg to about 1,840 mg per dose, from about 1,770 mg to about 1,830 mg per dose, from about 1,780 mg to about 1,820 mg per dose, from about 1,790 mg to about 1,810 mg per dose, from about 1,791 mg to about 1,809 mg per dose, from about 1,792 mg to about 1,808 mg per dose, from about 1,793 mg to about 1,807 mg per dose, from about 1,794 mg to about 1,806 mg per dose, from about 1,795 mg to about 1,805 mg per dose, from about 1,796 mg to about 1,804 mg per dose, from about 1,797 mg to about 1,803 mg per dose, from about 1,798 mg to about 1,802 mg per dose, or from about 1,799 mg to about 1,801 mg per dose, such as an amount of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg per dose, such as in an amount of about 1,800 mg per dose, or in an amount of from about 2,100 mg to about 2,700 mg per dose, from about 2,150 mg to about 2,650 mg per dose, from about 2,200 mg to about 2,600 mg per dose, from about 2,250 mg to about 2,550 mg per dose, from about 2,300 mg to about 2,500 mg per dose, from about 2,350 mg to about 2,450 mg per dose, from about 2,360 mg to about 2,440 mg per dose, from about 2,370 mg to about 2,430 mg per dose, from about 2,380 mg to about 2,420 mg per dose, from about 2,390 mg to about 2,410 mg per dose, from about 2,391 mg to about 2,409 mg per dose, from about 2,392 mg to about 2,408 mg per dose, from about 2,393 mg to about 2,407 mg per dose, from about 2,394 mg to about 2,406 mg per dose, from about 2,395 mg to about 2,405 mg per dose, from about 2,396 mg to about 2,404 mg per dose, from about 2,397 mg to about 2,403 mg per dose, from about 2,398 mg to about 2,402 mg per dose, or from about 2,399 mg to about 2,401 mg per dose, such as in an amount of about 2,400 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II))); and

wherein the oxytocin antagonist is administered to the subject following transfer of one or more embryos (e.g., one, two, three, or more embryos) to the uterus of the subject.

In another aspect, the disclosure provides a method of treating a subject undergoing embryo transfer therapy by administering to the subject a therapeutically effective amount of an oxytocin antagonist, such as a compound represented by formula (I)

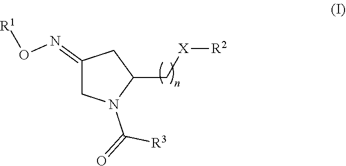

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in one or more doses (e.g., in 1, 2, 3, 4, 5, 6, 7, 8, 9, 10, or more doses) totaling from about 1,500 mg to about 2,700 mg (e.g., in one or more doses (e.g., in 1, 2, 3, 4, 5, 6, 7, 8, 9, 10, or more doses) totaling from about 1,500 mg to about 2,100 mg, from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg, such as in one or more doses (e.g., in 1, 2, 3, 4, 5, 6, 7, 8, 9, 10, or more doses) totaling 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg, such as in one or more doses (e.g., in 1, 2, 3, 4, 5, 6, 7, 8, 9, 10, or more doses) totaling about 1,800 mg, or in one or more doses (e.g., in 1, 2, 3, 4, 5, 6, 7, 8, 9, 10, or more doses) totaling from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg, such as in one or more doses (e.g., in 1, 2, 3, 4, 5, 6, 7, 8, 9, 10, or more doses) totaling about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in one or more doses (e.g., in 1, 2, 3, 4, 5, 6, 7, 8, 9, 10, or more doses) totaling about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II))); and

wherein the oxytocin antagonist is administered to the subject following transfer of one or more embryos (e.g., one, two, three, or more embryos) to the uterus of the subject.

In a further aspect, the disclosure provides a method of treating a subject undergoing embryo transfer therapy by administering to the subject a therapeutically effective amount of an oxytocin antagonist, such as a compound represented by formula (I)

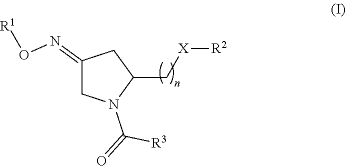

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in a single dose (e.g., on the day of the embryo transfer therapy) of from about 1,500 mg to about 2,700 mg (e.g., in a single dose (e.g., on the day of the embryo transfer therapy) of from about 1,500 mg to about 2,100 mg, from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg, such as in a single dose (e.g., on the day of the embryo transfer therapy) of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg, such as in a single dose (e.g., on the day of the embryo transfer therapy) of about 1,800 mg, or in a single dose (e.g., on the day of the embryo transfer therapy) of from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg, such as in a single dose (e.g., on the day of the embryo transfer therapy) of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in an amount of about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II))); and

wherein the oxytocin antagonist is administered to the subject following transfer of one or more embryos (e.g., one, two, three, or more embryos) to the uterus of the subject.

In another aspect, the disclosure features an oxytocin antagonist, such as a compound represented by formula (I), for use in the method described in any of the preceding aspects of the disclosure.

In some embodiments, the administering reduces the likelihood of embryo implantation failure and/or miscarriage.

In another aspect, the disclosure provides a method of treating a subject undergoing embryo transfer therapy by transferring one or more embryos (e.g., one, two, three, or more embryos) to the uterus of the subject, wherein the subject is subsequently administered a therapeutically effective amount of an oxytocin antagonist, such as a compound represented by formula (I)

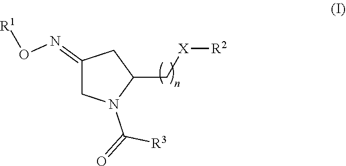

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in an amount of from about 1,500 mg to about 2,700 mg per dose. For example, the compound may be administered to the subject in an amount of from about 1,500 mg to about 2,100 mg per dose, from about 1,550 mg to about 2,050 mg per dose, from about 1,600 mg to about 2,000 mg per dose, from about 1,650 mg to about 1,950 mg per dose, from about 1,700 mg to about 1,900 mg per dose, from about 1,750 mg to about 1,850 mg per dose, from about 1,760 mg to about 1,840 mg per dose, from about 1,770 mg to about 1,830 mg per dose, from about 1,780 mg to about 1,820 mg per dose, from about 1,790 mg to about 1,810 mg per dose, from about 1,791 mg to about 1,809 mg per dose, from about 1,792 mg to about 1,808 mg per dose, from about 1,793 mg to about 1,807 mg per dose, from about 1,794 mg to about 1,806 mg per dose, from about 1,795 mg to about 1,805 mg per dose, from about 1,796 mg to about 1,804 mg per dose, from about 1,797 mg to about 1,803 mg per dose, from about 1,798 mg to about 1,802 mg per dose, or from about 1,799 mg to about 1,801 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of from about 2,100 mg to about 2,700 mg per dose, from about 2,150 mg to about 2,650 mg per dose, from about 2,200 mg to about 2,600 mg per dose, from about 2,250 mg to about 2,550 mg per dose, from about 2,300 mg to about 2,500 mg per dose, from about 2,350 mg to about 2,450 mg per dose, from about 2,360 mg to about 2,440 mg per dose, from about 2,370 mg to about 2,430 mg per dose, from about 2,380 mg to about 2,420 mg per dose, from about 2,390 mg to about 2,410 mg per dose, from about 2,391 mg to about 2,409 mg per dose, from about 2,392 mg to about 2,408 mg per dose, from about 2,393 mg to about 2,407 mg per dose, from about 2,394 mg to about 2,406 mg per dose, from about 2,395 mg to about 2,405 mg per dose, from about 2,396 mg to about 2,404 mg per dose, from about 2,397 mg to about 2,403 mg per dose, from about 2,398 mg to about 2,402 mg per dose, or from about 2,399 mg to about 2,401 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg per dose, such as in an amount of about 1,800 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg per dose, such as in an amount of about 2,400 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of from about 1,600 mg to about 2,600 mg per dose, such as in an amount of from about 1,610 mg to about 2,590 mg per dose, from about 1,620 mg to about 2,580 mg per dose, from about 1,630 mg to about 2,570 mg per dose, from about 1,640 mg to about 2,560 mg per dose, from about 1,650 mg to about 2,550 mg per dose, from about 1,660 mg to about 2,540 mg per dose, from about 1,670 mg to about 2,530 mg per dose, from about 1,680 mg to about 2,520 mg per dose, from about 1,690 mg to about 2,510 mg per dose, from about 1,700 mg to about 2,500 mg per dose, from about 1,710 mg to about 2,490 mg per dose, from about 1,720 mg to about 2,480 mg per dose, from about 1,730 mg to about 2,470 mg per dose, from about 1,740 mg to about 2,460 mg per dose, from about 1,750 mg to about 2,450 mg per dose, from about 1,760 mg to about 2,440 mg per dose, from about 1,770 mg to about 2,430 mg per dose, from about 1,780 mg to about 2,420 mg per dose, from about 1,790 mg to about 2,410 mg per dose, from about 1,800 mg to about 2,400 mg per dose, from about 1,810 mg to about 2,390 mg per dose, from about 1,820 mg to about 2,380 mg per dose, from about 1,830 mg to about 2,370 mg per dose, from about 1,840 mg to about 2,360 mg per dose, from about 1,850 mg to about 2,350 mg per dose, from about 1,860 mg to about 2,340 mg per dose, from about 1,870 mg to about 2,330 mg per dose, from about 1,880 mg to about 2,320 mg per dose, from about 1,890 mg to about 2,310 mg per dose, from about 1,900 mg to about 2,300 mg per dose, from about 1,910 mg to about 2,290 mg per dose, from about 1,920 mg to about 2,280 mg per dose, from about 1,930 mg to about 2,270 mg per dose, from about 1,940 mg to about 2,260 mg per dose, from about 1,950 mg to about 2,250 mg per dose, from about 1,960 mg to about 2,240 mg per dose, from about 1,970 mg to about 2,230 mg per dose, from about 1,980 mg to about 2,220 mg per dose, from about 1,990 mg to about 2,210 mg per dose, from about 2,000 mg to about 2,200 mg per dose, from about 2,010 mg to about 2,190 mg per dose, from about 2,020 mg to about 2,180 mg per dose, from about 2,030 mg to about 2,170 mg per dose, from about 2,040 mg to about 2,160 mg per dose, from about 2,050 mg to about 2,150 mg per dose, from about 2,060 mg to about 2,140 mg per dose, from about 2,070 mg to about 2,130 mg per dose, from about 2,080 mg to about 2,120 mg per dose, or from about 2,090 mg to about 2,110 mg per dose, such as about 2,100 mg per dose e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In another aspect, the disclosure provides a method of treating a subject undergoing embryo transfer therapy by transferring one or more embryos (e.g., one, two, three, or more embryos) to the uterus of the subject, wherein the subject is subsequently administered a therapeutically effective amount of an oxytocin antagonist, such as a compound represented by formula (I)

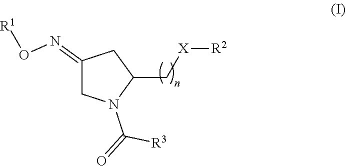

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in one or more doses (e.g., in 1, 2, 3, 4, 5, 6, 7, 8, 9, 10, or more doses) totaling from about 1,500 mg to about 2,700 mg. For example, the compound may be administered to the subject in one or more doses (e.g., in 1, 2, 3, 4, 5, 6, 7, 8, 9, 10, or more doses) totaling from about 1,500 mg to about 2,100 mg, from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in an amount of about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling from about 1,600 mg to about 2,600 mg, such as in one or more doses (e.g., in 1, 2, 3, 4, 5, 6, 7, 8, 9, 10, or more doses) totaling from about 1,610 mg to about 2,590 mg, from about 1,620 mg to about 2,580 mg, from about 1,630 mg to about 2,570 mg, from about 1,640 mg to about 2,560 mg, from about 1,650 mg to about 2,550 mg, from about 1,660 mg to about 2,540 mg, from about 1,670 mg to about 2,530 mg, from about 1,680 mg to about 2,520 mg, from about 1,690 mg to about 2,510 mg, from about 1,700 mg to about 2,500 mg, from about 1,710 mg to about 2,490 mg, from about 1,720 mg to about 2,480 mg, from about 1,730 mg to about 2,470 mg, from about 1,740 mg to about 2,460 mg, from about 1,750 mg to about 2,450 mg, from about 1,760 mg to about 2,440 mg, from about 1,770 mg to about 2,430 mg, from about 1,780 mg to about 2,420 mg, from about 1,790 mg to about 2,410 mg, from about 1,800 mg to about 2,400 mg, from about 1,810 mg to about 2,390 mg, from about 1,820 mg to about 2,380 mg, from about 1,830 mg to about 2,370 mg, from about 1,840 mg to about 2,360 mg, from about 1,850 mg to about 2,350 mg, from about 1,860 mg to about 2,340 mg, from about 1,870 mg to about 2,330 mg, from about 1,880 mg to about 2,320 mg, from about 1,890 mg to about 2,310 mg, from about 1,900 mg to about 2,300 mg, from about 1,910 mg to about 2,290 mg, from about 1,920 mg to about 2,280 mg, from about 1,930 mg to about 2,270 mg, from about 1,940 mg to about 2,260 mg, from about 1,950 mg to about 2,250 mg, from about 1,960 mg to about 2,240 mg, from about 1,970 mg to about 2,230 mg, from about 1,980 mg to about 2,220 mg, from about 1,990 mg to about 2,210 mg, from about 2,000 mg to about 2,200 mg, from about 2,010 mg to about 2,190 mg, from about 2,020 mg to about 2,180 mg, from about 2,030 mg to about 2,170 mg, from about 2,040 mg to about 2,160 mg, from about 2,050 mg to about 2,150 mg, from about 2,060 mg to about 2,140 mg, from about 2,070 mg to about 2,130 mg, from about 2,080 mg to about 2,120 mg, or from about 2,090 mg to about 2,110 mg, such as about 2,100 mg e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses, such as in a single dose) totaling about 1,800 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses, such as in a single dose) totaling about 2,100 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses, such as in a single dose) totaling about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In a further aspect, the disclosure provides a method of treating a subject undergoing embryo transfer therapy by transferring one or more embryos (e.g., one, two, three, or more embryos) to the uterus of the subject, wherein the subject is subsequently administered a therapeutically effective amount of an oxytocin antagonist, such as a compound represented by formula (I)

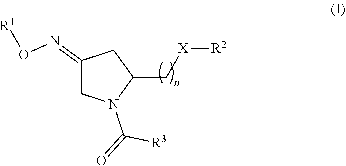

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in a single dose (e.g., on the day of the embryo transfer therapy) of from about 1,500 mg to about 2,700 mg. For example, the compound may be administered to the subject in a single dose (e.g., on the day of the embryo transfer therapy) of from about 1,500 mg to about 2,100 mg, from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in an amount of about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of from about 1,600 mg to about 2,600 mg, such as in a single dose (e.g., on the day of the embryo transfer therapy) of from about 1,610 mg to about 2,590 mg, from about 1,620 mg to about 2,580 mg, from about 1,630 mg to about 2,570 mg, from about 1,640 mg to about 2,560 mg, from about 1,650 mg to about 2,550 mg, from about 1,660 mg to about 2,540 mg, from about 1,670 mg to about 2,530 mg, from about 1,680 mg to about 2,520 mg, from about 1,690 mg to about 2,510 mg, from about 1,700 mg to about 2,500 mg, from about 1,710 mg to about 2,490 mg, from about 1,720 mg to about 2,480 mg, from about 1,730 mg to about 2,470 mg, from about 1,740 mg to about 2,460 mg, from about 1,750 mg to about 2,450 mg, from about 1,760 mg to about 2,440 mg, from about 1,770 mg to about 2,430 mg, from about 1,780 mg to about 2,420 mg, from about 1,790 mg to about 2,410 mg, from about 1,800 mg to about 2,400 mg, from about 1,810 mg to about 2,390 mg, from about 1,820 mg to about 2,380 mg, from about 1,830 mg to about 2,370 mg, from about 1,840 mg to about 2,360 mg, from about 1,850 mg to about 2,350 mg, from about 1,860 mg to about 2,340 mg, from about 1,870 mg to about 2,330 mg, from about 1,880 mg to about 2,320 mg, from about 1,890 mg to about 2,310 mg, from about 1,900 mg to about 2,300 mg, from about 1,910 mg to about 2,290 mg, from about 1,920 mg to about 2,280 mg, from about 1,930 mg to about 2,270 mg, from about 1,940 mg to about 2,260 mg, from about 1,950 mg to about 2,250 mg, from about 1,960 mg to about 2,240 mg, from about 1,970 mg to about 2,230 mg, from about 1,980 mg to about 2,220 mg, from about 1,990 mg to about 2,210 mg, from about 2,000 mg to about 2,200 mg, from about 2,010 mg to about 2,190 mg, from about 2,020 mg to about 2,180 mg, from about 2,030 mg to about 2,170 mg, from about 2,040 mg to about 2,160 mg, from about 2,050 mg to about 2,150 mg, from about 2,060 mg to about 2,140 mg, from about 2,070 mg to about 2,130 mg, from about 2,080 mg to about 2,120 mg, or from about 2,090 mg to about 2,110 mg, such as about 2,100 mg e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of about 1,800 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of about 2,100 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In another aspect, the disclosure features an oxytocin antagonist, such as a compound represented by formula (I), for use in the method described in any of the preceding aspects of the disclosure.

In some embodiments of any of the above aspects of the disclosure, administration of the oxytocin antagonist reduces the likelihood of embryo implantation failure and/or miscarriage.

In another aspect, the disclosure provides a method of treating a subject undergoing embryo transfer therapy by:

a. administering to the subject a therapeutically effective amount of an oxytocin antagonist, such as a compound represented by formula (I)

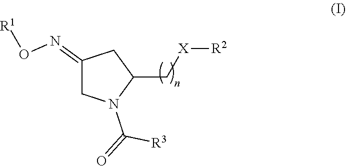

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in an amount of from about 1,500 mg to about 2,700 mg per dose (e.g., in an amount of from about 1,500 mg to about 2,100 mg per dose, from about 1,550 mg to about 2,050 mg per dose, from about 1,600 mg to about 2,000 mg per dose, from about 1,650 mg to about 1,950 mg per dose, from about 1,700 mg to about 1,900 mg per dose, from about 1,750 mg to about 1,850 mg per dose, from about 1,760 mg to about 1,840 mg per dose, from about 1,770 mg to about 1,830 mg per dose, from about 1,780 mg to about 1,820 mg per dose, from about 1,790 mg to about 1,810 mg per dose, from about 1,791 mg to about 1,809 mg per dose, from about 1,792 mg to about 1,808 mg per dose, from about 1,793 mg to about 1,807 mg per dose, from about 1,794 mg to about 1,806 mg per dose, from about 1,795 mg to about 1,805 mg per dose, from about 1,796 mg to about 1,804 mg per dose, from about 1,797 mg to about 1,803 mg per dose, from about 1,798 mg to about 1,802 mg per dose, or from about 1,799 mg to about 1,801 mg per dose, such as an amount of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg per dose, such as in an amount of about 1,800 mg per dose, or in an amount of from about 2,100 mg to about 2,700 mg per dose, from about 2,150 mg to about 2,650 mg per dose, from about 2,200 mg to about 2,600 mg per dose, from about 2,250 mg to about 2,550 mg per dose, from about 2,300 mg to about 2,500 mg per dose, from about 2,350 mg to about 2,450 mg per dose, from about 2,360 mg to about 2,440 mg per dose, from about 2,370 mg to about 2,430 mg per dose, from about 2,380 mg to about 2,420 mg per dose, from about 2,390 mg to about 2,410 mg per dose, from about 2,391 mg to about 2,409 mg per dose, from about 2,392 mg to about 2,408 mg per dose, from about 2,393 mg to about 2,407 mg per dose, from about 2,394 mg to about 2,406 mg per dose, from about 2,395 mg to about 2,405 mg per dose, from about 2,396 mg to about 2,404 mg per dose, from about 2,397 mg to about 2,403 mg per dose, from about 2,398 mg to about 2,402 mg per dose, or from about 2,399 mg to about 2,401 mg per dose, such as in an amount of about 2,400 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II))); and

b. transferring one or more embryos (e.g., one, two, three, or more embryos) to the uterus of the subject prior to administration of the oxytocin antagonist.

In another aspect, the disclosure provides a method of treating a subject undergoing embryo transfer therapy by:

a. administering to the subject a therapeutically effective amount of an oxytocin antagonist, such as a compound represented by formula (I)

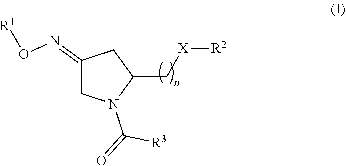

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in one or more doses (e.g., in 1, 2, 3, 4, 5, 6, 7, 8, 9, 10, or more doses) totaling from about 1,500 mg to about 2,700 mg (e.g., in one or more doses (e.g., in 1, 2, 3, 4, 5, 6, 7, 8, 9, 10, or more doses) totaling from about 1,500 mg to about 2,100 mg, from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg, such as in one or more doses (e.g., in 1, 2, 3, 4, 5, 6, 7, 8, 9, 10, or more doses) totaling 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg, such as in one or more doses (e.g., in 1, 2, 3, 4, 5, 6, 7, 8, 9, 10, or more doses) totaling about 1,800 mg, or in one or more doses (e.g., in 1, 2, 3, 4, 5, 6, 7, 8, 9, 10, or more doses) totaling from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg, such as in one or more doses (e.g., in 1, 2, 3, 4, 5, 6, 7, 8, 9, 10, or more doses) totaling about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in one or more doses (e.g., in 1, 2, 3, 4, 5, 6, 7, 8, 9, 10, or more doses) totaling about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II))); and

b. transferring one or more embryos (e.g., one, two, three, or more embryos) to the uterus of the subject prior to administration of the oxytocin antagonist.

In a further aspect, the disclosure provides a method of treating a subject undergoing embryo transfer therapy by:

a. administering to the subject a therapeutically effective amount of an oxytocin antagonist, such as a compound represented by formula (I)

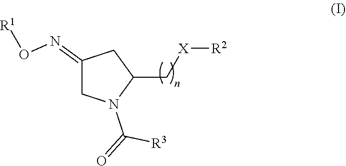

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,

wherein the compound is administered to the subject in a single dose (e.g., on the day of the embryo transfer therapy) of from about 1,500 mg to about 2,700 mg (e.g., in a single dose (e.g., on the day of the embryo transfer therapy) of from about 1,500 mg to about 2,100 mg, from about 1,550 mg to about 2,050 mg, from about 1,600 mg to about 2,000 mg, from about 1,650 mg to about 1,950 mg, from about 1,700 mg to about 1,900 mg, from about 1,750 mg to about 1,850 mg, from about 1,760 mg to about 1,840 mg, from about 1,770 mg to about 1,830 mg, from about 1,780 mg to about 1,820 mg, from about 1,790 mg to about 1,810 mg, from about 1,791 mg to about 1,809 mg, from about 1,792 mg to about 1,808 mg, from about 1,793 mg to about 1,807 mg, from about 1,794 mg to about 1,806 mg, from about 1,795 mg to about 1,805 mg, from about 1,796 mg to about 1,804 mg, from about 1,797 mg to about 1,803 mg, from about 1,798 mg to about 1,802 mg, or from about 1,799 mg to about 1,801 mg, such as in a single dose (e.g., on the day of the embryo transfer therapy) of about 1,500 mg, 1,510 mg, 1,520 mg, 1,530 mg, 1,540 mg, 1,550 mg, 1,560 mg, 1,570 mg, 1,580 mg, 1,590 mg, 1,600 mg, 1,610 mg, 1,620 mg, 1,630 mg, 1,640 mg, 1,650 mg, 1,660 mg, 1,670 mg, 1,680 mg, 1,690 mg, 1,700 mg, 1,710 mg, 1,720 mg, 1,730 mg, 1,740 mg, 1,750 mg, 1,760 mg, 1,770 mg, 1,780 mg, 1,790 mg, 1,800 mg, 1,810 mg, 1,820 mg, 1,830 mg, 1,840 mg, 1,850 mg, 1,860 mg, 1,870 mg, 1,880 mg, 1,890 mg, 1,900 mg, 1,910 mg, 1,920 mg, 1,930 mg, 1,940 mg, 1,950 mg, 1,960 mg, 1,970 mg, 1,980 mg, 1,990 mg, 2,000 mg, 2,010 mg, 2,020 mg, 2,030 mg, 2,040 mg, 2,050 mg, 2,060 mg, 2,070 mg, 2,080 mg, 2,090 mg, or 2,100 mg, such as in a single dose (e.g., on the day of the embryo transfer therapy) of about 1,800 mg, or in a single dose (e.g., on the day of the embryo transfer therapy) of from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg, such as in a single dose (e.g., on the day of the embryo transfer therapy) of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg, such as in an amount of about 2,400 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II))); and

b. transferring one or more embryos (e.g., one, two, three, or more embryos) to the uterus of the subject prior to administration of the oxytocin antagonist.

In another aspect, the disclosure features an oxytocin antagonist, such as a compound represented by formula (I), for use in the method described in any of the preceding aspects of the disclosure.

In some embodiments of any of the above aspects of the disclosure, the administering reduces the likelihood of embryo implantation failure and/or miscarriage.

In some embodiments, the oxytocin antagonist is administered to the subject within about 1 hour to about 24 hours following the transfer of the one or more embryos to the subject. For instance, in some embodiments, the oxytocin antagonist is administered to the subject within about 1 hour to about 12 hours following the transfer of the one or more embryos to the subject. In some embodiments, the oxytocin antagonist is administered to the subject within from about 12 hours to about 24 hours following the transfer of the one or more embryos to the subject. In some embodiments, the oxytocin antagonist is administered to the subject within from about 1 hour to about 10 hours following the transfer of the one or more embryos to the subject. In some embodiments, the oxytocin antagonist is administered to the subject within from about 1 hour to about 9 hours following the transfer of the one or more embryos to the subject. In some embodiments, the oxytocin antagonist is administered to the subject within from about 1 hour to about 8 hours following the transfer of the one or more embryos to the subject. In some embodiments, the oxytocin antagonist is administered to the subject within from about 1 hour to about 7 hours following the transfer of the one or more embryos to the subject. In some embodiments, the oxytocin antagonist is administered to the subject within from about 1 hour to about 6 hours following the transfer of the one or more embryos to the subject. In some embodiments, the oxytocin antagonist is administered to the subject within from about 1 hour to about 5 hours following the transfer of the one or more embryos to the subject. In some embodiments, the oxytocin antagonist is administered to the subject within from about 1 hour to about 4 hours following the transfer of the one or more embryos to the subject. In some embodiments, the oxytocin antagonist is administered to the subject within from about 2 hours to about 6 hours following the transfer of the one or more embryos to the subject. In some embodiments, the oxytocin antagonist is administered to the subject within from about 3 hours to about 5 hours following the transfer of the one or more embryos to the subject.

For instance, in some embodiments, the oxytocin antagonist is administered to the subject about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, 84 hours, 96 hours, 108 hours, 120 hours, 132 hours, 144 hours, 156 hours, 168 hours, or more, following the transfer of the one or more embryos to the subject.

In some embodiments, the oxytocin antagonist is administered to the subject after embryo transfer in a single dose.

In some embodiments, the oxytocin antagonist is administered to the subject in multiple doses following embryo transfer, such as in multiple periodic doses. In some embodiments, the oxytocin antagonist is administered to the subject in from 1 to 20 doses following embryo transfer, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is administered to the subject following embryo transfer in from 1 to 20 doses per 24 hours, such as 1 dose per 24 hours, 2 doses per 24 hours, 3 doses per 24 hours, 4 doses per 24 hours, 5 doses per 24 hours, 6 doses per 24 hours, 7 doses per 24 hours, 8 doses per 24 hours, 9 doses per 24 hours, 10 doses per 24 hours, 11 doses per 24 hours, 12 doses per 24 hours, 13 doses per 24 hours, 14 doses per 24 hours, 15 doses per 24 hours, 16 doses per 24 hours, 17 doses per 24 hours, 18 doses per 24 hours, 19 doses per 24 hours, 20 doses per 24 hours. In some embodiments, the oxytocin antagonist is administered to the subject following embryo transfer in more than 20 doses per 24 hours.

For instance, in some embodiments, the oxytocin antagonist is administered to the subject in from 1 to 10 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is administered to the subject following embryo transfer in from 1 to 10 doses per 24 hours, such as 1 dose per 24 hours, 2 doses per 24 hours, 3 doses per 24 hours, 4 doses per 24 hours, 5 doses per 24 hours, 6 doses per 24 hours, 7 doses per 24 hours, 8 doses per 24 hours, 9 doses per 24 hours, 10 doses per 24 hours.

In some embodiments, the oxytocin antagonist is administered to the subject in from 1 to 5 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is administered to the subject in from 10 to 20 doses, for instance, per 12 hours, per 24 hours, per 36 hours, per 48 hours, per 60 hours, per 72 hours, per 84 hours, per 96 hours, per 108 hours, per 120 hours, per 132 hours, per 144 hours, per 156 hours, per 168 hours, or longer, following embryo transfer. In some embodiments, the oxytocin antagonist is administered to the subject in from 10 to 15 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer.

In some embodiments, the oxytocin antagonist is administered to the subject in 1, 2, 3, 4, 5, 6, 7, 8, 9, 10, 11, 12, 13, 14, 15, 16, 17, 18, 19, 20 doses, or more, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer.

In some embodiments, the oxytocin antagonist is administered to the subject following embryo transfer in up to 7 doses (e.g., 1, 2, 3, 4, 5, 6, or 7 doses) per 24 hours. In some embodiments, the oxytocin antagonist is administered to the subject following embryo transfer in 1 dose per 24 hours, such as 1 dose per 24 hours of compound (II), below. In some embodiments, the oxytocin antagonist is administered to the subject following embryo transfer in 2 doses per 24 hours, such as 2 doses per 24 hours of compound (II), below. In some embodiments, the oxytocin antagonist is administered to the subject following embryo transfer in 3 doses per 24 hours, such as 3 doses per 24 hours of compound (II), below. In some embodiments, the oxytocin antagonist is administered to the subject following embryo transfer in 4 doses per 24 hours, such as 4 doses per 24 hours of compound (II), below. In some embodiments, the oxytocin antagonist is administered to the subject following embryo transfer in 5 doses per 24 hours, such as 5 doses per 24 hours of compound (II), below. In some embodiments, the oxytocin antagonist is administered to the subject following embryo transfer in 6 doses per 24 hours, such as 6 doses per 24 hours of compound (II), below. In some embodiments, the oxytocin antagonist is administered to the subject following embryo transfer in 7 doses per 24 hours, such as 7 doses per 24 hours of compound (II), below.

When the oxytocin antagonist is administered in multiple doses following embryo transfer, administration of the oxytocin antagonist may terminate, for instance, within from about 1 hour to about 14 days, or more, following embryo transfer. For instance, administration of the oxytocin antagonist may terminate within about 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, 96 hours, 108 hours, 120 hours, 132 hours, 144 hours, 156 hours, 168 hours, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more, following embryo transfer.

Thus, in some embodiments, the oxytocin antagonist is administered to the subject in daily doses following embryo transfer for about 1 day to about 14 days following embryo transfer. In some embodiments, the daily doses are administered to the subject for about 3 days to about 11 days following embryo transfer. In some embodiments, the daily doses are administered to the subject for 7 days following embryo transfer.

In some embodiments, administration of the oxytocin antagonist to the subject reduces the likelihood of the subject having a miscarriage. For instance, administration of the oxytocin antagonist may reduce the likelihood of the subject having a miscarriage following the embryo transfer process such that the subject gives birth to a live offspring (e.g., a live human baby), for example, at a gestational age of at least about 24 weeks.

In some embodiments, the oxytocin antagonist is administered to the subject in an amount sufficient to achieve a plasma concentration of the oxytocin antagonist in the subject of from about 1 μM to about 20 μM. In some embodiments, the oxytocin antagonist is a compound represented by formula (I) (e.g., a compound represented by formula (II) herein) and is administered to the subject such that the subject exhibits a plasma concentration of the compound of from about 1 μM to about 20 μM at the time of embryo transfer to the uterus of the subject. For instance, in some embodiments, the compound is administered to the subject in an amount sufficient to achieve a plasma concentration of the compound in the subject (e.g., at the time of embryo transfer) of from about 5 μM to about 19 μM, 10 μM to about 18 μM, 14 μM to about 17 μM, 15 μM to about 16 μM, 1 μM to about 19 μM, 2 μM to about 18 μM, 3 μM to about 17 μM, 4 μM to about 16 μM, 5 μM to about 15 μM, or more. In some embodiments, the plasma concentration, such as the maximum plasma concentration achieved from administration of a single dose of the compound, is achieved within from about 1 hour to about 3 hours (e.g., about 1 hour, 1.1 hours, 1.2 hours, 1.3 hours, 1.4 hours, 1.5 hours, 1.6 hours, 1.7 hours, 1.8 hours, 1.9 hours, 2 hours, 2.1 hours, 2.2 hours, 2.3 hours, 2.4 hours, 2.5 hours, 2.6 hours, 2.7 hours, 2.8 hours, 2.9 hours, or 3 hours) of administering the compound to the subject.

In some embodiments, from 1 to 3 embryos are transferred to the subject. In some embodiments, from 1 to 2 embryos are transferred to the subject. For instance, in some embodiments, 1 embryo is transferred to the subject. In some embodiments, 2 embryos are transferred to the subject. In some embodiments, 3 embryos are transferred to the subject.

In some embodiments, the subject has previously undergone one or more cycles (e.g., one, two, three, four, five, six, seven, eight, nine, ten, or more cycles) of failed embryo transfer therapy, such as by in vitro fertilization-embryo transfer (IVF-ET) or intracytoplasmic sperm injection-embryo transfer (ICSI-ET) therapy. In some embodiments, the subject has not previously undergone embryo transfer therapy.

In some embodiments, the subject is a mammal and the one or more embryos are mammalian embryos. For instance, in some embodiments, the mammal is a human and the one or more mammalian embryos are human embryos.

In some embodiments, the one or more embryos are produced ex vivo by in vitro fertilization (IVF), such as by IVF of one or more ova derived from the subject. In some embodiments, the one or more embryos are produced ex vivo by intracytoplasmic sperm injection (ICSI), such as by ICSI into one or more ova derived from the subject.

In some embodiments, the one or more ova are derived from one or more oocytes (one, two, three, four, five, six, seven, eight, nine, ten, or more oocytes) isolated from the subject. In some embodiments, the one or more oocytes include from 1 to 4 ova (mature oocytes). For instance, in some embodiments, the one or more oocytes include 1 mature oocyte. In some embodiments, the one or more oocytes include 2 mature oocytes. In some embodiments, the one or more oocytes include 3 mature oocytes. In some embodiments, the one or more oocytes include 4 mature oocytes.

In some embodiments, the one or more ova are isolated directly from the subject.

In some embodiments, the one or more oocytes or ova are isolated from the subject from about 1 day to about 7 days prior to the transfer of the one or more embryos to the subject. In some embodiments, the one or more oocytes or ova are isolated from the subject from about 2 days to about 6 days prior to the transfer of the one or more embryos to the subject. In some embodiments, the one or more oocytes or ova are isolated from the subject from about 3 days to about 5 days prior to the transfer of the one or more embryos to the subject. In some embodiments, the one or more oocytes or ova are isolated from the subject about 3 days prior to the transfer of the one or more embryos to the subject. In some embodiments, the one or more oocytes or ova are isolated from the subject about 4 days prior to the transfer of the one or more embryos to the subject. In some embodiments, the one or more oocytes or ova are isolated from the subject about 5 days prior to the transfer of the one or more embryos to the subject.

In some embodiments, a gonadotropin-releasing hormone (GnRH) antagonist is administered to the subject prior to isolation of the one or more oocytes (e.g., containing one or more mature oocytes) or ova from the subject. In some embodiments, human chorionic gonadotropin (hCG) is administered to the subject prior to isolation of the one or more oocytes or ova from the subject. For instance, the hCG can be administered to the subject in a single dose. In some embodiments, the hCG is administered to the subject in multiple doses. The hCG can be administered to the subject intravenously, such as by intravenous injection.

In some embodiments, progesterone is administered to the subject following isolation of the one or more oocytes or ova from the subject. The progesterone can be administered intravaginally, and may be administered at a dose of from about 300 mg to about 600 mg (for instance, about 300 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, or more). In some embodiments, 300 mg of progesterone per dose is administered to the subject following isolation of the one or more oocytes or ova from the subject. In some embodiments, 600 mg of progesterone per dose is administered to the subject following isolation of the one or more oocytes or ova from the subject.

In some embodiments, the progesterone is administered to the subject daily, preferably beginning within about 24 hours (e.g., within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours) of isolation of the one or more oocytes or ova from the subject and continuing for about 6 or more weeks (e.g., from about 6 weeks to about 10 weeks, such as about 6 weeks, about 7 weeks, about 8 weeks, about 9 weeks, about 10 weeks, or more) following the transfer of the one or more embryos to the subject.

In some embodiments, the one or more embryos are freshly transferred to the uterus of the subject (i.e., transferred to the uterus of the subject during the same menstrual cycle as isolation of the one or more oocytes or ova from the subject). For instance, the one or more embryos may be transferred to the uterus of the subject from about 1 day to about 7 days (e.g., from about 3 days to about 5 days, such as 3 days, 4 days, or 5 days) following the isolation of one or more oocytes or ova from the subject in preparation for IVF or ICSI.

In some embodiments, the one or more embryos are frozen and thawed prior to the transfer of the one or more embryos to the subject.

In some embodiments, the one or more embryos each contain from 6 to 8 blastomeres immediately prior to the transfer of the one or more embryos to the subject. The blastomeres may be of approximately equal sizes as assessed by visual microscopy prior to the transfer of the one or more embryos to the subject. In some embodiments, the one or more embryos comprise an embryo having the form of a morula. In some embodiments, the one or more embryos comprise an embryo having the form of a blastula (e.g., a mammalian blastocyst).

In some embodiments, the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)

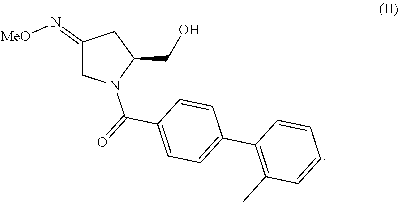 [SMILES: CO/N=C1/C[C@@H](CO)N(C(=O)c2ccc(-c3ccccc3C)cc2)C1]

In some embodiments, the compound represented by formula (II) (i.e., (3Z,5S)-5-(hydroxymethyl)-1-[(2′-methyl-1,1′-biphenyl-4-yl)carbonyl]pyrrolidin-3-one O-methyloxime) is substantially pure. For instance, in some embodiments, the compound represented by formula (II) has a purity of at least 85%, such as a purity of from 85% to 99.9% or more (e.g., a purity of 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more). The purity of the compound represented by formula (II) may be assessed, for instance, using nuclear magnetic resonance (NMR) techniques and/or chromatographic methods, such as high-performance liquid chromatography (HPLC) procedures, that are known in the art and described herein, such as those techniques that are described in U.S. Pat. No. 9,670,155, the disclosure of which is incorporated herein by reference in its entirety.

In some embodiments, the compound represented by formula (II) is substantially pure with respect to diastereomers of this compound and other by-products that may be formed during the synthesis of this compound. For instance, in some embodiments, the compound represented by formula (II) has a purity of at least 85%, such as a purity of from 85% to 99.9% or more (e.g., a purity of 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more) with respect to diastereomers of this compound and other by-products that may be formed during the synthesis of this compound, such as a by-product that is formed during the synthesis of this compound as described in U.S. Pat. No. 9,670,155. The purity of the compound represented by formula (II) may be assessed, for instance, using NMR techniques and/or chromatographic methods, such as HPLC procedures, that are known in the art and described herein, such as those techniques that are described in U.S. Pat. No. 9,670,155.

In some embodiments, the compound represented by formula (II) is substantially pure with respect to its (3E) diastereomer, (3E,5S)-5-(hydroxymethyl)-1-[(2′-methyl-1,1′-biphenyl-4-yl)carbonyl]pyrrolidin-3-one O-methyloxime. For instance, in some embodiments, the compound represented by formula (II) has a purity of at least 85%, such as a purity of from 85% to 99.9% or more (e.g., a purity of 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more) with respect to (3E,5S)-5-(hydroxymethyl)-1-[(2′-methyl-1,1′-biphenyl-4-yl)carbonyl]pyrrolidin-3-one O-methyloxime. For instance, compound (II) may be administered in the form of a composition (e.g., a tablet, such as a dispersible tablet, capsule, gel cap, powder, liquid solution, or liquid suspension) that contains less than 15% of the (3E) diastereomer. For example, compound (II) may be administered in the form of a composition (e.g., a tablet, such as a dispersible tablet, capsule, gel cap, powder, liquid solution, or liquid suspension) that contains less than 14%, less than 13%, less than 12%, less than 11%, less than 10%, less than 9%, less than 8%, less than 7%, less than 6%, less than 5%, less than 4%, less than 3%, less than 2%, less than 1%, less than 0.1%, less than 0.01%, less than 0.001%, or less of the (3E) diastereomer. The purity of the compound represented by formula (II) may be assessed, for instance, using NMR techniques and/or chromatographic methods, such as HPLC procedures, that are known in the art and described herein, such as those techniques that are described in U.S. Pat. No. 9,670,155.

In some embodiments, the compound is in a crystalline state. In some embodiments, the compound exhibits characteristic X-ray powder diffraction peaks at about 7.05° 2θ, about 13.13° 2θ, and about 23.34° 2θ. For instance, the compound may exhibit characteristic X-ray powder diffraction peaks at about 7.05° 2θ, about 12.25° 2θ, about 13.13° 2θ, about 16.54° 2θ, about 18.00° 2θ, about 21.84° 2θ, and about 23.34° 2θ. In some embodiments, the compound exhibits characteristic X-ray powder diffraction peaks as set forth in Table 1, below.

TABLE 1
Characteristic X-ray powder diffraction (XRPD)
peaks of crystal form of compound (II)

| XRPD | | Intensity |
|---|---|---|
| Peak (° 2θ) | d space (Å) | (%) |
| 7.05 ± 0.20 | 12.520 ± 0.354 | 45 |
| 12.25 ± 0.20 | 7.218 ± 0.117 | 36 |
| 13.13 ± 0.20 | 6.739 ± 0.102 | 55 |
| 14.16 ± 0.20 | 6.250 ± 0.088 | 8 |
| 16.54 ± 0.20 | 5.356 ± 0.064 | 38 |
| 18.00 ± 0.20 | 4.923 ± 0.054 | 36 |
| 18.77 ± 0.20 | 4.723 ± 0.050 | 34 |
| 21.32 ± 0.20 | 4.165 ± 0.039 | 5 |
| 21.84 ± 0.2 | 4.066 ± 0.037 | 36 |
| 23.34 ± 0.20 | 3.808 ± 0.032 | 100 |
| 24.08 ± 0.20 | 3.693 ± 0.030 | 14 |
| 24.67 ± 0.20 | 3.605 ± 0.029 | 1 |
| 25.45 ± 0.20 | 3.497 ± 0.027 | 27 |
| 25.69 ± 0.20 | 3.465 ± 0.027 | 8 |
| 26.45 ± 0.20 | 3.367 ± 0.025 | 10 |
| 27.09 ± 0.20 | 3.289 ± 0.024 | 2 |
| 28.05 ± 0.20 | 3.179 ± 0.022 | 14 |
| 28.56 ± 0.20 | 3.123 ± 0.021 | 3 |
| 29.26 ± 0.20 | 3.050 ± 0.020 | 16 |
| 30.72 ± 0.20 | 2.908 ± 0.018 | 2 |
| 31.00 ± 0.20 | 2.882 ± 0.018 | 3 |
| 31.19 ± 0.20 | 2.865 ± 0.018 | 5 |
| 33.19 ± 0.20 | 2.697 ± 0.016 | 2 |
| 33.60 ± 0.20 | 2.665 ± 0.015 | 6 |
| 34.36 ± 0.20 | 2.608 ± 0.015 | 4 |
| 34.75 ± 0.20 | 2.580 ± 0.014 | 2 |
| 35.91 ± 0.20 | 2.499 ± 0.013 | 2 |
| 36.52 ± 0.20 | 2.458 ± 0.013 | 3 |
| 37.38 ± 0.20 | 2.404 ± 0.012 | 2 |
| 37.70 ± 0.20 | 2.384 ± 0.012 | 1 |
| 38.73 ± 0.20 | 2.323 ± 0.012 | 3 |
| 39.11 ± 0.20 | 2.301 ± 0.011 | 2 |
| 39.80 ± 0.20 | 2.263 0.011 | 4 |

In some embodiments, the compound is administered orally to the subject. In some embodiments, the compound is administered intravenously to the subject. For instance, the compound may be administered to the subject in the form of a tablet, capsule, gel cap, powder, liquid solution, or liquid suspension. In some embodiments, the compound is administered to the subject in the form of a tablet, such as a dispersible tablet. The dispersible tablet may have, for example, one or more, or all, of the following components:

a. about 1-20% by weight of calcium silicate;

b. about 0.1-20% by weight of PVP30K;

c. about 0.01-5% by weight of poloxamer 188;

d. about 0.5-20% by weight of sodium croscarmellose;

e. about 1-90% by weight of microcrystalline cellulose 112;

f. about 1-90% by weight of lactose monohydrate;

g. about 0.01-0.5% by weight of sodium saccharine; and

h. about 0.1-10% by weight of glycerol dibehenate.

For instance, the dispersible tablet may have the following composition:

a. about 5% by weight of calcium silicate;

b. about 1% by weight of PVP30K;

c. about 2% by weight of poloxamer 188;

d. about 5% by weight of sodium croscarmellose;

e. about 1.5% by weight of microcrystalline cellulose 112;

f. about 47.8% by weight of lactose monohydrate;

g. about 0.2% by weight of sodium saccharine; and

h. about 4% by weight of glycerol dibehenate.

In some embodiments, the compound is administered to the subject in a unit dosage form containing from about 25 mg to about 250 mg of the compound, such as a unit dosage form containing about 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, or more, of the compound.

In some embodiments, the compound is administered to the subject in a unit dosage form containing from about 25 mg to about 75 mg of the compound, such as a unit dosage form containing about 50 mg of the compound. In some embodiments, the compound is administered to the subject in a unit dosage form containing from about 175 mg to about 225 mg of the compound, such as a unit dosage form containing about 200 mg of the compound.

In some embodiments, the oxytocin antagonist is administered to the subject in an amount of from 1,500 mg to 2,100 mg per dose, such as an amount of from 1,510 mg to 2,090 mg per dose, from 1,520 mg to 2,080 mg per dose, from 1,530 mg to 2,070 mg per dose, from 1,540 mg to 2,060 mg per dose, from 1,550 mg to 2,050 mg per dose, from 1,560 mg to 2,040 mg per dose, from 1,570 mg to 2,030 mg per dose, from 1,580 mg to 2,020 mg per dose, from 1,590 mg to 2,010 mg per dose, from 1,600 mg to 2,000 mg per dose, from 1,610 mg to 1,990 mg per dose, from 1,620 mg to 1,980 mg per dose, from 1,630 mg to 1,970 mg per dose, from 1,640 mg to 1,960 mg per dose, from 1,650 mg to 1,950 mg per dose, from 1,660 mg to 1,940 mg per dose, from 1,670 mg to 1,930 mg per dose, from 1,680 mg to 1,920 mg per dose, from 1,690 mg to 1,910 mg per dose, from 1,700 mg to 1,900 mg per dose, from 1,710 mg to 1,890 mg per dose, from 1,720 mg to 1,880 mg per dose, from 1,730 mg to 1,870 mg per dose, from 1,740 mg to 1,860 mg per dose, from 1,750 mg to 1,850 mg per dose, from 1,760 mg to 1,840 mg per dose, from 1,770 mg to 1,830 mg per dose, from 1,780 mg to 1,820 mg per dose, or from 1,790 mg to 1,810 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,501 mg to about 2,099 mg per dose, such as an amount of about 1,501 mg, 1,502 mg, 1,503 mg, 1,504 mg, 1,505 mg, 1,506 mg, 1,507 mg, 1,508 mg, 1,509 mg, 1,510 mg, 1,511 mg, 1,512 mg, 1,513 mg, 1,514 mg, 1,515 mg, 1,516 mg, 1,517 mg, 1,518 mg, 1,519 mg, 1,520 mg, 1,521 mg, 1,522 mg, 1,523 mg, 1,524 mg, 1,525 mg, 1,526 mg, 1,527 mg, 1,528 mg, 1,529 mg, 1,530 mg, 1,531 mg, 1,532 mg, 1,533 mg, 1,534 mg, 1,535 mg, 1,536 mg, 1,537 mg, 1,538 mg, 1,539 mg, 1,540 mg, 1,541 mg, 1,542 mg, 1,543 mg, 1,544 mg, 1,545 mg, 1,546 mg, 1,547 mg, 1,548 mg, 1,549 mg, 1,550 mg, 1,551 mg, 1,552 mg, 1,553 mg, 1,554 mg, 1,555 mg, 1,556 mg, 1,557 mg, 1,558 mg, 1,559 mg, 1,560 mg, 1,561 mg, 1,562 mg, 1,563 mg, 1,584 mg, 1,565 mg, 1,566 mg, 1,567 mg, 1,568 mg, 1,569 mg, 1,570 mg, 1,571 mg, 1,572 mg, 1,573 mg, 1,574 mg, 1,575 mg, 1,576 mg, 1,577 mg, 1,578 mg, 1,579 mg, 1,580 mg, 1,581 mg, 1,582 mg, 1,583 mg, 1,584 mg, 1,585 mg, 1,586 mg, 1,587 mg, 1,588 mg, 1,589 mg, 1,590 mg, 1,591 mg, 1,592 mg, 1,593 mg, 1,594 mg, 1,595 mg, 1,596 mg, 1,597 mg, 1,598 mg, 1,599 mg, 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, 2,000 mg, 2,001 mg, 2,002 mg, 2,003 mg, 2,004 mg, 2,005 mg, 2,006 mg, 2,007 mg, 2,008 mg, 2,009 mg, 2,010 mg, 2,011 mg, 2,012 mg, 2,013 mg, 2,014 mg, 2,015 mg, 2,016 mg, 2,017 mg, 2,018 mg, 2,019 mg, 2,020 mg, 2,021 mg, 2,022 mg, 2,023 mg, 2,024 mg, 2,025 mg, 2,026 mg, 2,027 mg, 2,028 mg, 2,029 mg, 2,030 mg, 2,031 mg, 2,032 mg, 2,033 mg, 2,034 mg, 2,035 mg, 2,036 mg, 2,037 mg, 2,038 mg, 2,039 mg, 2,040 mg, 2,041 mg, 2,042 mg, 2,043 mg, 2,044 mg, 2,045 mg, 2,046 mg, 2,047 mg, 2,048 mg, 2,049 mg, 2,050 mg, 2,051 mg, 2,052 mg, 2,053 mg, 2,054 mg, 2,055 mg, 2,056 mg, 2,057 mg, 2,058 mg, 2,059 mg, 2,060 mg, 2,061 mg, 2,062 mg, 2,063 mg, 2,064 mg, 2,065 mg, 2,066 mg, 2,067 mg, 2,068 mg, 2,069 mg, 2,070 mg, 2,071 mg, 2,072 mg, 2,073 mg, 2,074 mg, 2,075 mg, 2,076 mg, 2,077 mg, 2,078 mg, 2,079 mg, 2,080 mg, 2,081 mg, 2,082 mg, 2,083 mg, 2,084 mg, 2,085 mg, 2,086 mg, 2,087 mg, 2,088 mg, 2,089 mg, 2,090 mg, 2,091 mg, 2,092 mg, 2,093 mg, 2,094 mg, 2,095 mg, 2,096 mg, 2,097 mg, 2,098 mg, or 2,099 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,600 mg to about 2,000 mg per dose, such as an amount of about 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, or 2,000 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,700 mg to about 1,900 mg per dose, such as an amount of about 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, or 1,900 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,750 mg to about 1,850 mg per dose, such as an amount of about 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, or 1,850 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,760 mg to about 1,840 mg per dose, such as an amount of about 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, or 1,840 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (I)).

For example, in some embodiments, the oxytocin antagonist is administered to the subject in an amount of from about 1,770 mg to about 1,830 mg per dose, such as an amount of about 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, or 1,830 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,780 mg to about 1,820 mg per dose, such as an amount of about 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, or 1,820 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,790 mg to about 1,810 mg per dose, such as an amount of about 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, or 1,810 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of from about 2,100 mg to about 2,700 mg per dose, from about 2,150 mg to about 2,650 mg per dose, from about 2,200 mg to about 2,600 mg per dose, from about 2,250 mg to about 2,550 mg per dose, from about 2,300 mg to about 2,500 mg per dose, from about 2,350 mg to about 2,450 mg per dose, from about 2,360 mg to about 2,440 mg per dose, from about 2,370 mg to about 2,430 mg per dose, from about 2,380 mg to about 2,420 mg per dose, from about 2,390 mg to about 2,410 mg per dose, from about 2,391 mg to about 2,409 mg per dose, from about 2,392 mg to about 2,408 mg per dose, from about 2,393 mg to about 2,407 mg per dose, from about 2,394 mg to about 2,406 mg per dose, from about 2,395 mg to about 2,405 mg per dose, from about 2,396 mg to about 2,404 mg per dose, from about 2,397 mg to about 2,403 mg per dose, from about 2,398 mg to about 2,402 mg per dose, or from about 2,399 mg to about 2,401 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the compound is administered to the subject in an amount of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (I)).

In some embodiments, the oxytocin antagonist is administered to the subject in an amount of about 1,800 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in an amount of about 2,100 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in an amount of about 2,400 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in one or more doses (e.g., in 1, 2, 3, 4, 5, 6, 7, 8, 9, 10, or more doses) totaling from 1,500 mg to 2,100 mg, such as in one or more doses (e.g., in 1, 2, 3, 4, 5, 6, 7, 8, 9, 10, or more doses) totaling from 1,510 mg to 2,090 mg, from 1,520 mg to 2,080 mg, from 1,530 mg to 2,070 mg, from 1,540 mg to 2,060 mg, from 1,550 mg to 2,050 mg, from 1,560 mg to 2,040 mg, from 1,570 mg to 2,030 mg, from 1,580 mg to 2,020 mg, from 1,590 mg to 2,010 mg, from 1,600 mg to 2,000 mg, from 1,610 mg to 1,990 mg, from 1,620 mg to 1,980 mg, from 1,630 mg to 1,970 mg, from 1,640 mg to 1,960 mg, from 1,650 mg to 1,950 mg, from 1,660 mg to 1,940 mg, from 1,670 mg to 1,930 mg, from 1,680 mg to 1,920 mg, from 1,690 mg to 1,910 mg, from 1,700 mg to 1,900 mg, from 1,710 mg to 1,890 mg, from 1,720 mg to 1,880 mg, from 1,730 mg to 1,870 mg, from 1,740 mg to 1,860 mg, from 1,750 mg to 1,850 mg, from 1,760 mg to 1,840 mg, from 1,770 mg to 1,830 mg, from 1,780 mg to 1,820 mg, or from 1,790 mg to 1,810 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in in one or more doses (e.g., in 1, 2, 3, 4, 5, 6, 7, 8, 9, 10, or more doses) totaling from about 1,501 mg to about 2,099 mg, such as in one or more doses (e.g., in 1, 2, 3, 4, 5, 6, 7, 8, 9, 10, or more doses) totaling about 1,501 mg, 1,502 mg, 1,503 mg, 1,504 mg, 1,505 mg, 1,506 mg, 1,507 mg, 1,508 mg, 1,509 mg, 1,510 mg, 1,511 mg, 1,512 mg, 1,513 mg, 1,514 mg, 1,515 mg, 1,516 mg, 1,517 mg, 1,518 mg, 1,519 mg, 1,520 mg, 1,521 mg, 1,522 mg, 1,523 mg, 1,524 mg, 1,525 mg, 1,526 mg, 1,527 mg, 1,528 mg, 1,529 mg, 1,530 mg, 1,531 mg, 1,532 mg, 1,533 mg, 1,534 mg, 1,535 mg, 1,536 mg, 1,537 mg, 1,538 mg, 1,539 mg, 1,540 mg, 1,541 mg, 1,542 mg, 1,543 mg, 1,544 mg, 1,545 mg, 1,546 mg, 1,547 mg, 1,548 mg, 1,549 mg, 1,550 mg, 1,551 mg, 1,552 mg, 1,553 mg, 1,554 mg, 1,555 mg, 1,556 mg, 1,557 mg, 1,558 mg, 1,559 mg, 1,560 mg, 1,561 mg, 1,562 mg, 1,563 mg, 1,584 mg, 1,565 mg, 1,566 mg, 1,567 mg, 1,568 mg, 1,569 mg, 1,570 mg, 1,571 mg, 1,572 mg, 1,573 mg, 1,574 mg, 1,575 mg, 1,576 mg, 1,577 mg, 1,578 mg, 1,579 mg, 1,580 mg, 1,581 mg, 1,582 mg, 1,583 mg, 1,584 mg, 1,585 mg, 1,586 mg, 1,587 mg, 1,588 mg, 1,589 mg, 1,590 mg, 1,591 mg, 1,592 mg, 1,593 mg, 1,594 mg, 1,595 mg, 1,596 mg, 1,597 mg, 1,598 mg, 1,599 mg, 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, 2,000 mg, 2,001 mg, 2,002 mg, 2,003 mg, 2,004 mg, 2,005 mg, 2,006 mg, 2,007 mg, 2,008 mg, 2,009 mg, 2,010 mg, 2,011 mg, 2,012 mg, 2,013 mg, 2,014 mg, 2,015 mg, 2,016 mg, 2,017 mg, 2,018 mg, 2,019 mg, 2,020 mg, 2,021 mg, 2,022 mg, 2,023 mg, 2,024 mg, 2,025 mg, 2,026 mg, 2,027 mg, 2,028 mg, 2,029 mg, 2,030 mg, 2,031 mg, 2,032 mg, 2,033 mg, 2,034 mg, 2,035 mg, 2,036 mg, 2,037 mg, 2,038 mg, 2,039 mg, 2,040 mg, 2,041 mg, 2,042 mg, 2,043 mg, 2,044 mg, 2,045 mg, 2,046 mg, 2,047 mg, 2,048 mg, 2,049 mg, 2,050 mg, 2,051 mg, 2,052 mg, 2,053 mg, 2,054 mg, 2,055 mg, 2,056 mg, 2,057 mg, 2,058 mg, 2,059 mg, 2,060 mg, 2,061 mg, 2,062 mg, 2,063 mg, 2,064 mg, 2,065 mg, 2,066 mg, 2,067 mg, 2,068 mg, 2,069 mg, 2,070 mg, 2,071 mg, 2,072 mg, 2,073 mg, 2,074 mg, 2,075 mg, 2,076 mg, 2,077 mg, 2,078 mg, 2,079 mg, 2,080 mg, 2,081 mg, 2,082 mg, 2,083 mg, 2,084 mg, 2,085 mg, 2,086 mg, 2,087 mg, 2,088 mg, 2,089 mg, 2,090 mg, 2,091 mg, 2,092 mg, 2,093 mg, 2,094 mg, 2,095 mg, 2,096 mg, 2,097 mg, 2,098 mg, or 2,099 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (I)).

For example, in some embodiments, the oxytocin antagonist is administered to the subject in one or more doses (e.g., in 1, 2, 3, 4, 5, 6, 7, 8, 9, 10, or more doses) totaling from about 1,600 mg to about 2,000 mg, such as in one or more doses (e.g., in 1, 2, 3, 4, 5, 6, 7, 8, 9, 10, or more doses) totaling about 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,684 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, or 2,000 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in one or more doses (e.g., in 1, 2, 3, 4, 5, 6, 7, 8, 9, 10, or more doses) totaling from about 1,700 mg to about 1,900 mg, such as in one or more doses (e.g., in 1, 2, 3, 4, 5, 6, 7, 8, 9, 10, or more doses) totaling about 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, or 1,900 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in in one or more doses (e.g., in 1, 2, 3, 4, 5, 6, 7, 8, 9, 10, or more doses) totaling from about 1,750 mg to about 1,850 mg, such as an amount of about 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, or 1,850 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in one or more doses (e.g., in 1, 2, 3, 4, 5, 6, 7, 8, 9, 10, or more doses) totaling from about 1,760 mg to about 1,840 mg, such as in one or more doses (e.g., in 1, 2, 3, 4, 5, 6, 7, 8, 9, 10, or more doses) totaling about 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, or 1,840 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in one or more doses (e.g., in 1, 2, 3, 4, 5, 6, 7, 8, 9, 10, or more doses) totaling from about 1,770 mg to about 1,830 mg, such as an amount of about 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, or 1,830 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in one or more doses (e.g., in 1, 2, 3, 4, 5, 6, 7, 8, 9, 10, or more doses) totaling from about 1,780 mg to about 1,820 mg, such as an amount of about 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, or 1,820 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in one or more doses (e.g., in 1, 2, 3, 4, 5, 6, 7, 8, 9, 10, or more doses) totaling from about 1,790 mg to about 1,810 mg, such as an amount of about 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, or 1,810 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the compound is administered to the subject in one or more doses (e.g., in 1, 2, 3, 4, 5, 6, 7, 8, 9, 10, or more doses) totaling about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in one or more doses (e.g., in 1, 2, 3, 4, 5, 6, 7, 8, 9, 10, or more doses) totaling about 1,800 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in one or more doses (e.g., in 1, 2, 3, 4, 5, 6, 7, 8, 9, 10, or more doses) totaling about 2,100 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in one or more doses (e.g., in 1, 2, 3, 4, 5, 6, 7, 8, 9, 10, or more doses) totaling about 2,400 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in a single dose (e.g., on the day of the embryo transfer therapy) of from 1,500 mg to 2,100 mg, such as in a single dose (e.g., on the day of the embryo transfer therapy) of from 1,510 mg to 2,090 mg, from 1,520 mg to 2,080 mg, from 1,530 mg to 2,070 mg, from 1,540 mg to 2,060 mg, from 1,550 mg to 2,050 mg, from 1,560 mg to 2,040 mg, from 1,570 mg to 2,030 mg, from 1,580 mg to 2,020 mg, from 1,590 mg to 2,010 mg, from 1,600 mg to 2,000 mg, from 1,610 mg to 1,990 mg, from 1,620 mg to 1,980 mg, from 1,630 mg to 1,970 mg, from 1,640 mg to 1,960 mg, from 1,650 mg to 1,950 mg, from 1,660 mg to 1,940 mg, from 1,670 mg to 1,930 mg, from 1,680 mg to 1,920 mg, from 1,690 mg to 1,910 mg, from 1,700 mg to 1,900 mg, from 1,710 mg to 1,890 mg, from 1,720 mg to 1,880 mg, from 1,730 mg to 1,870 mg, from 1,740 mg to 1,860 mg, from 1,750 mg to 1,850 mg, from 1,760 mg to 1,840 mg, from 1,770 mg to 1,830 mg, from 1,780 mg to 1,820 mg, or from 1,790 mg to 1,810 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (I)).

For example, in some embodiments, the oxytocin antagonist is administered to the subject in a single dose (e.g., on the day of the embryo transfer therapy) of from about 1,501 mg to about 2,099 mg, such as in a single dose (e.g., on the day of the embryo transfer therapy) of about 1,501 mg, 1,502 mg, 1,503 mg, 1,504 mg, 1,505 mg, 1,506 mg, 1,507 mg, 1,508 mg, 1,509 mg, 1,510 mg, 1,511 mg, 1,512 mg, 1,513 mg, 1,514 mg, 1,515 mg, 1,516 mg, 1,517 mg, 1,518 mg, 1,519 mg, 1,520 mg, 1,521 mg, 1,522 mg, 1,523 mg, 1,524 mg, 1,525 mg, 1,526 mg, 1,527 mg, 1,528 mg, 1,529 mg, 1,530 mg, 1,531 mg, 1,532 mg, 1,533 mg, 1,534 mg, 1,535 mg, 1,536 mg, 1,537 mg, 1,538 mg, 1,539 mg, 1,540 mg, 1,541 mg, 1,542 mg, 1,543 mg, 1,544 mg, 1,545 mg, 1,546 mg, 1,547 mg, 1,548 mg, 1,549 mg, 1,550 mg, 1,551 mg, 1,552 mg, 1,553 mg, 1,554 mg, 1,555 mg, 1,556 mg, 1,557 mg, 1,558 mg, 1,559 mg, 1,560 mg, 1,561 mg, 1,562 mg, 1,563 mg, 1,564 mg, 1,565 mg, 1,566 mg, 1,567 mg, 1,568 mg, 1,569 mg, 1,570 mg, 1,571 mg, 1,572 mg, 1,573 mg, 1,574 mg, 1,575 mg, 1,576 mg, 1,577 mg, 1,578 mg, 1,579 mg, 1,580 mg, 1,581 mg, 1,582 mg, 1,583 mg, 1,584 mg, 1,585 mg, 1,586 mg, 1,587 mg, 1,588 mg, 1,589 mg, 1,590 mg, 1,591 mg, 1,592 mg, 1,593 mg, 1,594 mg, 1,595 mg, 1,596 mg, 1,597 mg, 1,598 mg, 1,599 mg, 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, 2,000 mg, 2,001 mg, 2,002 mg, 2,003 mg, 2,004 mg, 2,005 mg, 2,006 mg, 2,007 mg, 2,008 mg, 2,009 mg, 2,010 mg, 2,011 mg, 2,012 mg, 2,013 mg, 2,014 mg, 2,015 mg, 2,016 mg, 2,017 mg, 2,018 mg, 2,019 mg, 2,020 mg, 2,021 mg, 2,022 mg, 2,023 mg, 2,024 mg, 2,025 mg, 2,026 mg, 2,027 mg, 2,028 mg, 2,029 mg, 2,030 mg, 2,031 mg, 2,032 mg, 2,033 mg, 2,034 mg, 2,035 mg, 2,036 mg, 2,037 mg, 2,038 mg, 2,039 mg, 2,040 mg, 2,041 mg, 2,042 mg, 2,043 mg, 2,044 mg, 2,045 mg, 2,046 mg, 2,047 mg, 2,048 mg, 2,049 mg, 2,050 mg, 2,051 mg, 2,052 mg, 2,053 mg, 2,054 mg, 2,055 mg, 2,056 mg, 2,057 mg, 2,058 mg, 2,059 mg, 2,060 mg, 2,061 mg, 2,062 mg, 2,063 mg, 2,064 mg, 2,065 mg, 2,066 mg, 2,067 mg, 2,068 mg, 2,069 mg, 2,070 mg, 2,071 mg, 2,072 mg, 2,073 mg, 2,074 mg, 2,075 mg, 2,076 mg, 2,077 mg, 2,078 mg, 2,079 mg, 2,080 mg, 2,081 mg, 2,082 mg, 2,083 mg, 2,084 mg, 2,085 mg, 2,086 mg, 2,087 mg, 2,088 mg, 2,089 mg, 2,090 mg, 2,091 mg, 2,092 mg, 2,093 mg, 2,094 mg, 2,095 mg, 2,096 mg, 2,097 mg, 2,098 mg, or 2,099 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,600 mg to about 2,000 mg, such as in a single dose (e.g., on the day of the embryo transfer therapy) of about 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, or 2,000 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,700 mg to about 1,900 mg, such as in a single dose (e.g., on the day of the embryo transfer therapy) of about 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, or 1,900 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,750 mg to about 1,850 mg, such as in a single dose (e.g., on the day of the embryo transfer therapy) of about 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, or 1,850 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (I)).

For example, in some embodiments, the oxytocin antagonist is administered to the subject in a single dose (e.g., on the day of the embryo transfer therapy) of from about 1,760 mg to about 1,840 mg, such as in a single dose (e.g., on the day of the embryo transfer therapy) of about 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, or 1,840 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,770 mg to about 1,830 mg, such as in a single dose (e.g., on the day of the embryo transfer therapy) of about 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, or 1,830 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of about 1,780 mg to about 1,820 mg, such as in a single dose (e.g., on the day of the embryo transfer therapy) of about 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, or 1,820 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,790 mg to about 1,810 mg, such as in a single dose (e.g., on the day of the embryo transfer therapy) of about 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, or 1,810 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the compound is administered to the subject in a single dose (e.g., on the day of the embryo transfer therapy) of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in a single dose (e.g., on the day of the embryo transfer therapy) of about 1,800 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in a single dose (e.g., on the day of the embryo transfer therapy) of about 2,100 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in a single dose (e.g., on the day of the embryo transfer therapy) of about 2,400 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

Administration of the oxytocin antagonist may induce a reduction in uterine contractility. In some embodiments, the subject exhibits a reduction in the frequency of uterine contractions following administration of the oxytocin antagonist, such as a reduction of from about 1% to about 20% (e.g., a reduction of about 1%, 2%, 3%, 4%, 5%, 6%, 7%, 8%, 9%, 10%, 11%, 12%, 13%, 14%, 15%, 16%, 17%, 18%, 19%, 20%, or more) relative to a measurement of the frequency of uterine contractions in the subject recorded prior to administration of the oxytocin antagonist.

In some embodiments of any of the above aspects of the disclosure, the subject has been determined to exhibit a serum progesterone (P4) concentration of less than about 320 nM prior to the transfer of the one or more embryos to the subject. For instance, the subject may exhibit a serum P4 concentration of from about 200 nM to about 300 nM (e.g., a serum P4 concentration of about 200 nM, 205 nM, 210 nM, 215 nM, 220 nM, 225 nM, 230 nM, 235 nM, 240 nM, 245 nM, 250 nM, 255 nM, 260 nM, 265 nM, 270 nM, 275 nM, 280 nM, 285 nM, 290 nM, 295 nM, or 300 nM) prior to the transfer of the one or more embryos to the subject. In some embodiments, the subject has been determined to exhibit a serum P4 concentration of less than about 320 nM, for instance, within 24 hours prior to the transfer of the one or more embryos to the subject (e.g., within 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the transfer of the one or more embryos to the subject).

In some embodiments, the subject has been determined to exhibit a serum P4 concentration of from about 200 nM to about 300 nM, for instance, within 24 hours prior to the transfer of the one or more embryos to the subject (e.g., within 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the transfer of the one or more embryos to the subject).

In some embodiments, the subject has been determined to exhibit a serum P4 concentration of less than 2.0 ng/ml (e.g., of 1.54 ng/ml or less) prior to the transfer of the one or more embryos to the subject. In some embodiments, the subject has been determined to exhibit a serum P4 concentration of less than 2.0 ng/ml (e.g., of 1.54 ng/ml or less), for instance, from about 1 day to about 7 days prior to the transfer of the one or more embryos to the subject.

In some embodiments, the subject has been determined to exhibit a serum P4 concentration of less than 2.0 ng/ml (e.g., of 1.54 ng/ml or less) about 1 day prior to the transfer of the one or more embryos to the subject, such as within about 24 hours of, or immediately prior to, isolation of the one or more oocytes or ova from a subject undergoing IVF-ET or ICSI-ET.

In some embodiments, the subject has been determined to exhibit a serum P4 concentration of less than 2.0 ng/ml (e.g., of 1.54 ng/ml or less) about 2 days prior to the transfer of the one or more embryos to the subject, such as within about 24 hours of, or immediately prior to, isolation of the one or more oocytes or ova from a subject undergoing IVF-ET or ICSI-ET.

In some embodiments, the subject has been determined to exhibit a serum P4 concentration of less than 2.0 ng/ml (e.g., of 1.54 ng/ml or less) about 3 days prior to the transfer of the one or more embryos to the subject, such as within about 24 hours of, or immediately prior to, isolation of the one or more oocytes or ova from a subject undergoing IVF-ET or ICSI-ET.

In some embodiments, the subject has been determined to exhibit a serum P4 concentration of less than 2.0 ng/ml (e.g., of 1.54 ng/ml or less) about 4 days prior to the transfer of the one or more embryos to the subject, such as within about 24 hours of, or immediately prior to, isolation of the one or more oocytes or ova from a subject undergoing IVF-ET or ICSI-ET.

In some embodiments, the subject has been determined to exhibit a serum P4 concentration of less than 2.0 ng/ml (e.g., of 1.54 ng/ml or less) about 5 days prior to the transfer of the one or more embryos to the subject, such as within about 24 hours of, or immediately prior to, isolation of the one or more oocytes or ova from a subject undergoing IVF-ET or ICSI-ET.

In some embodiments, the subject has been determined to exhibit a serum P4 concentration of less than 2.0 ng/ml (e.g., of 1.54 ng/ml or less) about 6 days prior to the transfer of the one or more embryos to the subject, such as within about 24 hours of, or immediately prior to, isolation of the one or more oocytes or ova from a subject undergoing IVF-ET or ICSI-ET.

In some embodiments, the subject has been determined to exhibit a serum P4 concentration of less than 2.0 ng/ml (e.g., of 1.54 ng/ml or less) about 7 days prior to the transfer of the one or more embryos to the subject, such as within about 24 hours of, or immediately prior to, isolation of the one or more oocytes or ova from a subject undergoing IVF-ET or ICSI-ET.

In some embodiments, the subject has been determined to exhibit the serum P4 concentration of less than 2.0 ng/ml (e.g., of 1.54 ng/ml or less) within about 48 hours of administering hCG to the subject (e.g., so as to induce final follicular maturation), such as within about 47 hours, 46 hours, 45 hours, 44 hours, 43 hours, 42 hours, 41 hours, 40 hours, 39 hours, 38 hours, 37 hours, 36 hours, 35 hours, 34 hours, 33 hours, 32 hours, 31 hours, 30 hours, 29 hours, 28 hours, 27 hours, 26 hours, 25 hours, 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 1 hour, or less, prior to administration of hCG to the subject.

In some embodiments, the subject has been determined to exhibit a serum P4 concentration of less than 1.5 ng/ml prior to the transfer of the one or more embryos to the subject. In some embodiments, the subject has been determined to exhibit a serum P4 concentration of less than 1.5 ng/ml, for instance, from about 1 day to about 7 days prior to the transfer of the one or more embryos to the subject.

In some embodiments, the subject has been determined to exhibit a serum P4 concentration of less than 1.5 ng/ml about 1 day prior to the transfer of the one or more embryos to the subject, such as within about 24 hours of, or immediately prior to, isolation of the one or more oocytes or ova from a subject undergoing IVF-ET or ICSI-ET.

In some embodiments, the subject has been determined to exhibit a serum P4 concentration of less than 1.5 ng/ml about 2 days prior to the transfer of the one or more embryos to the subject, such as within about 24 hours of, or immediately prior to, isolation of the one or more oocytes or ova from a subject undergoing IVF-ET or ICSI-ET.

In some embodiments, the subject has been determined to exhibit a serum P4 concentration of less than 1.5 ng/ml about 3 days prior to the transfer of the one or more embryos to the subject, such as within about 24 hours of, or immediately prior to, isolation of the one or more oocytes or ova from a subject undergoing IVF-ET or ICSI-ET.

In some embodiments, the subject has been determined to exhibit a serum P4 concentration of less than 1.5 ng/ml about 4 days prior to the transfer of the one or more embryos to the subject, such as within about 24 hours of, or immediately prior to, isolation of the one or more oocytes or ova from a subject undergoing IVF-ET or ICSI-ET.

In some embodiments, the subject has been determined to exhibit a serum P4 concentration of less than 1.5 ng/ml about 5 days prior to the transfer of the one or more embryos to the subject, such as within about 24 hours of, or immediately prior to, isolation of the one or more oocytes or ova from a subject undergoing IVF-ET or ICSI-ET.

In some embodiments, the subject has been determined to exhibit a serum P4 concentration of less than 1.5 ng/ml about 6 days prior to the transfer of the one or more embryos to the subject, such as within about 24 hours of, or immediately prior to, isolation of the one or more oocytes or ova from a subject undergoing IVF-ET or ICSI-ET.

In some embodiments, the subject has been determined to exhibit a serum P4 concentration of less than 1.5 ng/ml about 7 days prior to the transfer of the one or more embryos to the subject, such as within about 24 hours of, or immediately prior to, isolation of the one or more oocytes or ova from a subject undergoing IVF-ET or ICSI-ET.

In some embodiments, the subject has been determined to exhibit the serum P4 concentration of less than 1.5 ng/ml within about 48 hours of administering hCG to the subject (e.g., so as to induce final follicular maturation), such as within about 47 hours, 46 hours, 45 hours, 44 hours, 43 hours, 42 hours, 41 hours, 40 hours, 39 hours, 38 hours, 37 hours, 36 hours, 35 hours, 34 hours, 33 hours, 32 hours, 31 hours, 30 hours, 29 hours, 28 hours, 27 hours, 26 hours, 25 hours, 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 1 hour, or less, prior to administration of hCG to the subject.

In some embodiments, the serum P4 concentration is assessed immediately following isolation of a sample (e.g., a blood serum sample) from the subject. In some embodiments, a sample (e.g., a blood serum sample) is withdrawn from a subject and is stored or preserved prior to progesterone analysis. In some embodiments, (i) the sample is withdrawn from the subject and (ii) the determination of the progesterone concentration in the sample is made immediately prior to the isolation of one or more oocytes or ova from the subject, such as a subject undergoing IVF-ET or ICSI-ET. For instance, in some embodiments, the sample is withdrawn from the subject and the serum P4 concentration is assessed from about 1 day to about 7 days prior to the transfer of the one or more embryos to the subject. In some embodiments, the sample is withdrawn from the subject and the serum P4 concentration is assessed about 3 days prior to the transfer of the one or more embryos to the subject. In some embodiments, the sample is withdrawn from the subject and the serum P4 concentration is assessed about 4 days prior to the transfer of the one or more embryos to the subject. In some embodiments, the sample is withdrawn from the subject and the serum P4 concentration is assessed about 5 days prior to the transfer of the one or more embryos to the subject. In some embodiments, the sample is withdrawn from the subject and the serum P4 concentration is assessed within about 48 hours of administering hCG to the subject, for instance, in preparation for oocyte or ovum retrieval, such as within about 47 hours, 46 hours, 45 hours, 44 hours, 43 hours, 42 hours, 41 hours, 40 hours, 39 hours, 38 hours, 37 hours, 36 hours, 35 hours, 34 hours, 33 hours, 32 hours, 31 hours, 30 hours, 29 hours, 28 hours, 27 hours, 26 hours, 25 hours, 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 1 hour, or less, prior to administration of hCG to the subject.

In some embodiments, the subject exhibits an increase in endometrial and/or myometrial prostaglandin E2 (PGE2) expression following administration of the oxytocin antagonist to the subject, for instance, as assessed by mass spectrometric and/or spectroscopic techniques described herein or known in the art. In some embodiments, the subject exhibits an increase in endometrial and/or myometrial prostaglandin F2α (PGF2α) expression following administration of the oxytocin antagonist to the subject, for instance, as assessed by mass spectrometric and/or spectroscopic techniques described herein or known in the art. In some embodiments, the subject exhibits a reduction in endometrial and/or myometrial PGF2α signaling following administration of the oxytocin antagonist, for instance, as assessed by detecting an increase in the concentration of phosphatidylinositol-4,5-bisphosphate (PIP₂) and/or a decrease in the concentration of one or more secondary messengers involved in PGF2α signal transduction, such as diacylglycerol (DAG), inositol-1,4,5-trisphosphate (IP₃), and/or intracellular calcium (Ca²⁺) released from Ca²⁺ stores, such as sarcoplasmic reticula. For instance, the subject may exhibit a transient increase in endometrial and/or myometrial PGF2α expression, followed by a reduction in PGF2α signalling in these tissues, as evidenced, for instance, by a reduction in endometrial and/or myometrial [DAG], [IP₃], and/or [Ca²⁺].

In some embodiments, the subject sustains pregnancy for at least about 14 days following the transfer of the one or more embryos to the subject, such as for about 14 days, 15 days, 16 days, 17 days, 18 days, 19 days, 20 days, 21 days, 22 days, 23 days, 24 days, 25 days, 26 days, 27 days, 28 days, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, or more, following the transfer of the one or more embryos to the subject. In some embodiments, the subject sustains pregnancy for at least about 6 weeks following the transfer of the one or more embryos to the subject, such as for about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, or more. In some embodiments, the subject sustains pregnancy for at least about 10 weeks following the transfer of the one or more embryos to the subject and/or following the retrieval of one or more oocytes or ova from the subject, such as for about 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, or more, following the transfer of the one or more embryos to the subject and/or following the retrieval of one or more oocytes or ova from the subject.

In some embodiments, pregnancy is assessed by a blood pregnancy test, such as by detecting the presence and/or quantity of hCG in a blood sample isolated from the subject. In some embodiments, pregnancy is assessed by detecting intrauterine embryo heartbeat, for instance, at about 6 weeks or more (e.g., about 6 weeks following the transfer of the one or more embryos to the subject and/or following the retrieval of one or more oocytes or ova from the subject, such as for about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, or more) following the transfer of the one or more embryos to the subject and/or following the retrieval of one or more oocytes or ova from the subject.

In some embodiments, the subject sustains pregnancy and exhibits a live birth following administration of the oxytocin antagonist to the subject. For instance, in some embodiments, the subject sustains pregnancy following administration of the oxytocin antagonist to the subject and exhibits a live birth at a gestational age of at least about 24 weeks, such as at a gestational age of about 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, or more.

In another aspect, the disclosure provides a kit including a package insert and an oxytocin antagonist, such as a compound represented by formula (I)

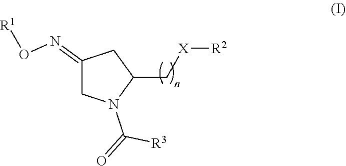

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring;

wherein the package insert instructs a user of the kit to perform the method of any of the foregoing aspects and embodiments of the disclosure. In some embodiments, the oxytocin antagonist is a compound represented by formula (II)

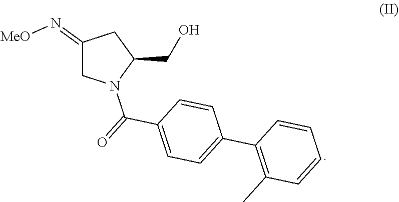 [SMILES: CO/N=C1/C[C@@H](CO)N(C(=O)c2ccc(-c3ccccc3C)cc2)C1]

In some embodiments, the compound represented by formula (II) (i.e., (3Z,5S)-5-(hydroxymethyl)-1-[(2′-methyl-1,1′-biphenyl-4-yl)carbonyl]pyrrolidin-3-one O-methyloxime) is substantially pure. For instance, in some embodiments, the compound represented by formula (II) has a purity of at least 85%, such as a purity of from 85% to 99.9% or more (e.g., a purity of 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more). The purity of the compound represented by formula (II) may be assessed, for instance, using NMR techniques and/or chromatographic methods, such as HPLC procedures, that are known in the art and described herein, such as those techniques that are described in U.S. Pat. No. 9,670,155, the disclosure of which is incorporated herein by reference in its entirety.

In some embodiments, the compound represented by formula (II) is substantially pure with respect to diastereomers of this compound and other by-products that may be formed during the synthesis of this compound. For instance, in some embodiments, the compound represented by formula (II) has a purity of at least 85%, such as a purity of from 85% to 99.9% or more (e.g., a purity of 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more) with respect to diastereomers of this compound and other by-products that may be formed during the synthesis of this compound, such as a by-product that is formed during the synthesis of this compound as described in U.S. Pat. No. 9,670,155. The purity of the compound represented by formula (II) may be assessed, for instance, using NMR techniques and/or chromatographic methods, such as HPLC procedures, that are known in the art and described herein, such as those techniques that are described in U.S. Pat. No. 9,670,155.

In some embodiments, the compound represented by formula (II) is substantially pure with respect to its (3E) diastereomer, (3E,5S)-5-(hydroxymethyl)-1-[(2′-methyl-1,1′-biphenyl-4-yl)carbonyl]pyrrolidin-3-one O-methyloxime. For instance, in some embodiments, the compound represented by formula (II) has a purity of at least 85%, such as a purity of from 85% to 99.9% or more (e.g., a purity of 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more) with respect to (3E,5S)-5-(hydroxymethyl)-1-[(2′-methyl-1,1′-biphenyl-4-yl)carbonyl]pyrrolidin-3-one O-methyloxime. For instance, compound (II) may be administered in the form of a composition (e.g., a tablet, such as a dispersible tablet, capsule, gel cap, powder, liquid solution, or liquid suspension) that contains less than 15% of the (3E) diastereomer. For example, compound (II) may be administered in the form of a composition (e.g., a tablet, such as a dispersible tablet, capsule, gel cap, powder, liquid solution, or liquid suspension) that contains less than 14%, less than 13%, less than 12%, less than 11%, less than 10%, less than 9%, less than 8%, less than 7%, less than 6%, less than 5%, less than 4%, less than 3%, less than 2%, less than 1%, less than 0.1%, less than 0.01%, less than 0.001%, or less of the (3E) diastereomer. The purity of the compound represented by formula (II) may be assessed, for instance, using NMR techniques and/or chromatographic methods, such as HPLC procedures, that are known in the art and described herein, such as those techniques that are described in U.S. Pat. No. 9,670,155.

In some embodiments, the compound is formulated for oral administration to the subject, and may be, for instance, in the form of a tablet, capsule, gel cap, powder, liquid solution, or liquid suspension. In some embodiments, the compound is formulated as a tablet, such as a dispersible tablet. The compound may be formulated in a unit dosage form containing from about 25 mg to about 250 mg of the compound, such as a unit dosage form containing about 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, or more, of the compound. In some embodiments, the compound is formulated in a unit dosage form containing from about 25 mg to about 75 mg of the compound, such as a unit dosage form containing about 50 mg of the compound. In some embodiments the compound is formulated in a unit dosage form containing from about 175 mg to about 225 mg of the compound, such as a unit dosage form containing about 200 mg of the compound.

In some embodiments, the oxytocin antagonist is epelsiban, or a salt, derivative, variant, crystal form, or formulation thereof, such as a salt, derivative, variant, crystal form, or formulation described in U.S. Pat. Nos. 7,514,437; 8,367,673; 8,541,579; 7,550,462; 7,919,492; 8,202,864; 8,742,099; 9,408,851; 8,716,286; or 8,815,856, the disclosures of each of which are incorporated herein by reference in their entirety.

In some embodiments, the oxytocin antagonist is retosiban, or a salt, derivative, variant, crystal form, or formulation thereof, such as a salt, derivative, variant, crystal form, or formulation described in U.S. Pat. Nos. 7,514,437; 8,367,673; 8,541,579; 8,071,594; 8,357,685; 8,937,179; or 9,452,169, the disclosures of each of which are incorporated herein by reference in their entirety.

In some embodiments, the oxytocin antagonist is barusiban, or a salt, derivative, variant, crystal 5 form, or formulation thereof, such as a salt, derivative, variant, crystal form, or formulation described in U.S. Pat. Nos. 6,143,722; 7,091,314; 7,816,489; or 9,579,305, or WO 2017/060339, the disclosures of each of which are incorporated herein by reference in their entirety.

In some embodiments, the oxytocin antagonist is atosiban, or a salt, derivative, variant, crystal form, or formulation thereof, such as a salt, derivative, variant, crystal form, or formulation described in U.S. Pat. No. 4,504,469 or 4,402,942, the disclosures of each of which are incorporated herein by reference in their entirety.

In another aspect, the disclosure features a method of treating a subject undergoing embryo transfer therapy, wherein the concentration of P4 in a sample isolated from the subject has been determined, by:

a. comparing the concentration of P4 to a P4 reference level; and

b. administering to the subject a therapeutically effective amount of an oxytocin antagonist if the concentration of P4 in the sample isolated from the subject is below the P4 reference level;

wherein one or more embryos are transferred to the uterus of the subject.

In another aspect, the disclosure features a method of treating a subject undergoing embryo transfer therapy by:

a. determining the concentration of P4 in a sample isolated from the subject;

b. comparing the concentration of P4 to a P4 reference level; and

c. administering to the subject a therapeutically effective amount of an oxytocin antagonist if the concentration of P4 in the sample isolated from the subject is below the P4 reference level;

wherein one or more embryos are transferred to the uterus of the subject.

In another aspect, the disclosure features an oxytocin antagonist for use in a method of treating a subject undergoing embryo transfer therapy, wherein the concentration of P4 in a sample isolated from the subject has been determined, wherein the method includes:

a. comparing the concentration of P4 to a P4 reference level; and

b. administering to the subject a therapeutically effective amount of an oxytocin antagonist if the concentration of P4 in the sample isolated from the subject is below the P4 reference level;

wherein one or more embryos are transferred to the uterus of the subject.

In another aspect, the disclosure features an oxytocin antagonist for use in a method of treating a subject undergoing embryo transfer therapy, wherein the method includes:

a. determining the concentration of P4 in a sample isolated from the subject;

b. comparing the concentration of P4 to a P4 reference level; and

c. administering to the subject a therapeutically effective amount of an oxytocin antagonist if the concentration of P4 in the sample isolated from the subject is below the P4 reference level;

wherein one or more embryos are transferred to the uterus of the subject.

In some embodiments, the subject is identified as having a concentration of P4 in the sample isolated from the subject that is less than the P4 reference level. Thus, in some embodiments, the method includes comparing the concentration of P4 in the sample isolated from the subject to a P4 reference level, determining that the concentration of P4 in the sample isolated from the subject is less than the P4 reference level, and administering a therapeutically effective amount of the oxytocin antagonist to the subject.

In some embodiments, the method includes the step of informing the subject that the subject has been identified as having a concentration of P4 in the sample isolated from the subject that is less than the P4 reference level.

In another aspect, the disclosure features a method of treating a subject undergoing embryo transfer therapy, wherein the concentration of P4 in a sample isolated from the subject has been determined, by:

a. comparing the concentration of P4 to a P4 reference level;

b. administering to the subject a therapeutically effective amount of an oxytocin antagonist if the concentration of P4 in the sample isolated from the subject is below the P4 reference level; and

c. transferring one or more embryos to the uterus of the subject.

In another aspect, the disclosure features a method of treating a subject undergoing embryo transfer therapy by:

a. determining the concentration of P4 in a sample isolated from the subject;

b. comparing the concentration of P4 to a P4 reference level;

c. administering to the subject a therapeutically effective amount of an oxytocin antagonist if the concentration of P4 in the sample isolated from the subject is below the P4 reference level; and

d. transferring one or more embryos to the uterus of the subject.

In another aspect, the disclosure features an oxytocin antagonist for use in a method of treating a subject undergoing embryo transfer therapy, wherein the concentration of P4 in a sample isolated from the subject has been determined, wherein the method includes:

a. comparing the concentration of P4 to a P4 reference level;

b. administering to the subject a therapeutically effective amount of an oxytocin antagonist if the concentration of P4 in the sample isolated from the subject is below the P4 reference level; and

c. transferring one or more embryos to the uterus of the subject.

In another aspect, the disclosure features an oxytocin antagonist for use in a method of treating a subject undergoing embryo transfer therapy, wherein the method includes:

a. determining the concentration of P4 in a sample isolated from the subject;

b. comparing the concentration of P4 to a P4 reference level;

c. administering to the subject a therapeutically effective amount of an oxytocin antagonist if the concentration of P4 in the sample isolated from the subject is below the P4 reference level; and

d. transferring one or more embryos to the uterus of the subject.

In some embodiments, the subject is identified as having a concentration of P4 in the sample isolated from the subject that is less than the P4 reference level. Thus, in some embodiments, the method includes comparing the concentration of P4 in the sample isolated from the subject to a P4 reference level, determining that the concentration of P4 in the sample isolated from the subject is less than the P4 reference level, administering a therapeutically effective amount of the oxytocin antagonist to the subject, and transferring one or more embryos to the uterus of the subject.

In some embodiments, the method includes the step of informing the subject that the subject has been identified as having a concentration of P4 in the sample isolated from the subject that is less than the P4 reference level.

In another aspect, the disclosure features a method of determining whether a subject undergoing embryo transfer therapy is likely to benefit from oxytocin antagonist treatment, wherein the concentration of P4 in a sample isolated from the subject has been determined, the method including comparing the concentration of P4 to a P4 reference level, wherein a reduced concentration of P4 in the sample isolated from the subject relative to the P4 reference level identifies the subject as likely to benefit from oxytocin antagonist treatment prior to, concurrently with, and/or following transfer of one or more embryos to the subject.

In another aspect, the disclosure features a method of determining whether a subject undergoing embryo transfer therapy is likely to benefit from oxytocin antagonist treatment, the method including determining the concentration of P4 in a sample isolated from the subject and comparing the concentration of P4 to a P4 reference level, wherein a reduced concentration of P4 in the sample isolated from the subject relative to the P4 reference level identifies the subject as likely to benefit from oxytocin antagonist treatment prior to, concurrently with, and/or following transfer of one or more embryos to the subject.

In another aspect, the disclosure features a method of collecting data for determining whether a subject undergoing embryo transfer therapy is likely to benefit from oxytocin antagonist treatment, wherein the concentration of P4 in a sample isolated from the subject has been determined, the method including comparing the concentration of P4 to a P4 reference level, wherein a reduced concentration of P4 in the sample isolated from the subject relative to the P4 reference level identifies the subject as likely to benefit from oxytocin antagonist treatment prior to, concurrently with, and/or following transfer of one or more embryos to the subject.

In another aspect, the disclosure features a method of collecting data for determining whether a subject undergoing embryo transfer therapy is likely to benefit from oxytocin antagonist treatment, the method including determining the concentration of P4 in a sample isolated from the subject and comparing the concentration of P4 to a P4 reference level, wherein a reduced concentration of P4 in the sample isolated from the subject relative to the P4 reference level identifies the subject as likely to benefit from oxytocin antagonist treatment prior to, concurrently with, and/or following transfer of one or more embryos to the subject.

In another aspect, the disclosure features a probe for specifically detecting P4 in the manufacture of a kit for use in a method of determining whether a subject undergoing embryo transfer therapy is likely to benefit from oxytocin antagonist treatment, wherein the concentration of P4 in a sample isolated from the subject has been determined, the method including comparing the concentration of P4 to a P4 reference level, wherein a reduced concentration of P4 in the sample isolated from the subject relative to the P4 reference level identifies the subject as likely to benefit from oxytocin antagonist treatment prior to, concurrently with, and/or following transfer of one or more embryos to the subject.

In another aspect, the disclosure features a probe for specifically detecting P4 in the manufacture of a kit for use in a method of determining whether a subject undergoing embryo transfer therapy is likely to benefit from oxytocin antagonist treatment, the method including determining the concentration of P4 in a sample isolated from the subject and comparing the concentration of P4 to a P4 reference level, wherein a reduced concentration of P4 in the sample isolated from the subject relative to the P4 reference level identifies the subject as likely to benefit from oxytocin antagonist treatment prior to, concurrently with, and/or following transfer of one or more embryos to the subject.

In another aspect, the disclosure features a method of determining whether a subject undergoing embryo transfer therapy is likely to exhibit enhanced endometrial receptivity in response to oxytocin antagonist treatment, wherein the concentration of P4 in a sample isolated from the subject has been determined, the method including comparing the concentration of P4 to a P4 reference level, wherein a reduced concentration of P4 in the sample isolated from the subject relative to the P4 reference level identifies the subject as likely to exhibit enhanced endometrial receptivity in response to oxytocin antagonist treatment prior to, concurrently with, and/or following transfer of one or more embryos to the subject.

In another aspect, the disclosure features a method of determining whether a subject undergoing embryo transfer therapy is likely to exhibit enhanced endometrial receptivity in response to oxytocin antagonist treatment, the method including determining the concentration of P4 in a sample isolated from the subject and comparing the concentration of P4 to a P4 reference level, wherein a reduced concentration of P4 in the sample isolated from the subject relative to the P4 reference level identifies the subject as likely to exhibit enhanced endometrial receptivity in response to oxytocin antagonist treatment prior to, concurrently with, and/or following transfer of one or more embryos to the subject.

In another aspect, the disclosure features a method of collecting data for determining whether a subject undergoing embryo transfer therapy is likely to exhibit enhanced endometrial receptivity in response to oxytocin antagonist treatment, wherein the concentration of P4 in a sample isolated from the subject has been determined, the method including comparing the concentration of P4 to a P4 reference level, wherein a reduced concentration of P4 in the sample isolated from the subject relative to the P4 reference level identifies the subject as likely to exhibit enhanced endometrial receptivity in response to oxytocin antagonist treatment prior to, concurrently with, and/or following transfer of one or more embryos to the subject.

In another aspect, the disclosure features a method of collecting data for determining whether a subject undergoing embryo transfer therapy is likely to exhibit enhanced endometrial receptivity in response to oxytocin antagonist treatment, the method including determining the concentration of P4 in a sample isolated from the subject and comparing the concentration of P4 to a P4 reference level, wherein a reduced concentration of P4 in the sample isolated from the subject relative to the P4 reference level identifies the subject as likely to exhibit enhanced endometrial receptivity in response to oxytocin antagonist treatment prior to, concurrently with, and/or following transfer of one or more embryos to the subject.

In another aspect, the disclosure features a probe for specifically detecting progesterone in the manufacture of a kit for use in a method of determining whether a subject undergoing embryo transfer therapy is likely to exhibit enhanced endometrial receptivity in response to oxytocin antagonist treatment, wherein the concentration of P4 in a sample isolated from the subject has been determined, the method including comparing the concentration of P4 to a P4 reference level, wherein a reduced concentration of P4 in the sample isolated from the subject relative to the P4 reference level identifies the subject as likely to exhibit enhanced endometrial receptivity in response to oxytocin antagonist treatment prior to, concurrently with, and/or following transfer of one or more embryos to the subject.

In another aspect, the disclosure features a probe for specifically detecting progesterone in the manufacture of a kit for use in a method of determining whether a subject undergoing embryo transfer therapy is likely to exhibit enhanced endometrial receptivity in response to oxytocin antagonist treatment, the method including determining the concentration of P4 in a sample isolated from the subject and comparing the concentration of P4 to a P4 reference level, wherein a reduced concentration of P4 in the sample isolated from the subject relative to the P4 reference level identifies the subject as likely to exhibit enhanced endometrial receptivity in response to oxytocin antagonist treatment prior to, concurrently with, and/or following transfer of one or more embryos to the subject.

In some embodiments of the foregoing twelve aspects of the disclosure, the subject is identified as having a concentration of P4 in the sample isolated from the subject that is less than the P4 reference level.

In some embodiments, the method includes the step of informing the subject that the subject has been identified as having a concentration of P4 in the sample isolated from the subject that is less than the P4 reference level. Thus, in some embodiments, the method includes the step of informing the subject that the subject has been identified as likely to benefit from oxytocin antagonist treatment. In some embodiments, the method includes the step of informing the subject that the subject has been identified as likely to exhibit enhanced endometrial receptivity in response to oxytocin antagonist treatment.

In some embodiments, the method includes administering a therapeutically effective amount of an oxytocin antagonist to the subject if a reduced concentration of P4 in the sample isolated from the subject relative to the P4 reference level is detected. Thus, in some embodiments, the method includes comparing the concentration of P4 to a P4 reference level, determining that the concentration of P4 in the sample isolated from the subject is less than the P4 reference level, identifying the subject as likely to benefit from oxytocin antagonist treatment and/or identifying the subject as likely to exhibit enhanced endometrial receptivity in response to oxytocin antagonist treatment, and administering a therapeutically effective amount of an oxytocin antagonist to the subject.

In some embodiments of any of the above aspects of the disclosure, administering of the oxytocin antagonist reduces the likelihood of embryo implantation failure and/or miscarriage.

In some embodiments of any of the above aspects of the disclosure, the oxytocin antagonist is administered to the subject prior to the transfer of the one or more embryos to the uterus of the subject.

In some embodiments of any of the above aspects of the disclosure, the oxytocin antagonist is administered to the subject from about 1 hour to about 24 hours prior to the transfer of the one or more embryos to the subject. In some embodiments, the oxytocin antagonist is administered to the subject from about 1 hour to about 12 hours prior the transfer of the one or more embryos to the subject. In some embodiments, the oxytocin antagonist is administered to the subject from about 12 hours to about 24 hours prior the transfer of the one or more embryos to the subject.

In some embodiments of any of the above aspects of the disclosure, the oxytocin antagonist is administered to the subject from about 1 hour to about 10 hours prior the transfer of the one or more embryos to the subject. In some embodiments, the oxytocin antagonist is administered to the subject from about 1 hour to about 9 hours prior the transfer of the one or more embryos to the subject. In some embodiments, the oxytocin antagonist is administered to the subject from about 1 hour to about 8 hours prior the transfer of the one or more embryos to the subject. In some embodiments, the oxytocin antagonist is administered to the subject from about 1 hour to about 7 hours prior the transfer of the one or more embryos to the subject. In some embodiments, the oxytocin antagonist is administered to the subject from about 1 hour to about 6 hours prior the transfer of the one or more embryos to the subject. In some embodiments, the oxytocin antagonist is administered to the subject from about 1 hour to about 5 hours prior the transfer of the one or more embryos to the subject. In some embodiments, the oxytocin antagonist is administered to the subject from about 1 hour to about 4 hours prior the transfer of the one or more embryos to the subject.

In some embodiments of any of the above aspects of the disclosure, the oxytocin antagonist is administered to the subject from about 2 hours to about 6 hours prior the transfer of the one or more embryos to the subject. In some embodiments, the oxytocin antagonist is administered to the subject from about 3 hours to about 5 hours prior the transfer of the one or more embryos to the subject. In some embodiments, the oxytocin antagonist is administered to the subject about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours, or more prior to the transfer of the one or more embryos to the subject.

In some embodiments of any of the above aspects of the disclosure, the oxytocin antagonist is administered to the subject about 4 hours prior to the transfer of the one or more embryos to the subject.

In some embodiments of any of the above aspects of the disclosure, the oxytocin antagonist is administered to the subject prior to embryo transfer in a single dose.

In some embodiments of any of the above aspects of the disclosure, the oxytocin antagonist is administered to the subject prior to embryo transfer (i.e., prior to the transfer of the one or more embryos to the uterus of the subject) in multiple doses (for instance, in multiple periodic doses), such as from 1 to 20 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, prior to embryo transfer. In some embodiments, the oxytocin antagonist is administered to the subject prior to embryo transfer in from 1 to 20 doses per 24 hours, such as 1 dose per 24 hours, 2 doses per 24 hours, 3 doses per 24 hours, 4 doses per 24 hours, 5 doses per 24 hours, 6 doses per 24 hours, 7 doses per 24 hours, 8 doses per 24 hours, 9 doses per 24 hours, 10 doses per 24 hours, 11 doses per 24 hours, 12 doses per 24 hours, 13 doses per 24 hours, 14 doses per 24 hours, 15 doses per 24 hours, 16 doses per 24 hours, 17 doses per 24 hours, 18 doses per 24 hours, 19 doses per 24 hours, 20 doses per 24 hours. In some embodiments, the oxytocin antagonist is administered to the subject prior to embryo transfer in more than 20 doses per 24 hours.

For instance, in some embodiments, the oxytocin antagonist is administered to the subject in from 1 to 10 doses, for example, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, prior to embryo transfer. In some embodiments, the oxytocin antagonist is administered to the subject prior to embryo transfer in from 1 to 10 doses per 24 hours, such as 1 dose per 24 hours, 2 doses per 24 hours, 3 doses per 24 hours, 4 doses per 24 hours, 5 doses per 24 hours, 6 doses per 24 hours, 7 doses per 24 hours, 8 doses per 24 hours, 9 doses per 24 hours, 10 doses per 24 hours.

In some embodiments, the oxytocin antagonist is administered to the subject in from 1 to 5 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, prior to embryo transfer. In some embodiments, the oxytocin antagonist is administered to the subject in from 10 to 20 doses, for instance, per 12 hours, per 24 hours, per 36 hours, per 48 hours, per 60 hours, per 72 hours, per 84 hours, per 96 hours, per 108 hours, per 120 hours, per 132 hours, per 144 hours, per 156 hours, per 168 hours, or longer, prior to embryo transfer. In some embodiments, the oxytocin antagonist is administered to the subject in from 10 to 15 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, prior to embryo transfer.

In some embodiments, the oxytocin antagonist is administered to the subject in 1, 2, 3, 4, 5, 6, 7, 8, 9, 10, 11, 12, 13, 14, 15, 16, 17, 18, 19, 20, or more, doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, prior to embryo transfer.

In some embodiments, the oxytocin antagonist is administered to the subject prior to embryo transfer in up to 7 doses (e.g., 1, 2, 3, 4, 5, 6, or 7 doses) per 24 hours. In some embodiments, the oxytocin antagonist is administered to the subject prior to embryo transfer in 1 dose per 24 hours, such as 1 dose per 24 hours of compound (II). In some embodiments, the oxytocin antagonist is administered to the subject prior to embryo transfer in 2 doses per 24 hours, such as 2 doses per 24 hours of compound (II). In some embodiments, the oxytocin antagonist is administered to the subject prior to embryo transfer in 3 doses per 24 hours, such as 3 doses per 24 hours of compound (II). In some embodiments, the oxytocin antagonist is administered to the subject prior to embryo transfer in 4 doses per 24 hours, such as 4 doses per 24 hours of compound (II). In some embodiments, the oxytocin antagonist is administered to the subject prior to embryo transfer in 5 doses per 24 hours, such as 5 doses per 24 hours of compound (II). In some embodiments, the oxytocin antagonist is administered to the subject prior to embryo transfer in 6 doses per 24 hours, such as 6 doses per 24 hours of compound (II). In some embodiments, the oxytocin antagonist is administered to the subject prior to embryo transfer in 7 doses per 24 hours, such as 7 doses per 24 hours of compound (II).

The multiple doses may be administered, for example, starting at from about 1 hour to about 14 days, or more, prior to embryo transfer. In some embodiments, the multiple doses are administered starting at from about 1 hour to about 7 days, or more, prior to embryo transfer. In some embodiments, the multiple doses may be administered starting at from about 1 day to about 14 days prior to embryo transfer. In some embodiments, the multiple doses may be administered starting at from about 3 days to about 11 days prior to embryo transfer. In some embodiments, the multiple doses may be administered starting at from about 1 day to about 7 days prior to embryo transfer. In some embodiments, the multiple doses may be administered starting at from about 2 days to about 5 days prior to embryo transfer. In some embodiments, the multiple doses may be administered starting at from about 3 days to about 4 days prior to embryo transfer. For instance, the multiple doses may be administered starting at 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, 96 hours, 108 hours, 120 hours, 132 hours, 144 hours, 156 hours, 168 hours, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more, prior to embryo transfer to the subject.

In some embodiments, the multiple doses are administered starting at about 2 days prior to embryo transfer.

In some embodiments, the multiple doses are administered starting at about 3 days prior to embryo transfer.

In some embodiments, the multiple doses are administered starting at about 4 days prior to embryo transfer.

In some embodiments, the multiple doses are administered starting at about 5 days prior to embryo transfer.

In some embodiments, the multiple doses are administered starting at about 6 days prior to embryo transfer.

In some embodiments, the multiple doses are administered starting at about 7 days prior to embryo transfer.

In some embodiments, the multiple doses terminate on the day of embryo transfer to the subject.

In some embodiments, the multiple doses terminate with a final dose of the oxytocin antagonist that is administered concurrently with (e.g., within 60 minutes of) transfer of the one or more embryos to the subject.

In some embodiments of any of the above aspects of the disclosure, the multiple doses continue following embryo transfer. For instance, the oxytocin antagonist may be administered to the subject in one or more additional doses concurrently with embryo transfer. In some embodiments, the oxytocin antagonist is administered to the subject in one or more additional doses following embryo transfer (for instance, in multiple periodic doses), such as in one or more additional doses administered within about 1 hour to about 1 week, or longer (e.g., within about 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, 96 hours, 108 hours, 120 hours, 132 hours, 144 hours, 156 hours, 168 hours, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more) following the transfer of the one or more embryos to the subject.

For example, in some embodiments, the oxytocin antagonist is administered to the subject in one or more additional doses within about 1 hour to about 24 hours following the transfer of the one or more embryos to the subject. In some embodiments, the oxytocin antagonist is administered to the subject in one or more additional doses within about 1 hour to about 12 hours following the transfer of the one or more embryos to the subject. In some embodiments, the oxytocin antagonist is administered to the subject in one or more additional doses within from about 12 hours to about 24 hours following the transfer of the one or more embryos to the subject. In some embodiments, the oxytocin antagonist is administered to the subject in one or more additional doses within from about 1 hour to about 10 hours following the transfer of the one or more embryos to the subject. In some embodiments, the oxytocin antagonist is administered to the subject in one or more additional doses within from about 1 hour to about 9 hours following the transfer of the one or more embryos to the subject. In some embodiments, the oxytocin antagonist is administered to the subject in one or more additional doses within from about 1 hour to about 8 hours following the transfer of the one or more embryos to the subject. In some embodiments, the oxytocin antagonist is administered to the subject in one or more additional doses within from about 1 hour to about 7 hours following the transfer of the one or more embryos to the subject. In some embodiments, the oxytocin antagonist is administered to the subject in one or more additional doses within from about 1 hour to about 6 hours following the transfer of the one or more embryos to the subject. In some embodiments, the oxytocin antagonist is administered to the subject in one or more additional doses within from about 1 hour to about 5 hours following the transfer of the one or more embryos to the subject. In some embodiments, the oxytocin antagonist is administered to the subject in one or more additional doses within from about 1 hour to about 4 hours following the transfer of the one or more embryos to the subject. In some embodiments, the oxytocin antagonist is administered to the subject in one or more additional doses within from about 2 hours to about 6 hours following the transfer of the one or more embryos to the subject. In some embodiments, the oxytocin antagonist is administered to the subject in one or more additional doses within from about 3 hours to about 5 hours following the transfer of the one or more embryos to the subject.

In some embodiments, the oxytocin antagonist is administered to the subject in one or more additional doses starting at about 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, 96 hours, 108 hours, 120 hours, 132 hours, 144 hours, 156 hours, 168 hours, or more, following the transfer of the one or more embryos to the subject.

In some embodiments, the oxytocin antagonist is administered to the subject in multiple additional doses following embryo transfer, such as in from 1 to 20 additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is additionally administered to the subject following embryo transfer in from 1 to 20 doses per 24 hours, such as 1 dose per 24 hours, 2 doses per 24 hours, 3 doses per 24 hours, 4 doses per 24 hours, 5 doses per 24 hours, 6 doses per 24 hours, 7 doses per 24 hours, 8 doses per 24 hours, 9 doses per 24 hours, 10 doses per 24 hours, 11 doses per 24 hours, 12 doses per 24 hours, 13 doses per 24 hours, 14 doses per 24 hours, 15 doses per 24 hours, 16 doses per 24 hours, 17 doses per 24 hours, 18 doses per 24 hours, 19 doses per 24 hours, 20 doses per 24 hours. In some embodiments, the oxytocin antagonist is additionally administered to the subject following embryo transfer in more than 20 doses per 24 hours.

For instance, in some embodiments, the oxytocin antagonist is administered to the subject in from 1 to 10 additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is additionally administered to the subject following embryo transfer in from 1 to 10 doses per 24 hours, such as 1 dose per 24 hours, 2 doses per 24 hours, 3 doses per 24 hours, 4 doses per 24 hours, 5 doses per 24 hours, 6 doses per 24 hours, 7 doses per 24 hours, 8 doses per 24 hours, 9 doses per 24 hours, 10 doses per 24 hours.

In some embodiments, the oxytocin antagonist is administered to the subject in from 1 to 5 additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is administered to the subject in from 10 to 20 additional doses, for instance, per 12 hours, per 24 hours, per 36 hours, per 48 hours, per 60 hours, per 72 hours, per 84 hours, per 96 hours, per 108 hours, per 120 hours, per 132 hours, per 144 hours, per 156 hours, per 168 hours, or longer, following embryo transfer. In some embodiments, the oxytocin antagonist is administered to the subject in from 10 to 15 additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer.

In some embodiments, the oxytocin antagonist is administered to the subject in 1, 2, 3, 4, 5, 6, 7, 8, 9, 10, 11, 12, 13, 14, 15, 16, 17, 18, 19, 20, or more, additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer.

In some embodiments, the oxytocin antagonist is administered to the subject following embryo transfer in up to 7 additional doses (e.g., 1, 2, 3, 4, 5, 6, or 7 doses) per 24 hours. In some embodiments, the oxytocin antagonist is additionally administered to the subject following embryo transfer in 1 dose per 24 hours, such as 1 additional dose per 24 hours of compound (II). In some embodiments, the oxytocin antagonist is additionally administered to the subject following embryo transfer in 2 doses per 24 hours, such as 2 additional doses per 24 hours of compound (II). In some embodiments, the oxytocin antagonist is additionally administered to the subject following embryo transfer in 3 doses per 24 hours, such as 3 additional doses per 24 hours of compound (II). In some embodiments, the oxytocin antagonist is additionally administered to the subject following embryo transfer in 4 doses per 24 hours, such as 4 additional doses per 24 hours of compound (II). In some embodiments, the oxytocin antagonist is additionally administered to the subject following embryo transfer in 5 doses per 24 hours, such as 5 additional doses per 24 hours of compound (II). In some embodiments, the oxytocin antagonist is additionally administered to the subject following embryo transfer in 6 doses per 24 hours, such as 6 additional doses per 24 hours of compound (II). In some embodiments, the oxytocin antagonist is additionally administered to the subject following embryo transfer in 7 doses per 24 hours, such as 7 additional doses per 24 hours of compound (II).

When one or more additional doses of the oxytocin antagonist are administered to the subject following embryo transfer, administration of the oxytocin antagonist may terminate, for instance, within from about 1 hour to about 14 days, or more, following embryo transfer. For instance, administration of the oxytocin antagonist may terminate within about 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, 96 hours, 108 hours, 120 hours, 132 hours, 144 hours, 156 hours, 168 hours, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more, following embryo transfer.

Thus, in some embodiments, the oxytocin antagonist is administered to the subject in additional daily doses following embryo transfer for about 1 day to about 14 days following embryo transfer. In some embodiments, the additional daily doses are administered to the subject for about 3 days to about 11 days following embryo transfer. In some embodiments, the additional daily doses are administered to the subject for 7 days following embryo transfer.

In some embodiments of any of the above aspects of the disclosure, the oxytocin antagonist is administered to the subject concurrently with the transfer of the one or more embryos to the uterus of the subject

In some embodiments, the oxytocin antagonist is administered to the subject concurrently with embryo transfer in a single dose.

In some embodiments, the oxytocin antagonist is administered to the subject in multiple doses beginning during embryo transfer (for instance, in multiple periodic doses) and continuing after embryo transfer, such as from 1 to 20 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, beginning during embryo transfer and continuing following embryo transfer. For instance, in some embodiments, the oxytocin antagonist is administered to the subject in from 1 to 10 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, beginning during embryo transfer and continuing following embryo transfer. In some embodiments, the oxytocin antagonist is administered to the subject in from 1 to 5 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, beginning during embryo transfer and continuing following embryo transfer. In some embodiments, the oxytocin antagonist is administered to the subject in from 10 to 20 doses, for instance, per 12 hours, per 24 hours, per 36 hours, per 48 hours, per 60 hours, per 72 hours, per 84 hours, per 96 hours, per 108 hours, per 120 hours, per 132 hours, per 144 hours, per 156 hours, per 168 hours, or longer, beginning during embryo transfer and continuing following embryo transfer. In some embodiments, the oxytocin antagonist is administered to the subject in from 10 to 15 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, beginning during embryo transfer and continuing following embryo transfer. In some embodiments, the oxytocin antagonist is administered to the subject in 1, 2, 3, 4, 5, 6, 7, 8, 9, 10, 11, 12, 13, 14, 15, 16, 17, 18, 19, 20, or more, doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is administered to the subject beginning during embryo transfer and continuing following embryo transfer in up to 7 doses (e.g., 1, 2, 3, 4, 5, 6, or 7 doses) per 24 hours.

For example, in some embodiments, the oxytocin antagonist is first administered to the subject concurrently with the transfer of the one or more embryos to the uterus of the subject, and the oxytocin antagonist is subsequently administered to the subject in one or more additional doses within about 1 hour to about 24 hours following the transfer of the one or more embryos to the subject. For instance, in some embodiments, the oxytocin antagonist is administered to the subject in one or more additional doses within about 1 hour to about 12 hours following the transfer of the one or more embryos to the subject. In some embodiments, the oxytocin antagonist is administered to the subject in one or more additional doses within from about 12 hours to about 24 hours following the transfer of the one or more embryos to the subject. In some embodiments, the oxytocin antagonist is administered to the subject in one or more additional doses within from about 1 hour to about 10 hours following the transfer of the one or more embryos to the subject. In some embodiments, the oxytocin antagonist is administered to the subject in one or more additional doses within from about 1 hour to about 9 hours following the transfer of the one or more embryos to the subject. In some embodiments, the oxytocin antagonist is administered to the subject in one or more additional doses within from about 1 hour to about 8 hours following the transfer of the one or more embryos to the subject. In some embodiments, the oxytocin antagonist is administered to the subject in one or more additional doses within from about 1 hour to about 7 hours following the transfer of the one or more embryos to the subject. In some embodiments, the oxytocin antagonist is administered to the subject in one or more additional doses within from about 1 hour to about 6 hours following the transfer of the one or more embryos to the subject. In some embodiments, the oxytocin antagonist is administered to the subject in one or more additional doses within from about 1 hour to about 5 hours following the transfer of the one or more embryos to the subject. In some embodiments, the oxytocin antagonist is administered to the subject in one or more additional doses within from about 1 hour to about 4 hours following the transfer of the one or more embryos to the subject. In some embodiments, the oxytocin antagonist is administered to the subject in one or more additional doses within from about 2 hours to about 6 hours following the transfer of the one or more embryos to the subject. In some embodiments, the oxytocin antagonist is administered to the subject in one or more additional doses within from about 3 hours to about 5 hours following the transfer of the one or more embryos to the subject.

In some embodiments, the oxytocin antagonist is first administered to the subject concurrently with the transfer of the one or more embryos to the uterus of the subject, and the oxytocin antagonist is subsequently administered to the subject in one or more additional doses starting at about 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, 96 hours, 108 hours, 120 hours, 132 hours, 144 hours, 156 hours, 168 hours, or more, following the transfer of the one or more embryos to the subject.

In some embodiments, the oxytocin antagonist is first administered to the subject concurrently with the transfer of the one or more embryos to the uterus of the subject, and the oxytocin antagonist is subsequently administered to the subject in multiple additional doses following embryo transfer, such as in from 1 to 20 additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is additionally administered to the subject following embryo transfer in from 1 to 20 doses per 24 hours, such as 1 dose per 24 hours, 2 doses per 24 hours, 3 doses per 24 hours, 4 doses per 24 hours, 5 doses per 24 hours, 6 doses per 24 hours, 7 doses per 24 hours, 8 doses per 24 hours, 9 doses per 24 hours, 10 doses per 24 hours, 11 doses per 24 hours, 12 doses per 24 hours, 13 doses per 24 hours, 14 doses per 24 hours, 15 doses per 24 hours, 16 doses per 24 hours, 17 doses per 24 hours, 18 doses per 24 hours, 19 doses per 24 hours, 20 doses per 24 hours. In some embodiments, the oxytocin antagonist is additionally administered to the subject following embryo transfer in more than 20 doses per 24 hours.

For instance, in some embodiments, the oxytocin antagonist is first administered to the subject concurrently with the transfer of the one or more embryos to the uterus of the subject, and the oxytocin antagonist is subsequently administered to the subject in from 1 to 10 additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is additionally administered to the subject following embryo transfer in from 1 to 10 doses per 24 hours, such as 1 dose per 24 hours, 2 doses per 24 hours, 3 doses per 24 hours, 4 doses per 24 hours, 5 doses per 24 hours, 6 doses per 24 hours, 7 doses per 24 hours, 8 doses per 24 hours, 9 doses per 24 hours, 10 doses per 24 hours.

In some embodiments, the oxytocin antagonist is first administered to the subject concurrently with the transfer of the one or more embryos to the uterus of the subject, and the oxytocin antagonist is subsequently administered to the subject in from 1 to 5 additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is administered to the subject in from 10 to 20 additional doses, for instance, per 12 hours, per 24 hours, per 36 hours, per 48 hours, per 60 hours, per 72 hours, per 84 hours, per 96 hours, per 108 hours, per 120 hours, per 132 hours, per 144 hours, per 156 hours, per 168 hours, or longer, following embryo transfer. In some embodiments, the oxytocin antagonist is administered to the subject in from 10 to 15 additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer.

In some embodiments, the oxytocin antagonist is first administered to the subject concurrently with the transfer of the one or more embryos to the uterus of the subject, and the oxytocin antagonist is subsequently administered to the subject in 1, 2, 3, 4, 5, 6, 7, 8, 9, 10, 11, 12, 13, 14, 15, 16, 17, 18, 19, 20, or more, additional doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer.

In some embodiments, the oxytocin antagonist is first administered to the subject concurrently with the transfer of the one or more embryos to the uterus of the subject, and the oxytocin antagonist is subsequently administered to the subject following embryo transfer in up to 7 additional doses (e.g., 1, 2, 3, 4, 5, 6, or 7 doses) per 24 hours. In some embodiments, the oxytocin antagonist is additionally administered to the subject following embryo transfer in 1 dose per 24 hours, such as 1 additional dose per 24 hours of compound (II). In some embodiments, the oxytocin antagonist is additionally administered to the subject following embryo transfer in 2 doses per 24 hours, such as 2 additional doses per 24 hours of compound (II). In some embodiments, the oxytocin antagonist is additionally administered to the subject following embryo transfer in 3 doses per 24 hours, such as 3 additional doses per 24 hours of compound (II). In some embodiments, the oxytocin antagonist is additionally administered to the subject following embryo transfer in 4 doses per 24 hours, such as 4 additional doses per 24 hours of compound (II). In some embodiments, the oxytocin antagonist is additionally administered to the subject following embryo transfer in 5 doses per 24 hours, such as 5 additional doses per 24 hours of compound (II). In some embodiments, the oxytocin antagonist is additionally administered to the subject following embryo transfer in 6 doses per 24 hours, such as 6 additional doses per 24 hours of compound (II). In some embodiments, the oxytocin antagonist is additionally administered to the subject following embryo transfer in 7 doses per 24 hours, such as 7 additional doses per 24 hours of compound (II).

When one or more additional doses of the oxytocin antagonist are administered to the subject following embryo transfer, administration of the oxytocin antagonist may terminate, for instance, within from about 1 hour to about 14 days, or more, following embryo transfer. For instance, administration of the oxytocin antagonist may terminate within about 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, 96 hours, 108 hours, 120 hours, 132 hours, 144 hours, 156 hours, 168 hours, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more, following embryo transfer.

Thus, in some embodiments, the oxytocin antagonist is first administered to the subject concurrently with the transfer of the one or more embryos to the uterus of the subject, and the oxytocin antagonist is subsequently administered to the subject in additional daily doses following embryo transfer for about 1 day to about 14 days following embryo transfer. In some embodiments, the additional daily doses are administered to the subject for about 3 days to about 11 days following embryo transfer. In some embodiments, the additional daily doses are administered to the subject for 7 days following embryo transfer.

In some embodiments of any of the above aspects of the disclosure, the oxytocin antagonist is administered to the subject following the transfer of the one or more embryos to the uterus of the subject

In some embodiments, the oxytocin antagonist is administered to the subject within about 1 hour to about 24 hours following the transfer of the one or more embryos to the subject. For instance, in some embodiments, the oxytocin antagonist is administered to the subject within about 1 hour to about 12 hours following the transfer of the one or more embryos to the subject. In some embodiments, the oxytocin antagonist is administered to the subject within from about 12 hours to about 24 hours following the transfer of the one or more embryos to the subject. In some embodiments, the oxytocin antagonist is administered to the subject within from about 1 hour to about 10 hours following the transfer of the one or more embryos to the subject. In some embodiments, the oxytocin antagonist is administered to the subject within from about 1 hour to about 9 hours following the transfer of the one or more embryos to the subject. In some embodiments, the oxytocin antagonist is administered to the subject within from about 1 hour to about 8 hours following the transfer of the one or more embryos to the subject. In some embodiments, the oxytocin antagonist is administered to the subject within from about 1 hour to about 7 hours following the transfer of the one or more embryos to the subject. In some embodiments, the oxytocin antagonist is administered to the subject within from about 1 hour to about 6 hours following the transfer of the one or more embryos to the subject. In some embodiments, the oxytocin antagonist is administered to the subject within from about 1 hour to about 5 hours following the transfer of the one or more embryos to the subject. In some embodiments, the oxytocin antagonist is administered to the subject within from about 1 hour to about 4 hours following the transfer of the one or more embryos to the subject. In some embodiments, the oxytocin antagonist is administered to the subject within from about 2 hours to about 6 hours following the transfer of the one or more embryos to the subject. In some embodiments, the oxytocin antagonist is administered to the subject within from about 3 hours to about 5 hours following the transfer of the one or more embryos to the subject.

For instance, in some embodiments, the oxytocin antagonist is administered to the subject about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, 84 hours, 96 hours, 108 hours, 120 hours, 132 hours, 144 hours, 156 hours, 168 hours, or more, following the transfer of the one or more embryos to the subject.

In some embodiments, the oxytocin antagonist is administered to the subject after embryo transfer in a single dose.

In some embodiments, the oxytocin antagonist is administered to the subject in multiple doses following embryo transfer, such as in multiple periodic doses. In some embodiments, the oxytocin antagonist is administered to the subject in from 1 to 20 doses following embryo transfer, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is administered to the subject following embryo transfer in from 1 to 20 doses per 24 hours, such as 1 dose per 24 hours, 2 doses per 24 hours, 3 doses per 24 hours, 4 doses per 24 hours, 5 doses per 24 hours, 6 doses per 24 hours, 7 doses per 24 hours, 8 doses per 24 hours, 9 doses per 24 hours, 10 doses per 24 hours, 11 doses per 24 hours, 12 doses per 24 hours, 13 doses per 24 hours, 14 doses per 24 hours, 15 doses per 24 hours, 16 doses per 24 hours, 17 doses per 24 hours, 18 doses per 24 hours, 19 doses per 24 hours, 20 doses per 24 hours. In some embodiments, the oxytocin antagonist is administered to the subject following embryo transfer in more than 20 doses per 24 hours.

For instance, in some embodiments, the oxytocin antagonist is administered to the subject in from 1 to 10 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is administered to the subject following embryo transfer in from 1 to 10 doses per 24 hours, such as 1 dose per 24 hours, 2 doses per 24 hours, 3 doses per 24 hours, 4 doses per 24 hours, 5 doses per 24 hours, 6 doses per 24 hours, 7 doses per 24 hours, 8 doses per 24 hours, 9 doses per 24 hours, 10 doses per 24 hours.

In some embodiments, the oxytocin antagonist is administered to the subject in from 1 to 5 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer. In some embodiments, the oxytocin antagonist is administered to the subject in from 10 to 20 doses, for instance, per 12 hours, per 24 hours, per 36 hours, per 48 hours, per 60 hours, per 72 hours, per 84 hours, per 96 hours, per 108 hours, per 120 hours, per 132 hours, per 144 hours, per 156 hours, per 168 hours, or longer, following embryo transfer. In some embodiments, the oxytocin antagonist is administered to the subject in from 10 to 15 doses, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer.

In some embodiments, the oxytocin antagonist is administered to the subject in 1, 2, 3, 4, 5, 6, 7, 8, 9, 10, 11, 12, 13, 14, 15, 16, 17, 18, 19, 20 doses, or more, for instance, per 12 hours, per 24 hours, per 36 hours, per 48 hours, per 60 hours, per 72 hours, per 84 hours, per 96 hours, per 108 hours, 120 hours, per 132 hours, per 144 hours, per 156 hours, per 168 hours, or longer, following embryo transfer.

In some embodiments, the oxytocin antagonist is administered to the subject following embryo transfer in up to 7 doses (e.g., 1, 2, 3, 4, 5, 6, or 7 doses) per 24 hours. In some embodiments, the oxytocin antagonist is administered to the subject following embryo transfer in 1 dose per 24 hours, such as 1 dose per 24 hours of compound (II). In some embodiments, the oxytocin antagonist is administered to the subject following embryo transfer in 2 doses per 24 hours, such as 2 doses per 24 hours of compound (II). In some embodiments, the oxytocin antagonist is administered to the subject following embryo transfer in 3 doses per 24 hours, such as 3 doses per 24 hours of compound (II). In some embodiments, the oxytocin antagonist is administered to the subject following embryo transfer in 4 doses per 24 hours, such as 4 doses per 24 hours of compound (II). In some embodiments, the oxytocin antagonist is administered to the subject following embryo transfer in 5 doses per 24 hours, such as 5 doses per 24 hours of compound (II). In some embodiments, the oxytocin antagonist is administered to the subject following embryo transfer in 6 doses per 24 hours, such as 6 doses per 24 hours of compound (II). In some embodiments, the oxytocin antagonist is administered to the subject following embryo transfer in 7 doses per 24 hours, such as 7 doses per 24 hours of compound (II).

When the oxytocin antagonist is administered in multiple doses following embryo transfer, administration of the oxytocin antagonist may terminate, for instance, within from about 1 hour to about 14 days, or more, following embryo transfer. For instance, administration of the oxytocin antagonist may terminate within about 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, 96 hours, 108 hours, 120 hours, 132 hours, 144 hours, 156 hours, 168 hours, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more, following embryo transfer.

Thus, in some embodiments, the oxytocin antagonist is administered to the subject in daily doses following embryo transfer for about 1 day to about 14 days following embryo transfer. In some embodiments, the daily doses are administered to the subject for about 3 days to about 11 days following embryo transfer. In some embodiments, the daily doses are administered to the subject for 7 days following embryo transfer.

In some embodiments of any of the above aspects of the disclosure, administration of the oxytocin antagonist to the subject reduces the likelihood of the subject having a miscarriage following the transfer of the one or more embryos to the subject.

In some embodiments, the sample is a blood sample.

In some embodiments, the embryo transfer therapy includes the transfer of from 1 to 2 embryos to the subject. In some embodiments, the embryo transfer therapy includes the transfer of 1 embryo to the subject. In some embodiments, the embryo transfer therapy includes the transfer of 2 embryos to the subject.

In some embodiments, the subject is a mammal and the one or more embryos are mammalian embryos. In some embodiments, the mammal is a human and the one or more embryos are human embryos.

In some embodiments, the one or more embryos are produced ex vivo by IVF, such as by IVF of one or more ova derived from the subject.

In some embodiments, the one or more embryos are produced ex vivo by ICSI, such as by ICSI into one or more ova derived from the subject.

In some embodiments, the one or more ova are derived from one or more oocytes isolated from the subject. In some embodiments, the one or more oocytes are isolated from the subject from about 1 day to about 7 days prior to the transfer of the one or more embryos to the subject. In some embodiments, the one or more oocytes are isolated from the subject about 2 days prior to the transfer of the one or more embryos to the subject. In some embodiments, the one or more oocytes are isolated from the subject about 3 days prior to the transfer of the one or more embryos to the subject. In some embodiments, the one or more oocytes are isolated from the subject about 4 days prior to the transfer of the one or more embryos to the subject. In some embodiments, the one or more oocytes are isolated from the subject about 5 days prior to the transfer of the one or more embryos to the subject.

In some embodiments, the one or more oocytes include from 1 to 4 mature oocytes (i.e., 1 to 4 ova).

In some embodiments, a GnRH antagonist is administered to the subject prior to isolation of the one or more oocytes (e.g., containing one or more mature oocytes) from the subject.

In some embodiments, hCG is administered to the subject prior to isolation of the one or more oocytes (e.g., containing one or more mature oocytes) from the subject, such as by a single intravenous injection, for instance, to induce final follicular maturation.

In some embodiments, progesterone is administered to the subject following isolation of the one or more oocytes from the subject. The progesterone may be administered intravaginally. In some embodiments, about 300 mg to about 600 mg of progesterone per dose is administered to the subject. In some embodiments, the progesterone is administered to the subject daily, such as beginning within about 24 hours of isolation of the one or more oocytes from the subject and continuing for about 6 or more weeks following the transfer of the one or more embryos to the subject.

In some embodiments, the one or more ova are isolated directly from the subject. In some embodiments, the one or more ova are isolated from the subject from about 1 day to about 7 days prior to the transfer of the one or more embryos to the subject. In some embodiments, the one or more ova are isolated from the subject about 2 days prior to the transfer of the one or more embryos to the subject. In some embodiments, the one or more ova are isolated from the subject about 3 days prior to the transfer of the one or more embryos to the subject. In some embodiments, the one or more ova are isolated from the subject about 4 days prior to the transfer of the one or more embryos to the subject. In some embodiments, the one or more ova are isolated from the subject about 5 days prior to the transfer of the one or more embryos to the subject.

In some embodiments, a GnRH antagonist is administered to the subject prior to isolation of the one or more ova from the subject, such as in a single intravenous injection.

In some embodiments, hCG is administered to the subject prior to isolation of the one or more ova from the subject, such as by a single intravenous injection, for instance, to induce final follicular maturation.

In some embodiments, progesterone is administered to the subject following isolation of the one or more ova from the subject. The progesterone may be administered intravaginally. In some embodiments, about 300 mg to about 600 mg of progesterone per dose is administered to the subject. In some embodiments, the progesterone is administered to the subject daily, such as beginning within about 24 hours of isolation of the one or more ova from the subject and continuing for about 6 or more weeks following the transfer of the one or more embryos to the subject.

In some embodiments, the one or more embryos are transferred to the subject during the same menstrual cycle as isolation of the one or more oocytes from the subject.

In some embodiments, the one or more embryos are transferred to the subject during the same menstrual cycle as isolation of the one or more ova from the subject.

In some embodiments, the one or more embryos are frozen and thawed prior to the transfer of the one or more embryos to the subject.

In some embodiments, the one or more embryos each contain from 6 to 8 blastomeres immediately prior to the transfer of the one or more embryos to the subject. The blastomeres may be of approximately equal sizes as assessed by visual microscopy prior to the transfer of the one or more embryos to the subject. In some embodiments, the one or more embryos comprise an embryo having the form of a morula. In some embodiments, the one or more embryos comprise an embryo having the form of a blastula (e.g., a mammalian blastocyst).

In some embodiments, the oxytocin antagonist is a compound represented by formula (I)

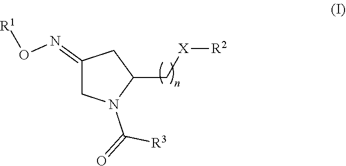

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring.

In some embodiments, the compound is (3Z,5S)-5-(hydroxymethyl)-1-[(2′-methyl-1,1′-biphenyl-4 yl)carbonyl]pyrrolidin-3-one O-methyloxime, represented by formula (I)

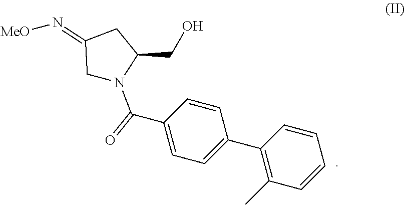 [SMILES: CO/N=C1/C[C@@H](CO)N(C(=O)c2ccc(-c3ccccc3C)cc2)C1]

In some embodiments, the compound represented by formula (II) (i.e., (3Z,5S)-5-(hydroxymethyl)-1-[(2′-methyl-1,1′-biphenyl-4-yl)carbonyl]pyrrolidin-3-one O-methyloxime) is substantially pure. For instance, in some embodiments, the compound represented by formula (II) has a purity of at least 85%, such as a purity of from 85% to 99.9% or more (e.g., a purity of 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more). The purity of the compound represented by formula (II) may be assessed, for instance, using NMR techniques and/or chromatographic methods, such as HPLC procedures, that are known in the art and described herein, such as those techniques that are described in U.S. Pat. No. 9,670,155, the disclosure of which is incorporated herein by reference in its entirety.

In some embodiments, the compound represented by formula (II) is substantially pure with respect to diastereomers of this compound and other by-products that may be formed during the synthesis of this compound. For instance, in some embodiments, the compound represented by formula (II) has a purity of at least 85%, such as a purity of from 85% to 99.9% or more (e.g., a purity of 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more) with respect to diastereomers of this compound and other by-products that may be formed during the synthesis of this compound, such as a by-product that is formed during the synthesis of this compound as described in U.S. Pat. No. 9,670,155. The purity of the compound represented by formula (II) may be assessed, for instance, using NMR techniques and/or chromatographic methods, such as HPLC procedures, that are known in the art and described herein, such as those techniques that are described in U.S. Pat. No. 9,670,155.

In some embodiments, the compound represented by formula (II) is substantially pure with respect to its (3E) diastereomer, (3E,5S)-5-(hydroxymethyl)-1-[(2′-methyl-1,1′-biphenyl-4-yl)carbonyl]pyrrolidin-3-one O-methyloxime. For instance, in some embodiments, the compound represented by formula (II) has a purity of at least 85%, such as a purity of from 85% to 99.9% or more (e.g., a purity of 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more) with respect to (3E,5S)-5-(hydroxymethyl)-1-[(2′-methyl-1,1′-biphenyl-4-yl)carbonyl]pyrrolidin-3-one O-methyloxime. For instance, compound (II) may be administered in the form of a composition (e.g., a tablet, such as a dispersible tablet, capsule, gel cap, powder, liquid solution, or liquid suspension) that contains less than 15% of the (3E) diastereomer. For example, compound (II) may be administered in the form of a composition (e.g., a tablet, such as a dispersible tablet, capsule, gel cap, powder, liquid solution, or liquid suspension) that contains less than 14%, less than 13%, less than 12%, less than 11%, less than 10%, less than 9%, less than 8%, less than 7%, less than 6%, less than 5%, less than 4%, less than 3%, less than 2%, less than 1%, less than 0.1%, less than 0.01%, less than 0.001%, or less of the (3E) diastereomer. The purity of the compound represented by formula (II) may be assessed, for instance, using NMR techniques and/or chromatographic methods, such as HPLC procedures, that are known in the art and described herein, such as those techniques that are described in U.S. Pat. No. 9,670,155.

In some embodiments, the compound is in a crystalline state. In some embodiments, the compound exhibits characteristic X-ray powder diffraction peaks at about 7.05° 2θ, about 13.13° 2θ, and about 23.34° 2θ. For instance, the compound may exhibit characteristic X-ray powder diffraction peaks at about 7.05° 2θ, about 12.25° 2θ, about 13.13° 2θ, about 16.54° 2θ, about 18.00° 2θ, about 21.84° 2θ, and about 23.34° 2θ. In some embodiments, the compound exhibits characteristic X-ray powder diffraction peaks as set forth in Table 1, above.

In some embodiments, the compound is administered orally to the subject. In some embodiments, the compound is administered intravenously to the subject. For instance, the compound may be administered to the subject in the form of a tablet, capsule, gel cap, powder, liquid solution, or liquid suspension. In some embodiments, the compound is administered to the subject in the form of a tablet, such as a dispersible tablet. The dispersible tablet may have, for example, one or more, or all, of the following components:

a. about 1-20% by weight of calcium silicate;

b. about 0.1-20% by weight of PVP30K;

c. about 0.01-5% by weight of poloxamer 188;

d. about 0.5-20% by weight of sodium croscarmellose;

e. about 1-90% by weight of microcrystalline cellulose 112;

f. about 1-90% by weight of lactose monohydrate;

g. about 0.01-0.5% by weight of sodium saccharine; and

h. about 0.1-10% by weight of glycerol dibehenate.

For instance, the dispersible tablet may have the following composition:

a. about 5% by weight of calcium silicate;

b. about 1% by weight of PVP30K;

c. about 2% by weight of poloxamer 188;

d. about 5% by weight of sodium croscarmellose;

e. about 1.5% by weight of microcrystalline cellulose 112;

f. about 47.8% by weight of lactose monohydrate;

g. about 0.2% by weight of sodium saccharine; and

h. about 4% by weight of glycerol dibehenate.

In some embodiments, the compound is administered to the subject in a unit dosage form containing from about 25 mg to about 250 mg of the compound, such as a unit dosage form containing about 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, or more, of the compound.

In some embodiments, the compound is administered to the subject in a unit dosage form containing from about 25 mg to about 75 mg of the compound, such as a unit dosage form containing about 50 mg of the compound. In some embodiments, the compound is administered to the subject in a unit dosage form containing from about 175 mg to about 225 mg of the compound, such as a unit dosage form containing about 200 mg of the compound.

In some embodiments, the oxytocin antagonist is administered to the subject in an amount of from 1,500 mg to 2,100 mg per dose, such as an amount of from 1,510 mg to 2,090 mg per dose, from 1,520 mg to 2,080 mg per dose, from 1,530 mg to 2,070 mg per dose, from 1,540 mg to 2,060 mg per dose, from 1,550 mg to 2,050 mg per dose, from 1,560 mg to 2,040 mg per dose, from 1,570 mg to 2,030 mg per dose, from 1,580 mg to 2,020 mg per dose, from 1,590 mg to 2,010 mg per dose, from 1,600 mg to 2,000 mg per dose, from 1,610 mg to 1,990 mg per dose, from 1,620 mg to 1,980 mg per dose, from 1,630 mg to 1,970 mg per dose, from 1,640 mg to 1,960 mg per dose, from 1,650 mg to 1,950 mg per dose, from 1,660 mg to 1,940 mg per dose, from 1,670 mg to 1,930 mg per dose, from 1,680 mg to 1,920 mg per dose, from 1,690 mg to 1,910 mg per dose, from 1,700 mg to 1,900 mg per dose, from 1,710 mg to 1,890 mg per dose, from 1,720 mg to 1,880 mg per dose, from 1,730 mg to 1,870 mg per dose, from 1,740 mg to 1,860 mg per dose, from 1,750 mg to 1,850 mg per dose, from 1,760 mg to 1,840 mg per dose, from 1,770 mg to 1,830 mg per dose, from 1,780 mg to 1,820 mg per dose, or from 1,790 mg to 1,810 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,501 mg to about 2,099 mg per dose, such as an amount of about 1,501 mg, 1,502 mg, 1,503 mg, 1,504 mg, 1,505 mg, 1,506 mg, 1,507 mg, 1,508 mg, 1,509 mg, 1,510 mg, 1,511 mg, 1,512 mg, 1,513 mg, 1,514 mg, 1,515 mg, 1,516 mg, 1,517 mg, 1,518 mg, 1,519 mg, 1,520 mg, 1,521 mg, 1,522 mg, 1,523 mg, 1,524 mg, 1,525 mg, 1,526 mg, 1,527 mg, 1,528 mg, 1,529 mg, 1,530 mg, 1,531 mg, 1,532 mg, 1,533 mg, 1,534 mg, 1,535 mg, 1,536 mg, 1,537 mg, 1,538 mg, 1,539 mg, 1,540 mg, 1,541 mg, 1,542 mg, 1,543 mg, 1,544 mg, 1,545 mg, 1,546 mg, 1,547 mg, 1,548 mg, 1,549 mg, 1,550 mg, 1,551 mg, 1,552 mg, 1,553 mg, 1,554 mg, 1,555 mg, 1,556 mg, 1,557 mg, 1,558 mg, 1,559 mg, 1,560 mg, 1,561 mg, 1,562 mg, 1,563 mg, 1,564 mg, 1,565 mg, 1,566 mg, 1,567 mg, 1,568 mg, 1,569 mg, 1,570 mg, 1,571 mg, 1,572 mg, 1,573 mg, 1,574 mg, 1,575 mg, 1,576 mg, 1,577 mg, 1,578 mg, 1,579 mg, 1,580 mg, 1,581 mg, 1,582 mg, 1,583 mg, 1,584 mg, 1,585 mg, 1,586 mg, 1,587 mg, 1,588 mg, 1,589 mg, 1,590 mg, 1,591 mg, 1,592 mg, 1,593 mg, 1,594 mg, 1,595 mg, 1,596 mg, 1,597 mg, 1,598 mg, 1,599 mg, 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, 2,000 mg, 2,001 mg, 2,002 mg, 2,003 mg, 2,004 mg, 2,005 mg, 2,006 mg, 2,007 mg, 2,008 mg, 2,009 mg, 2,010 mg, 2,011 mg, 2,012 mg, 2,013 mg, 2,014 mg, 2,015 mg, 2,016 mg, 2,017 mg, 2,018 mg, 2,019 mg, 2,020 mg, 2,021 mg, 2,022 mg, 2,023 mg, 2,024 mg, 2,025 mg, 2,026 mg, 2,027 mg, 2,028 mg, 2,029 mg, 2,030 mg, 2,031 mg, 2,032 mg, 2,033 mg, 2,034 mg, 2,035 mg, 2,036 mg, 2,037 mg, 2,038 mg, 2,039 mg, 2,040 mg, 2,041 mg, 2,042 mg, 2,043 mg, 2,044 mg, 2,045 mg, 2,046 mg, 2,047 mg, 2,048 mg, 2,049 mg, 2,050 mg, 2,051 mg, 2,052 mg, 2,053 mg, 2,054 mg, 2,055 mg, 2,056 mg, 2,057 mg, 2,058 mg, 2,059 mg, 2,060 mg, 2,061 mg, 2,062 mg, 2,063 mg, 2,064 mg, 2,065 mg, 2,066 mg, 2,067 mg, 2,068 mg, 2,069 mg, 2,070 mg, 2,071 mg, 2,072 mg, 2,073 mg, 2,074 mg, 2,075 mg, 2,076 mg, 2,077 mg, 2,078 mg, 2,079 mg, 2,080 mg, 2,081 mg, 2,082 mg, 2,083 mg, 2,084 mg, 2,085 mg, 2,086 mg, 2,087 mg, 2,088 mg, 2,089 mg, 2,090 mg, 2,091 mg, 2,092 mg, 2,093 mg, 2,094 mg, 2,095 mg, 2,096 mg, 2,097 mg, 2,098 mg, or 2,099 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,600 mg to about 2,000 mg per dose, such as an amount of about 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, or 2,000 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,700 mg to about 1,900 mg per dose, such as an amount of about 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, or 1,900 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,750 mg to about 1,850 mg per dose, such as an amount of about 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, or 1,850 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,760 mg to about 1,840 mg per dose, such as an amount of about 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, or 1,840 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (I)).

For example, in some embodiments, the oxytocin antagonist is administered to the subject in an amount of from about 1,770 mg to about 1,830 mg per dose, such as an amount of about 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, or 1,830 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (I)).

For example, in some embodiments, the oxytocin antagonist is administered to the subject in an amount of from about 1,780 mg to about 1,820 mg per dose, such as an amount of about 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, or 1,820 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,790 mg to about 1,810 mg per dose, such as an amount of about 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, or 1,810 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in an amount of from about 2,100 mg to about 2,700 mg per dose, from about 2,150 mg to about 2,650 mg per dose, from about 2,200 mg to about 2,600 mg per dose, from about 2,250 mg to about 2,550 mg per dose, from about 2,300 mg to about 2,500 mg per dose, from about 2,350 mg to about 2,450 mg per dose, from about 2,360 mg to about 2,440 mg per dose, from about 2,370 mg to about 2,430 mg per dose, from about 2,380 mg to about 2,420 mg per dose, from about 2,390 mg to about 2,410 mg per dose, from about 2,391 mg to about 2,409 mg per dose, from about 2,392 mg to about 2,408 mg per dose, from about 2,393 mg to about 2,407 mg per dose, from about 2,394 mg to about 2,406 mg per dose, from about 2,395 mg to about 2,405 mg per dose, from about 2,396 mg to about 2,404 mg per dose, from about 2,397 mg to about 2,403 mg per dose, from about 2,398 mg to about 2,402 mg per dose, or from about 2,399 mg to about 2,401 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the compound is administered to the subject in an amount of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg per dose (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (I)).

In some embodiments, the oxytocin antagonist is administered to the subject in an amount of about 1,800 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in an amount of about 2,100 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in an amount of about 2,400 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in one or more doses (e.g., in 1, 2, 3, 4, 5, 6, 7, 8, 9, 10, or more doses) totaling from 1,500 mg to 2,100 mg, such as in one or more doses (e.g., in 1, 2, 3, 4, 5, 6, 7, 8, 9, 10, or more doses) totaling from 1,510 mg to 2,090 mg, from 1,520 mg to 2,080 mg, from 1,530 mg to 2,070 mg, from 1,540 mg to 2,060 mg, from 1,550 mg to 2,050 mg, from 1,560 mg to 2,040 mg, from 1,570 mg to 2,030 mg, from 1,580 mg to 2,020 mg, from 1,590 mg to 2,010 mg, from 1,600 mg to 2,000 mg, from 1,610 mg to 1,990 mg, from 1,620 mg to 1,980 mg, from 1,630 mg to 1,970 mg, from 1,640 mg to 1,960 mg, from 1,650 mg to 1,950 mg, from 1,660 mg to 1,940 mg, from 1,670 mg to 1,930 mg, from 1,680 mg to 1,920 mg, from 1,690 mg to 1,910 mg, from 1,700 mg to 1,900 mg, from 1,710 mg to 1,890 mg, from 1,720 mg to 1,880 mg, from 1,730 mg to 1,870 mg, from 1,740 mg to 1,860 mg, from 1,750 mg to 1,850 mg, from 1,760 mg to 1,840 mg, from 1,770 mg to 1,830 mg, from 1,780 mg to 1,820 mg, or from 1,790 mg to 1,810 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in in one or more doses (e.g., in 1, 2, 3, 4, 5, 6, 7, 8, 9, 10, or more doses) totaling from about 1,501 mg to about 2,099 mg, such as in one or more doses (e.g., in 1, 2, 3, 4, 5, 6, 7, 8, 9, 10, or more doses) totaling about 1,501 mg, 1,502 mg, 1,503 mg, 1,504 mg, 1,505 mg, 1,506 mg, 1,507 mg, 1,508 mg, 1,509 mg, 1,510 mg, 1,511 mg, 1,512 mg, 1,513 mg, 1,514 mg, 1,515 mg, 1,516 mg, 1,517 mg, 1,518 mg, 1,519 mg, 1,520 mg, 1,521 mg, 1,522 mg, 1,523 mg, 1,524 mg, 1,525 mg, 1,526 mg, 1,527 mg, 1,528 mg, 1,529 mg, 1,530 mg, 1,531 mg, 1,532 mg, 1,533 mg, 1,534 mg, 1,535 mg, 1,536 mg, 1,537 mg, 1,538 mg, 1,539 mg, 1,540 mg, 1,541 mg, 1,542 mg, 1,543 mg, 1,544 mg, 1,545 mg, 1,546 mg, 1,547 mg, 1,548 mg, 1,549 mg, 1,550 mg, 1,551 mg, 1,552 mg, 1,553 mg, 1,554 mg, 1,555 mg, 1,556 mg, 1,557 mg, 1,558 mg, 1,559 mg, 1,560 mg, 1,561 mg, 1,562 mg, 1,563 mg, 1,564 mg, 1,565 mg, 1,566 mg, 1,567 mg, 1,568 mg, 1,569 mg, 1,570 mg, 1,571 mg, 1,572 mg, 1,573 mg, 1,574 mg, 1,575 mg, 1,576 mg, 1,577 mg, 1,578 mg, 1,579 mg, 1,580 mg, 1,581 mg, 1,582 mg, 1,583 mg, 1,584 mg, 1,585 mg, 1,586 mg, 1,587 mg, 1,588 mg, 1,589 mg, 1,590 mg, 1,591 mg, 1,592 mg, 1,593 mg, 1,594 mg, 1,595 mg, 1,596 mg, 1,597 mg, 1,598 mg, 1,599 mg, 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, 2,000 mg, 2,001 mg, 2,002 mg, 2,003 mg, 2,004 mg, 2,005 mg, 2,006 mg, 2,007 mg, 2,008 mg, 2,009 mg, 2,010 mg, 2,011 mg, 2,012 mg, 2,013 mg, 2,014 mg, 2,015 mg, 2,016 mg, 2,017 mg, 2,018 mg, 2,019 mg, 2,020 mg, 2,021 mg, 2,022 mg, 2,023 mg, 2,024 mg, 2,025 mg, 2,026 mg, 2,027 mg, 2,028 mg, 2,029 mg, 2,030 mg, 2,031 mg, 2,032 mg, 2,033 mg, 2,034 mg, 2,035 mg, 2,036 mg, 2,037 mg, 2,038 mg, 2,039 mg, 2,040 mg, 2,041 mg, 2,042 mg, 2,043 mg, 2,044 mg, 2,045 mg, 2,046 mg, 2,047 mg, 2,048 mg, 2,049 mg, 2,050 mg, 2,051 mg, 2,052 mg, 2,053 mg, 2,054 mg, 2,055 mg, 2,056 mg, 2,057 mg, 2,058 mg, 2,059 mg, 2,060 mg, 2,061 mg, 2,062 mg, 2,063 mg, 2,064 mg, 2,065 mg, 2,066 mg, 2,067 mg, 2,068 mg, 2,069 mg, 2,070 mg, 2,071 mg, 2,072 mg, 2,073 mg, 2,074 mg, 2,075 mg, 2,076 mg, 2,077 mg, 2,078 mg, 2,079 mg, 2,080 mg, 2,081 mg, 2,082 mg, 2,083 mg, 2,084 mg, 2,085 mg, 2,086 mg, 2,087 mg, 2,088 mg, 2,089 mg, 2,090 mg, 2,091 mg, 2,092 mg, 2,093 mg, 2,094 mg, 2,095 mg, 2,096 mg, 2,097 mg, 2,098 mg, or 2,099 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (I)).

For example, in some embodiments, the oxytocin antagonist is administered to the subject in one or more doses (e.g., in 1, 2, 3, 4, 5, 6, 7, 8, 9, 10, or more doses) totaling from about 1,600 mg to about 2,000 mg, such as in one or more doses (e.g., in 1, 2, 3, 4, 5, 6, 7, 8, 9, 10, or more doses) totaling about 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, or 2,000 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in one or more doses (e.g., in 1, 2, 3, 4, 5, 6, 7, 8, 9, 10, or more doses) totaling from about 1,700 mg to about 1,900 mg, such as in one or more doses (e.g., in 1, 2, 3, 4, 5, 6, 7, 8, 9, 10, or more doses) totaling about 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, or 1,900 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in in one or more doses (e.g., in 1, 2, 3, 4, 5, 6, 7, 8, 9, 10, or more doses) totaling from about 1,750 mg to about 1,850 mg, such as an amount of about 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, or 1,850 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in one or more doses (e.g., in 1, 2, 3, 4, 5, 6, 7, 8, 9, 10, or more doses) totaling from about 1,760 mg to about 1,840 mg, such as in one or more doses (e.g., in 1, 2, 3, 4, 5, 6, 7, 8, 9, 10, or more doses) totaling about 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, or 1,840 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in one or more doses (e.g., in 1, 2, 3, 4, 5, 6, 7, 8, 9, 10, or more doses) totaling from about 1,770 mg to about 1,830 mg, such as an amount of about 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, or 1,830 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in one or more doses (e.g., in 1, 2, 3, 4, 5, 6, 7, 8, 9, 10, or more doses) totaling from about 1,780 mg to about 1,820 mg, such as an amount of about 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, or 1,820 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in one or more doses (e.g., in 1, 2, 3, 4, 5, 6, 7, 8, 9, 10, or more doses) totaling from about 1,790 mg to about 1,810 mg, such as an amount of about 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, or 1,810 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in one or more doses (e.g., in 1, 2, 3, 4, 5, 6, 7, 8, 9, 10, or more doses) totaling from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the compound is administered to the subject in one or more doses (e.g., in 1, 2, 3, 4, 5, 6, 7, 8, 9, 10, or more doses) totaling about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in one or more doses (e.g., in 1, 2, 3, 4, 5, 6, 7, 8, 9, 10, or more doses) totaling about 1,800 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in one or more doses (e.g., in 1, 2, 3, 4, 5, 6, 7, 8, 9, 10, or more doses) totaling about 2,100 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in one or more doses (e.g., in 1, 2, 3, 4, 5, 6, 7, 8, 9, 10, or more doses) totaling about 2,400 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in a single dose (e.g., on the day of the embryo transfer therapy) of from 1,500 mg to 2,100 mg, such as in a single dose (e.g., on the day of the embryo transfer therapy) of from 1,510 mg to 2,090 mg, from 1,520 mg to 2,080 mg, from 1,530 mg to 2,070 mg, from 1,540 mg to 2,060 mg, from 1,550 mg to 2,050 mg, from 1,560 mg to 2,040 mg, from 1,570 mg to 2,030 mg, from 1,580 mg to 2,020 mg, from 1,590 mg to 2,010 mg, from 1,600 mg to 2,000 mg, from 1,610 mg to 1,990 mg, from 1,620 mg to 1,980 mg, from 1,630 mg to 1,970 mg, from 1,640 mg to 1,960 mg, from 1,650 mg to 1,950 mg, from 1,660 mg to 1,940 mg, from 1,670 mg to 1,930 mg, from 1,680 mg to 1,920 mg, from 1,690 mg to 1,910 mg, from 1,700 mg to 1,900 mg, from 1,710 mg to 1,890 mg, from 1,720 mg to 1,880 mg, from 1,730 mg to 1,870 mg, from 1,740 mg to 1,860 mg, from 1,750 mg to 1,850 mg, from 1,760 mg to 1,840 mg, from 1,770 mg to 1,830 mg, from 1,780 mg to 1,820 mg, or from 1,790 mg to 1,810 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,501 mg to about 2,099 mg, such as in a single dose (e.g., on the day of the embryo transfer therapy) of about 1,501 mg, 1,502 mg, 1,503 mg, 1,504 mg, 1,505 mg, 1,506 mg, 1,507 mg, 1,508 mg, 1,509 mg, 1,510 mg, 1,511 mg, 1,512 mg, 1,513 mg, 1,514 mg, 1,515 mg, 1,516 mg, 1,517 mg, 1,518 mg, 1,519 mg, 1,520 mg, 1,521 mg, 1,522 mg, 1,523 mg, 1,524 mg, 1,525 mg, 1,526 mg, 1,527 mg, 1,528 mg, 1,529 mg, 1,530 mg, 1,531 mg, 1,532 mg, 1,533 mg, 1,534 mg, 1,535 mg, 1,536 mg, 1,537 mg, 1,538 mg, 1,539 mg, 1,540 mg, 1,541 mg, 1,542 mg, 1,543 mg, 1,544 mg, 1,545 mg, 1,546 mg, 1,547 mg, 1,548 mg, 1,549 mg, 1,550 mg, 1,551 mg, 1,552 mg, 1,553 mg, 1,554 mg, 1,555 mg, 1,556 mg, 1,557 mg, 1,558 mg, 1,559 mg, 1,560 mg, 1,561 mg, 1,562 mg, 1,563 mg, 1,564 mg, 1,565 mg, 1,566 mg, 1,567 mg, 1,568 mg, 1,569 mg, 1,570 mg, 1,571 mg, 1,572 mg, 1,573 mg, 1,574 mg, 1,575 mg, 1,576 mg, 1,577 mg, 1,578 mg, 1,579 mg, 1,580 mg, 1,581 mg, 1,582 mg, 1,583 mg, 1,584 mg, 1,585 mg, 1,586 mg, 1,587 mg, 1,588 mg, 1,589 mg, 1,590 mg, 1,591 mg, 1,592 mg, 1,593 mg, 1,594 mg, 1,595 mg, 1,596 mg, 1,597 mg, 1,598 mg, 1,599 mg, 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, 2,000 mg, 2,001 mg, 2,002 mg, 2,003 mg, 2,004 mg, 2,005 mg, 2,006 mg, 2,007 mg, 2,008 mg, 2,009 mg, 2,010 mg, 2,011 mg, 2,012 mg, 2,013 mg, 2,014 mg, 2,015 mg, 2,016 mg, 2,017 mg, 2,018 mg, 2,019 mg, 2,020 mg, 2,021 mg, 2,022 mg, 2,023 mg, 2,024 mg, 2,025 mg, 2,026 mg, 2,027 mg, 2,028 mg, 2,029 mg, 2,030 mg, 2,031 mg, 2,032 mg, 2,033 mg, 2,034 mg, 2,035 mg, 2,036 mg, 2,037 mg, 2,038 mg, 2,039 mg, 2,040 mg, 2,041 mg, 2,042 mg, 2,043 mg, 2,044 mg, 2,045 mg, 2,046 mg, 2,047 mg, 2,048 mg, 2,049 mg, 2,050 mg, 2,051 mg, 2,052 mg, 2,053 mg, 2,054 mg, 2,055 mg, 2,056 mg, 2,057 mg, 2,058 mg, 2,059 mg, 2,060 mg, 2,061 mg, 2,062 mg, 2,063 mg, 2,064 mg, 2,065 mg, 2,066 mg, 2,067 mg, 2,068 mg, 2,069 mg, 2,070 mg, 2,071 mg, 2,072 mg, 2,073 mg, 2,074 mg, 2,075 mg, 2,076 mg, 2,077 mg, 2,078 mg, 2,079 mg, 2,080 mg, 2,081 mg, 2,082 mg, 2,083 mg, 2,084 mg, 2,085 mg, 2,086 mg, 2,087 mg, 2,088 mg, 2,089 mg, 2,090 mg, 2,091 mg, 2,092 mg, 2,093 mg, 2,094 mg, 2,095 mg, 2,096 mg, 2,097 mg, 2,098 mg, or 2,099 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,600 mg to about 2,000 mg, such as in a single dose (e.g., on the day of the embryo transfer therapy) of about 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, or 2,000 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,700 mg to about 1,900 mg, such as in a single dose (e.g., on the day of the embryo transfer therapy) of about 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, or 1,900 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,750 mg to about 1,850 mg, such as in a single dose (e.g., on the day of the embryo transfer therapy) of about 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, or 1,850 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,760 mg to about 1,840 mg, such as in a single dose (e.g., on the day of the embryo transfer therapy) of about 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, or 1,840 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,770 mg to about 1,830 mg, such as in a single dose (e.g., on the day of the embryo transfer therapy) of about 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, or 1,830 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of about 1,780 mg to about 1,820 mg, such as in a single dose (e.g., on the day of the embryo transfer therapy) of about 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, or 1,820 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,790 mg to about 1,810 mg, such as in a single dose (e.g., on the day of the embryo transfer therapy) of about 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, or 1,810 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the compound is administered to the subject in a single dose (e.g., on the day of the embryo transfer therapy) of from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the compound is administered to the subject in a single dose (e.g., on the day of the embryo transfer therapy) of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg (e.g., wherein the compound is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in a single dose (e.g., on the day of the embryo transfer therapy) of about 1,800 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in a single dose (e.g., on the day of the embryo transfer therapy) of about 2,100 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in a single dose (e.g., on the day of the embryo transfer therapy) of about 2,400 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is epelsiban, or a salt, derivative, variant, crystal form, or formulation thereof, such as a salt, derivative, variant, crystal form, or formulation described in U.S. Pat. Nos. 7,514,437; 8,367,673; 8,541,579; 7,550,462; 7,919,492; 8,202,864; 8,742,099; 9,408,851; 8,716,286; or 8,815,856, the disclosures of each of which are incorporated herein by reference in their entirety.

In some embodiments, the oxytocin antagonist is retosiban, or a salt, derivative, variant, crystal form, or formulation thereof, such as a salt, derivative, variant, crystal form, or formulation described in U.S. Pat. Nos. 7,514,437; 8,367,673; 8,541,579; 8,071,594; 8,357,685; 8,937,179; or 9,452,169, the disclosures of each of which are incorporated herein by reference in their entirety.

In some embodiments, the oxytocin antagonist is barusiban, or a salt, derivative, variant, crystal form, or formulation thereof, such as a salt, derivative, variant, crystal form, or formulation described in U.S. Pat. Nos. 6,143,722; 7,091,314; 7,816,489; or 9,579,305, or WO 2017/060339, the disclosures of each of which are incorporated herein by reference in their entirety.

In some embodiments, the oxytocin antagonist is atosiban, or a salt, derivative, variant, crystal form, or formulation thereof, such as a salt, derivative, variant, crystal form, or formulation described in U.S. Pat. No. 4,504,469 or 4,402,942, the disclosures of each of which are incorporated herein by reference in their entirety.

In some embodiments, the oxytocin antagonist is administered orally.

In some embodiments, the oxytocin antagonist is administered parenterally.

In some embodiments, the oxytocin antagonist is administered intravenously.

In some embodiments, the P4 reference level is from about 1.0 ng/ml to about 2.0 ng/ml. For instance, the P4 reference level may be 1.0 ng/ml, 1.1 ng/ml, 1.2 ng/ml, 1.3 ng/ml, 1.4 ng/ml, 1.5 ng/ml, 1.6 ng/ml, 1.7 ng/ml, 1.8 mg/ml, 1.9 ng/ml, or 2.0 ng/ml, among others. In some embodiments, the P4 reference level is 1.5 ng/ml. In some embodiments, the P4 reference level is 1.54 ng/ml.

In some embodiments, the sample is isolated from the subject from about 1 day to about 7 days prior to the transfer of the one or more embryos to the subject. For instance, in some embodiments, the sample is isolated from the subject about 2 days prior to the transfer of the one or more embryos to the subject. In some embodiments, the sample is isolated from the subject about 3 days prior to the transfer of the one or more embryos to the subject. In some embodiments, the sample is isolated from the subject about 4 days prior to the transfer of the one or more embryos to the subject. In some embodiments, the sample is isolated from the subject about 5 days prior to the transfer of the one or more embryos to the subject.

In some embodiments, the sample is isolated from the subject up to 24 hours prior (e.g., 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior) to isolation of one or more oocytes (e.g., containing one or more mature oocytes) from the subject. In some embodiments, the sample is isolated from the subject immediately prior to isolation of one or more oocytes from the subject.

In some embodiments, the sample is isolated from the subject up to 24 hours prior (e.g., 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior) to isolation of one or more ova from the subject. In some embodiments, the sample is isolated from the subject immediately prior to isolation of one or more ova from the subject.

In some embodiments, the sample is isolated from the subject within about 48 hours of administering hCG to the subject (e.g., so as to induce final follicular maturation), such as within about 47 hours, 46 hours, 45 hours, 44 hours, 43 hours, 42 hours, 41 hours, 40 hours, 39 hours, 38 hours, 37 hours, 36 hours, 35 hours, 34 hours, 33 hours, 32 hours, 31 hours, 30 hours, 29 hours, 28 hours, 27 hours, 26 hours, 25 hours, 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 1 hour, or less, prior to administration of hCG to the subject.

In some embodiments, the P4 reference level is from about 200 nM to about 400 nM. In some embodiments, the P4 reference level is 320 nM. In some embodiments, the sample is isolated from the subject up to 24 hours prior to transfer of the one or more embryos to the subject, such as from 1 hour to 24 hours prior to embryo transfer, from 1 hour to 12 hours prior to embryo transfer, from 1 hour to 8 hours prior to embryo transfer, from 1 hour to 4 hours prior to embryo transfer, or from immediately prior to embryo transfer to 1 hour prior to embryo transfer. In some embodiments, the sample is isolated from the subject immediately prior to transfer of the one or more embryos to the subject (i.e., up to 60 minutes prior to the scheduled transfer of one or more embryos to the subject).

In some embodiments, the subject exhibits an increase in endometrial and/or myometrial PGE2 expression following administration of the oxytocin antagonist to the subject, for instance, as assessed by mass spectrometric and/or spectroscopic techniques described herein or known in the art. In some embodiments, the subject exhibits an increase in endometrial and/or myometrial PGF2α expression following administration of the oxytocin antagonist to the subject, for instance, as assessed by mass spectrometric and/or spectroscopic techniques described herein or known in the art. In some embodiments, the subject exhibits a reduction in endometrial and/or myometrial PGF2α signaling following administration of the oxytocin antagonist, for instance, as assessed by detecting an increase in the concentration of PIP₂ and/or a decrease in the concentration of one or more secondary messengers involved in PGF2α signal transduction, such as DAG, IP₃, and/or intracellular Ca²⁺ released from Ca²⁺ stores, such as sarcoplasmic reticula. For instance, the subject may exhibit a transient increase in endometrial and/or myometrial PGF2α expression, followed by a reduction in PGF2α signalling in these tissues, as evidenced, for instance, by a reduction in endometrial and/or myometrial [DAG], [IP₃], and/or [Ca²⁺].

In some embodiments, the subject sustains pregnancy for at least about 14 days following the transfer of the one or more embryos to the subject, such as for about 14 days, 15 days, 16 days, 17 days, 18 days, 19 days, 20 days, 21 days, 22 days, 23 days, 24 days, 25 days, 26 days, 27 days, 28 days, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, or more, following the transfer of the one or more embryos to the subject. In some embodiments, the subject sustains pregnancy for at least about 6 weeks following the transfer of the one or more embryos to the subject, such as for about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, or more. In some embodiments, the subject sustains pregnancy for at least about 10 weeks following the transfer of the one or more embryos to the subject and/or following the retrieval of one or more oocytes or ova from the subject, such as for about 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, or more, following the transfer of the one or more embryos to the subject and/or following the retrieval of one or more oocytes or ova from the subject.

In some embodiments, pregnancy is assessed by a blood pregnancy test, such as by detecting the presence and/or quantity of hCG in a blood sample isolated from the subject. In some embodiments, pregnancy is assessed by detecting intrauterine embryo heartbeat, for instance, at about 6 weeks or more (e.g., about 6 weeks following the transfer of the one or more embryos to the subject and/or following the retrieval of one or more oocytes or ova from the subject, such as for about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, or more) following the transfer of the one or more embryos to the subject and/or following the retrieval of one or more oocytes or ova from the subject.

In some embodiments, the subject sustains pregnancy and exhibits a live birth following administration of the oxytocin antagonist to the subject. For instance, in some embodiments, the subject sustains pregnancy following administration of the oxytocin antagonist to the subject and exhibits a live birth at a gestational age of at least about 24 weeks, such as at a gestational age of about 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, or more.

In another aspect, the disclosure provides a kit including a package insert and an oxytocin antagonist, wherein the package insert instructs a user of the kit to perform the method of any of the foregoing aspects of the disclosure. In some embodiments of this aspect, the oxytocin antagonist is a compound represented by formula (I)

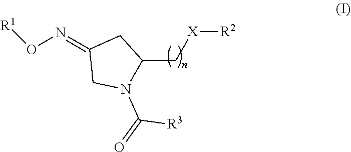

or a geometric isomer, enantiomer, diastereomer, racemate, or sat thereof, wherein PGP-5:

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring;

In some embodiments, the compound is represented by formula (II)

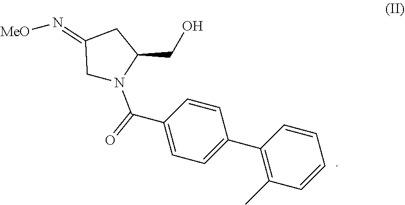 [SMILES: CO/N=C1/C[C@@H](CO)N(C(=O)c2ccc(-c3ccccc3C)cc2)C1]

In some embodiments, the compound represented by formula (II) (i.e., (3Z,5S)-5-(hydroxymethyl)-1-[(2′-methyl-1,1′-biphenyl-4-yl)carbonyl]pyrrolidin-3-one O-methyloxime) is substantially pure. For instance, in some embodiments, the compound represented by formula (II) has a purity of at least 85%, such as a purity of from 85% to 99.9% or more (e.g., a purity of 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more). The purity of the compound represented by formula (II) may be assessed, for instance, using NMR techniques and/or chromatographic methods, such as HPLC procedures, that are known in the art and described herein, such as those techniques that are described in U.S. Pat. No. 9,670,155, the disclosure of which is incorporated herein by reference in its entirety.

In some embodiments, the compound represented by formula (II) is substantially pure with respect to diastereomers of this compound and other by-products that may be formed during the synthesis of this compound. For instance, in some embodiments, the compound represented by formula (II) has a purity of at least 85%, such as a purity of from 85% to 99.9% or more (e.g., a purity of 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more) with respect to diastereomers of this compound and other by-products that may be formed during the synthesis of this compound, such as a by-product that is formed during the synthesis of this compound as described in U.S. Pat. No. 9,670,155. The purity of the compound represented by formula (II) may be assessed, for instance, using NMR techniques and/or chromatographic methods, such as HPLC procedures, that are known in the art and described herein, such as those techniques that are described in U.S. Pat. No. 9,670,155.

In some embodiments, the compound represented by formula (II) is substantially pure with respect to its (3E) diastereomer, (3E,5S)-5-(hydroxymethyl)-1-[(2′-methyl-1,1′-biphenyl-4-yl)carbonyl]pyrrolidin-3-one O-methyloxime. For instance, in some embodiments, the compound represented by formula (II) has a purity of at least 85%, such as a purity of from 85% to 99.9% or more (e.g., a purity of 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more) with respect to (3E,5S)-5-(hydroxymethyl)-1-[(2′-methyl-1,1′-biphenyl-4-yl)carbonyl]pyrrolidin-3-one O-methyloxime. For instance, compound (II) may be administered in the form of a composition (e.g., a tablet, such as a dispersible tablet, capsule, gel cap, powder, liquid solution, or liquid suspension) that contains less than 15% of the (3E) diastereomer. For example, compound (II) may be administered in the form of a composition (e.g., a tablet, such as a dispersible tablet, capsule, gel cap, powder, liquid solution, or liquid suspension) that contains less than 14%, less than 13%, less than 12%, less than 11%, less than 10%, less than 9%, less than 8%, less than 7%, less than 6%, less than 5%, less than 4%, less than 3%, less than 2%, less than 1%, less than 0.1%, less than 0.01%, less than 0.001%, or less of the (3E) diastereomer. The purity of the compound represented by formula (II) may be assessed, for instance, using NMR techniques and/or chromatographic methods, such as HPLC procedures, that are known in the art and described herein, such as those techniques that are described in U.S. Pat. No. 9,670,155.

In some embodiments, the oxytocin antagonist is epelsiban, or a salt, derivative, variant, crystal form, or formulation thereof, such as a salt, derivative, variant, crystal form, or formulation described in U.S. Pat. Nos. 7,514,437; 8,367,673; 8,541,579; 7,550,462; 7,919,492; 8,202,864; 8,742,099; 9,408,851; 8,716,286; or 8,815,856, the disclosures of each of which are incorporated herein by reference in their entirety.

In some embodiments, the oxytocin antagonist is retosiban, or a salt, derivative, variant, crystal form, or formulation thereof, such as a salt, derivative, variant, crystal form, or formulation described in U.S. Pat. Nos. 7,514,437; 8,367,673; 8,541,579; 8,071,594; 8,357,685; 8,937,179; or 9,452,169, the disclosures of each of which are incorporated herein by reference in their entirety.

In some embodiments, the oxytocin antagonist is barusiban, or a salt, derivative, variant, crystal form, or formulation thereof, such as a salt, derivative, variant, crystal form, or formulation described in U.S. Pat. Nos. 6,143,722; 7,091,314; 7,816,489; or 9,579,305, or WO 2017/060339, the disclosures of each of which are incorporated herein by reference in their entirety.

In some embodiments, the oxytocin antagonist is atosiban, or a salt, derivative, variant, crystal form, or formulation thereof, such as a salt, derivative, variant, crystal form, or formulation described in U.S. Pat. No. 4,504,469 or 4,402,942, the disclosures of each of which are incorporated herein by reference in their entirety.

In some embodiments of any of the above aspects of the disclosure, the subject is a human female subject, such as a human female subject of up to 44 years of age, such as a human female subject of from 18 to 44 years of age, such as a human female subject of 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, or 44 years of age. In some embodiments of any of the above aspects of the disclosure, the subject is a human female subject of up to 42 years of age, such as a human female subject of from 18 to 42 years of age, such as a human female subject of 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, or 42 years of age. In some embodiments of any of the above aspects of the disclosure, the subject is a human female subject of up to 36 years of age, such as a human female subject of from 18 to 36 years of age, such as a female subject of 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, or 36 years of age.

### Definitions

As used herein, the term “about” refers to a value that is within 10% above or below the value being described. For instance, the phrase “about 50 mg” refers to a value between and including 45 mg and 55 mg.

As used herein, the term “affinity” refers to the strength of a binding interaction between two molecules, such as a ligand and a receptor. The term “Kᵢ”, as used herein, is intended to refer to the inhibition constant of an antagonist for a particular molecule of interest, and can be expressed as a molar concentration (M). K values for antagonist-target interactions can be determined, e.g., using methods established in the art. Methods that can be used to determine the K of an antagonist for a molecular target include competitive binding experiments, such as competitive radioligand binding assays, for instance, as described in U.S. Pat. No. 9,670,155, the disclosure of which is incorporated herein by reference in its entirety. The term “K_{d}”, as used herein, is intended to refer to the dissociation constant, which can be obtained, for example, from the ratio of the rate constant for the dissociation of the two molecules (k_{d}) to the rate constant for the association of the two molecules (kₐ) and is expressed as a molar concentration (M). K_{d} values for receptor-ligand interactions can be determined, e.g., using methods established in the art. Methods that can be used to determine the K_{d} of a receptor-ligand interaction include surface plasmon resonance, e.g., through the use of a biosensor system such as a BIACORE® system.

As used herein, the term “assisted reproductive technology” or “ART” refers to a fertility treatment in which one or more female gametes (ova) and male gametes (sperm cells) are manipulated ex vivo so as to promote ovum fertilization and formation of a zygote or embryo. The zygote or embryo is then transferred to the uterus of a female subject, for instance, using the compositions and methods described herein. Exemplary assisted reproductive technology procedures include in vitro fertilization (IVF) and intracytoplasmic sperm injection (ICSI) techniques described herein and known in the art.

As used herein, the term “benefit” in the context of a subject undergoing embryo transfer therapy refers to any clinical improvement in the subject's condition or ability to undergo successful embryo implantation and development. Exemplary benefits in this context, such as in the context of a subject treated with an oxytocin antagonist prior to, concurrently with, and/or after the transfer of one or more embryos to the subject, include, without limitation, an increase in the subject's endometrial receptivity, as well as the prevention of a miscarriage in a subject following transfer of one or more embryos to the subject. A subject can be determined to benefit, for instance, from oxytocin antagonist treatment as described herein by observing an elevated endometrial receptivity in the subject (for instance, as assessed by detecting a reduction in prostaglandin F2α (PGF2α) signal transduction as described herein and/or by assessing the subject's ability to sustain a pregnancy for at least 14 days, 6 weeks, 10 weeks, or more, following the transfer of one or more embryos to the subject and/or following the retrieval of one or more oocytes or ova from the subject, and/or by detecting the ability of the subject to give birth to a live offspring at least 24 weeks following the transfer of one or more embryos to the subject. Additionally or alternatively, a subject can be determined to benefit from oxytocin antagonist treatment as described herein by monitoring the subject for a miscarriage following the transfer of one or more embryos to the subject and observing that the subject has not undergone a miscarriage.

As used herein, the term “concurrently with” in the context of administration of a therapeutic agent, such as an oxytocin antagonist described herein, during embryo transfer therapy describes a process in which the therapeutic agent is administered to a subject at substantially the same time as one or more embryos are transferred to the uterus of the subject. For instance, a therapeutic agent is considered to be administered to the subject concurrently with the transfer of one or more embryos if the therapeutic agent is administered to the subject within 1 hour or less (e.g., 60 minutes, 55 minutes, 50 minutes, 45 minutes, 40 minutes, 35 minutes, 30 minutes, 25 minutes, 20 minutes, 15 minutes, 10 minutes, 5 minutes, or less) of the transfer of the one or more embryos to the uterus of the subject.

As used herein, the term “controlled ovarian hyperstimulation” refers to a procedure in which ovulation is induced in a subject, such as a human subject, prior to oocyte or ovum retrieval for use in embryo formation, for instance, by in vitro fertilization (IVF) or intracytoplasmic sperm injection (ICSI). Controlled ovarian hyperstimulation procedures may involve administration of human chorionic gonadotropin (hCG) and/or a gonadotropin-releasing hormone (GnRH) antagonist to the subject so as to promote follicular maturation. Controlled ovarian hyperstimulation methods are known in the art and are described, for instance, in U.S. Pat. Nos. 7,405,197 and 7,815,912, the disclosures of each of which are incorporated herein by reference as they pertain to methods for inducing follicular maturation and ovulation in conjunction with assisted reproductive technology.

As used herein, the term “crystalline” or“crystalline form” means having a physical state that is a regular three-dimensional array of atoms, ions, molecules or molecular assemblies. Crystalline forms have lattice arrays of building blocks called asymmetric units that are arranged according to well-defined symmetries into unit cells that are repeated in three-dimensions. In contrast, the term “amorphous” or “amorphous form” refers to an unorganized (no orderly) structure. The physical state of a therapeutic compound may be determined by exemplary techniques such as x-ray diffraction, polarized light microscopy, thermal gravimetric analysis, and/or differential scanning calorimetry.

As used herein, the term “derived from” in the context of a cell derived from a subject refers to a cell, such as a mammalian ovum, that is either isolated from the subject or obtained from expansion, division, maturation, or manipulation (e.g., ex vivo expansion, division, maturation, or manipulation) of one or more cells isolated from the subject. For instance, an ovum is “derived from” a subject or an oocyte as described herein if the ovum is directly isolated from the subject or obtained from the maturation of an oocyte isolated from the subject, such as an oocyte isolated from the subject from about 1 day to about 7 days prior to the subject undergoing an embryo transfer procedure (e.g., an oocyte isolated from the subject from about 3 days to about 5 days prior to the subject undergoing an embryo transfer procedure). As used herein, the term “dispersible tablet” refers to a tablet capable of rapidly disintegrating in water and that is swallowed by a subject, or that is intended to be disintegrated rapidly in water and subsequently swallowed by a subject, such as a subject undergoing embryo transfer therapy as described herein.

As used herein, the term “dose” refers to the quantity of a therapeutic agent, such as an oxytocin antagonist described herein, that is administered to a subject for the treatment of a disorder or condition, such as to enhance endometrial receptivity and promote successful embryo implantation in the context of assisted reproductive technology. A therapeutic agent as described herein may be administered in a single dose or in multiple doses. In each case, the therapeutic agent may be administered using one or more unit dosage forms of the therapeutic agent. For instance, a single dose of 100 mg of a therapeutic agent may be administered using, e.g., two 50 mg unit dosage forms of the therapeutic agent. Similarly, a single dose of 300 mg of a therapeutic agent may be administered using, e.g., six 50 mg unit dosage forms of the therapeutic agent or two 50 mg unit dosage forms of the therapeutic agent and one 200 mg unit dosage form of the therapeutic agent, among other combinations. Similarly, a single dose of 900 mg of a therapeutic agent may be administered using, e.g., six 50 mg unit dosage forms of the therapeutic agent and three 200 mg unit dosage forms of the therapeutic agent or ten 50 mg unit dosage form of the therapeutic agent and two 200 mg unit dosage forms of the therapeutic agent, among other combinations.

As used herein, the term “embryo” refers to a multicellular, post-zygotic derivative of a fertilized ovum. An embryo may contain two or more blastomeres. For instance, embryos for use with the compositions and methods of the disclosure include those that contain from 6 to 8 blastomeres. Embryos may be produced ex vivo, for instance, by in vitro fertilization (IVF) of an ovum, such as an ovum isolated from a subject undergoing embryo transfer therapy or from a donor, or an ovum produced by maturation of an oocyte isolated from a subject undergoing embryo transfer therapy or from a donor. Embryos may be produced ex vivo, for instance, by intracytoplasmic sperm injection (ICSI) of an ovum, such as an ovum isolated from a subject undergoing embryo transfer therapy or from a donor, or an ovum produced by maturation of an oocyte isolated from a subject undergoing embryo transfer therapy or from a donor. An embryo may have a variety of multicellular forms resulting from ovum fertilization and mitosis of the ensuing zygote. For instance, an embryo may have the form of a morula, which is typically formed from about 3 days to about 4 days following ovum fertilization, and contains two or more cells (such as from 2 to 16 cells, for instance, from 6 to 8 cells) packed contiguously in a spherical arrangement. An embryo may have the form of a blastula (e.g., a mammalian blastocyst), which is typically formed from about 5 days to about 7 days following ovum fertilization, characterized by a spherical morphology containing an outer lining of cells (e.g., a mammalian trophoblast or trophectoderm) surrounding an inner cell mass and a fluid-filled cavity (e.g., a mammalian blastocoele). A blastocyst may contain, for instance, from about 20 to about 300 cells (e.g., about 20 cells, 25 cells, 30 cells, 35 cells, 40 cells, 45 cells, 50 cells, 55 cells, 60 cells, 65 cells, 70 cells, 75 cells, 80 cells, 85 cells, 90 cells, 95 cells, 100 cells, 105 cells, 110 cells, 115 cells, 120 cells, 125 cells, 130 cells, 135 cells, 140 cells, 145 cells, 150 cells, 155 cells, 160 cells, 165 cells, 170 cells, 175 cells, 180 cells, 185 cells, 190 cells, 195 cells, 200 cells, 205 cells, 210 cells, 215 cells, 220 cells, 225 cells, 230 cells, 235 cells, 240 cells, 255 cells, 265 cells, 270 cells, 275 cells, 280 cells, 285 cells, 290 cells, 295 cells, or 300 cells) or more.

As used herein, the term “embryo transfer therapy” refers to a procedure in which one or more embryos are administered to the uterus of a subject, such as a mammalian subject (e.g., a human subject) so as to promote implantation of the one or more embryos into the endometrium of the subject. The embryo may be produced ex vivo, for instance, by in vitro fertilization (IVF) or by intracytoplasmic sperm injection (ICSI), optionally using one or more ova derived from the subject (e.g., one or more ova obtained from maturation of one or more oocytes isolated from the subject) or using one or more ova derived from a donor (e.g., one or more ova obtained from maturation of one or more oocytes isolated from a donor). The embryo may be freshly transferred to the subject, for example, by performing intrauterine embryo transfer using one or more embryos produced by fertilization within about 1 day to about 7 days, such as within about 3 days to about 5 days, of oocyte retrieval from the subject or donor. Embryo transfer is considered “fresh” when ovarian hyperstimulation and ovum/oocyte retrieval from the subject are performed during the same menstrual cycle as embryo transfer to the subject. Alternatively, the embryo may be cryopreserved for long-term storage and subsequently thawed prior to embryo transfer. This process is referred to herein as frozen embryo transfer (FET).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “endometrial receptivity” refers to the ability of the uterus to provide optimal conditions to promote proper implantation and development of an embryo, such as an embryo produced ex vivo by in vitro fertilization of, or intracytoplasmic sperm injection into, an ovum (e.g., an ovum obtained directly from a subject undergoing an embryo transfer procedure therapy or by maturation of one or more oocytes obtained from a subject undergoing an embryo transfer procedure, or an ovum obtained directly from a donor not undergoing an embryo transfer procedure or by maturation of one or more oocytes obtained from a donor not undergoing an embryo transfer procedure). Endometrial receptivity may be enhanced (i.e., increased) using the compositions and methods described herein, for instance, by administration of an oxytocin antagonist to a subject undergoing embryo transfer therapy prior to, concurrently with, and/or following the transfer of one or more embryos to the subject. Enhanced endometrial receptivity may manifest clinically in one or more ways. For instance, a subject exhibiting enhanced endometrial receptivity (e.g., in response to treatment with an oxytocin antagonist prior to, concurrently with, and/or following the transfer of one or more embryos to the subject) may exhibit decreased prostaglandin F2α (PGF2α) signaling in the subject's endometrial and/or myometrial tissue.

For instance, a subject can be determined to exhibit enhanced endometrial receptivity in response to oxytocin antagonist administration if the subject demonstrates a reduced concentration of one or more secondary messengers involved in PGF2α signal transduction, such as diacylglycerol (DAG), inositol-1,4,5-trisphosphate (IP₃), and/or intracellular calcium (Ca²⁺) released from Ca²⁺ stores, such as sarcoplasmic reticula. For instance, a subject can be determined to exhibit enhanced endometrial receptivity in response to oxytocin antagonist treatment as described herein by detecting a decrease in the concentration of one or more of the foregoing secondary messengers in a tissue sample, cell sample, or blood sample isolated from the subject's endometrium and/or myometrium of 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, or more, relative to a measure of the secondary messenger prior to administration of the oxytocin antagonist. Enhanced endometrial receptivity in a subject undergoing embryo transfer therapy can also be observed by assessing the ability of the subject to sustain pregnancy for a period of time following embryo transfer to the uterus of the subject. For instance, a subject exhibiting enhanced endometrial receptivity in response to oxytocin antagonist therapy may sustain pregnancy for at least 14 days following transfer of one or more embryos to the subject, as assessed, for instance, by a blood pregnancy test, such as by detecting the presence and/or quantity of human chorionic gonadotropin (hCG) in a blood sample isolated from the subject using hCG tests known in the art and/or described herein. A subject exhibiting enhanced endometrial receptivity in response to oxytocin antagonist therapy may sustain pregnancy for at least 6 weeks, such as for 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, or 40 weeks, following transfer of one or more embryos to the subject and/or following the retrieval of one or more oocytes or ova from the subject, as assessed, for instance, by detecting intrauterine embryo heartbeat. A subject exhibiting enhanced endometrial receptivity in response to oxytocin antagonist therapy may give birth to a live offspring at a gestational age of at least 24 weeks, for instance, at a gestational age of 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, or 40 weeks.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “gestational age” describes how far along a particular pregnancy is, and is measured from the first day of a pregnant female subject's last menstrual cycle to the current date. As used herein, the term “labor” (which may also be termed birth) relates to the expulsion of the fetus and placenta from the uterus of a pregnant female subject. For a normal pregnancy, labor may occur at a gestational age of about 40 weeks. “Preterm labor” as used herein refers to a condition in which labor commences more than three weeks before the full gestation period, which is typically about 40 weeks. That is, preterm labor occurs at any stage prior to, e.g., 38 weeks of gestation. Preterm labor typically leads to the occurrence of labor, or physiological changes associated with labor in a pregnant female subject, if not treated. Preterm labor may or may not be associated with vaginal bleeding or rupture of uterine membranes. Preterm labor may also be referred to as premature labor. The avoidance of preterm labor in a subject will prolong the term of pregnancy and may therefore avoid preterm delivery, thus reducing the risk of neonatal mortality and morbidity.

As used herein, the term “gonadotropin-releasing hormone antagonist” or “GnRH antagonist” refers to a compound capable of inhibiting the gonadotropin-releasing hormone receptor, e.g., such that release of one or more gonadotropins (such as follicle stimulating hormone and luteinizing hormone) is inhibited. GnRH antagonists include 2-phenylethylpyrimidine-2,4(1H,3H)-dione derivatives, such as those described in U.S. Pat. Nos. 7,056,927; 7,176,211; and 7,419,983; the disclosures of each of which are incorporated herein by reference in their entirety. Exemplary GnRH antagonists include elagolix, relugolix, ASP-1707, and SK12670, among others.

As used herein, the term “IC₅₀” refers to the concentration of a substance (antagonist) that reduces the efficacy of a reference agonist or the constitutive activity of a biological target by 50%, for instance, as measured in a competitive ligand binding assay or in a cell-based functional assay, such as a Ca²⁺ mobilization assay. Exemplary Ca²⁺ mobilization assays that can be used to determine the IC₅₀ of oxytocin antagonist include fluorimetric imaging assays, such as those described in U.S. Pat. No. 9,670,155, the disclosure of which is incorporated herein by reference in its entirety.

As used herein, the term “in vitro fertilization” (IVF) refers to a process in which an ovum, such as a human ovum, is contacted ex vivo with one or more sperm cells so as to promote fertilization of the ovum and zygote formation. The ovum can be derived from a subject, such as a human subject, undergoing embryo transfer therapy. For instance, the ovum may be obtained from maturation of one or more oocytes isolated from the subject, e.g., from about 1 day to about 7 days prior to embryo transfer to the subject (such as from about 3 days to about 5 days prior to embryo transfer to the subject). The ovum may also be retrieved directly from the subject, for instance, by transvaginal ovum retrieval procedures known in the art. Alternatively, the ovum may be derived or isolated from a donor.

As used herein, the term “intracytoplasmic sperm injection” (ICSI) refers to a process in which a sperm cell is injected directly into an ovum, such as a human ovum, so as to promote fertilization of the ovum and zygote formation. The sperm cell may be injected into the ovum, for instance, by piercing the oolemma with a microinjector so as to deliver the sperm cell directly to the cytoplasm of the ovum. ICSI procedures useful in conjunction with the compositions and methods described herein are known in the art and are described, for instance, in WO 2013/158658, WO 2008/051620, and WO 2000/009674, among others, the disclosures of which are incorporated herein by reference as they pertain to compositions and methods for performing intracytoplasmic sperm injection.

As used herein, the term “miscarriage” refers to a naturally-occurring, spontaneous termination of a pregnancy at a stage in which the embryo or fetus is incapable of surviving independently of the mother. For instance, in human subjects, an embryo or fetus may be incapable of surviving independently of the mother at a gestational age of less than about 20 weeks (e.g., a gestational age of less than about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, or 20 weeks).

As used herein, the term “oral bioavailability” refers to the fraction of a compound administered to a subject, such as a mammal (e.g., a human) that reaches systemic circulation in the subject, and that is not sequestered in a non-target organ or excreted without absorption via the gastrointestinal tract. The term refers to a blood plasma concentration that is integrated overtime and is typically expressed as a percentage of the orally administered dose.

As used herein, the terms “ovum” and “mature oocyte” refer to a mature haploid female reproductive cell or gamete. In the context of assisted reproductive technology as described herein, ova may be produced ex vivo by maturation of one or more oocytes isolated from a subject undergoing embryo transfer therapy. Ova may also be isolated directly from the subject, for example, by transvaginal ovum retrieval methods described herein or known in the art.

As used herein, the terms “oxytocin antagonist,” “OT antagonist,” “oxytocin receptor antagonist,” and “OTR antagonist” are used interchangeably and refer to a compound capable of inhibiting the oxytocin receptor, for example, such that activity of one or more downstream signaling molecules in the oxytocin signal transduction cascade is inhibited. Oxytocin antagonists for use with the compositions and methods described herein include pyrrolidin-3-one oxime derivatives, such as those described in U.S. Pat. No. 7,115,754, the disclosure of which is incorporated herein by reference in its entirety. For instance, oxytocin antagonists include (3Z,5S)-5-(hydroxymethyl)-1-[(2′-methyl-1,1′-biphenyl-4-yl)carbonyl]pyrrolidin-3-one O-methyloxime, as described, for instance, in U.S. Pat. No. 9,670,155, the disclosure of which is incorporated herein by reference in its entirety. Additional examples of oxytocin antagonists include atosiban, retosiban, barusiban, and epelsiban, as well as derivatives thereof, among others. For instance, oxytocin antagonists that may be used in conjunction with the compositions and methods described herein include epelsiban, as well as salts, derivatives, variants, crystal forms, and formulations thereof, such as a salt, derivative, variant, crystal form, or formulation described in U.S. Pat. Nos. 7,514,437; 8,367,673; 8,541,579; 7,550,462; 7,919,492; 8,202,864; 8,742,099; 9,408,851; 8,716,286; or 8,815,856, the disclosures of each of which are incorporated herein by reference in their entirety. Additional oxytocin antagonists that may be used in conjunction with the compositions and methods described herein include retosiban, as well as salts, derivatives, variants, crystal forms, and formulations thereof, such as a salt, derivative, variant, crystal form, or formulation described in U.S. Pat. Nos. 7,514,437; 8,367,673; 8,541,579; 8,071,594; 8,357,685; 8,937,179; or 9,452,169, the disclosures of each of which are incorporated herein by reference in their entirety. Oxytocin antagonists useful in conjunction with the compositions and methods described herein further include barusiban, as well as salts, derivatives, variants, crystal forms, and formulations thereof, such as a salt, derivative, variant, crystal form, or formulation described in U.S. Pat. Nos. 6,143,722; 7,091,314; 7,816,489; or 9,579,305, or WO 2017/060339, the disclosures of each of which are incorporated herein by reference in their entirety. Oxytocin antagonists useful in conjunction with the compositions and methods described herein additionally include atosiban, as well as salts, derivatives, variants, crystal forms, and formulations thereof, such as a salt, derivative, variant, crystal form, or formulation described in U.S. Pat. No. 4,504,469 or 4,402,942, the disclosures of each of which are incorporated herein by reference in their entirety.

As used herein, the term “pharmaceutical composition” refers to a mixture containing a therapeutic compound, such as an oxytocin antagonist described herein, to be administered to a subject, such as a mammal, e.g., a human, in order to prevent, treat or control a particular disease or condition affecting or that may affect the mammal, such as to reduce the likelihood of embryo implantation failure in a subject undergoing embryo transfer therapy.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “probe” refers to an agent, such as an antibody, capable of specifically binding to, and detecting the presence of, an analyte of interest. Exemplary probes for use in the detection of progesterone include monoclonal antibodies described herein and known in the art, such as those produced and released by ATCC Accession Number HB 8886 as described in U.S. Pat. No. 4,720,455, the disclosure of which is incorporated herein by reference in its entirety.

As used herein, the term “prostaglandin F2α signaling” or “PGF2α signaling” refers to the endogenous signal transduction cascade by which PGF2α potentiates the intracellular activity of the PGF2α receptor so as to effect one or more biological responses. PGF2α signaling encompasses the PGF2α-mediated stimulation of the PGF2α receptor (FP), a G protein-coupled receptor, which leads to the activation of the G_{q} protein and, in turn phospholipase C (PLC), phosphatidylinositol-3-kinase (PI3K), and extracellular signal-regulated kinases (ERK) 1 and 2. PGF2α signaling can be detected by observing an increase in the concentration of phosphatidylinsolitol-4,5-bisphosphate (PIP₂) and/or a decrease in the concentration of one or more secondary messengers involved in PGF2α signal transduction, such as diacylglycerol (DAG), inositol-1,4,5-tisphosphate (IP₃), and/or intracellular calcium (Ca²⁺) released from Ca²⁺ stores, such as sarcoplasmic reticula. The PGF2α signal transduction cascade is described in detail, for instance, in Xu et al., Reproduction 149:139-146 (2015), the disclosure of which is incorporated herein by reference as it pertains to the proteins and messengers involved in PGF2α signaling.

As used herein, the terms “progesterone reference level” and “P4 reference level” refer to a concentrations of progesterone present within a mammalian subject (e.g., a human subject undergoing an embryo transfer procedure) or within a sample isolated therefrom (such as a serum sample) that, below which, indicates that the subject is likely to benefit from oxytocin antagonist treatment prior to, concurrently with, and/or following the transfer of one or more embryos to the uterus of the subject. P4 reference levels, as described herein, may have different values depending on the point in time during which the serum progesterone level of the patient is assessed. For instance, a P4 reference level of about 320 nM may be used in conjunction with the compositions and methods described herein when being compared to the concentration of P4 present in the serum of a human subject on the day of the embryo transfer procedure. In another example, a P4 reference level of about 1.5 ng/ml may be used in conjunction with the compositions and methods described herein when being compared to the concentration of P4 present in the serum of a human subject the day of oocyte or ovum retrieval from the subject.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and/or cells) isolated from a subject.

As used herein, the phrases “specifically binds” and “binds” refer to a binding reaction which is determinative of the presence of a particular protein in a heterogeneous population of proteins and other biological molecules that is recognized, e.g., by a ligand with particularity. A ligand (e.g., a protein, peptide, or small molecule) that specifically binds to a protein will bind to the protein, e.g., with a K_{D} of less than 100 nM. For example, a ligand that specifically binds to a protein may bind to the protein with a K_{D} of up to 100 nM (e.g., between 1 μM and 100 nM). A ligand that does not exhibit specific binding to a protein or a domain thereof may exhibit a K_{D} of greater than 100 nM (e.g., greater than 200 nM, 300 nM, 400 nM, 500 nM, 600 nm, 700 nM, 800 nM, 900 nM, 1 μM, 100 μM, 500 μM, or 1 mM) for that particular protein or domain thereof. A variety of assay formats may be used to determine the affinity of a ligand for a specific protein. For example, solid-phase ELISA assays are routinely used to identify ligands that specifically bind a target protein. See, e.g., Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of assay formats and conditions that can be used to determine specific protein binding.

As used herein, the terms “subject” and “patient” are interchangeable and refer to an organism that receives treatment for a particular disease or condition as described herein. Examples of subjects and patients include mammals, such as humans, receiving treatment for diseases or conditions, such as to reduce the likelihood of embryo implantation failure before, during, or after embryo transfer therapy sex hormone-dependent diseases.

As used herein, the term “substantially pure” refers to a compound that has a purity of at least 85%, as assessed, for instance, using nuclear magnetic resonance (NMR) and/or high-performance liquid chromatography (HPLC) techniques described herein or known in the art.

As used herein, the term “tₘₐₓ” refers to the time following administration of a compound to a subject at which the compound exhibits a maximum concentration in the blood (e.g., serum or plasma) of the subject.

A compound, salt form, crystal polymorph, therapeutic agent, or other composition described herein may be referred to as being characterized by graphical data “substantially as depicted in” a figure. Such data may include, without limitation, powder X-ray diffractograms, NMR spectra, differential scanning calorimetry curves, and thermogravimetric analysis curves, among others. As is known in the art, such graphical data may provide additional technical information to further define the compound, salt form, crystal polymorph, therapeutic agent, or other composition. As is understood by one of skill in the art, such graphical representations of data may be subject to small variations, e.g., in peak relative intensities and peak positions due to factors such as variations in instrument response and variations in sample concentration and purity. Nonetheless, one of skill in the art will readily be capable of comparing the graphical data in the figures herein with graphical data generated for a compound, salt form, crystal polymorph, therapeutic agent, or other composition and confirm whether the two sets of graphical data are characterizing the same material or two different materials. For instance, a crystal form of (3Z,5S)-5-(hydroxymethyl)-1-[(2′-methyl-1,1′-biphenyl-4-yl)carbonyl]pyrrolidin-3-one O-methyloxime referred to herein as being characterized by graphical data “substantially as depicted in” a figure will thus be understood to include any crystal form of (3Z,5S)-5-(hydroxymethyl)-1-[(2′-methyl-1,1′-biphenyl-4 yl)carbonyl]pyrrolidin-3-one O-methyloxime characterized by the graphical data, optionally having one or more of small variations, e.g., one or more variations described above or known to one of skill in the art.

As used herein, the terms “treat” or “treatment” in the context of a subject undergoing embryo transfer therapy refer to treatment, for instance, by administration of an oxytocin antagonist, with the intention of enhancing endometrial receptivity thereby reducing the likelihood of embryo implantation failure and promoting pregnancy in the subject. Those in need of treatment include, for example, female mammalian subjects, such as female human subjects, that are undergoing embryo transfer therapy, such as subjects undergoing oocyte or ovum retrieval followed by in vitro fertilization or intracytoplasmic sperm injection and subsequent embryo transfer. Those in need of treatment also include, for example, female mammalian subjects, such as female human subjects, that are undergoing embryo transfer therapy, for example, using embryos produced ex vivo by in vitro fertilization or intracytoplasmic sperm injections of one or more ova derived from a donor (e.g., isolated directly from a donor by transvaginal ovum retrieval or by maturation of one or more oocytes obtained directly from the donor). The subject may be undergoing fresh embryo transfer or frozen embryo transfer, and may be transferred, for instance, one, two, three, or more embryos according to the methods described herein. The subject may be one that has previously undergone embryo transfer therapy, either successfully or unsuccessfully, including subjects that have previously undergone one or more cycles (for instance, one, two, three, four, five, six, seven, eight, nine, ten, or more cycles) of failed embryo transfer therapy. A subject can be considered to have been treated, for instance, by administration of an oxytocin antagonist according to the methods described herein, if the subject exhibits endometrial receptivity following administration of the therapeutic agent. Endometrial receptivity can be observed in a variety of clinical manifestations, including a reduction in prostaglandin F2α (PGF2α) signal transduction following oxytocin antagonist administration, successful implantation of the embryo into the endometrium of the subject, as well as the subject's capacity to achieve and sustain pregnancy following embryo transfer, such as for about 14 days, 15 days, 16 days, 17 days, 18 days, 19 days, 20 days, 21 days, 22 days, 23 days, 24 days, 25 days, 26 days, 27 days, 28 days, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, or more, following the transfer of one or more embryos to the subject and/or following the retrieval of one or more oocytes or ova from the subject. Pregnancy can be assessed using methods described herein or known in the art, such as by detecting and/or quantifying human chorionic gonadotropin (hCG) in a blood sample isolated from the subject and/or by detecting intrauterine embryo heartbeat.

As used herein, the term “unit dosage form” refers to a single composition containing a therapeutic agent, such as an oxytocin antagonist described herein, formulated in a manner appropriate for administration to a subject, such as a subject undergoing embryo transfer therapy as described herein. Unit dosage forms include solid and liquid formulations, such as tablets (e.g., dispersible tablets), capsules, gel caps, powders, liquid solutions, and liquid suspensions. A subject may be administered a single dose of a therapeutic agent by administration of one or more unit dosage forms. For instance, a single dose of 100 mg of a therapeutic agent can be administered using two 50 mg unit dosage forms of the therapeutic agent.

As used herein, the term “acyl” refers to the chemical moiety —C(O)R in which R is C₁-C₆ alkyl, aryl, heteroaryl, C₁-C₆ alkyl aryl, or C₁-C₆ alkyl heteroaryl.

As used herein, the term “acylamino” refers to the chemical moiety —NRC(O)R′ in which each of R and R′ is independently hydrogen, C₁-C₆-alkyl, aryl, heteroaryl, C₁-C₆ alkyl aryl, or C₁-C₆ alkyl heteroaryl.

As used herein, the term “acyloxy” refers to the chemical moiety —OC(O)R in which R is C₁-C₆ alkyl, aryl, heteroaryl, C₁-C₆ alkyl aryl, or C₁-C₆ alkyl heteroaryl.

As used herein, the term “alkoxy” refers to the chemical moiety —O—R in which R is C₁-C₆ alkyl, aryl, heteroaryl, C₁-C₆ alkyl aryl, or C₁-C₆ alkyl heteroaryl. Exemplary alkoxy groups include methoxy, ethoxy, phenoxy, and the like.

As used herein, the term “alkoxycarbonyl” refers to the chemical moiety —C(O)OR in which R is hydrogen, C₁-C₆ alkyl, aryl, heteroaryl, C₁-C₆ alkyl aryl, or C₁-C₆ alkyl heteroaryl.

As used herein, the term “amino” refers to the chemical moiety —NRR′ in which each of R and R′ is independently hydrogen, C₁-C₆ alkyl, aryl, heteroaryl, C₁-C₆ alkyl aryl, C₁-C₆ alkyl heteroaryl, cycloalkyl, or heterocycloalkyl, or R and R′, together with the nitrogen atom to which they are bound, can optionally form a 3-8-membered heterocycloalkyl ring.

As used herein, the term “aminocarbonyl” refers to the chemical moiety —C(O)NRR′ in which each of R and R′ is independently hydrogen, C₁-C₆ alkyl, aryl, heteroaryl, C₁-C₆ alkyl aryl, or C₁-C₆ alkyl heteroaryl.

As used herein, the term “aryl” refers to an unsaturated aromatic carbocyclic group of from 6 to 14 carbon atoms having a single ring (e.g., optionally substituted phenyl) or multiple condensed rings (e.g., optionally substituted naphthyl). Exemplary aryl groups include phenyl, naphthyl, phenanthrenyl, and the like. As used herein, the term “aryl” includes substituted aryl substituents, such as an aryl moiety containing a C₁-C₆ alkyl, C₂-C₆ alkenyl, C₂-C₆ alkynyl, cycloalkyl, heterocycloalkyl, C₁-C₆ alkyl aryl, C₁-C₆ alkyl heteroaryl, C₁-C₆ alkyl cycloalkyl, C₁-C₆-alkyl heterocycloalkyl, amino, ammonium, acyl, acyloxy, acylamino, aminocarbonyl, alkoxycarbonyl, ureido, carbamate, aryl, heteroaryl, sulfinyl, sulfonyl, alkoxy, sulfanyl, halogen, carboxy, trihalomethyl, cyano, hydroxy, mercapto, or nitro substituent, or the like.

Exemplary substituted aryl groups include biphenyl and substituted biphenyl substituents.

As used herein, the term “C₁-C₆ alkyl” refers to an optionally branched alkyl moiety having from 1 to 6 carbon atoms, such as methyl, ethyl, propyl, isopropyl, butyl, isobutyl, sec-butyl, tert-butyl, pentyl, isopentyl, neopentyl, tert-pentyl, hexyl, and the like.

As used herein, the term “C₂-C₆ alkenyl” refers to an optionally branched alkenyl moiety having from 2 to 6 carbon atoms, such as vinyl, allyl, 1-propenyl, isopropenyl, 1-butenyl, 2-butenyl, 2-methylallyl, and the like.

As used herein, the term “C₂-C₆ alkynyl” refers to an optionally branched alkynyl moiety having from 2 to 6 carbon atoms, such as ethynyl, 2-propynyl, and the like.

As used herein, the term “carboxy” refers to the chemical moiety —C(O)OH, as well as the ionized form thereof, —C(O)O⁻, and salts thereof.

As used herein, the term “cycloalkyl” refers to a monocyclic cycloalkyl group having, for instance, from 3 to 8 carbon atoms, such as cyclopropyl, cyclobutyl, cyclopentyl, cyclohexyl, cycloheptyl, cyclooctyl, and the like.

As used herein, the term “halogen” refers to a fluorine atom, a chlorine atom, a bromine atom, or an iodine atom.

As used herein, the term “heteroaryl” refers to a monocyclic heteroaromatic, or a bicyclic or a tricyclic fused-ring heteroaromatic group. Exemplary heteroaryl groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, phthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl, benzoquinolyl, and the like.

As used herein, the term “heterocycloalky” refers to a 3 to 8-membered heterocycloalkyl group having one or more heteroatoms, such as a nitrogen atom, an oxygen atom, a sulfur atom, and the like, and optionally having one or more oxo groups. Exemplary heterocycloalkyl substituents include pyrrolidinyl, piperidinyl, oxopiperidinyl, morpholinyl, piperazinyl, 1-methylpiperazinyl, oxopiperazinyl, thiomorpholinyl, azepanyl, diazepanyl, oxazepanyl, thiazepanyl, dioxothiazepanyl, azokanyl, tetrahydrofuranyl, tetrahydropyranyl, and the like.

As used herein, the term “sulfanyl” refers to the chemical moiety —S—R in which R is C₁-C₆ alkyl, aryl, heteroaryl, C₁-C₆ alkyl aryl, or C₁-C₆ alkyl heteroaryl. Exemplary sulfanyl groups include methylsulfanyl, ethylsulfanyl, and the like.

As used herein, the term “sulfinyl” refers to the chemical moiety —S(O)—R in which R is hydrogen, C₁-C₆ alkyl, C₁-C₆ alkyl substituted with one or more halogens, such as a —SO—CF₃ substituent, aryl, heteroaryl, C₁-C₆ alkyl aryl, or C₁-C₆ alkyl heteroaryl.

As used herein, the term “sulfonyl” refers to chemical moiety —SO₂—R in which R is hydrogen, aryl, heteroaryl, C₁-C₆ alkyl, C₁-C₆ alkyl substituted with one or more halogens, such as a —SO₂—CF₃ substituent, C₁-C₆ alkyl aryl, or C₁-C₆ alkyl heteroaryl.

As used herein, the term “sulfonylamino” refers to the chemical moiety —NRSO₂—R′ in which each of R and R′ is independently hydrogen, C₁-C₆ alkyl, aryl, heteroaryl, C₁-C₆ alkyl aryl, or C₁-C₆ alkyl heteroaryl.

As used herein, the term “sulfonyloxy” refers to the chemical moiety —OSO₂—R in which R is hydrogen, C₁-C₆ alkyl, C₁-C₆ alkyl substituted with one or more halogens, such as a —OSO₂—CF₃ substituent, aryl, heteroaryl, C₁-C₆ alkyl aryl, or C₁-C₆ alkyl heteroaryl.

As used herein, the term “ureido” refers to the chemical moiety —NRC(O)NR′R″ where each of R, R′, and R″ is independently hydrogen, C₁-C₆ alkyl, C₂-C₆ alkenyl, C₂-C₆ alkynyl, C₃-C₈ cycloalkyl, heterocycloalkyl, aryl, heteroaryl, C₁-C₆ alkyl aryl, C₁-C₆ alkyl heteroaryl, C₂-C₆ alkenyl aryl, C₂-C₆ alkenyl heteroaryl, C₂-C₆ alkynyl aryl, C₂-C₆ alkynyl heteroaryl, C₁-C₆ alkyl cycloalkyl, or C₁-C₆ alkyl heterocycloalkyl, or R′ and R″, together with the nitrogen atom to which they are bound, can optionally form a 3-8-membered heterocycloalkyl ring.

Unless otherwise constrained by the definition of the individual substituent, the foregoing chemical moieties, such as “alkyl”, “alkenyl”, “alkynyl”, “aryl,” and “heteroaryl” groups can optionally be substituted with, for example, from 1 to 5 substituents selected from the group consisting of C₁-C₆ alkyl, C₂-C₆ alkenyl, C₂-C₆ alkynyl, cycloalkyl, heterocycloalkyl, C₁-C₆ alkyl aryl, C₁-C₆ alkyl heteroaryl, C₁-C₆ alkyl cycloalkyl, C₁-C₆-alkyl heterocycloalkyl, amino, ammonium, acyl, acyloxy, acylamino, aminocarbonyl, alkoxycarbonyl, ureido, carbamate, aryl, heteroaryl, sulfinyl, sulfonyl, alkoxy, sulfanyl, halogen, carboxy, trihalomethyl, cyano, hydroxy, mercapto, nitro, and the like. The substitution may include situations in which neighboring substituents have undergone ring closure, such as ring closure of vicinal functional substituents, to form, for instance, lactams, lactones, cyclic anhydrides, acetals, hemiacetals, thioacetals, aminals, and hemiaminals, formed by ring closure, for example, to furnish a protecting group.

### BRIEF DESCRIPTION OF THE FIGURES

FIG. 1 is a graph showing calculated plasma concentrations of compound (II) following administration of 100 mg (third curve from the top), 300 mg (second curve from the top), and 900 mg (first curve from the top) of this compound to a human subject three days following oocyte retrieval from the subject in preparation for embryo transfer therapy. These pharmacokinetic profiles are contrasted with the calculated plasma concentration of atosiban (first curve from the bottom) in a human subject following administration of atosiban three days after oocyte retrieval in preparation for embryo transfer therapy. The indicated doses of compound (II) were administered orally to the human subject. Atosiban was administered to the human subject intravenously as a 6.75 mg bolus infusion, followed by an 18 mg/hr infusion for 0-1 hours and a subsequent 6 mg/hr infusion for 1-3 hours.

FIG. 2 is a magnified representation of the calculated pharmacokinetic profiles shown in FIG. 1. For clarity, the x-axis is restricted to values from 2.9 days to 3.5 days following oocyte retrieval.

FIG. 3 is a chart showing the quantity of human subjects that did (filled-in circles) and did not (empty circles) exhibit a live birth at the end of pregnancy following treatment with placebo (left column) or 100 mg, 300 mg, or 900 mg of compound (II) (first, second, and third columns on the right, respectively) about 4 hours prior to embryo transfer as described in Example 1, below. The quantity of subjects that did and did not exhibit a live birth are plotted in relation to each subject's pre-treatment serum progesterone concentration on the day of embryo transfer, shown on the y-axis in units of nM. Horizontal lines through each column designate the first (25^{th} percentile), second (median), and third (75^{th} percentiles) quartiles of pre-dose serum progesterone concentrations on the day of embryo transfer among all subjects.

FIG. 4 is a graph showing the percentage of human subjects that tested positive for ongoing pregnancy at 10 weeks following oocyte retrieval (black bars) and subjects that exhibited a live birth at a gestational age of at least 24 weeks (grey bars) in the study described in Example 1. The proportions of subjects that demonstrated these characteristics are plotted as a function of pre-treatment serum progesterone concentration quartile as measured on the day of embryo transfer, which is shown on the x-axis.

FIG. 5 is a graph showing the percentage of human subjects that tested positive for ongoing pregnancy at 10 weeks following oocyte retrieval (black bars) and subjects that exhibited a live birth at a gestational age of at least 24 weeks (grey bars) in the study described in Example 1. The proportions plotted in FIG. 5 exclude data from subjects that exhibited a pre-treatment serum progesterone concentration on the day of embryo transfer in the upper quartile of this metric.

FIG. 6 is a graph showing the average plasma percentage of compound (II) upon administration to human subjects that underwent hormonal treatment mimicking that of patients undergoing a frozen-thawed embryo transfer in the study described in Example 4. Data points denote the mean concentration of compound (II) in plasma following a single oral administration of compound (II) at a dose of 1,800 mg (top) and 900 mg (bottom).

FIG. 7 is a graph showing the effect of compound (II) on uterine contractility in human subjects that underwent hormonal treatment mimicking that of patients undergoing a frozen-thawed embryo transfer in the study described in Example 4. Values along the y-axis represent the quantity of uterine contractions (UC) per minute. Values along the x-axis represent time from administration of a single, oral dose of 1,800 mg of compound (II) (bottom), a single, oral dose of 900 mg of compound (II) (middle), or a single, oral dose of placebo (top).

FIG. 8 is a graph showing the effect of compound (II) on endometrial blood flow in human subjects that underwent hormonal treatment mimicking that of patients undergoing a frozen-thawed embryo transfer in the study described in Example 4. Values along the y-axis represent endometrial flow index (FI), a parameter that is proportional to the volumetric quantity of blood that circulates through the endometrium in a given period of time. Values along the x-axis represent time from administration of a single, oral dose of 1,800 mg of compound (II) (middle), a single, oral dose of 900 mg of compound (II) (top), or a single, oral dose of placebo (bottom). § denotes a p value of less than 0.10.

FIG. 9 is another graph showing the effect of compound (II) on endometrial blood flow in human subjects that underwent hormonal treatment mimicking that of patients undergoing a frozen-thawed embryo transfer in the study described in Example 4. Values along the y-axis represent endometrial vascularity index (VI), a parameter that is proportional to the total quantity of blood vessels in a subject's endometrium. Values along the x-axis represent time from administration of a single, oral dose of 1,800 mg of compound (II), a single, oral dose of 900 mg of compound (II), or a single, oral dose of placebo. § denotes a p value of less than 0.10.

FIG. 10 is a further graph showing the effect of compound (II) on endometrial blood flow in human subjects that underwent hormonal treatment mimicking that of patients undergoing a frozen-thawed embryo transfer in the study described in Example 4. Values along the y-axis represent vascularity flow index (VFI), a parameter that is proportional to the total quantity of blood that circulates through a given volume of a subject's endometrium. Values along the x-axis represent time from administration of a single, oral dose of 1,800 mg of compound (II), a single, oral dose of 900 mg of compound (II), or a single, oral dose of placebo. § denotes a p value of less than 0.10.

FIG. 11 is a graph showing the effect of compound (II) on the endometrial expression of various genes in human subjects that underwent hormonal treatment mimicking that of patients undergoing a frozen-thawed embryo transfer in the study described in Example 4. An RNA-Seq assay was used in order to compare pre-treatment expression with post-treatment expression for a variety of genes. Each gene is represented by a single point on the graph. Values along the x-axis represent the logarithm of the fold change for each gene. Values along the y-axis represent the inverse of the false discovery rate (FDR). Genes that were determined to exhibit a substantially low FDR and a substantially high fold change (either in the positive or negative direction) are shown in the box at the top of FIG. 11.

FIG. 12 is a heatmap showing the effect of compound (II) on the endometrial expression of various genes in human subjects that underwent hormonal treatment mimicking that of patients undergoing a frozen-thawed embryo transfer in the study described in Example 4. An RNA-Seq assay was used in order to compare pre-treatment expression with post-treatment expression for a variety of genes. Each column in the heatmap represents the unique gene expression pattern of a particular subject in the study following administration of either compound (II) or placebo.

FIGS. 13A-13C are graphs showing the relationship between in vivo exposure of compound (II) and the likelihood of a subject undergoing embryo transfer therapy to exhibit a beneficial response to this oxytocin receptor antagonist. FIG. 13A is a graph showing the relationship between the responsiveness to compound (II) of human subjects undergoing embryo transfer therapy as a function of simulated compound (II) Cmax, which is expressed in units of ng/ml. FIG. 13B is a graph showing the relationship between the responsiveness to compound (II) of human subjects undergoing embryo transfer therapy as a function of simulated compound (II) exposure, which is expressed in area under the curve (AUC) units of ng/ml*hr. The data displayed in FIG. 13A and FIG. 13B were obtained from a human clinical trial of patients undergoing embryo transfer therapy that were administered compound (II) in accordance with the protocol described in Example 1, below. In both FIG. 13A and FIG. 13B, responsiveness is a binary variable having a value of either 0 or 1. A response of 1 represents a positive pregnancy test at 14 days following embryo implantation. A response of 0 represents a negative pregnancy test. Each dot represents a the responsiveness of a single patient. The dots are scattered about the y-axis values of 0 and 1 for the sake of visual clarity. The curves in FIG. 13A and FIG. 13B are locally estimated scatterplot smoothing (LOESS) regression curves showing the correlation between compound (II) exposure and patient responsiveness. FIG. 13C shows the correlation between the simulated compound (II) exposure values (used as input variables in FIGS. 13A and 13B) and observed compound (II) exposure. The line shown in FIG. 13C is a LOESS regression line indicating a high degree of correlation between simulated compound (II) exposure values and observed compound (II) exposure values.

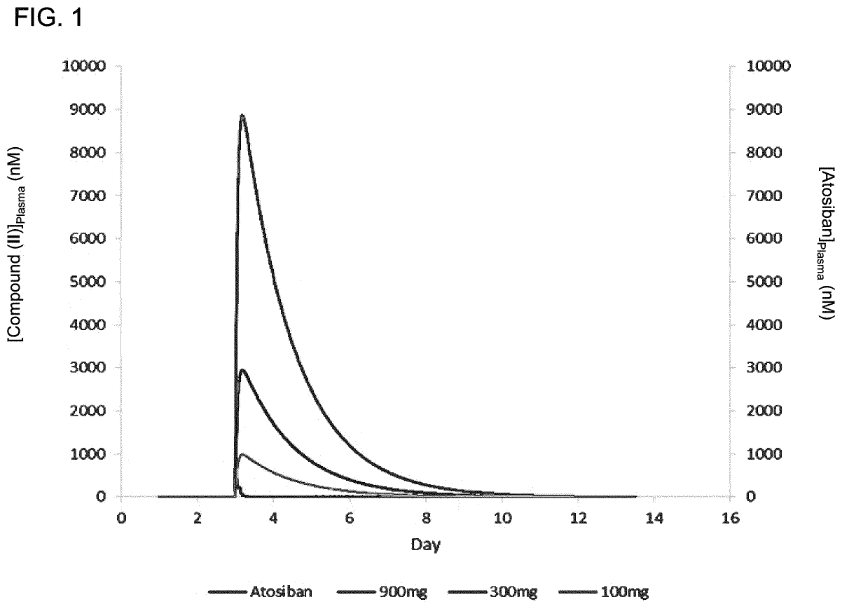

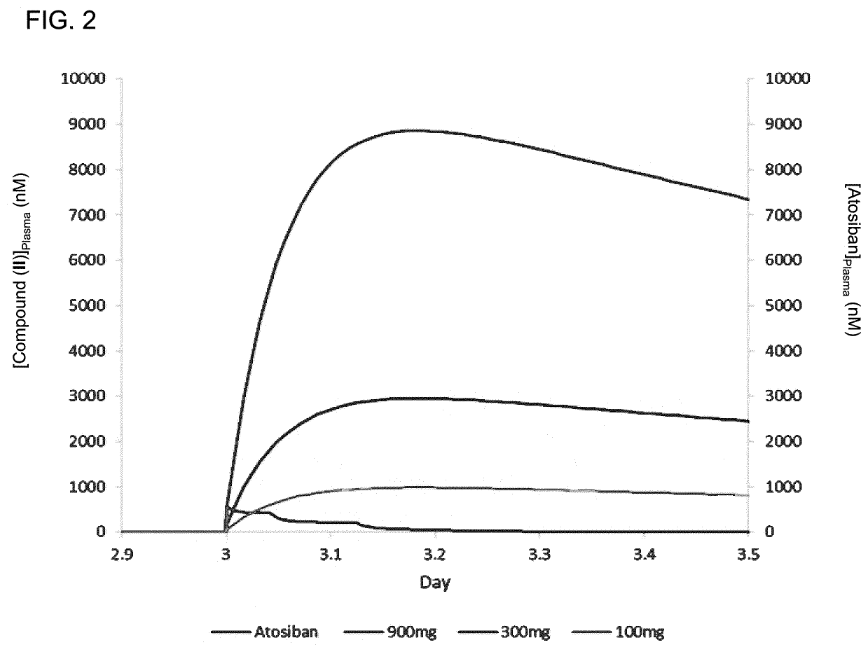

### DETAILED DESCRIPTION

The disclosure features compositions and methods for use in conjunction with assisted reproductive technology. For instance, the compositions and methods described herein can be used to treat subjects undergoing embryo transfer therapy by administering to the subject an oxytocin antagonist so as to enhance the endometrial receptivity of the subject and to reduce the likelihood of embryo implantation failure. The compositions and methods described herein can similarly reduce the likelihood of miscarriage in a subject that has undergone embryo transfer therapy. Using the methods described herein, an oxytocin antagonist can be administered to the subject before, during, and/or after the transfer of one or more embryos to the uterus of the subject so as to promote successful embryo implantation and a sustained pregnancy. The oxytocin antagonist can be administered in a single dose or in multiple doses, such as doses of varying strength or repeat doses of the same strength. For instance, the oxytocin antagonist may be administered in a single high dose or in multiple, lower-strength doses so as to achieve a maximal plasma concentration of the oxytocin antagonist. Oxytocin antagonists useful in conjunction with the compositions and methods described herein include pyrrolidin-3-one oxime compounds represented by formula (I)

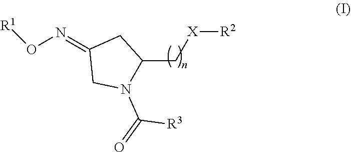

or a geometric isomer, enantiomer, diastereomer, racemate, or salt thereof, wherein

n is an integer from 1 to 3;

R¹ is selected from the group consisting of hydrogen and C₁-C₆ alkyl;

R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;

R³ is selected from the group consisting of aryl and heteroaryl;

X is selected from the group consisting of oxygen and NR⁴; and

R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring. Compounds of this genus are described, for example, in U.S. Pat. No. 7,115,754, the disclosure of which is incorporated herein by reference in its entirety. For instance, oxytocin antagonists that can be used in conjunction with the compositions and methods described herein include (3Z,5S)-5-(hydroxymethyl)-1-[(2′-methyl-1,1′-biphenyl-4-yl)carbonyl]pyrrolidin-3-one O-methyloxime, represented by formula (II), below.

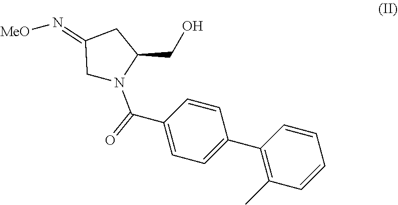 [SMILES: CO/N=C1/C[C@@H](CO)N(C(=O)c2ccc(-c3ccccc3C)cc2)C1]

Using the methods described herein, one can administer an oxytocin antagonist, such as compound (1) or compound (II), to a subject, such as a mammalian subject (e.g., a female human subject) in order to promote enhanced endometrial receptivity, reduce the likelihood of embryo implantation failure, and/or prevent miscarriage in a subject following the transfer of one or more embryos to the uterus of the subject. According to the methods described herein, a compound of formula (I), such as compound (II), may be administered to a subject prior to, concurrently with, and/or following the transfer of one or more embryos to the uterus of the subject so as to achieve a serum concentration of the compound of, for example, from about 1 μM to about 20 μM.

Additional oxytocin antagonists that may be used in conjunction with the compositions and methods described herein include epelsiban, retosiban, barusiban, and atosiban, as well as derivatives thereof, among others. For instance, oxytocin antagonists that may be used in conjunction with the compositions and methods described herein include epelsiban, as well as salts, derivatives, variants, crystal forms, and formulations thereof, such as a salt, derivative, variant, crystal form, or formulation described in U.S. Pat. Nos. 7,514,437; 8,367,673; 8,541,579; 7,550,462; 7,919,492; 8,202,864; 8,742,099; 9,408,851; 8,716,286; or 8,815,856, the disclosures of each of which are incorporated herein by reference in their entirety. Additional oxytocin antagonists that may be used in conjunction with the compositions and methods described herein include retosiban, as well as salts, derivatives, variants, crystal forms, and formulations thereof, such as a salt, derivative, variant, crystal form, or formulation described in U.S. Pat. Nos. 7,514,437; 8,367,673; 8,541,579; 8,071,594; 8,357,685; 8,937,179; or 9,452,169, the disclosures of each of which are incorporated herein by reference in their entirety. Oxytocin antagonists useful in conjunction with the compositions and methods described herein further include barusiban, as well as salts, derivatives, variants, crystal forms, and formulations thereof, such as a salt, derivative, variant, crystal form, or formulation described in U.S. Pat. Nos. 6,143,722; 7,091,314; 7,816,489; or 9,579,305, or WO 2017/060339, the disclosures of each of which are incorporated herein by reference in their entirety. Oxytocin antagonists useful in conjunction with the compositions and methods described herein additionally include atosiban, as well as salts, derivatives, variants, crystal forms, and formulations thereof, such as a salt, derivative, variant, crystal form, or formulation described in U.S. Pat. No. 4,504,469 or 4,402,942, the disclosures of each of which are incorporated herein by reference in their entirety. Using the methods described herein, one can administer one of the foregoing oxytocin antagonists, to a subject, such as a mammalian subject (e.g., a female human subject) in order to reduce the likelihood of embryo implantation failure. According to the methods described herein, one of the foregoing oxytocin antagonists may be administered to a subject prior to, concurrently with, and/or following the transfer of one or more embryos to the uterus of the subject so as to promote enhanced endometrial receptivity, reduce the likelihood of embryo implantation failure, and/or prevent miscarriage in a subject following the transfer of one or more embryos to the uterus of the subject.

The subject may be one that has previously undergone one or more successful or unsuccessful embryo implantation procedures. Alternatively, the subject may be one that has not undergone a previous embryo transfer cycle. According to the methods described herein, the one or more embryos that are ultimately transferred to the subject can be obtained, for instance, by in vitro fertilization (IFV) or intracytoplasmic sperm injection (ICSI) of an ovum isolated or derived from the subject or from a donor. For instances in which the ovum is isolated or derived from the subject, the ovum may be isolated from the subject directly or may be produced ex vivo by inducing maturation of one or more oocytes isolated from the subject. The ova or oocytes may be isolated from the subject, for instance, from about 1 day to about 7 days prior to embryo transfer. In some embodiments, the ova or oocytes are isolated from the subject from about 2 days to about 5 days prior to embryo transfer (e.g., 2 days, 3 days, 4 days, or 5 days prior to embryo transfer). Following fertilization of the ovum by contact with one or more sperm cells, the subsequently formed zygote can be matured ex vivo so as to produce an embryo, such as a morula or blastula (e.g., a mammalian blastocyst), which can then be transferred to the uterus of the subject for implantation into the endometrium. Embryo transfers that can be performed using the methods described herein include fresh embryo transfers, in which the ovum or oocyte used for embryo generation is retrieved from the subject and the ensuing embryo is transferred to the subject during the same menstrual cycle. The embryo can alternatively be produced and cryopreserved for long-term storage prior to transfer to the subject.

The present disclosure additionally features dosing regimens that can be applied to a subject undergoing embryo transfer therapy with an oxytocin antagonist, such as a compound of formula (I) or formula (II) or another oxytocin antagonist described herein, such as epelsiban, retosiban, barusiban, and atosiban, or a salt, derivative, variant, crystal form, or formulation thereof. Using the methods described herein, an oxytocin antagonist such as one of the foregoing agents can be administered to a subject before, during, or after embryo transfer in order to enhance endometrial receptivity, promote successful embryo implantation, and/or prevent the occurrence of a miscarriage in the subject.

For instance, a compound of formula (I) or formula (II) can be administered to a subject hours prior to embryo transfer, such as from about 1 hour to about 24 hours prior to the transfer of one or more embryos to the uterus of the subject, for instance, in a single dose of from about 1,500 mg to about 2,700 mg (e.g., in an amount of from 1,510 mg to 2,090 mg per dose, from 1,520 mg to 2,080 mg per dose, from 1,530 mg to 2,070 mg per dose, from 1,540 mg to 2,060 mg per dose, from 1,550 mg to 2,050 mg per dose, from 1,560 mg to 2,040 mg per dose, from 1,570 mg to 2,030 mg per dose, from 1,580 mg to 2,020 mg per dose, from 1,590 mg to 2,010 mg per dose, from 1,600 mg to 2,000 mg per dose, from 1,610 mg to 1,990 mg per dose, from 1,620 mg to 1,980 mg per dose, from 1,630 mg to 1,970 mg per dose, from 1,640 mg to 1,960 mg per dose, from 1,650 mg to 1,950 mg per dose, from 1,660 mg to 1,940 mg per dose, from 1,670 mg to 1,930 mg per dose, from 1,680 mg to 1,920 mg per dose, from 1,690 mg to 1,910 mg per dose, from 1,700 mg to 1,900 mg per dose, from 1,710 mg to 1,890 mg per dose, from 1,720 mg to 1,880 mg per dose, from 1,730 mg to 1,870 mg per dose, from 1,740 mg to 1,860 mg per dose, from 1,750 mg to 1,850 mg per dose, from 1,760 mg to 1,840 mg per dose, from 1,770 mg to 1,830 mg per dose, from 1,780 mg to 1,820 mg per dose, or from 1,790 mg to 1,810 mg per dose, such as an amount of about 1,500 mg, 1,501 mg, 1,502 mg, 1,503 mg, 1,504 mg, 1,505 mg, 1,506 mg, 1,507 mg, 1,508 mg, 1,509 mg, 1,510 mg, 1,511 mg, 1,512 mg, 1,513 mg, 1,514 mg, 1,515 mg, 1,516 mg, 1,517 mg, 1,518 mg, 1,519 mg, 1,520 mg, 1,521 mg, 1,522 mg, 1,523 mg, 1,524 mg, 1,525 mg, 1,526 mg, 1,527 mg, 1,528 mg, 1,529 mg, 1,530 mg, 1,531 mg, 1,532 mg, 1,533 mg, 1,534 mg, 1,535 mg, 1,536 mg, 1,537 mg, 1,538 mg, 1,539 mg, 1,540 mg, 1,541 mg, 1,542 mg, 1,543 mg, 1,544 mg, 1,545 mg, 1,546 mg, 1,547 mg, 1,548 mg, 1,549 mg, 1,550 mg, 1,551 mg, 1,552 mg, 1,553 mg, 1,554 mg, 1,555 mg, 1,556 mg, 1,557 mg, 1,558 mg, 1,559 mg, 1,560 mg, 1,561 mg, 1,562 mg, 1,563 mg, 1,564 mg, 1,565 mg, 1,566 mg, 1,567 mg, 1,568 mg, 1,569 mg, 1,570 mg, 1,571 mg, 1,572 mg, 1,573 mg, 1,574 mg, 1,575 mg, 1,576 mg, 1,577 mg, 1,578 mg, 1,579 mg, 1,580 mg, 1,581 mg, 1,582 mg, 1,583 mg, 1,584 mg, 1,585 mg, 1,586 mg, 1,587 mg, 1,588 mg, 1,589 mg, 1,590 mg, 1,591 mg, 1,592 mg, 1,593 mg, 1,594 mg, 1,595 mg, 1,596 mg, 1,597 mg, 1,598 mg, 1,599 mg, 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, 2,000 mg, 2,001 mg, 2,002 mg, 2,003 mg, 2,004 mg, 2,005 mg, 2,006 mg, 2,007 mg, 2,008 mg, 2,009 mg, 2,010 mg, 2,011 mg, 2,012 mg, 2,013 mg, 2,014 mg, 2,015 mg, 2,016 mg, 2,017 mg, 2,018 mg, 2,019 mg, 2,020 mg, 2,021 mg, 2,022 mg, 2,023 mg, 2,024 mg, 2,025 mg, 2,026 mg, 2,027 mg, 2,028 mg, 2,029 mg, 2,030 mg, 2,031 mg, 2,032 mg, 2,033 mg, 2,034 mg, 2,035 mg, 2,036 mg, 2,037 mg, 2,038 mg, 2,039 mg, 2,040 mg, 2,041 mg, 2,042 mg, 2,043 mg, 2,044 mg, 2,045 mg, 2,046 mg, 2,047 mg, 2,048 mg, 2,049 mg, 2,050 mg, 2,051 mg, 2,052 mg, 2,053 mg, 2,054 mg, 2,055 mg, 2,056 mg, 2,057 mg, 2,058 mg, 2,059 mg, 2,060 mg, 2,061 mg, 2,062 mg, 2,063 mg, 2,064 mg, 2,065 mg, 2,066 mg, 2,067 mg, 2,068 mg, 2,069 mg, 2,070 mg, 2,071 mg, 2,072 mg, 2,073 mg, 2,074 mg, 2,075 mg, 2,076 mg, 2,077 mg, 2,078 mg, 2,079 mg, 2,080 mg, 2,081 mg, 2,082 mg, 2,083 mg, 2,084 mg, 2,085 mg, 2,086 mg, 2,087 mg, 2,088 mg, 2,089 mg, 2,090 mg, 2,091 mg, 2,092 mg, 2,093 mg, 2,094 mg, 2,095 mg, 2,096 mg, 2,097 mg, 2,098 mg, 2,099 mg, or 2,100 mg per dose, such as an amount of about 1,800 mg per dose; or in an amount of from about 2,100 mg to about 2,700 mg per dose, such as an amount of about 2,100 mg, 2,101 mg, 2,102 mg, 2,103 mg, 2,104 mg, 2,105 mg, 2,106 mg, 2,107 mg, 2,108 mg, 2,109 mg, 2,110 mg, 2,111 mg, 2,112 mg, 2,113 mg, 2,114 mg, 2,115 mg, 2,116 mg, 2,117 mg, 2,118 mg, 2,119 mg, 2,120 mg, 2,121 mg, 2,122 mg, 2,123 mg, 2,124 mg, 2,125 mg, 2,126 mg, 2,127 mg, 2,128 mg, 2,129 mg, 2,130 mg, 2,131 mg, 2,132 mg, 2,133 mg, 2,134 mg, 2,135 mg, 2,136 mg, 2,137 mg, 2,138 mg, 2,139 mg, 2,140 mg, 2,141 mg, 2,142 mg, 2,143 mg, 2,144 mg, 2,145 mg, 2,146 mg, 2,147 mg, 2,148 mg, 2,149 mg, 2,150 mg, 2,151 mg, 2,152 mg, 2,153 mg, 2,154 mg, 2,155 mg, 2,156 mg, 2,157 mg, 2,158 mg, 2,159 mg, 2,160 mg, 2,161 mg, 2,162 mg, 2,163 mg, 2,164 mg, 2,165 mg, 2,166 mg, 2,167 mg, 2,168 mg, 2,169 mg, 2,170 mg, 2,171 mg, 2,172 mg, 2,173 mg, 2,174 mg, 2,175 mg, 2,176 mg, 2,177 mg, 2,178 mg, 2,179 mg, 2,180 mg, 2,181 mg, 2,182 mg, 2,183 mg, 2,184 mg, 2,185 mg, 2,186 mg, 2,187 mg, 2,188 mg, 2,189 mg, 2,190 mg, 2,191 mg, 2,192 mg, 2,193 mg, 2,194 mg, 2,195 mg, 2,196 mg, 2,197 mg, 2,198 mg, 2,199 mg, 2,200 mg, 2,201 mg, 2,202 mg, 2,203 mg, 2,204 mg, 2,205 mg, 2,206 mg, 2,207 mg, 2,208 mg, 2,209 mg, 2,210 mg, 2,211 mg, 2,212 mg, 2,213 mg, 2,214 mg, 2,215 mg, 2,216 mg, 2,217 mg, 2,218 mg, 2,219 mg, 2,220 mg, 2,221 mg, 2,222 mg, 2,223 mg, 2,224 mg, 2,225 mg, 2,226 mg, 2,227 mg, 2,228 mg, 2,229 mg, 2,230 mg, 2,231 mg, 2,232 mg, 2,233 mg, 2,234 mg, 2,235 mg, 2,236 mg, 2,237 mg, 2,238 mg, 2,239 mg, 2,240 mg, 2,241 mg, 2,242 mg, 2,243 mg, 2,244 mg, 2,245 mg, 2,246 mg, 2,247 mg, 2,248 mg, 2,249 mg, 2,250 mg, 2,251 mg, 2,252 mg, 2,253 mg, 2,254 mg, 2,255 mg, 2,256 mg, 2,257 mg, 2,258 mg, 2,259 mg, 2,260 mg, 2,261 mg, 2,262 mg, 2,263 mg, 2,264 mg, 2,265 mg, 2,266 mg, 2,267 mg, 2,268 mg, 2,269 mg, 2,270 mg, 2,271 mg, 2,272 mg, 2,273 mg, 2,274 mg, 2,275 mg, 2,276 mg, 2,277 mg, 2,278 mg, 2,279 mg, 2,280 mg, 2,281 mg, 2,282 mg, 2,283 mg, 2,284 mg, 2,285 mg, 2,286 mg, 2,287 mg, 2,288 mg, 2,289 mg, 2,290 mg, 2,291 mg, 2,292 mg, 2,293 mg, 2,294 mg, 2,295 mg, 2,296 mg, 2,297 mg, 2,298 mg, 2,299 mg, 2,300 mg, 2,301 mg, 2,302 mg, 2,303 mg, 2,304 mg, 2,305 mg, 2,306 mg, 2,307 mg, 2,308 mg, 2,309 mg, 2,310 mg, 2,311 mg, 2,312 mg, 2,313 mg, 2,314 mg, 2,315 mg, 2,316 mg, 2,317 mg, 2,318 mg, 2,319 mg, 2,320 mg, 2,321 mg, 2,322 mg, 2,323 mg, 2,324 mg, 2,325 mg, 2,326 mg, 2,327 mg, 2,328 mg, 2,329 mg, 2,330 mg, 2,331 mg, 2,332 mg, 2,333 mg, 2,334 mg, 2,335 mg, 2,336 mg, 2,337 mg, 2,338 mg, 2,339 mg, 2,340 mg, 2,341 mg, 2,342 mg, 2,343 mg, 2,344 mg, 2,345 mg, 2,346 mg, 2,347 mg, 2,348 mg, 2,349 mg, 2,350 mg, 2,351 mg, 2,352 mg, 2,353 mg, 2,354 mg, 2,355 mg, 2,356 mg, 2,357 mg, 2,358 mg, 2,359 mg, 2,360 mg, 2,361 mg, 2,362 mg, 2,363 mg, 2,364 mg, 2,365 mg, 2,366 mg, 2,367 mg, 2,368 mg, 2,369 mg, 2,370 mg, 2,371 mg, 2,372 mg, 2,373 mg, 2,374 mg, 2,375 mg, 2,376 mg, 2,377 mg, 2,378 mg, 2,379 mg, 2,380 mg, 2,381 mg, 2,382 mg, 2,383 mg, 2,384 mg, 2,385 mg, 2,386 mg, 2,387 mg, 2,388 mg, 2,389 mg, 2,390 mg, 2,391 mg, 2,392 mg, 2,393 mg, 2,394 mg, 2,395 mg, 2,396 mg, 2,397 mg, 2,398 mg, 2,399 mg, 2,400 mg, 2,401 mg, 2,402 mg, 2,403 mg, 2,404 mg, 2,405 mg, 2,406 mg, 2,407 mg, 2,408 mg, 2,409 mg, 2,410 mg, 2,411 mg, 2,412 mg, 2,413 mg, 2,414 mg, 2,415 mg, 2,416 mg, 2,417 mg, 2,418 mg, 2,419 mg, 2,420 mg, 2,421 mg, 2,422 mg, 2,423 mg, 2,424 mg, 2,425 mg, 2,426 mg, 2,427 mg, 2,428 mg, 2,429 mg, 2,430 mg, 2,431 mg, 2,432 mg, 2,433 mg, 2,434 mg, 2,435 mg, 2,436 mg, 2,437 mg, 2,438 mg, 2,439 mg, 2,440 mg, 2,441 mg, 2,442 mg, 2,443 mg, 2,444 mg, 2,445 mg, 2,446 mg, 2,447 mg, 2,448 mg, 2,449 mg, 2,450 mg, 2,451 mg, 2,452 mg, 2,453 mg, 2,454 mg, 2,455 mg, 2,456 mg, 2,457 mg, 2,458 mg, 2,459 mg, 2,460 mg, 2,461 mg, 2,462 mg, 2,463 mg, 2,464 mg, 2,465 mg, 2,466 mg, 2,467 mg, 2,468 mg, 2,469 mg, 2,470 mg, 2,471 mg, 2,472 mg, 2,473 mg, 2,474 mg, 2,475 mg, 2,476 mg, 2,477 mg, 2,478 mg, 2,479 mg, 2,480 mg, 2,481 mg, 2,482 mg, 2,483 mg, 2,484 mg, 2,485 mg, 2,486 mg, 2,487 mg, 2,488 mg, 2,489 mg, 2,490 mg, 2,491 mg, 2,492 mg, 2,493 mg, 2,494 mg, 2,495 mg, 2,496 mg, 2,497 mg, 2,498 mg, 2,499 mg, 2,500 mg, 2,501 mg, 2,502 mg, 2,503 mg, 2,504 mg, 2,505 mg, 2,506 mg, 2,507 mg, 2,508 mg, 2,509 mg, 2,510 mg, 2,511 mg, 2,512 mg, 2,513 mg, 2,514 mg, 2,515 mg, 2,516 mg, 2,517 mg, 2,518 mg, 2,519 mg, 2,520 mg, 2,521 mg, 2,522 mg, 2,523 mg, 2,524 mg, 2,525 mg, 2,526 mg, 2,527 mg, 2,528 mg, 2,529 mg, 2,530 mg, 2,531 mg, 2,532 mg, 2,533 mg, 2,534 mg, 2,535 mg, 2,536 mg, 2,537 mg, 2,538 mg, 2,539 mg, 2,540 mg, 2,541 mg, 2,542 mg, 2,543 mg, 2,544 mg, 2,545 mg, 2,546 mg, 2,547 mg, 2,548 mg, 2,549 mg, 2,550 mg, 2,551 mg, 2,552 mg, 2,553 mg, 2,554 mg, 2,555 mg, 2,556 mg, 2,557 mg, 2,558 mg, 2,559 mg, 2,560 mg, 2,561 mg, 2,562 mg, 2,563 mg, 2,564 mg, 2,565 mg, 2,566 mg, 2,567 mg, 2,568 mg, 2,569 mg, 2,570 mg, 2,571 mg, 2,572 mg, 2,573 mg, 2,574 mg, 2,575 mg, 2,576 mg, 2,577 mg, 2,578 mg, 2,579 mg, 2,580 mg, 2,581 mg, 2,582 mg, 2,583 mg, 2,584 mg, 2,585 mg, 2,586 mg, 2,587 mg, 2,588 mg, 2,589 mg, 2,590 mg, 2,591 mg, 2,592 mg, 2,593 mg, 2,594 mg, 2,595 mg, 2,596 mg, 2,597 mg, 2,598 mg, 2,599 mg, 2,600 mg, 2,601 mg, 2,602 mg, 2,603 mg, 2,604 mg, 2,605 mg, 2,606 mg, 2,607 mg, 2,608 mg, 2,609 mg, 2,610 mg, 2,611 mg, 2,612 mg, 2,613 mg, 2,614 mg, 2,615 mg, 2,616 mg, 2,617 mg, 2,618 mg, 2,619 mg, 2,620 mg, 2,621 mg, 2,622 mg, 2,623 mg, 2,624 mg, 2,625 mg, 2,626 mg, 2,627 mg, 2,628 mg, 2,629 mg, 2,630 mg, 2,631 mg, 2,632 mg, 2,633 mg, 2,634 mg, 2,635 mg, 2,636 mg, 2,637 mg, 2,638 mg, 2,639 mg, 2,640 mg, 2,641 mg, 2,642 mg, 2,643 mg, 2,644 mg, 2,645 mg, 2,646 mg, 2,647 mg, 2,648 mg, 2,649 mg, 2,650 mg, 2,651 mg, 2,652 mg, 2,653 mg, 2,654 mg, 2,655 mg, 2,656 mg, 2,657 mg, 2,658 mg, 2,659 mg, 2,660 mg, 2,661 mg, 2,662 mg, 2,663 mg, 2,664 mg, 2,665 mg, 2,666 mg, 2,667 mg, 2,668 mg, 2,669 mg, 2,670 mg, 2,671 mg, 2,672 mg, 2,673 mg, 2,674 mg, 2,675 mg, 2,676 mg, 2,677 mg, 2,678 mg, 2,679 mg, 2,680 mg, 2,681 mg, 2,682 mg, 2,683 mg, 2,684 mg, 2,685 mg, 2,686 mg, 2,687 mg, 2,688 mg, 2,689 mg, 2,690 mg, 2,691 mg, 2,692 mg, 2,693 mg, 2,694 mg, 2,695 mg, 2,696 mg, 2,697 mg, 2,698 mg, 2,699 mg, or 2,700 mg) to the subject or in multiple doses of lower strength that total one of the foregoing dosage quantities (e.g., in two or more doses of lower strength that total about 1,800 mg, about 2,100 mg, or about 2,400 mg).

In some embodiments, the compound is administered to the subject from about 1 hour to about 12 hours prior to embryo transfer, such as about 4 hours prior to embryo transfer. Using the methods described herein, the oxytocin antagonist, such as a compound of formula (I) or formula (II), can be administered to the subject concurrently with the transfer of one or more embryos to the uterus of the subject, such as within 60 minutes of embryo transfer, for instance, in a single dose of from about 1,500 mg to about 2,700 mg, such as in a single dose of about 1,800 mg, about 2,100 mg, or about 2,400 mg, or in multiple doses of lower strength that total about 1,800 mg, about 2,100 mg, or about 2,400 mg. Additionally or alternatively, the oxytocin antagonist can be administered to the subject following embryo transfer, such as from about 1 hour to about 24 hours following embryo transfer. For instance, the oxytocin antagonist can be administered following embryo transfer in a single dose of from about 1,500 mg to about 2,700 mg, such as in a single dose of about 1,800 mg, about 2,100 mg, or about 2,400 mg, or in multiple doses of lower strength that total about 1,800 mg, about 2,100 mg, or about 2,400 mg. In dosing regimens in which the oxytocin antagonist is administered in multiple doses, the compound (e.g., compound (1) or compound (11)) may be administered in multiple doses per day, such as in from 1 dose to 7 doses per day. The dosing may terminate, for instance, on the day of embryo transfer to the subject, or may continue following embryo transfer.

The sections that follow provide a description of various oxytocin antagonists useful in conjunction with the compositions and methods provided by the disclosure, as well as a description of dosing regimens that may guide the administration of oxytocin antagonists to a subject so as to enhance endometrial receptivity upon embryo transfer, reduce the likelihood of embryo implantation failure, and/or prevent the occurrence of a miscarriage in a subject undergoing an assisted reproduction procedure.

### (3Z,5S)-5-hydroxymethyl)-1-[(2′-methyl-1,1′-biphenyl-4-yl)carbonyl]pyrrolidin-3-one O-methyloxime (Compound II)

Compounds of formula (I), such as (3Z,5S)-5-(hydroxymethyl)-1-[(2′-methyl-1,1′-biphenyl-4-yl)carbonyl]pyrrolidin-3-one O-methyloxime, represented by formula (II), above, are non-peptide oxytocin antagonists that can be used to enhance endometrial receptivity, promote successful embryo implantation, and reduce the likelihood of miscarriage in subjects undergoing or that have undergone embryo transfer therapy. Compound (II), in particular, is an orally-active oxytocin antagonist capable of inhibiting human oxytocin receptor with a K of 52 nM and suppressing Ca²⁺ mobilization in cultured HEK293EBNA cells with an IC₅₀ of 81 nM. Additionally, compound (II) selectively inhibits the oxytocin receptor over the vasopressin VIa receptor, as compound (II) inhibits the vasopressin VIa receptor with a K of 120 nM. Compound (II) additionally demonstrates a variety of favorable pharmacokinetic properties, as this compound exhibits an oral bioavailability of from 42-100%, with a serum half-life of from 11-12 hours and a tₘₐₓ of from about 1-4 hours. The foregoing biochemical properties of compound (II), as well as methods for the synthesis and purification of this compound, are described in detail, for instance, in U.S. Pat. No. 9,670,155, the disclosure of which is incorporated herein by reference in its entirety.

### Synthesis of Compound (II)

An exemplary procedure for the synthesis of compound (II) is shown in Scheme 1, below.

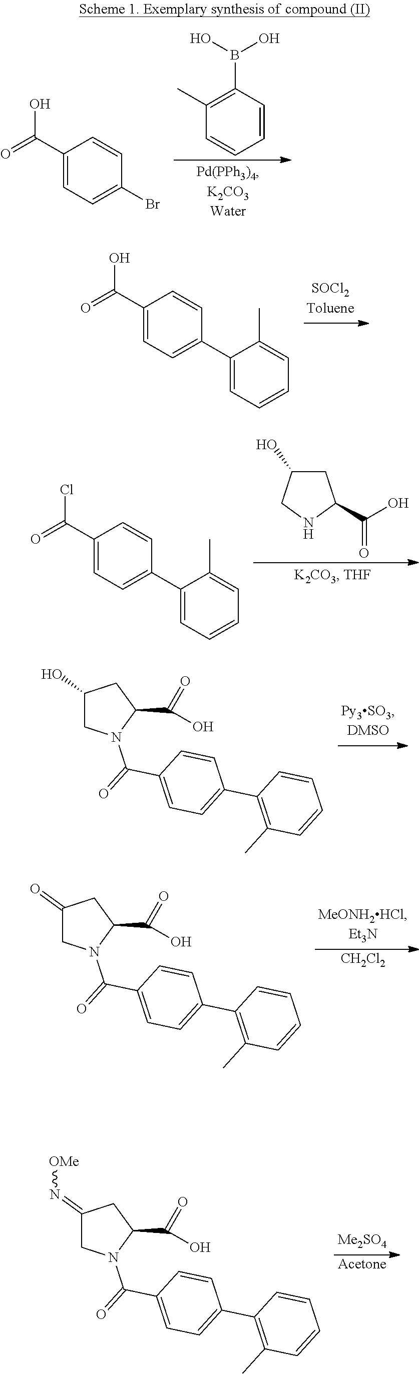

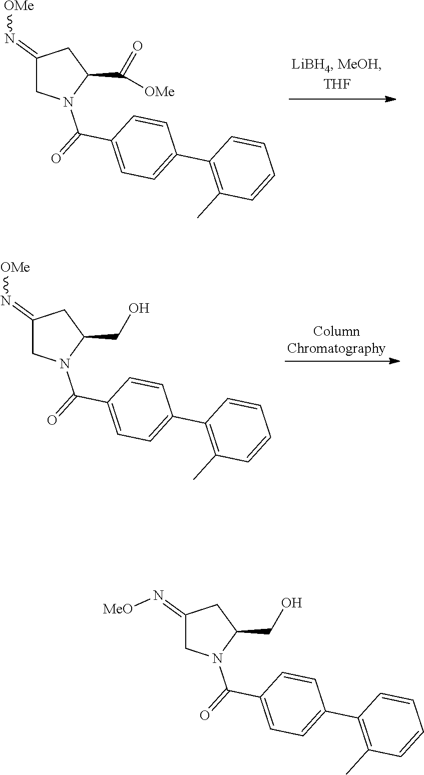 [SMILES: CON=C1C[C@@H](C(=O)OC)N(C(=O)c2ccc(-c3ccccc3C)cc2)C1 ; CON=C1C[C@@H](CO)N(C(=O)c2ccc(-c3ccccc3C)cc2)C1 ; CO/N=C1/C[C@@H](CO)N(C(=O)c2ccc(-c3ccccc3C)cc2)C1]

### Purity of Compound (II)

In some embodiments, the compound represented by formula (II) (i.e., (3Z,5S′)-5-(hydroxymethyl)-1-[(2′-methyl-1,1′-biphenyl-4-yl)carbonyl]pyrrolidin-3-one O-methyloxime) is substantially pure. For instance, in some embodiments, the compound represented by formula (II) has a purity of at least 85%, such as a purity of from 85% to 99.9% or more (e.g., a purity of 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more). The purity of the compound represented by formula (II) may be assessed, for instance, using NMR techniques and/or chromatographic methods, such as HPLC procedures, that are known in the art and described herein, such as those techniques that are described in U.S. Pat. No. 9,670,155, the disclosure of which is incorporated herein by reference in its entirety.

In some embodiments, the compound represented by formula (II) is substantially pure with respect to diastereomers of this compound and other by-products that may be formed during the synthesis of this compound. For instance, in some embodiments, the compound represented by formula (II) has a purity of at least 85%, such as a purity of from 85% to 99.9% or more (e.g., a purity of 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more) with respect to diastereomers of this compound and other by-products that may be formed during the synthesis of this compound, such as a by-product that is formed during the synthesis of this compound as described in U.S. Pat. No. 9,670,155. The purity of the compound represented by formula (II) may be assessed, for instance, using NMR techniques and/or chromatographic methods, such as HPLC procedures, that are known in the art and described herein, such as those techniques that are described in U.S. Pat. No. 9,670,155.

In some embodiments, the compound represented by formula (II) is substantially pure with respect to its (3E) diastereomer, (3E,5S)-5-(hydroxymethyl)-1-[(2′-methyl-1,1′-biphenyl-4-yl)carbonyl]pyrrolidin-3-one O-methyloxime. For instance, in some embodiments, the compound represented by formula (II) has a purity of at least 85%, such as a purity of from 85% to 99.9% or more (e.g., a purity of 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or more) with respect to (3E,5S)-5-(hydroxymethyl)-1-[(2′-methyl-1,1′-biphenyl-4-yl)carbonyl]pyrrolidin-3-one O-methyloxime. For instance, compound (II) may be administered in the form of a composition (e.g., a tablet, such as a dispersible tablet, capsule, gel cap, powder, liquid solution, or liquid suspension) that contains less than 15% of the (3E) diastereomer. For example, compound (II) may be administered in the form of a composition (e.g., a tablet, such as a dispersible tablet, capsule, gel cap, powder, liquid solution, or liquid suspension) that contains less than 14%, less than 13%, less than 12%, less than 11%, less than 10%, less than 9%, less than 8%, less than 7%, less than 6%, less than 5%, less than 4%, less than 3%, less than 2%, less than 1%, less than 0.1%, less than 0.01%, less than 0.001%, or less of the (3E) diastereomer. The purity of the compound represented by formula (II) may be assessed, for instance, using NMR techniques and/or chromatographic methods, such as HPLC procedures, that are known in the art and described herein, such as those techniques that are described in U.S. Pat. No. 9,670,155.

### Therapeutic Activity

The present disclosure is based in part on the discovery that compounds of formula (I), such as compound (II), is capable of promoting successful endometrial implantation of a transferred embryo in female human subjects and prolonging the duration of pregnancy relative to subjects not treated with this compound. Specifically, compound (II) has been found to reduce the risk of embryo implantation failure in clinical studies conducted with human subjects that previously underwent ovarian hyperstimulation and oocyte retrieval. It has been discovered that compounds of formula (I), such as compound (II), increase the rate of successful embryo implantation as assessed by a variety of metrics. These manifestations have been found to include an increase in the rate of positive pregnancy tests at 14 days, 6 weeks, and 10 weeks following embryo transfer and/or oocyte retrieval, as well as an increase in the rate of live births at a gestational age of at least 24 weeks.

Oxytocin antagonists, such as compounds of formula (I) and (II) and other oxytocin antagonists described herein, are particularly effective in subjects that do not exhibit elevated serum concentrations of progesterone (P4). For instance, as is described in detail in Example 1, below, compound (II) was found to improve successful embryo implantation rate (for example, as assessed by the above metrics) in a dose-dependent manner. This dose-dependent response was found to be particularly strong in subjects that exhibited a pre-treatment serum P4 concentration of less than 320 nM, such as from about 200 nM to about 300 nM or less. The foregoing P4 concentrations were measured on the day of transfer of one or more embryos to the subject. These heightened P4 levels are indicative of an elevated P4 concentration within about 48 hours of final follicular maturation (e.g., by way of hCG administration) and/or on the day of oocyte or ovum retrieval from the subject, such as a P4 concentration of from 1.0 ng/ml to 2.0 ng/ml (e.g., a P4 concentration of 1.0 ng/ml, 1.1 ng/ml, 1.2 ng/ml, 1.3 ng/ml, 1.4 ng/ml, 1.5 ng/ml, 1.6 ng/ml, 1.7 ng/ml, 1.8 ng/ml, 1.9 ng/ml, or 2.0 ng/ml, and particularly, 1.5 ng/ml). Thus, it has been discovered that a subject's propensity to benefit from treatment with an oxytocin antagonist, such as a compound of formula (I) or formula (II), or another oxytocin antagonist described herein, such as epelsiban, retosiban, barusiban, or atosiban, or a salt, derivative, variant, crystal form, or formulation thereof, can be determined based on the subject's pre-treatment serum level of P4.

Using the compositions and methods described herein, one of skill in the art can assess a patient's likelihood of benefitting from (e.g., experiencing enhanced (i.e., increased) endometrial receptivity in response to) oxytocin antagonist treatment by determining the subject's serum P4 concentration prior to treatment with an oxytocin antagonist. If the subject exhibits a serum P4 concentration below a reference level, such as a serum P4 concentration of below 320 nM on the day of embryo transfer (e.g., up to 24 hours prior to a scheduled embryo transfer, such as immediately prior to a scheduled embryo transfer) or a serum P4 concentration of less than 1.5 ng/ml within about 48 hours of final follicular maturation (e.g., by way of hCG administration) and/or on the day of oocyte or ovum retrieval (e.g., from 1 to 7 days prior to embryo transfer for a patient undergoing an IVF-ET procedure, such as from 3 to 5 days prior to embryo transfer for a patient undergoing an IVF-ET procedure), the subject may be administered an oxytocin antagonist, for instance, prior to, concurrently with, and/or following the transfer of one or more embryos to the subject. If the subject exhibits a serum P4 concentration above a reference level, such as a serum P4 concentration of above 320 nM on the day of embryo transfer (e.g., up to 24 hours prior to a scheduled embryo transfer, such as immediately prior to a scheduled embryo transfer) or a serum P4 concentration of greater than 1.5 ng/ml on the day of oocyte or ovum retrieval (e.g., from 1 to 7 days prior to embryo transfer for a patient undergoing an IVF-ET procedure, such as from 3 to 5 days prior to embryo transfer for a patient undergoing an IVF-ET procedure), a physician of skill in the art may determine that the subject will not be administered an oxytocin antagonist, and/or that the subject will be re-scheduled for oocyte or ovum retrieval or embryo transfer until such a time as the subject's serum P4 concentration declines to beneath the P4 reference level.

Additionally, without being limited by mechanism, it has been discovered that oxytocin antagonists such as compounds of formula (I) and (II), and other oxytocin antagonists described herein, may promote the transient overexpression of prostaglandin F2α (PGF2α) and prostaglandin E2 (PGE2) and subsequently inhibit the propagation of PGF2α signal transduction. The attenuation of PGF2α signalling may occur, for instance, by desensitization of the PGF2α receptor in response to the initial flare in PGF2α secretion. This pattern of (i) transiently heightened expression of PGF2α followed by (ii) the reduction in PGF2α signaling induced by oxytocin antagonists such as compounds of formula (I) and (II), as well as other oxytocin antagonists described herein, can in turn enhance the receptivity of the endometrium to one or more exogenous embryos, thereby promoting endometrial implantation and reducing the likelihood of embryo implantation failure. Notably, P4 is a negative regulator of PGF2α expression, which may explain why oxytocin antagonists such as compounds of formula (I) and (II), among other oxytocin antagonists described herein, can have a particularly robust therapeutic effect on subjects that do not exhibit elevated pre-treatment serum P4 concentrations. Such subjects include those that do not exhibit pre-treatment serum P4 concentrations of 320 nM or greater on the day of embryo transfer and/or pre-treatment serum P4 concentrations of 1.5 ng/ml or greater on the day of oocyte or ovum retrieval, as described in Examples 1 and 2, below.

The foregoing discoveries form important bases for the oxytocin antagonist dosing regimens described herein. To optimally enhance endometrial receptivity to one or more transferred embryos, compounds of formulas (I) and (II), as well as additional oxytocin antagonists described herein and known in the art, such as epelsiban, retosiban, barusiban, and atosiban, or a salt, derivative, variant, crystal form, or formulation thereof, can be administered to a subject so as to saturate the oxytocin receptor and achieve complete (i.e., 100%) inhibition of the receptor at the time of embryo implantation. This can be achieved, for instance, by administering compounds of formula (I) or (II) or another oxytocin antagonist described herein or known in the art, such as epelsiban, retosiban, barusiban, and atosiban, or a salt, derivative, variant, crystal form, or formulation thereof, to a subject undergoing embryo transfer therapy such that a maximum plasma concentration of the compound is reached at the time of embryo transfer.

For instance, compounds of formula (I) or (II) can be administered to a subject from about 1 hour to about 24 hours prior to embryo transfer, such as from about 1 hour to about 8 hours prior to embryo transfer so as to achieve a maximum plasma concentration of the compound at the time of embryo transfer. In some embodiments, the compound is administered about 4 hours prior to embryo transfer, as it has been discovered that oral administration of various doses of compound (II) results in a peak plasma concentration of the compound at from about 1 hour to about 4 hours following administration of the compound. Compounds of formula (I) or (II) may be administered prior to, during, and/or after embryo transfer in order to enhance endometrial receptivity and promote successful embryo implantation, for instance, as described below.

The sections that follow describe in further detail additional oxytocin antagonists that may be used in conjunction with the compositions and methods of the disclosure, as well as dosing schedules for the administration of oxytocin antagonists to subjects undergoing embryo transfer therapy and methods of assessing whether a subject is likely to benefit from oxytocin antagonist treatment on the basis of the subject's pre-treatment progesterone level(s).

### Oxytocin Antagonist Dosing Regimens

To promote endometrial receptivity and successful embryo implantation and to reduce the likelihood of miscarriage in a subject undergoing or that has undergone embryo transfer therapy, compounds of formula (I) or (II), or another oxytocin antagonist described herein, may be administered to a subject (e.g., a human subject) before, during, or after embryo transfer. In each case, compounds of formula (I) or (II), or another oxytocin antagonist described herein, may be administered to the subject so as to saturate the oxytocin receptor and achieve inhibition (e.g., 50%, 55%, 60%, 65%, 70%, 75%, 80%, 85%, 90%, 95%, 96%, 97%, 98%, 99%, or 100% inhibition) of the receptor prior to embryo transfer, at the time of embryo transfer, and/or following embryo transfer.

### Administrant Beginning Prior to Embryo Transfer Therapy

Compounds of formula (I) or (II) or another oxytocin antagonist described herein, such as epelsiban, retosiban, barusiban, and atosiban, or a salt, derivative, variant, crystal form, or formulation thereof, may be administered to the subject prior to embryo transfer, such as from about 1 hour to about 24 hours prior to the transfer of the one or more embryos to the subject. In some embodiments, the compound is administered to the subject so as to achieve a maximum plasma concentration of the compound at the time of embryo transfer. For instance, in some embodiments, the compound is administered to the subject from about 1 hour to about 8 hours prior to embryo transfer, such as about four hours prior to embryo transfer.

Compounds of formula (I) or (II) may be administered to a subject undergoing embryo transfer therapy in a single dose, such as in a single dose of from about 1,500 mg to about 2,700 mg, such as in a single dose of from 1,500 mg to 2,100 mg, from 1,510 mg to 2,090 mg, from 1,520 mg to 2,080 mg per dose, from 1,530 mg to 2,070 mg per dose, from 1,540 mg to 2,060 mg per dose, from 1,550 mg to 2,050 mg per dose, from 1,560 mg to 2,040 mg per dose, from 1,570 mg to 2,030 mg per dose, from 1,580 mg to 2,020 mg per dose, from 1,590 mg to 2,010 mg per dose, from 1,600 mg to 2,000 mg per dose, from 1,610 mg to 1,990 mg per dose, from 1,620 mg to 1,980 mg per dose, from 1,630 mg to 1,970 mg per dose, from 1,640 mg to 1,960 mg per dose, from 1,650 mg to 1,950 mg per dose, from 1,660 mg to 1,940 mg per dose, from 1,670 mg to 1,930 mg per dose, from 1,680 mg to 1,920 mg per dose, from 1,690 mg to 1,910 mg per dose, from 1,700 mg to 1,900 mg per dose, from 1,710 mg to 1,890 mg per dose, from 1,720 mg to 1,880 mg per dose, from 1,730 mg to 1,870 mg per dose, from 1,740 mg to 1,860 mg per dose, from 1,750 mg to 1,850 mg per dose, from 1,760 mg to 1,840 mg per dose, from 1,770 mg to 1,830 mg per dose, from 1,780 mg to 1,820 mg per dose, or from 1,790 mg to 1,810 mg per dose.

For example, in some embodiments, the oxytocin antagonist is administered to the subject in an amount of from 1,500 mg to 2,100 mg per dose, such as an amount of from 1,510 mg to 2,090 mg per dose, from 1,520 mg to 2,080 mg per dose, from 1,530 mg to 2,070 mg per dose, from 1,540 mg to 2,060 mg per dose, from 1,550 mg to 2,050 mg per dose, from 1,560 mg to 2,040 mg per dose, from 1,570 mg to 2,030 mg per dose, from 1,580 mg to 2,020 mg per dose, from 1,590 mg to 2,010 mg per dose, from 1,600 mg to 2,000 mg per dose, from 1,610 mg to 1,990 mg per dose, from 1,620 mg to 1,980 mg per dose, from 1,630 mg to 1,970 mg per dose, from 1,640 mg to 1,960 mg per dose, from 1,650 mg to 1,950 mg per dose, from 1,660 mg to 1,940 mg per dose, from 1,670 mg to 1,930 mg per dose, from 1,680 mg to 1,920 mg per dose, from 1,690 mg to 1,910 mg per dose, from 1,700 mg to 1,900 mg per dose, from 1,710 mg to 1,890 mg per dose, from 1,720 mg to 1,880 mg per dose, from 1,730 mg to 1,870 mg per dose, from 1,740 mg to 1,860 mg per dose, from 1,750 mg to 1,850 mg per dose, from 1,760 mg to 1,840 mg per dose, from 1,770 mg to 1,830 mg per dose, from 1,780 mg to 1,820 mg per dose, or from 1,790 mg to 1,810 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,501 mg to about 2,099 mg per dose, such as an amount of about 1,501 mg, 1,502 mg, 1,503 mg, 1,504 mg, 1,505 mg, 1,506 mg, 1,507 mg, 1,508 mg, 1,509 mg, 1,510 mg, 1,511 mg, 1,512 mg, 1,513 mg, 1,514 mg, 1,515 mg, 1,516 mg, 1,517 mg, 1,518 mg, 1,519 mg, 1,520 mg, 1,521 mg, 1,522 mg, 1,523 mg, 1,524 mg, 1,525 mg, 1,526 mg, 1,527 mg, 1,528 mg, 1,529 mg, 1,530 mg, 1,531 mg, 1,532 mg, 1,533 mg, 1,534 mg, 1,535 mg, 1,536 mg, 1,537 mg, 1,538 mg, 1,539 mg, 1,540 mg, 1,541 mg, 1,542 mg, 1,543 mg, 1,544 mg, 1,545 mg, 1,546 mg, 1,547 mg, 1,548 mg, 1,549 mg, 1,550 mg, 1,551 mg, 1,552 mg, 1,553 mg, 1,554 mg, 1,555 mg, 1,556 mg, 1,557 mg, 1,558 mg, 1,559 mg, 1,560 mg, 1,561 mg, 1,562 mg, 1,563 mg, 1,584 mg, 1,565 mg, 1,566 mg, 1,567 mg, 1,568 mg, 1,569 mg, 1,570 mg, 1,571 mg, 1,572 mg, 1,573 mg, 1,574 mg, 1,575 mg, 1,576 mg, 1,577 mg, 1,578 mg, 1,579 mg, 1,580 mg, 1,581 mg, 1,582 mg, 1,583 mg, 1,584 mg, 1,585 mg, 1,586 mg, 1,587 mg, 1,588 mg, 1,589 mg, 1,590 mg, 1,591 mg, 1,592 mg, 1,593 mg, 1,594 mg, 1,595 mg, 1,596 mg, 1,597 mg, 1,598 mg, 1,599 mg, 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, 2,000 mg, 2,001 mg, 2,002 mg, 2,003 mg, 2,004 mg, 2,005 mg, 2,006 mg, 2,007 mg, 2,008 mg, 2,009 mg, 2,010 mg, 2,011 mg, 2,012 mg, 2,013 mg, 2,014 mg, 2,015 mg, 2,016 mg, 2,017 mg, 2,018 mg, 2,019 mg, 2,020 mg, 2,021 mg, 2,022 mg, 2,023 mg, 2,024 mg, 2,025 mg, 2,026 mg, 2,027 mg, 2,028 mg, 2,029 mg, 2,030 mg, 2,031 mg, 2,032 mg, 2,033 mg, 2,034 mg, 2,035 mg, 2,036 mg, 2,037 mg, 2,038 mg, 2,039 mg, 2,040 mg, 2,041 mg, 2,042 mg, 2,043 mg, 2,044 mg, 2,045 mg, 2,046 mg, 2,047 mg, 2,048 mg, 2,049 mg, 2,050 mg, 2,051 mg, 2,052 mg, 2,053 mg, 2,054 mg, 2,055 mg, 2,056 mg, 2,057 mg, 2,058 mg, 2,059 mg, 2,060 mg, 2,061 mg, 2,062 mg, 2,063 mg, 2,064 mg, 2,065 mg, 2,066 mg, 2,067 mg, 2,068 mg, 2,069 mg, 2,070 mg, 2,071 mg, 2,072 mg, 2,073 mg, 2,074 mg, 2,075 mg, 2,076 mg, 2,077 mg, 2,078 mg, 2,079 mg, 2,080 mg, 2,081 mg, 2,082 mg, 2,083 mg, 2,084 mg, 2,085 mg, 2,086 mg, 2,087 mg, 2,088 mg, 2,089 mg, 2,090 mg, 2,091 mg, 2,092 mg, 2,093 mg, 2,094 mg, 2,095 mg, 2,096 mg, 2,097 mg, 2,098 mg, or 2,099 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,600 mg to about 2,000 mg per dose, such as an amount of about 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, or 2,000 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,700 mg to about 1,900 mg per dose, such as an amount of about 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, or 1,900 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,750 mg to about 1,850 mg per dose, such as an amount of about 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, or 1,850 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,760 mg to about 1,840 mg per dose, such as an amount of about 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, or 1,840 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (I)).

For example, in some embodiments, the oxytocin antagonist is administered to the subject in an amount of from about 1,770 mg to about 1,830 mg per dose, such as an amount of about 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, or 1,830 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (I)).

For example, in some embodiments, the oxytocin antagonist is administered to the subject in an amount of from about 1,780 mg to about 1,820 mg per dose, such as an amount of about 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, or 1,820 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in an amount of from about 1,790 mg to about 1,810 mg per dose, such as an amount of about 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, or 1,810 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in an amount of from about 2,100 mg to about 2,700 mg per dose, from about 2,150 mg to about 2,650 mg per dose, from about 2,200 mg to about 2,600 mg per dose, from about 2,250 mg to about 2,550 mg per dose, from about 2,300 mg to about 2,500 mg per dose, from about 2,350 mg to about 2,450 mg per dose, from about 2,360 mg to about 2,440 mg per dose, from about 2,370 mg to about 2,430 mg per dose, from about 2,380 mg to about 2,420 mg per dose, from about 2,390 mg to about 2,410 mg per dose, from about 2,391 mg to about 2,409 mg per dose, from about 2,392 mg to about 2,408 mg per dose, from about 2,393 mg to about 2,407 mg per dose, from about 2,394 mg to about 2,406 mg per dose, from about 2,395 mg to about 2,405 mg per dose, from about 2,396 mg to about 2,404 mg per dose, from about 2,397 mg to about 2,403 mg per dose, from about 2,398 mg to about 2,402 mg per dose, or from about 2,399 mg to about 2,401 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (I)).

For example, in some embodiments, the oxytocin antagonist is administered to the subject in an amount of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in an amount of about 1,800 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in an amount of about 2,100 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in an amount of about 2,400 mg per dose (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in one or more doses (e.g., in 1, 2, 3, 4, 5, 6, 7, 8, 9, 10, or more doses) totaling from 1,500 mg to 2,700 mg, such as in one or more doses (e.g., in 1, 2, 3, 4, 5, 6, 7, 8, 9, 10, or more doses) totaling from 1,500 mg to 2,100 mg, from 1,510 mg to 2,090 mg, from 1,520 mg to 2,080 mg, from 1,530 mg to 2,070 mg, from 1,540 mg to 2,060 mg, from 1,550 mg to 2,050 mg, from 1,560 mg to 2,040 mg, from 1,570 mg to 2,030 mg, from 1,580 mg to 2,020 mg, from 1,590 mg to 2,010 mg, from 1,600 mg to 2,000 mg, from 1,610 mg to 1,990 mg, from 1,620 mg to 1,980 mg, from 1,630 mg to 1,970 mg, from 1,640 mg to 1,960 mg, from 1,650 mg to 1,950 mg, from 1,660 mg to 1,940 mg, from 1,670 mg to 1,930 mg, from 1,680 mg to 1,920 mg, from 1,690 mg to 1,910 mg, from 1,700 mg to 1,900 mg, from 1,710 mg to 1,890 mg, from 1,720 mg to 1,880 mg, from 1,730 mg to 1,870 mg, from 1,740 mg to 1,860 mg, from 1,750 mg to 1,850 mg, from 1,760 mg to 1,840 mg, from 1,770 mg to 1,830 mg, from 1,780 mg to 1,820 mg, or from 1,790 mg to 1,810 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in in one or more doses (e.g., in 1, 2, 3, 4, 5, 6, 7, 8, 9, 10, or more doses) totaling from about 1,501 mg to about 2,099 mg, such as in one or more doses (e.g., in 1, 2, 3, 4, 5, 6, 7, 8, 9, 10, or more doses) totaling about 1,501 mg, 1,502 mg, 1,503 mg, 1,504 mg, 1,505 mg, 1,506 mg, 1,507 mg, 1,508 mg, 1,509 mg, 1,510 mg, 1,511 mg, 1,512 mg, 1,513 mg, 1,514 mg, 1,515 mg, 1,516 mg, 1,517 mg, 1,518 mg, 1,519 mg, 1,520 mg, 1,521 mg, 1,522 mg, 1,523 mg, 1,524 mg, 1,525 mg, 1,526 mg, 1,527 mg, 1,528 mg, 1,529 mg, 1,530 mg, 1,531 mg, 1,532 mg, 1,533 mg, 1,534 mg, 1,535 mg, 1,536 mg, 1,537 mg, 1,538 mg, 1,539 mg, 1,540 mg, 1,541 mg, 1,542 mg, 1,543 mg, 1,544 mg, 1,545 mg, 1,546 mg, 1,547 mg, 1,548 mg, 1,549 mg, 1,550 mg, 1,551 mg, 1,552 mg, 1,553 mg, 1,554 mg, 1,555 mg, 1,556 mg, 1,557 mg, 1,558 mg, 1,559 mg, 1,560 mg, 1,561 mg, 1,562 mg, 1,563 mg, 1,564 mg, 1,565 mg, 1,566 mg, 1,567 mg, 1,568 mg, 1,569 mg, 1,570 mg, 1,571 mg, 1,572 mg, 1,573 mg, 1,574 mg, 1,575 mg, 1,576 mg, 1,577 mg, 1,578 mg, 1,579 mg, 1,580 mg, 1,581 mg, 1,582 mg, 1,583 mg, 1,584 mg, 1,585 mg, 1,586 mg, 1,587 mg, 1,588 mg, 1,589 mg, 1,590 mg, 1,591 mg, 1,592 mg, 1,593 mg, 1,594 mg, 1,595 mg, 1,596 mg, 1,597 mg, 1,598 mg, 1,599 mg, 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, 2,000 mg, 2,001 mg, 2,002 mg, 2,003 mg, 2,004 mg, 2,005 mg, 2,006 mg, 2,007 mg, 2,008 mg, 2,009 mg, 2,010 mg, 2,011 mg, 2,012 mg, 2,013 mg, 2,014 mg, 2,015 mg, 2,016 mg, 2,017 mg, 2,018 mg, 2,019 mg, 2,020 mg, 2,021 mg, 2,022 mg, 2,023 mg, 2,024 mg, 2,025 mg, 2,026 mg, 2,027 mg, 2,028 mg, 2,029 mg, 2,030 mg, 2,031 mg, 2,032 mg, 2,033 mg, 2,034 mg, 2,035 mg, 2,036 mg, 2,037 mg, 2,038 mg, 2,039 mg, 2,040 mg, 2,041 mg, 2,042 mg, 2,043 mg, 2,044 mg, 2,045 mg, 2,046 mg, 2,047 mg, 2,048 mg, 2,049 mg, 2,050 mg, 2,051 mg, 2,052 mg, 2,053 mg, 2,054 mg, 2,055 mg, 2,056 mg, 2,057 mg, 2,058 mg, 2,059 mg, 2,060 mg, 2,061 mg, 2,062 mg, 2,063 mg, 2,064 mg, 2,065 mg, 2,066 mg, 2,067 mg, 2,068 mg, 2,069 mg, 2,070 mg, 2,071 mg, 2,072 mg, 2,073 mg, 2,074 mg, 2,075 mg, 2,076 mg, 2,077 mg, 2,078 mg, 2,079 mg, 2,080 mg, 2,081 mg, 2,082 mg, 2,083 mg, 2,084 mg, 2,085 mg, 2,086 mg, 2,087 mg, 2,088 mg, 2,089 mg, 2,090 mg, 2,091 mg, 2,092 mg, 2,093 mg, 2,094 mg, 2,095 mg, 2,096 mg, 2,097 mg, 2,098 mg, or 2,099 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (I)).

For example, in some embodiments, the oxytocin antagonist is administered to the subject in one or more doses (e.g., in 1, 2, 3, 4, 5, 6, 7, 8, 9, 10, or more doses) totaling from about 1,600 mg to about 2,000 mg, such as in one or more doses (e.g., in 1, 2, 3, 4, 5, 6, 7, 8, 9, 10, or more doses) totaling about 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, or 2,000 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in one or more doses (e.g., in 1, 2, 3, 4, 5, 6, 7, 8, 9, 10, or more doses) totaling from about 1,700 mg to about 1,900 mg, such as in one or more doses (e.g., in 1, 2, 3, 4, 5, 6, 7, 8, 9, 10, or more doses) totaling about 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, or 1,900 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in in one or more doses (e.g., in 1, 2, 3, 4, 5, 6, 7, 8, 9, 10, or more doses) totaling from about 1,750 mg to about 1,850 mg, such as an amount of about 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, or 1,850 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in one or more doses (e.g., in 1, 2, 3, 4, 5, 6, 7, 8, 9, 10, or more doses) totaling from about 1,760 mg to about 1,840 mg, such as in one or more doses (e.g., in 1, 2, 3, 4, 5, 6, 7, 8, 9, 10, or more doses) totaling about 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, or 1,840 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in one or more doses (e.g., in 1, 2, 3, 4, 5, 6, 7, 8, 9, 10, or more doses) totaling from about 1,770 mg to about 1,830 mg, such as an amount of about 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, or 1,830 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in one or more doses (e.g., in 1, 2, 3, 4, 5, 6, 7, 8, 9, 10, or more doses) totaling from about 1,780 mg to about 1,820 mg, such as an amount of about 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, or 1,820 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in one or more doses (e.g., in 1, 2, 3, 4, 5, 6, 7, 8, 9, 10, or more doses) totaling from about 1,790 mg to about 1,810 mg, such as an amount of about 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, or 1,810 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in one or more doses (e.g., in 1, 2, 3, 4, 5, 6, 7, 8, 9, 10, or more doses) totaling from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in one or more doses (e.g., in 1, 2, 3, 4, 5, 6, 7, 8, 9, 10, or more doses) totaling about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in one or more doses (e.g., in 1, 2, 3, 4, 5, 6, 7, 8, 9, 10, or more doses) totaling about 1,800 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in one or more doses (e.g., in 1, 2, 3, 4, 5, 6, 7, 8, 9, 10, or more doses) totaling about 2,100 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in one or more doses (e.g., in 1, 2, 3, 4, 5, 6, 7, 8, 9, 10, or more doses) totaling about 2,400 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in a single dose (e.g., on the day of the embryo transfer therapy) of from 1,500 mg to 2,700 mg, such as in a single dose (e.g., on the day of the embryo transfer therapy) of from 1,500 mg to 2,100 mg, from 1,510 mg to 2,090 mg, from 1,520 mg to 2,080 mg, from 1,530 mg to 2,070 mg, from 1,540 mg to 2,060 mg, from 1,550 mg to 2,050 mg, from 1,560 mg to 2,040 mg, from 1,570 mg to 2,030 mg, from 1,580 mg to 2,020 mg, from 1,590 mg to 2,010 mg, from 1,600 mg to 2,000 mg, from 1,610 mg to 1,990 mg, from 1,620 mg to 1,980 mg, from 1,630 mg to 1,970 mg, from 1,640 mg to 1,960 mg, from 1,650 mg to 1,950 mg, from 1,660 mg to 1,940 mg, from 1,670 mg to 1,930 mg, from 1,680 mg to 1,920 mg, from 1,690 mg to 1,910 mg, from 1,700 mg to 1,900 mg, from 1,710 mg to 1,890 mg, from 1,720 mg to 1,880 mg, from 1,730 mg to 1,870 mg, from 1,740 mg to 1,860 mg, from 1,750 mg to 1,850 mg, from 1,760 mg to 1,840 mg, from 1,770 mg to 1,830 mg, from 1,780 mg to 1,820 mg, or from 1,790 mg to 1,810 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,501 mg to about 2,099 mg, such as in a single dose (e.g., on the day of the embryo transfer therapy) of about 1,501 mg, 1,502 mg, 1,503 mg, 1,504 mg, 1,505 mg, 1,506 mg, 1,507 mg, 1,508 mg, 1,509 mg, 1,510 mg, 1,511 mg, 1,512 mg, 1,513 mg, 1,514 mg, 1,515 mg, 1,516 mg, 1,517 mg, 1,518 mg, 1,519 mg, 1,520 mg, 1,521 mg, 1,522 mg, 1,523 mg, 1,524 mg, 1,525 mg, 1,526 mg, 1,527 mg, 1,528 mg, 1,529 mg, 1,530 mg, 1,531 mg, 1,532 mg, 1,533 mg, 1,534 mg, 1,535 mg, 1,536 mg, 1,537 mg, 1,538 mg, 1,539 mg, 1,540 mg, 1,541 mg, 1,542 mg, 1,543 mg, 1,544 mg, 1,545 mg, 1,546 mg, 1,547 mg, 1,548 mg, 1,549 mg, 1,550 mg, 1,551 mg, 1,552 mg, 1,553 mg, 1,554 mg, 1,555 mg, 1,556 mg, 1,557 mg, 1,558 mg, 1,559 mg, 1,560 mg, 1,561 mg, 1,562 mg, 1,563 mg, 1,564 mg, 1,565 mg, 1,566 mg, 1,567 mg, 1,568 mg, 1,569 mg, 1,570 mg, 1,571 mg, 1,572 mg, 1,573 mg, 1,574 mg, 1,575 mg, 1,576 mg, 1,577 mg, 1,578 mg, 1,579 mg, 1,580 mg, 1,581 mg, 1,582 mg, 1,583 mg, 1,584 mg, 1,585 mg, 1,586 mg, 1,587 mg, 1,588 mg, 1,589 mg, 1,590 mg, 1,591 mg, 1,592 mg, 1,593 mg, 1,594 mg, 1,595 mg, 1,596 mg, 1,597 mg, 1,598 mg, 1,599 mg, 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, 2,000 mg, 2,001 mg, 2,002 mg, 2,003 mg, 2,004 mg, 2,005 mg, 2,006 mg, 2,007 mg, 2,008 mg, 2,009 mg, 2,010 mg, 2,011 mg, 2,012 mg, 2,013 mg, 2,014 mg, 2,015 mg, 2,016 mg, 2,017 mg, 2,018 mg, 2,019 mg, 2,020 mg, 2,021 mg, 2,022 mg, 2,023 mg, 2,024 mg, 2,025 mg, 2,026 mg, 2,027 mg, 2,028 mg, 2,029 mg, 2,030 mg, 2,031 mg, 2,032 mg, 2,033 mg, 2,034 mg, 2,035 mg, 2,036 mg, 2,037 mg, 2,038 mg, 2,039 mg, 2,040 mg, 2,041 mg, 2,042 mg, 2,043 mg, 2,044 mg, 2,045 mg, 2,046 mg, 2,047 mg, 2,048 mg, 2,049 mg, 2,050 mg, 2,051 mg, 2,052 mg, 2,053 mg, 2,054 mg, 2,055 mg, 2,056 mg, 2,057 mg, 2,058 mg, 2,059 mg, 2,060 mg, 2,061 mg, 2,062 mg, 2,063 mg, 2,064 mg, 2,065 mg, 2,066 mg, 2,067 mg, 2,068 mg, 2,069 mg, 2,070 mg, 2,071 mg, 2,072 mg, 2,073 mg, 2,074 mg, 2,075 mg, 2,076 mg, 2,077 mg, 2,078 mg, 2,079 mg, 2,080 mg, 2,081 mg, 2,082 mg, 2,083 mg, 2,084 mg, 2,085 mg, 2,086 mg, 2,087 mg, 2,088 mg, 2,089 mg, 2,090 mg, 2,091 mg, 2,092 mg, 2,093 mg, 2,094 mg, 2,095 mg, 2,096 mg, 2,097 mg, 2,098 mg, or 2,099 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,600 mg to about 2,000 mg, such as in a single dose (e.g., on the day of the embryo transfer therapy) of about 1,600 mg, 1,601 mg, 1,602 mg, 1,603 mg, 1,604 mg, 1,605 mg, 1,606 mg, 1,607 mg, 1,608 mg, 1,609 mg, 1,610 mg, 1,611 mg, 1,612 mg, 1,613 mg, 1,614 mg, 1,615 mg, 1,616 mg, 1,617 mg, 1,618 mg, 1,619 mg, 1,620 mg, 1,621 mg, 1,622 mg, 1,623 mg, 1,624 mg, 1,625 mg, 1,626 mg, 1,627 mg, 1,628 mg, 1,629 mg, 1,630 mg, 1,631 mg, 1,632 mg, 1,633 mg, 1,634 mg, 1,635 mg, 1,636 mg, 1,637 mg, 1,638 mg, 1,639 mg, 1,640 mg, 1,641 mg, 1,642 mg, 1,643 mg, 1,644 mg, 1,645 mg, 1,646 mg, 1,647 mg, 1,648 mg, 1,649 mg, 1,650 mg, 1,651 mg, 1,652 mg, 1,653 mg, 1,654 mg, 1,655 mg, 1,656 mg, 1,657 mg, 1,658 mg, 1,659 mg, 1,660 mg, 1,661 mg, 1,662 mg, 1,663 mg, 1,664 mg, 1,665 mg, 1,666 mg, 1,667 mg, 1,668 mg, 1,669 mg, 1,670 mg, 1,671 mg, 1,672 mg, 1,673 mg, 1,674 mg, 1,675 mg, 1,676 mg, 1,677 mg, 1,678 mg, 1,679 mg, 1,680 mg, 1,681 mg, 1,682 mg, 1,683 mg, 1,684 mg, 1,685 mg, 1,686 mg, 1,687 mg, 1,688 mg, 1,689 mg, 1,690 mg, 1,691 mg, 1,692 mg, 1,693 mg, 1,694 mg, 1,695 mg, 1,696 mg, 1,697 mg, 1,698 mg, 1,699 mg, 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, 1,900 mg, 1,901 mg, 1,902 mg, 1,903 mg, 1,904 mg, 1,905 mg, 1,906 mg, 1,907 mg, 1,908 mg, 1,909 mg, 1,910 mg, 1,911 mg, 1,912 mg, 1,913 mg, 1,914 mg, 1,915 mg, 1,916 mg, 1,917 mg, 1,918 mg, 1,919 mg, 1,920 mg, 1,921 mg, 1,922 mg, 1,923 mg, 1,924 mg, 1,925 mg, 1,926 mg, 1,927 mg, 1,928 mg, 1,929 mg, 1,930 mg, 1,931 mg, 1,932 mg, 1,933 mg, 1,934 mg, 1,935 mg, 1,936 mg, 1,937 mg, 1,938 mg, 1,939 mg, 1,940 mg, 1,941 mg, 1,942 mg, 1,943 mg, 1,944 mg, 1,945 mg, 1,946 mg, 1,947 mg, 1,948 mg, 1,949 mg, 1,950 mg, 1,951 mg, 1,952 mg, 1,953 mg, 1,954 mg, 1,955 mg, 1,956 mg, 1,957 mg, 1,958 mg, 1,959 mg, 1,960 mg, 1,961 mg, 1,962 mg, 1,963 mg, 1,964 mg, 1,965 mg, 1,966 mg, 1,967 mg, 1,968 mg, 1,969 mg, 1,970 mg, 1,971 mg, 1,972 mg, 1,973 mg, 1,974 mg, 1,975 mg, 1,976 mg, 1,977 mg, 1,978 mg, 1,979 mg, 1,980 mg, 1,981 mg, 1,982 mg, 1,983 mg, 1,984 mg, 1,985 mg, 1,986 mg, 1,987 mg, 1,988 mg, 1,989 mg, 1,990 mg, 1,991 mg, 1,992 mg, 1,993 mg, 1,994 mg, 1,995 mg, 1,996 mg, 1,997 mg, 1,998 mg, 1,999 mg, or 2,000 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,700 mg to about 1,900 mg, such as in a single dose (e.g., on the day of the embryo transfer therapy) of about 1,700 mg, 1,701 mg, 1,702 mg, 1,703 mg, 1,704 mg, 1,705 mg, 1,706 mg, 1,707 mg, 1,708 mg, 1,709 mg, 1,710 mg, 1,711 mg, 1,712 mg, 1,713 mg, 1,714 mg, 1,715 mg, 1,716 mg, 1,717 mg, 1,718 mg, 1,719 mg, 1,720 mg, 1,721 mg, 1,722 mg, 1,723 mg, 1,724 mg, 1,725 mg, 1,726 mg, 1,727 mg, 1,728 mg, 1,729 mg, 1,730 mg, 1,731 mg, 1,732 mg, 1,733 mg, 1,734 mg, 1,735 mg, 1,736 mg, 1,737 mg, 1,738 mg, 1,739 mg, 1,740 mg, 1,741 mg, 1,742 mg, 1,743 mg, 1,744 mg, 1,745 mg, 1,746 mg, 1,747 mg, 1,748 mg, 1,749 mg, 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, 1,850 mg, 1,851 mg, 1,852 mg, 1,853 mg, 1,854 mg, 1,855 mg, 1,856 mg, 1,857 mg, 1,858 mg, 1,859 mg, 1,860 mg, 1,861 mg, 1,862 mg, 1,863 mg, 1,864 mg, 1,865 mg, 1,866 mg, 1,867 mg, 1,868 mg, 1,869 mg, 1,870 mg, 1,871 mg, 1,872 mg, 1,873 mg, 1,874 mg, 1,875 mg, 1,876 mg, 1,877 mg, 1,878 mg, 1,879 mg, 1,880 mg, 1,881 mg, 1,882 mg, 1,883 mg, 1,884 mg, 1,885 mg, 1,886 mg, 1,887 mg, 1,888 mg, 1,889 mg, 1,890 mg, 1,891 mg, 1,892 mg, 1,893 mg, 1,894 mg, 1,895 mg, 1,896 mg, 1,897 mg, 1,898 mg, 1,899 mg, or 1,900 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,750 mg to about 1,850 mg, such as in a single dose (e.g., on the day of the embryo transfer therapy) of about 1,750 mg, 1,751 mg, 1,752 mg, 1,753 mg, 1,754 mg, 1,755 mg, 1,756 mg, 1,757 mg, 1,758 mg, 1,759 mg, 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, 1,840 mg, 1,841 mg, 1,842 mg, 1,843 mg, 1,844 mg, 1,845 mg, 1,846 mg, 1,847 mg, 1,848 mg, 1,849 mg, or 1,850 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (I)).

For example, in some embodiments, the oxytocin antagonist is administered to the subject in a single dose (e.g., on the day of the embryo transfer therapy) of from about 1,760 mg to about 1,840 mg, such as in a single dose (e.g., on the day of the embryo transfer therapy) of about 1,760 mg, 1,761 mg, 1,762 mg, 1,763 mg, 1,764 mg, 1,765 mg, 1,766 mg, 1,767 mg, 1,768 mg, 1,769 mg, 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, 1,830 mg, 1,831 mg, 1,832 mg, 1,833 mg, 1,834 mg, 1,835 mg, 1,836 mg, 1,837 mg, 1,838 mg, 1,839 mg, or 1,840 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,770 mg to about 1,830 mg, such as in a single dose (e.g., on the day of the embryo transfer therapy) of about 1,770 mg, 1,771 mg, 1,772 mg, 1,773 mg, 1,774 mg, 1,775 mg, 1,776 mg, 1,777 mg, 1,778 mg, 1,779 mg, 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, 1,820 mg, 1,821 mg, 1,822 mg, 1,823 mg, 1,824 mg, 1,825 mg, 1,826 mg, 1,827 mg, 1,828 mg, 1,829 mg, or 1,830 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of about 1,780 mg to about 1,820 mg, such as in a single dose (e.g., on the day of the embryo transfer therapy) of about 1,780 mg, 1,781 mg, 1,782 mg, 1,783 mg, 1,784 mg, 1,785 mg, 1,786 mg, 1,787 mg, 1,788 mg, 1,789 mg, 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, 1,810 mg, 1,811 mg, 1,812 mg, 1,813 mg, 1,814 mg, 1,815 mg, 1,816 mg, 1,817 mg, 1,818 mg, 1,819 mg, or 1,820 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

For example, in some embodiments, the oxytocin antagonist is administered to the subject in a single dose (e.g., on the day of the embryo transfer therapy) of from about 1,790 mg to about 1,810 mg, such as in a single dose (e.g., on the day of the embryo transfer therapy) of about 1,790 mg, 1,791 mg, 1,792 mg, 1,793 mg, 1,794 mg, 1,795 mg, 1,796 mg, 1,797 mg, 1,798 mg, 1,799 mg, 1,800 mg, 1,801 mg, 1,802 mg, 1,803 mg, 1,804 mg, 1,805 mg, 1,806 mg, 1,807 mg, 1,808 mg, 1,809 mg, or 1,810 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in a single dose (e.g., on the day of the embryo transfer therapy) of from about 2,100 mg to about 2,700 mg, from about 2,150 mg to about 2,650 mg, from about 2,200 mg to about 2,600 mg, from about 2,250 mg to about 2,550 mg, from about 2,300 mg to about 2,500 mg, from about 2,350 mg to about 2,450 mg, from about 2,360 mg to about 2,440 mg, from about 2,370 mg to about 2,430 mg, from about 2,380 mg to about 2,420 mg, from about 2,390 mg to about 2,410 mg, from about 2,391 mg to about 2,409 mg, from about 2,392 mg to about 2,408 mg, from about 2,393 mg to about 2,407 mg, from about 2,394 mg to about 2,406 mg, from about 2,395 mg to about 2,405 mg, from about 2,396 mg to about 2,404 mg, from about 2,397 mg to about 2,403 mg, from about 2,398 mg to about 2,402 mg, or from about 2,399 mg to about 2,401 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (I)).

For example, in some embodiments, the oxytocin antagonist is administered to the subject in a single dose (e.g., on the day of the embryo transfer therapy) of about 2,100 mg, 2,110 mg, 2,120 mg, 2,130 mg, 2,140 mg, 2,150 mg, 2,160 mg, 2,170 mg, 2,180 mg, 2,190 mg, 2,200 mg, 2,210 mg, 2,220 mg, 2,230 mg, 2,240 mg, 2,250 mg, 2,260 mg, 2,270 mg, 2,280 mg, 2,290 mg, 2,300 mg, 2,310 mg, 2,320 mg, 2,330 mg, 2,340 mg, 2,350 mg, 2,360 mg, 2,370 mg, 2,380 mg, 2,390 mg, 2,400 mg, 2,410 mg, 2,420 mg, 2,430 mg, 2,440 mg, 2,450 mg, 2,460 mg, 2,470 mg, 2,480 mg, 2,490 mg, 2,500 mg, 2,510 mg, 2,520 mg, 2,530 mg, 2,540 mg, 2,550 mg, 2,560 mg, 2,570 mg, 2,580 mg, 2,590 mg, 2,600 mg, 2,610 mg, 2,620 mg, 2,630 mg, 2,640 mg, 2,650 mg, 2,660 mg, 2,670 mg, 2,680 mg, 2,690 mg, or 2,700 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in a single dose (e.g., on the day of the embryo transfer therapy) of about 1,800 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in a single dose (e.g., on the day of the embryo transfer therapy) of about 2,100 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

In some embodiments, the oxytocin antagonist is administered to the subject in a single dose (e.g., on the day of the embryo transfer therapy) of about 2,400 mg (e.g., wherein the oxytocin antagonist is (3Z,5S)-5-(hydroxymethyl)-1-[(2′-methyl-1,1′-biphenyl-4-yl)carbonyl]pyrrolidin-3-one O-methyloxime, represented by formula (II)).

### Administration Beginning During Embryo Transfer Therapy

Compounds of formula (I) or (II) or another oxytocin antagonist described herein, such as epelsiban, retosiban, barusiban, and atosiban, or a salt, derivative, variant, crystal form, or formulation thereof, may be administered to a subject during embryo transfer, such as within about 60 minutes or less of transfer of the embryo to the uterus of the subject. In such instances, the compound may be administered to the subject in a single dose, such as in a single dose of from about 1,500 mg to about 2,700 mg as described herein (e.g., a single dose of about 1,800 mg, about 2,100 mg, or about 2,400 mg of the compound of formula (I) or (11)) or in multiple doses of lower strength totaling from about 1,500 mg to about 2,700 mg, such as about 1,800 mg, about 2,100 mg, or about 2,400 mg. A single dose of the compound can be administered, for instance, at the initiation of the embryo transfer procedure. For example, a compound of formula (I) or (II) may be administered to the subject upon entrance of an embryo delivery device, such as a catheter containing the one or more embryos to be transferred to the subject, into the vaginal canal of the subject. Additionally or alternatively, the compound may be administered to the subject upon entrance of the embryo delivery device beyond the cervix and into the uterus of the subject. The compound may be administered to the subject upon expulsion of the one or more embryos to be transferred from the embryo delivery device, and/or upon removal of the embryo delivery device from the uterus or vaginal canal of the subject. In some embodiments, multiple doses of the compound are administered throughout the duration of the embryo transfer process. Compounds of formula (I) or (II) may be administered continuously throughout the embryo transfer process, for instance, by continuous intravenous administration.

Dosing of the oxytocin antagonist that has begun during embryo transfer (e.g., within 60 minutes or less of the embryo transfer) may continue following embryo transfer. For instance, the compound may be administered to the subject in one or more additional doses following embryo transfer, for instance, in multiple repeat doses or doses of varying strength. The compound may be administered to the subject in one or more additional doses administered within, for instance, from about 1 hour to about 1 week, or longer (e.g., within about 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, 96 hours, 108 hours, 120 hours, 132 hours, 144 hours, 156 hours, 168 hours, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more) following the transfer of the one or more embryos to the subject. When multiple doses of compound (1) or compound (II) are administered to a subject following embryo transfer, the subject may be administered the additional doses, for instance, in regular intervals. Compounds of formula (I) or formula (II) may be administered to the subject following embryo transfer therapy, for example, in from 1 to 20 additional doses per day, per week, per month, or longer. For instance, the compound may be administered to the subject following embryo transfer in up to 7 doses (e.g., 1, 2, 3, 4, 5, 6, or 7 doses) per 24 hours.

### Administration Beginning Following Embryo Transfer Therapy

Dosing of the oxytocin antagonist (e.g., a compound of formula (I) or (II) or another oxytocin antagonist described herein, such as epelsiban, retosiban, barusiban, and atosiban, or a salt, derivative, variant, crystal form, or formulation thereof) may begin following the completion of the embryo transfer process. For instance, the compound of formula (I) or (II) may be administered to the subject following embryo transfer in a single dose, such as a single dose of from about 1,500 mg to about 2,700 mg (e.g., a single dose of about 1,800 mg, about 2,100 mg, or about 2,400 mg of the compound of formula (I) or (11)) or in multiple doses of lower strength, such as two or more doses of lower strength totaling from about 1,500 mg to about 2,700 mg, (e.g., totaling about 1,800 mg, about 2,100 mg, or about 2,400 mg).

The compound may be administered to the subject in one or more doses administered within, for instance, from about 1 hour to about 1 week, or longer (e.g., within about 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, 96 hours, 108 hours, 120 hours, 132 hours, 144 hours, 156 hours, 168 hours, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more) following the transfer of the one or more embryos to the subject. When multiple doses of compound (1) or compound (II) are administered to a subject following embryo transfer, the subject may be administered the doses, for instance, in regular intervals. Compounds of formula (I) or formula (II) may be administered to the subject following embryo transfer therapy, for example, in from 1 to 20 doses per day, per week, per month, or longer. For instance, the compound may be administered to the subject following embryo transfer in up to 7 doses (e.g., 1, 2, 3, 4, 5, 6, or 7 doses) per 24 hours.

### Additional Oxytocin Antagonists

In addition to compounds of formula (I) and (II), oxytocin antagonists that may be used in conjunction with the compositions and methods described herein include epelsiban, retosiban, barusiban, and atosiban, as well as salts, derivative, variants, crystal forms, and formulations thereof. The sections that follow provide a description of these agents, as well as synthetic methods for the preparation of these oxytocin antagonists.

### Epelsiban

Oxytocin antagonists useful in conjunction with the compositions and methods described herein include epelsiban ((3R,6R)-3-(2,3-dihydro-1H-inden-2-yl)-1-[(1R)-1-(2,6-dimethyl-3-pyridinyl)-2-(4-morpholinyl)-2-oxoethyl]-6-[(1S)-1-methylpropyl]-2,5-piperazinedione), as well as salts, derivatives, variants, crystal forms, and formulations thereof, such as a salt, derivative, variant, crystal form, or formulation described in U.S. Pat. Nos. 7,514,437; 8,367,673; 8,541,579; 7,550,462; 7,919,492; 8,202,864; 8,742,099; 9,408,851; 8,716,286; or 8,815,856, the disclosures of each of which are incorporated herein by reference in their entirety. Epelsiban is shown graphically in structural formula (III), below.

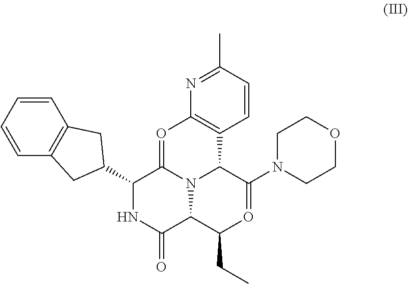

Exemplary methods for the preparation of epelsiban are described, for instance, in U.S. Pat. No. 8,742,099, and are depicted in Scheme 2, below.

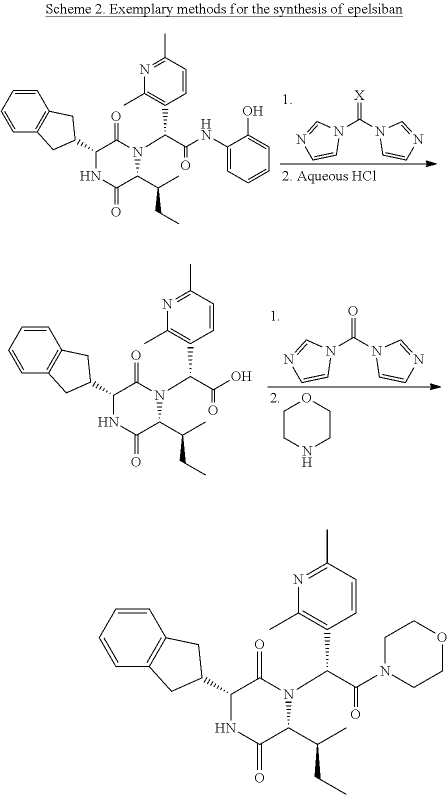

wherein X represents oxygen or sulfur. It is to be understood that the foregoing compound can be synthesized by alternative methods, for instance, by substituting one of the amide-bond forming agents shown in the foregoing scheme with another amide-bond forming agent described herein or known in the art.

### Retosiban

Oxytocin antagonists useful in conjunction with the compositions and methods described herein include retosiban ((3R,6R)-3-(2,3-dihydro-1H-inden-2-yl)-1-[(1R)-1-(2-methyl-1,3-oxazol-4-yl)-2-(4-morpholinyl)-2-oxoethyl]-8-[(1S)-1-methylpropyl]-2,5-piperazinedione), as well as salts, derivatives, variants, crystal forms, and formulations thereof, such as a salt, derivative, variant, crystal form, or formulation described in U.S. Pat. Nos. 7,514,437; 8,367,673; 8,541,579; 8,071,594; 8,357,685; 8,937,179; or 9,452,169, the disclosures of each of which are incorporated herein by reference in their entirety. Retosiban is shown graphically in structural formula (IV), below.

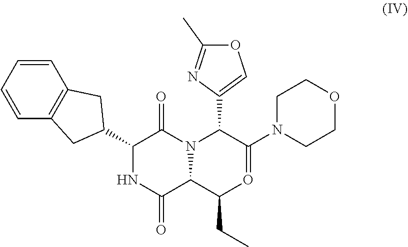

Exemplary methods for the preparation of retosiban are described, for instance, in U.S. Pat. No. 8,937,139, and are depicted in Scheme 3, below.

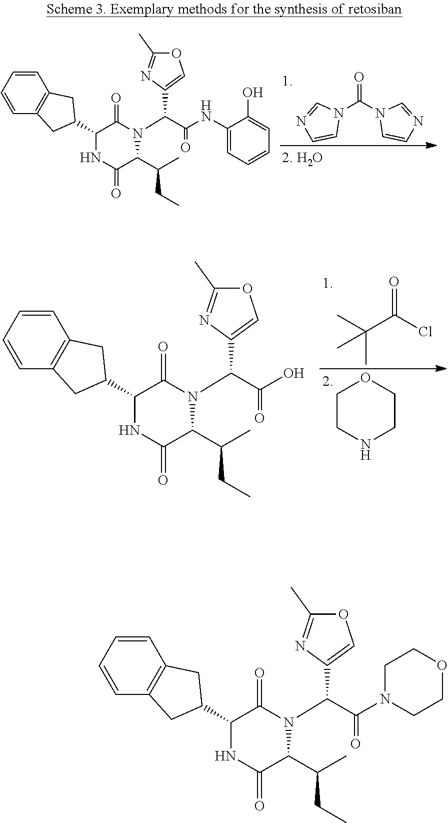

It is to be understood that the foregoing compound can be synthesized by alternative methods, for instance, by substituting one of the amide-bond forming agents shown in the foregoing scheme with another amide-bond forming agent described herein or known in the art.

### Barusiban

Oxytocin antagonists useful in conjunction with the compositions and methods described herein include barusiban, as well as salts, derivatives, variants, crystal forms, and formulations thereof, such as a salt, derivative, variant, crystal form, or formulation described in U.S. Pat. Nos. 6,143,722; 7,091,314; 7,816,489; or 9,579,305, or WO 2017/060339, the disclosures of each of which are incorporated herein by reference in their entirety. Barusiban is shown graphically in structural formula (V), below.

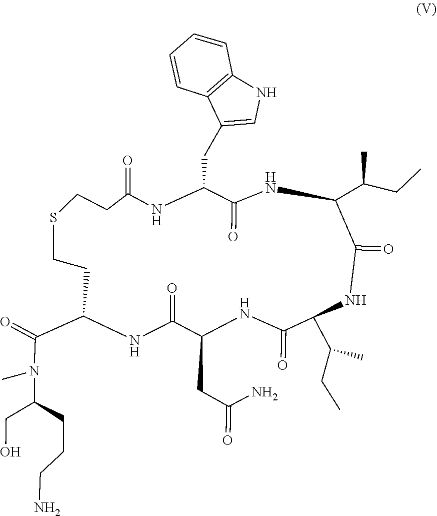 [SMILES: CC[C@@H](C)[C@@H]1NC(=O)[C@H]([C@@H](C)CC)NC(=O)[C@@H](Cc2c[nH]c3ccccc23)NC(=O)CCSCC[C@@H](C(=O)N(C)[C@H](CO)CCCN)NC(=O)[C@H](CC(N)=O)NC1=O]

Exemplary methods for the preparation of barusiban are described, for instance, in WO 2017/060339, and may involve solid-phase peptide synthesis as well as solution-phase cyclization, for instance, by thioetherification. It is to be understood that the foregoing compound can be synthesized by alternative methods, for instance, by substituting one of the amide-bond forming agents shown WO 2017/060339 with another amide-bond forming agent described herein or known in the art.

### Atosiban

Oxytocin antagonists useful in conjunction with the compositions and methods described herein include atosiban, as well as salts, derivatives, variants, crystal forms, and formulations thereof, such as a salt, derivative, variant, crystal form, or formulation described in U.S. Pat. No. 4,504,469 or 4,402,942, the disclosures of each of which are incorporated herein by reference in their entirety. Atosiban is shown graphically in structural formula (VI), below.

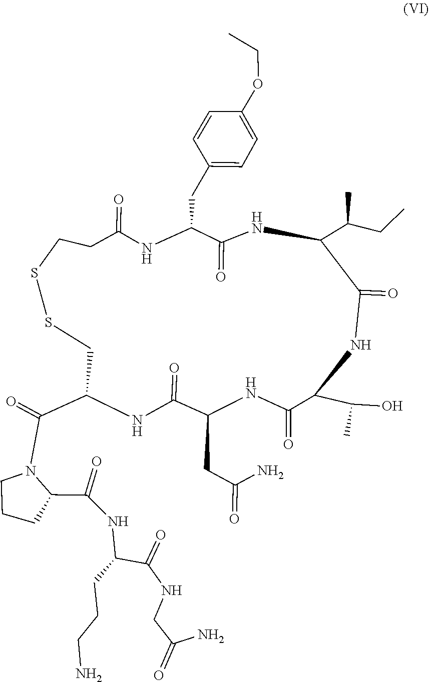 [SMILES: CCOc1ccc(C[C@H]2NC(=O)CCSSC[C@@H](C(=O)N3CCC[C@H]3C(=O)N[C@@H](CCCN)C(=O)NCC(N)=O)NC(=O)[C@H](CC(N)=O)NC(=O)[C@H]([C@@H](C)O)NC(=O)[C@H]([C@@H](C)CC)NC2=O)cc1]

Exemplary methods for the preparation of atosiban are described, for instance, in U.S. Pat. Nos. 4,504,469 and 4,402,942, and may involve solid-phase peptide synthesis as well as solution-phase cyclization, for instance, by disulfide bond formation. It is to be understood that the foregoing compound can be synthesized by alternative methods, for instance, by substituting one of the amide-bond forming agents shown U.S. Pat. No. 4,504,469 or 4,402,942 with another amide-bond forming agent described herein or known in the art.

### Methods of Assessing Serum Progesterone Levels

Using the compositions and methods described herein, one of skill in the art can assess the likelihood that a subject (e.g., a human subject) undergoing embryo transfer therapy will benefit from oxytocin antagonist treatment by comparing the serum progesterone concentration of a subject to a progesterone reference level. For instance, a physician of skill in the art can withdraw a sample from a subject undergoing embryo transfer therapy at one of multiple time points during an assisted reproductive technology process. Upon comparing the subject's serum progesterone concentration to that of an appropriate progesterone reference level, a determination that the subject exhibits a reduced serum progesterone concentration relative to the progesterone reference level indicates that the subject is particularly well suited for, and likely to benefit from (e.g., likely to exhibit enhanced endometrial receptivity in response to) treatment with an oxytocin antagonist, such as a compound of formula (I) or (II), or another oxytocin antagonist described herein or known in the art, such as epelsiban, retosiban, barusiban, and atosiban, prior to, concurrently with, and/or following the transfer of one or more embryos to the uterus of the subject.

For example, the sample may be withdrawn from the subject on the day of oocyte or ovum retrieval in the case of a subject using an autologous gamete for the ex vivo production of an embryo. In such instances, the progesterone reference level may be from 1.0 ng/ml to 2.0 ng/ml, such as 1.0 ng/ml, 1.1 ng/ml, 1.2 ng/ml, 1.3 ng/ml, 1.4 ng/ml, 1.5 ng/ml, 1.6 ng/ml, 1.7 ng/ml, 1.8 ng/ml, 1.9 ng/ml, or 2.0 ng/ml. The progesterone reference level may be, for instance, 1.5 ng/ml in such instances. The physician may then compare the progesterone level in the sample (e.g., serum sample) isolated from the subject to that of the progesterone reference level. A determination that the subject exhibits a reduced serum progesterone concentration relative to the progesterone reference level indicates that the subject is particularly well suited for, and likely to benefit from (e.g., likely to exhibit enhanced endometrial receptivity in response to), treatment with an oxytocin antagonist.

Additionally or alternatively, the sample may be withdrawn from the subject on the day of the embryo transfer procedure (e.g., following oocyte or ovum retrieval in the case of a subject using an autologous gamete for the ex vivo production of an embryo). In such instances, the progesterone reference level may be from 200 nM to 300 nM or more, such as 320 nM. The physician may then compare the progesterone level in the sample (e.g., serum sample) isolated from the subject to that of the progesterone reference level. A determination that the subject exhibits a reduced serum progesterone concentration relative to the progesterone reference level indicates that the subject is particularly well suited for, and likely to benefit from (e.g., likely to exhibit enhanced endometrial receptivity in response to), treatment with an oxytocin antagonist.

Methods of quantitating the concentration of progesterone in a sample (e.g., serum sample) isolated from a subject are known in the art and include, for instance, competitive enzyme-linked immunosorbant assays (ELISA), such as those described in U.S. Pat. No. 9,201,077, the disclosure of which is incorporated herein by reference in its entirety. Antibodies capable of specifically binding to progesterone and that may be used in conjunction with progesterone detection assays include those produced and released by ATCC Accession Number HB 8886 as described in U.S. Pat. No. 4,720,455, the disclosure of which is incorporated herein by reference in its entirety.

### Follicular Maturation and Oocyte/Ovum Retrieval

A variety of methods can be used in order to induce follicular maturation and to perform oocyte (e.g., mature oocyte) retrieval in conjunction with the compositions and methods described herein. In some embodiments, ova or oocytes are isolated from the subject from about 1 day to about 7 days prior to the transfer of the one or more embryos to the subject, such as from about 2 days to about 5 days prior to embryo transfer. The ova or oocytes isolated from the subject may include mature oocytes, such as from 1 to 4 mature oocytes that are ready for fertilization upon contact with one or more sperm cells. The ova or oocytes may be isolated from a subject undergoing embryo transfer therapy or from a donor, such as a familial donor.

A subject undergoing embryo transfer therapy or a donor may be prepared for ovum or oocyte retrieval by controlled ovarian hyperstimulation, for instance, according to methods described herein or known in the art. For example, a subject or donor may be administered a GnRH antagonist so as to prevent a premature increase in the serum concentration of luteinizing hormone (LH). Additionally or alternatively, final follicular maturation can be achieved by administration of hCG to the subject or donor prior to isolation of the one or more ova or oocytes. For instance, the hCG can be administered to the subject in a single dose or in multiple doses, for instance, by intravenous injection according to procedures known in the art.

In some embodiments, a luteal support is provided to the subject or donor following ovum or oocyte retrieval. This may be performed, for instance, by administering progesterone to the subject or donor following the retrieval procedure. For example, progesterone may be administered to the subject or donor intravaginally at a dose of from about 300 mg to about 600 mg. The progesterone may be administered to the subject in a single dose or in multiple doses. For instance, progesterone may be administered to the subject in regularly spaced intervals beginning within about 24 hours of isolation of the one or more ova or oocytes, such as within 12 hours of retrieval, and continuing for about 6 or more weeks following the transfer of the one or more embryos to the subject.

### Embryo Quality and Condition

Embryos for use in conjunction with the compositions and methods described herein include those that are at, for example, the morula or the blastula stage of embryonic development. For instance, embryos that may be transferred to a subject as described herein include those that contain from 6 to 8 blastomeres immediately prior to transfer of the one or more embryos to the subject. The blastomeres may be of approximately equal sizes as assessed by visual microscopy prior to the transfer of the one or more embryos to the subject.

Embryos for use in conjunction with the compositions and methods described herein include those that are formed, for instance, by IVF or ICSI methods known in the art. In some embodiments, the embryos are freshly transferred to the uterus of the subject, for instance, from about 1 day to about 7 days (e.g., from about 2 days to about 5 days) following the isolation of one or more oocytes or ova from the subject for IVF or ICSI. In some embodiments, the one or more embryos are frozen and cryopreserved for long-term storage prior to thaw and transfer to the subject. Methods for the cryopreservation of embryos are known in the art and have been described, for instance, in WO 1991/003935 and WO 2010/011766, the disclosures of each of which are incorporated herein by reference as they pertain to compositions and procedures for cryopreserving embryos for long-term storage.

### Methods of Assessing Pregnancy

Techniques for assessing pregnancy for use in conjunction with the compositions and methods described herein include qualitative and quantitative assessments of a sample isolated from a subject, such as a sample of blood or urine. Methods for assessing pregnancy include detecting the presence and/or quantity of hCG in a sample isolated from a subject. This can be achieved, for instance, using conventional receptor-ligand binding assays known in the art, such as through the use of competitive radioligand binding assays, which are described for the detection of hCG in U.S. Pat. No. 4,094,963, the disclosure of which is incorporated herein by reference as it pertains to methods of detecting hCG in subject samples to assess pregnancy. Additionally or alternatively, test strips may be used to determine hCG concentrations, as described, for instance, in U.S. Pat. No. 7,989,217, the disclosure of which is incorporated herein by reference as it pertains to methods of detecting hCG in subject samples to assess pregnancy. Urine samples isolated from a subject can additionally be analyzed in order to determine pregnancy, as described, for instance, in U.S. Pat. No. 4,315,908, the disclosure of which is incorporated herein by reference as it pertains to methods of detecting hCG in subject samples to assess pregnancy.

Additionally or alternatively, pregnancy may be assessed by detecting intrauterine heartbeat, such as the heartbeat of the embryo or developing fetus following successful embryo implantation. Compositions and methods for detecting embryonic and fetal heartbeat are known in the art and are described, for instance, in U.S. Pat. Nos. 3,780,725 and 4,437,467, the disclosures of each of which are incorporated herein by reference as they pertain to methods of detecting heartbeat to assess pregnancy in a subject.

Following embryo transfer, for instance, as described herein, a subject may be subject to one or more pregnancy tests, for example, using one or more of the foregoing procedures. The subject may be tested for pregnancy at one or more points following embryo transfer therapy, such as at about 14 days, about 6 weeks, about 10 weeks, or longer, following embryo transfer and/or oocyte retrieval.

### Pharmaceutical Compositions

Oxytocin antagonists for use with the compositions and methods of the disclosure can be formulated into a pharmaceutical composition for administration to a subject, such as a female human subject, in a biologically compatible form suitable for administration in vivo. A pharmaceutical composition containing an oxytocin antagonist (e.g., a compound of formula (I) or (II), above) may additionally contain a suitable diluent, carrier, or excipient. Oxytocin antagonists can be administered to a subject, for example, orally or by intravenous injection. Under ordinary conditions of storage and use, a pharmaceutical composition may contain a preservative, e.g., to prevent the growth of microorganisms. Conventional procedures and ingredients for the selection and preparation of suitable formulations are described, for example, in Remington: The Science and Practice of Pharmacy (2012, 22^{nd} ed.) and in The United States Pharmacopeia: The National Formulary (2015, USP 38 NF 33), the disclosures of each of which are incorporated herein by reference as they pertain to pharmaceutically acceptable formulations for therapeutic compositions.

In some embodiments, compound (II) is administered to a subject according to the methods described herein in crystalline form. For instance, compound (II) may be administered to a subject undergoing embryo transfer therapy in a crystalline form that exhibits characteristic X-ray powder diffraction peaks at about 7.05° 2θ, about 13.13° 2θ, and about 23.34° 2θ. For instance, the compound may exhibit characteristic X-ray powder diffraction peaks at about 7.05° 2θ, about 12.25° 2θ, about 13.13° 2θ, about 16.54° 2θ, about 18.00° 2θ, about 21.54° 2θ, and about 23.34° 2θ. In some embodiments, the compound exhibits characteristic X-ray powder diffraction peaks as set forth in Table 1, below.

TABLE 1
Characteristic X-ray powder diffraction (XRPD)
peaks of crystal form of compound (II)

| XRPD | | Intensity |
|---|---|---|
| Peak (° 2θ) | d space (Å) | (%) |
| 7.05 ± 0.20 | 12.520 ± 0.354 | 45 |
| 12.25 ± 0.20 | 7.218 ± 0.117 | 36 |
| 13.13 ± 0.20 | 6.739 ± 0.102 | 55 |
| 14.16 ± 0.20 | 6.250 ± 0.088 | 8 |
| 16.54 ± 0.20 | 5.356 ± 0.064 | 38 |
| 18.00 ± 0.20 | 4.923 ± 0.054 | 36 |
| 18.77 ± 0.20 | 4.723 ± 0.050 | 34 |
| 21.32 ± 0.20 | 4.165 ± 0.039 | 5 |
| 21.84 ± 0.2 | 4.066 ± 0.037 | 36 |
| 23.34 ± 0.20 | 3.808 ± 0.032 | 100 |
| 24.08 ± 0.20 | 3.693 ± 0.030 | 14 |
| 24.67 ± 0.20 | 3.605 ± 0.029 | 1 |
| 25.45 ± 0.20 | 3.497 ± 0.027 | 27 |
| 25.69 ± 0.20 | 3.465 ± 0.027 | 8 |
| 26.45 ± 0.20 | 3.367 ± 0.025 | 10 |
| 27.09 ± 0.20 | 3.289 ± 0.024 | 2 |
| 28.05 ± 0.20 | 3.179 ± 0.022 | 14 |
| 28.56 ± 0.20 | 3.123 ± 0.021 | 3 |
| 29.26 ± 0.20 | 3.050 ± 0.020 | 16 |
| 30.72 ± 0.20 | 2.908 ± 0.018 | 2 |
| 31.00 ± 0.20 | 2.882 ± 0.018 | 3 |
| 31.19 ± 0.20 | 2.865 ± 0.018 | 5 |
| 33.19 ± 0.20 | 2.697 ± 0.016 | 2 |
| 33.60 ± 0.20 | 2.665 ± 0.015 | 6 |
| 34.36 ± 0.20 | 2.608 ± 0.015 | 4 |
| 34.75 ± 0.20 | 2.580 ± 0.014 | 2 |
| 35.91 ± 0.20 | 2.499 ± 0.013 | 2 |
| 36.52 ± 0.20 | 2.458 ± 0.013 | 3 |
| 37.38 ± 0.20 | 2.404 ± 0.012 | 2 |
| 37.70 ± 0.20 | 2.384 ± 0.012 | 1 |
| 38.73 ± 0.20 | 2.323 ± 0.012 | 3 |
| 39.11 ± 0.20 | 2.301 ± 0.011 | 2 |
| 39.80 ± 0.20 | 2.264 0.011 | 4 |

The foregoing crystal form has been shown to exhibit enhanced stability to aqueous media and physical stress, and is described in detail, for instance, in US 2016/0002160, the disclosure of which is incorporated herein by reference in its entirety.

Compounds of formula (I) or (II) can be administered by a variety of mutes, such as orally or intravenously. When formulated for oral administration, for instance, the compound may be administered in the form of a tablet, capsule, gel cap, powder, liquid solution, or liquid suspension. In some embodiments, the compound is administered to the subject in the form of a tablet, such as a dispersible tablet. The dispersible tablet may have, for example, one or more, or all, of the following components:

a. about 1-20% by weight of calcium silicate;

b. about 0.1-20% by weight of PVP30K;

c. about 0.01-5% by weight of poloxamer 188;

d. about 0.5-20% by weight of sodium croscarmellose;

e. about 1-90% by weight of microcrystalline cellulose 112;

f. about 1-90% by weight of lactose monohydrate;

g. about 0.01-0.5% by weight of sodium saccharine; and

h. about 0.1-10% by weight of glycerol dibehenate.

For instance, the dispersible tablet may have the following composition:

a. about 5% by weight of calcium silicate;

b. about 1% by weight of PVP30K;

c. about 2% by weight of poloxamer 188;

d. about 5% by weight of sodium croscarmellose;

e. about 1.5% by weight of microcrystalline cellulose 112;

f. about 47.8% by weight of lactose monohydrate;

g. about 0.2% by weight of sodium saccharine; and

h. about 4% by weight of glycerol dibehenate.

The foregoing formulations of compound (II) have been shown to exhibit rapid absorption kinetics upon administration to a subject, and are described in detail, for instance, in US 2015/0164859, the disclosure of which is incorporated herein by reference in its entirety.

Pharmaceutical compositions of compound (1) or (II) may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a subject in need of treatment.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Oral Administration of Compound (II) Promotes Successful Embryo Implantation and Prolongs Pregnancy in Subjects Undergoing Embryo Transfer Therapy

### Materials and Methods

In a randomized, double-blind, parallel groups, Phase 2 clinical study of the efficacy of compound (II) in enhancing endometrial receptivity and promoting successful embryo implantation in humans, this compound was orally administered to subjects undergoing embryo transfer therapy in doses of varying strength. A total of 247 female subjects were selected for treatment based on a variety of inclusion criteria. Of these, 244 subjects completed the study. The study was open to healthy female volunteers from 18 to 36 years of age that had previously undergone up to one IVF or ICSI cycle that resulting in a negative pregnancy test as assessed by hCG detection, despite the transfer of at least one embryo of good quality, which was defined as an embryo having from six to eight blastomeres of uniform size and shape on the day of embryo transfer, ooplasm having no granularity, absence of multinucleation, and a maximum fragmentation of 10%. Subjects included in the study had at least one functional ovary and were capable of communicating with the investigator and research staff and complying with the requirements of the study protocol. A demographic summary of the subjects included in the study is shown in Table 2, below. Data are presented in the form of mean (standard deviation).

TABLE 2
Demographic summary of subjects included in study

| | | | | Compound (II) | | | | | |
|---|---|---|---|---|---|---|---|---|---|
| | | Placebo | | 100 mg dose | | 300 mg dose | | 900 mg dose | |
| Parameter | Unit | n = 65 | | n = 62 | | n = 60 | | n = 60 | |
| Age | years | 31.5 | (3.3) | 31.5 | (3.1) | 31.8 | (3.1) | 31.1 | (3.3) |
| Body mass index | kg/m² | 23.37 | (4.15) | 23.86 | (3.72) | 23.72 | (6.29) | 23.65 | (3.99) |
| Endometrium thickness | mm | 7.0 | (2.9) | 7.0 | (2.8) | 6.8 | (2.4) | 6.9 | (2.9) |
| Oocytes retrieved | n | 11.0 | (5.2) | 10.3 | (4.4) | 11.7 | (5.6) | 10.2 | (4.2) |
| Embryos generated | n | 6.6 | (3.1) | 6.2 | (3.7) | 7.3 | (4.1) | 5.9 | (3.2) |
| Good quality embryos generated | n | 3.7 | (2.3) | 3.6 | (3.5) | 4.4 | (3.6) | 3.5 | (2.9) |
| Embryos transferred | % n = 1 | 60.0% | | 62.9% | | 60.0% | | 59.3% | |
| | % n = 2 | 40.0% | | 37.1% | | 40.0% | | 40.7% | |
| Embryo transfer difficult | % | 1.50% | | 3.20% | | 1.70% | | 0.0% | |
| Uterine contraction rate | n/min | 2.01 | (0.68) | 2.05 | (0.49) | 1.97 | (0.56) | 2.12 | (0.48) |
| at time of embryo transfer | | | | | | | | | |
| Serum P4 level at time | nM | 287 | (156) | 256 | (155) | 321 | (155) | 238 | (130) |
| of embryo transfer | | | | | | | | | |
| Serum E2 level at time | pM | 4255 | (2790) | 3833 | (2127) | 4988 | (2913) | 4265 | (2781) |

| of embryo transfer | | | | | | | | |
|---|---|---|---|---|---|---|---|---|
| Serum compound (II) level | ng/mL | N/A | 484.1 | (159.8) | 1453.1 | (453.8) | 4159.0 | (1367.7) |

at time of embryo transfer

Subjects included in the study underwent an initial screening period beginning up to 12 weeks prior to the day of oocyte retrieval from the subject. During this 12-week period, subjects underwent physical and gynecological examination in preparation for oocyte retrieval. This analysis included a recordation of the subjects' vital signs, hematology and biochemistry analysis of blood samples withdrawn from the subjects, urinary analysis, and a comprehensive review of the subjects' medical histories.

At the conclusion of the screening period, subjects underwent controlled ovarian hyperstimulation by administration of a GnRH antagonist so as to prevent a premature rise in serum LH concentration. Concurrent pre-treatment with an oral contraceptive prior to controlled ovarian hyperstimulation was allowed, but not required. Final follicular maturation was performed with a single administration of hCG to the subject. Luteal support was performed by intravaginal administration of micronized natural progesterone at a dose of 600 mg (3×200 mg dosage forms) daily, commencing within 6-24 hours of oocyte retrieval. Progesterone administration continued for at least 6 weeks following embryo transfer for subjects testing positive for pregnancy at 14 days following embryo transfer. Retrieved oocytes contained at least 1-4 mature oocytes (i.e., ova), which were subsequently used for IVF or ICSI for embryo generation.

The embryo transfer procedure was conducted three days after the oocyte retrieval day (OPU+3 days). Subjects undergoing embryo transfer were monitored prior to initiating the procedure. This analysis included a recordation of vital signs, as well as a transvaginal ultrasound to assess uterine contraction rate and endometrial thickness. Subjects were considered eligible for embryo transfer if the uterine contraction rate was found to be greater than or equal to 1.5 contractions per minute. Eligible subjects subsequently underwent blood sample analysis to determine pre-treatment levels of serum E2 and P4.

Upon confirming eligibility, subjects were randomized to one of four treatment arms: those receiving a single 100 mg dose of compound (II), a single 300 mg dose of compound (II), a single 900 mg dose of compound (II), or placebo. Subjects receiving a 100 mg dose of compound (II) received 2×50 mg dispersible tablets. Subjects receiving a 300 mg dose of compound (II) received 2×50 mg dispersible tablets and 1×200 mg dispersible tablet. Subjects receiving a 900 mg dose of compound (II) received 2×50 mg dispersible tablets and 4×200 mg dispersible tablets. Subjects not treated with compound (II) were administered a placebo, for instance, in 2×50 mg dispersible tablets and 4×200 mg dispersible tablets. Subjects did not consume food or fluids, with the exception of water, for 2 hours prior to administration and for 1 hour following administration.

Subjects were administered the indicated dose of compound (II) or placebo approximately 4 hours prior to embryo transfer. About 30 minutes prior to embryo transfer (approximately 3.5 hours following administration of compound (II) or placebo), a transvaginal ultrasound was conducted so as to record uterine contraction rate, and blood sample analysis was performed to obtain a post-treatment measurement of serum concentrations of compound (II), E2, and P4. At 4 hours following treatment with compound (II) or placebo, subjects underwent an ultrasound-guided embryo transfer according to conventional procedures. From one to two embryos of good quality were transferred to each subject. To reduce uterine contractions at the time of embryo transfer, soft or ultra-soft catheters were used and contact with the uterine fundus was avoided. Any difficulties that occurred during the embryo transfer procedure were recorded, including instances in which uterine sounding or cervical dilation were required, instances in which a harder catheter was required, or instances in which blood was found in any part of the catheter.

Approximately 1 hour following embryo transfer, subjects underwent a final physical examination and were subsequently discharged from the clinical unit until the first follow-up visit, which occurred at about 14 days following oocyte retrieval (OPU+14 days). At this time, subjects underwent a physical examination as well as a blood sample analysis to assess pregnancy by detection of hCG. Subjects testing positive for pregnancy continued the study and were scheduled for follow-up examinations at about 6 weeks following embryo transfer and at about 10 weeks following oocyte retrieval (OPU+10 weeks). Subjects that returned for examination at about 6 weeks following embryo transfer underwent ultrasound analysis. Pregnancy status was monitored by detecting embryo heartbeat. Subjects that exhibited a live birth during the study were scheduled for follow-up consultations to assess the subjects' physical state.

### Statistical Analysis

A two-sided type I error rate of 0.1 (corresponding to a one-sided type I error rate of 0.05) was used for analysis of data collected from this study. Subjects with a negative blood pregnancy test at 14 days following oocyte retrieval were considered as negative for the subsequent efficacy endpoints (e.g., pregnancy tests at 6 weeks following embryo transfer and 10 weeks following oocyte retrieval, as well as live birth rate).

Analysis of pregnancy rate at 6 weeks following embryo transfer was conducted via the Cochran-Armitage test of a linear trend in proportions, using all of the treatment arms as an ordinal scaled variable. A secondary analysis was conducted by fitting a logistic regression model with dose as a covariate and testing whether the slope was equal to zero. As higher pregnancy rates may occur with increasing number of transferred embryos, any potential effect of the number of embryos transferred on efficacy was explored, for example, by using the embryo transfer rate as a covariate. In addition, a potential dose time embryo transfer rate interaction was explored. Any potential effect of the embryo transfer difficulty on efficacy was also explored. Any possible site to site effect was explored as well.

Individual dose versus placebo comparisons were tested via Fisher's exact test and as contrasts within the logistic regression models. Corresponding confidence intervals were produced. No multiplicity adjustment was planned for these individual comparisons.

Positive blood pregnancy test at 14 days following oocyte retrieval and positive embryo heartbeat at 10 weeks following oocyte retrieval were assessed in the same manner as described above. Change from baseline to the time of embryo transfer in uterine contraction rate was analyzed by the Wilcoxon Rank Sum Test by comparing the uterine contraction rate associated with each dose to that observed with placebo-treated subjects.

For descriptive statistics of plasma concentrations of compound (II), E2, and P4, concentrations below the limit of quantification (LOQ) were assigned a value of zero, and results were provided if at least ⅔ of the plasma values per time point were above the LOQ.

### Results

A summary of the results of the clinical study over the entirety of the subjects that participated in the trial is shown in Table 3, below. The primary parameters of interest included the relative change in uterine contractility, positive pregnancy rates at about 14 days and 6 weeks following embryo transfer, positive pregnancy rates at 10 weeks following oocyte retrieval, as well as the live birth rate at a gestational age of at least 24 weeks.

TABLE 3
Results of compound (II) treatment among all subjects that participated in clinical trial

| | | Compound (II) | | | |
|---|---|---|---|---|---|
| | Placebo | 100 mg dose | 300 mg dose | 900 mg dose | All doses |
| Parameter | n = 65 | n = 62 | n = 60 | n = 60 | n = 182 |
| Relative changes | 0.0% | −8.7% | −4.0% | −13.3% | |
| in uterine | Wilcoxon | p = 0.30 | p = 0.72% | p = 0.05 | p = 0.14 |
| contractions | Rank Test | | | | |
| Positive pregnancy | 50.8% | 56.5% | 50.0% | 53.3% | |
| test at | Fisher Exact | p = 0.59 | p = 1.00 | p = 0.86 | |
| 14 days post | Test | | | | |
| embryo transfer | Logistic | p = 0.52 | p = 0.93 | p = 0.77 | Trend test |
| | Model* | | | | p = 0.96 |
| Ongoing pregnancy | 33.8% | 46.8% | 35.0% | 46.7% | 42.9% |
| rate at 6 weeks | Fisher Exact | p = 0.15 | p = 1.00 | p = 0.15 | p = 0.24 |
| post embryo transfer | Test | | | | |
| | Logistic Model | p = 0.09 | p = 0.99 | p = 0.12 | Trend test |
| | II** | | | | p = 0.33 |
| Ongoing pregnancy | 29.2% | 43.5% | 35.0% | 45.0% | 41.2% |
| rate at 10 weeks | Fisher Exact | p = 0.10 | p = 0.57 | p = 0.09 | p = 0.10 |
| post oocyte retrieval | Test | | | | |
| | Logistic | p = 0.10 | p = 0.49 | p = 0.07 | Trend test |
| | Model | | | | p = 0.15 |
| Live birth rate | 29.2% | 40.3% | 35.0% | 43.3% | 39.6% |
| at gestational age | Fisher Exact | p = 0.20 | p = 0.57 | p = 0.14 | p = 0.18 |
| of at least 24 weeks | Test | | | | |
| | Logistic | p = 0.19 | p = 0.49 | p = 0.10 | Trend test |
| | Model | | | | p = 0.20 |

*Logistic Model: Endpoint as dependent variable and treatment, site, and embryo transfer rate as independent variable
**Logistic Model II: Endpoint as dependent variable and treatment as independent variable

During the course of the analysis, it was noted that subjects in the 300 mg compound (II) treatment arm exhibited elevated pre-treatment serum P4 concentrations relative to the remainder of the subjects studied (Table 2). These heightened P4 levels are indicative of an elevated P4 concentration on the day of oocyte retrieval from the subject, and can reflect a P4 concentration of from 1.0 ng/ml to 2.0 ng/ml, such as a P4 concentration of 1.5 ng/ml on the day of oocyte retrieval. It was discovered that the effect of compound (II) was particularly robust among subjects that did not exhibit an elevated serum P4 concentration at the time of embryo transfer, and thus, likely did not exhibit a P4 concentration at or above a level of 1.5 ng/ml on the day of oocyte retrieval. Table 4, below, provides a summary of the pregnancy rate at 6 weeks following embryo transfer exhibited by subjects from each pre-treatment serum P4 concentration quartile.

TABLE 4
Pregnancy rate at about 6 weeks following embryo
transfer by pre-treatment serum P4 concentration quartile

| Ongoing pregnancy | | | | | |
|---|---|---|---|---|---|
| rate at 6 weeks | | | | | |
| post embryo transfer | Pre-dose Serum [P4] Quartile | | | | |
| Frequency | 1 | 2 | 3 | 4 | Total |
| Negative | 32 | 37 | 33 | 45 | 147 |
| | (51.61%) | (59.68%) | (54.10%) | (72.58%) | |
| Positive | 30 | 25 | 28 | 17 | 100 |
| | (48.39%) | (40.32%) | (45.90%) | (27.42%) | |
| Total | 62 | 62 | 61 | 62 | 247 |

Table 5, below, provides a summary of the live birth rate at a gestational age of at least 24 weeks (i) exhibited by all subjects and (ii) excluding subjects that exhibited a pre-treatment serum P4 concentration from the upper quartile of this metric.

TABLE 5
Live birth rate at a gestational age of at least 24 weeks
among subjects from all pre-treatment serum P4 quartiles
and excluding subjects from the upper quartile of this metric

| Live birth rate at a | Frequency | | | | | |
|---|---|---|---|---|---|---|
| gestational age of at | No. of | | | | | |
| least 24 weeks | embryos | | 100 mg | 300 mg | 900 mg | All |
| Subject Population | transferred | Placebo | dose | dose | dose | doses |
| All P4 | 1 | 11/39 | 13/39 | 12/36 | 14/35 | 39/110 |
| quartiles | | (28.21%) | (33.33%) | (33.33%) | (40.00%) | (35.45%) |
| | | Fisher | p = 0.81 | p = 0.80 | p = 0.33 | p = 0.44 |
| | | Exact | | | | Trend |
| | | Test | | | | test: |
| | | | | | | p = 0.24 |
| | 2 | 8/26 | 12/23 | 9/24 | 12/24 | 33/71 |
| | | (30.77%) | (52.17%) | (37.50%) | (50.00%) | (46.48%) |
| | | Fisher | p = 0.15 | p = 0.77 | p = 0.25 | p = 0.25 |
| | | Exact | | | | Trend |
| | | Test | | | | test: |
| | | | | | | p = 0.31 |
| Excluding | 1 | 9/30 | 11/34 | 10/22 | 13/29 | 34/85 |
| upper P4 | | (30.00%) | (32.35%) | (45.45%) | (44.83%) | (40.00%) |
| quartile | | Fisher | p = 1.00 | p = 0.38 | p = 0.29 | p = 0.39 |
| | | Exact | | | | Trend |
| | | Test | | | | test: |
| | | | | | | p = 0.16 |
| | 2 | 6/19 | 8/16 | 7/13 | 12/20 | 27/49 |
| | | (31.58%) | (50.00%) | (53.85%) | (60.00%) | (55.10%) |
| | | Fisher | p = 0.32 | p = 0.28 | p = 0.11 | p = 0.11 |
| | | Exact | | | | Trend |
| | | Test | | | | test: |
| | | | | | | p = 0.08 |

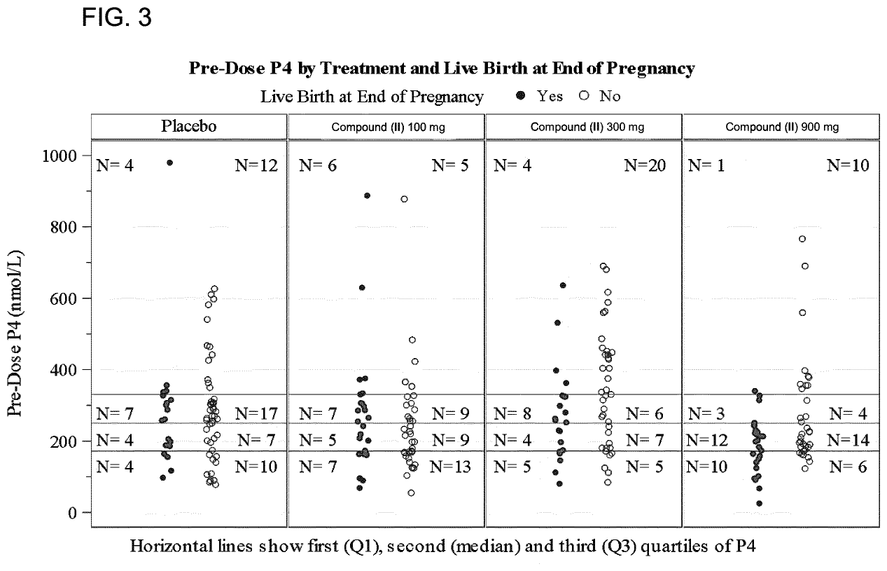

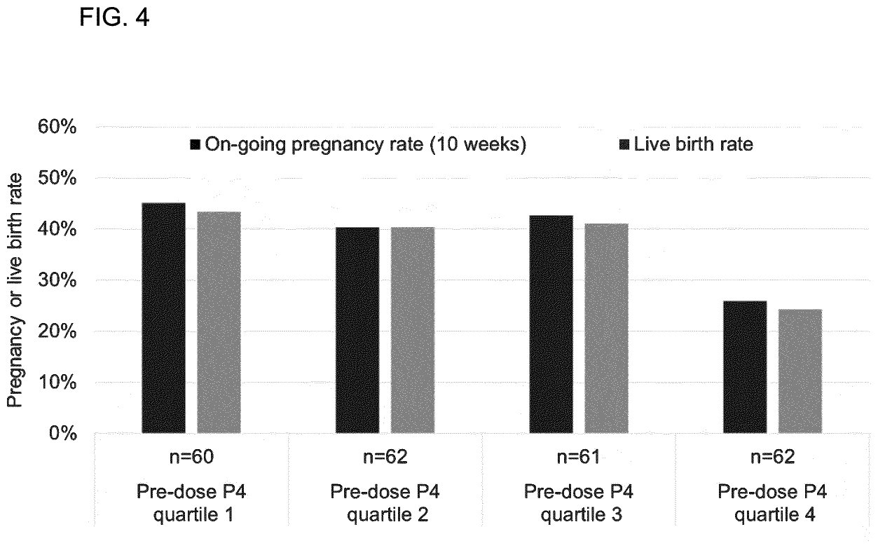

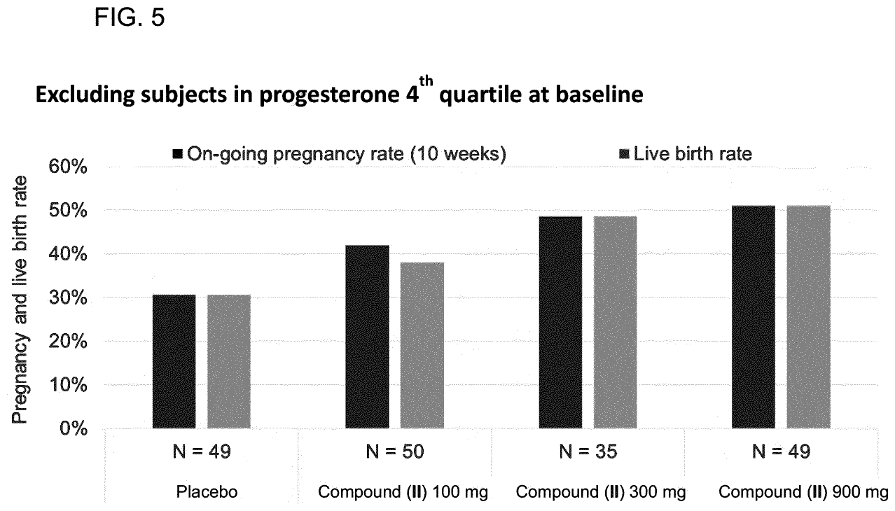

Collectively, these data demonstrate that lower overall pregnancy rates were observed among subjects with elevated pre-dose serum P4 concentrations. Upon analyzing, post hoc, the collected data with respect to subjects from pre-dose serum P4 concentration quartiles 1-3, an enhanced therapeutic effect of compound (II) was observed (FIGS. 3-5). This analysis is summarized in Table 6, below. Collectively, these data demonstrate that treatment with compound (II) lead to an overall increase in pregnancy and live-birth rates in the treatment arms versus the placebo group with in a significant dose-dependent fashion (p<0.02).

TABLE 6
Results of compound (II) treatment excluding subjects from pre-treatment serum P4 concentration Q4

| | | Compound (II) | | | |
|---|---|---|---|---|---|
| | Placebo | 100 mg dose | 300 mg dose | 900 mg dose | All doses |
| Parameter | n = 49 | n = 50 | n = 35 | n = 49 | n = 134 |
| Positive pregnancy | 53.1% | 54.0% | 62.9% | 59.2% | |
| test at 14 days post | Fisher Exact | p = 1.00 | p = 0.50 | p = 0.68 | p = 0.61 |
| embryo transfer | Test | | | | |
| | | | | | Trend test |
| | | | | | p = 0.42 |
| Ongoing pregnancy | 36.7% | 44.0% | 48.6% | 53.1% | 48.5% |
| rate at 6 weeks post | Fisher Exact | p = 0.54 | p = 0.37 | p = 0.15 | p = 0.18 |
| embryo transfer | Test | | | | |
| | | | | | Trend test |
| | | | | | p = 0.095 |
| Ongoing pregnancy | 30.6% | 42.0% | 48.6% | 51.0% | 47.0% |
| rate at 10 weeks post | Fisher Exact | p = 0.30 | p = 0.11 | p = 0.064 | p = 0.063 |
| oocyte retrieval | Test | | | | |
| | | | | | Trend test |
| | | | | | p = 0.035 |
| Live birth rate at | 15/49; | 19/50; | 17/35; | 25/49; | 61/134; |
| gestational age | 30.6% | 38.0% | 48.6% | 51.0% | 45.5% |
| of at least 24 weeks | Fisher Exact | p = 0.53 | p = 0.11 | p = 0.064 | p = 0.090 |
| | Test | | | | |
| | | | | | Trend test |
| | | | | | p = 0.025 |

| | | | | |
|---|---|---|---|---|
| Absolute increase in live birth rate vs placebo | 7.4% | 18.0% | 20.4% | 14.9% |
| Relative increase in live birth rate vs placebo | 24.2% | 58.8% | 66.7% | 48.7% |

This post hoc analysis revealed that subjects exhibiting an elevated serum P4 concentration on the day of embryo transfer also exhibited an elevated serum P4 concentration on the day of oocyte retrieval, such as a serum P4 concentration above the threshold level of 1.5 ng/ml. Table 7, below, summarizes the quantity of subjects for which data were available that exhibited a serum P4 concentration above 1.5 ng/ml on the day of oocyte retrieval prior to administration of hCG to induce final follicular maturation.

TABLE 7
Subjects that exhibited a serum P4
concentration above 1.5 ng/ml on the day of
oocyte retrieval prior to hCG administration

| | | Compound (II) | | | |
|---|---|---|---|---|---|
| Subject | | 100 | 300 | 900 | |
| Population | Placebo | mg dose | mg dose | mg dose | Total |
| Serum P4 greater | 1/24 | 1/23 | 5/24 | 2/23 | 9/94 |
| than 1.5 ng/ml | (4.17%) | (4.35%) | (20.83%) | (8.70%) | (9.57%) |
| on the day of | | | | | |
| oocyte retrieval | | | | | |

As shown in Table 7, the 300 mg treatment arm contained the highest proportion of subjects having a serum P4 concentration of greater than 1.5 ng/ml on the day of oocyte retrieval prior to hCG administration. Table 2, above, demonstrates that subjects in the 300 mg treatment arm exhibited an elevated serum P4 concentration on the day of embryo transfer as well (e.g., an average serum P4 concentration of about 320 nM). Taken together, these data demonstrate that subjects exhibiting an elevated serum P4 concentration on the day of embryo transfer, such as 320 nM or greater, also exhibited a heightened serum P4 concentration on the day of oocyte retrieval, such as 1.5 ng/ml or greater.

As described above, removal of subjects from the upper serum P4 quartile from the analysis revealed a particularly robust therapeutic effect of compound (II). A regression analysis was conducted to quantify the ability of pre-treatment serum progesterone concentration on the day of embryo transfer to serve as a negative predictor of clinical pregnancy. This regression analysis is summarized in Table 8, below.

TABLE 8
Regression model of utility of pre-treatment serum
P4 as a negative predictor of clinical pregnancy

| | Variable | | |
|---|---|---|---|
| | Pre- | Pre- | No. of |
| Regression | treatment | treatment | embryos |
| Parameter | serum E2 | serum P4 | transferred |
| N | 245 | 245 | 245 |
| Adjusted odds ratio | 1.05 | 0.78 | 1.72 |
| 90% Confidence | 0.66-1.44 | 0.65-0.93 | 1.10-3.69 |
| interval of | | | |
| adjusted odds ratio | | | |
| p-value | 0.40 | 0.020 | 0.045 |

As shown in Table 8, a significant negative relationship was identified between pre-treatment serum progesterone concentration and clinical pregnancy rate.

It has been presently discovered that compound (II) may promote the transient overexpression of PGF2α and the subsequent downregulation of PGF2α signaling, for instance, by desensitization of the PGF2α receptor. This heightened expression of PGF2α and subsequent attenuation of PGF2α signaling can in turn enhance the receptivity of the endometrium to exogenously administered embryos. Notably, P4 is a negative regulator of PGF2α expression, which may explain why compound (II) has a particularly strong therapeutic effect on subjects that do not exhibit elevated pre-treatment serum P4 concentrations.

Taken together, the data obtained from this study demonstrate the ability of compound (II) to promote endometrial receptivity, reduce the likelihood of embryo implantation failure in subjects undergoing embryo transfer therapy, and prolong pregnancy in such subjects through various gestational ages, as well as the ability of pre-treatment serum P4 concentration to serve as a predictive indicator of a subject's propensity to benefit from oxytocin antagonist treatment during the course of an assisted reproductive technology procedure.

### Example 2. Administration of an Oxytocin Antagonist to a Subject Undergoing Embryo Transfer Therapy on the Basis of the Subject's Pre-Treatment Serum Progesterone Level

Using the compositions and methods described herein, a skilled practitioner can assess the likelihood that a human subject undergoing embryo transfer therapy will benefit from oxytocin antagonist treatment by comparing the serum progesterone concentration of a subject to a progesterone reference level. For example, on the basis of a subject's pre-treatment serum progesterone concentration, a practitioner of skill in the art can determine whether the subject is likely to exhibit increased endometrial receptivity in response to oxytocin antagonist treatment. This determination can subsequently inform the practitioner's decision of whether to administer to the subject an oxytocin antagonist, such as a pyrrolidine-3-one oxime compound of formula (I) or (11) or another oxytocin antagonist described herein or known in the art, such as epelsiban, retosiban, barusiban, and atosiban, or a salt, derivative, variant, crystal form, or formulation thereof.

For instance, a physician of skill in the art can withdraw a sample from a subject undergoing embryo transfer therapy on the day of oocyte or ovum retrieval in the case of a subject using an autologous gamete for the ex vivo production of an embryo. In such instances, the progesterone reference level may be from 1.0 ng/ml to 2.0 ng/ml, such as 1.0 ng/ml, 1.1 ng/ml, 1.2 ng/ml, 1.3 ng/ml, 1.4 ng/ml, 1.5 ng/ml, 1.6 ng/ml, 1.7 ng/ml, 1.8 ng/ml, 1.9 ng/ml, or 2.0 ng/ml. The progesterone reference level may be, for instance, 1.5 ng/ml in such instances. The physician may then compare the progesterone level in the sample (e.g., serum sample) isolated from the subject to that of the progesterone reference level. A determination that the subject exhibits a reduced serum progesterone concentration relative to the progesterone reference level indicates that the subject is particularly well suited for, and likely to benefit from (e.g., likely to exhibit enhanced endometrial receptivity in response to) treatment with an oxytocin antagonist. Upon making such a determination, the physician may subsequently administer an oxytocin antagonist to the subject. The oxytocin antagonist may be administered to the subject prior to, concurrently with, and/or after the transfer of one or more embryos to the subject.

Additionally or alternatively, the physician may withdraw a sample (e.g., a serum sample) from the subject on the day of the embryo transfer procedure (e.g., following oocyte or ovum retrieval in the case of a subject using an autologous gamete for the ex vivo production of an embryo). In such instances, the progesterone reference level may be from 200 nM to 300 nM or more, such as 320 nM. The physician may then compare the progesterone level in the sample (e.g., serum sample) isolated from the subject to that of the progesterone reference level. A determination that the subject exhibits a reduced serum progesterone concentration relative to the progesterone reference level indicates that the subject is particularly well suited for, and likely to benefit from (e.g., likely to exhibit enhanced endometrial receptivity in response to), treatment with an oxytocin antagonist. Upon making such a determination, the physician may subsequently administer an oxytocin antagonist to the subject. The oxytocin antagonist may be administered to the subject prior to, concurrently with, and/or after the transfer of one or more embryos to the subject.

### Example 3. Beneficial Oxytocin Antagonistic Effects and Metabolic Profile of Compound (II)

Using the compositions and methods described herein, one of skill in the art can administer an oxytocin antagonist to a subject undergoing an embryo transfer procedure, such as an oxytocin antagonist represented by formula (I), e.g., compound (II), so as to promote enhanced endometrial receptivity, reduce the likelihood of embryo implantation failure, and/or prevent miscarriage in a subject following the transfer of one or more embryos to the uterus of the subject. When compound (II) is administered as the oxytocin antagonist, it can be particularly advantageous to administer compound (II) in a substantially pure form with respect to its (3E) diastereomer, (3E,5S)-5-(hydroxymethyl)-1-[(2′-methyl-1,1′-biphenyl-4-yl)carbonyl]pyrrolidin-3-one O-methyloxime, such as in a form containing less than 15%, less than 10%, less than 5%, less than 1%, or less than 0.1% of the (3E) diastereomer. This advantage derives from the discovery that substantially pure compound (II) exhibits a superior ability to inhibit spontaneous uterine contractions relative to the substantially pure (3E) diastereomer. Uterine contractility is one component of endometrial receptivity, and elevated uterine contractility can lead to the expulsion of an embryo from the uterus and failed embryo implantation. This surprising disparity in uterine contractility inhibition between compound (II) and its (3E) diastereomer is described, for instance, in U.S. Pat. No. 9,670,155. As described therein, there is a dose-dependent reduction in spontaneous uterine contractions when substantially pure compound (II) is administered at 10, 30, and 60 mg/kg to anesthetized late-term pregnant rats. Inhibition of spontaneous uterine contractions of from about 10% to about 20% was observed from 5 to 15 minutes after oral administration of the substantially pure compound (II), and inhibition of about 42% was observed from 170 to 180 minutes after oral administration of the substantially pure compound (II) at a dose of 60 mg/kg. The inhibitory activity of the substantially pure compound (II) with respect to uterine contraction was found to be markedly higher than that of the substantially pure (3E) diastereomer using the same vehicle and in the same model organism.

This difference in inhibitory activity leads to an important clinical benefit, as the substantially pure compound (II) may be administered to a subject at a lower therapeutically effective dosage relative to the (3E) diastereomer or an isomeric mixture of both compounds.

In addition to exhibiting different inhibitory potencies, the substantially pure compound (II) exhibits superior metabolic properties relative to its (3E) diastereomer. It has been discovered that the substantially pure compound (II) is preferentially metabolized by cytochrome P450 isoform 3A4 (CYP3A4), while the substantially pure (3E) diastereomer is preferentially metabolized by cytochrome P450 isoforms 2D6 (CYP2D6) and 2C19 (CYP2C19).

To measure the metabolic properties of the substantially pure compound (II) and its (3E) diastereomer, microsomal stability assays were conducted. These experiments were designed to investigate the metabolism of the substantially pure compound (II) and its (3E) diastereomer by cytochrome P450 alone (CYP) or in combination with uridine 5′-diphosphoglucuronosyl transferase (UGT). The substantially pure compound (II) and its (3E) diastereomer were each incubated at a concentration of 3 μM with pooled liver microsomes and with appropriate co-factors for either cytochrome P450 alone or in combination with UGT. At five time points over the course of a 45 minute experiment, the compounds were analyzed by liquid chromatography and tandem mass spectrometry (LC-MS/MS). Intrinsic clearance values (CLᵢₙₜ) with standard error (SE CLᵢₙₜ) and metabolic half-life (t_{1/2}) were calculated and are indicated in Table 9, below.

TABLE 9
Metabolism of substantially pure (3Z) and (3E) isomers
by cytochrome P450 alone or in combination with UGT

| | Metabolic | | | Metabolic | | | | |
|---|---|---|---|---|---|---|---|---|
| | Stability—CYP | | | Stabilit—CYP/UGT | | | | |
| | CLᵢₙₜ | | | | CLᵢₙₜ | | | |
| | (μL/ | | | | (μL/ | | | |
| | min/mg | SE | t_{1/2} | | min/mg | SE | t_{1/2} | |
| Compound | protein) | CLᵢₙₜ | (min) | n | protein) | CLᵢₙₜ | (min) | n |
| Compound | 11.6 | 3.74 | 120 | 5 | 9.33 | 3.87 | 149 | 5 |
| (II) | | | | | | | | |
| (3E) | 6.40 | 3.49 | 216 | 5 | 5.38 | 3.32 | 258 | 5 |
| isomer | | | | | | | | |
| Z/E ratio | | | 0.56 | | | | 0.58 | |

As shown in Table 9, the metabolic stability of each of the substantially pure compound (II) and its (3E) diastereomer in the presence of co-factors required for cytochrome P450 activity is similar to that of each isomer in the presence of co-factors required for combined cytochrome P450 and UGT activity, indicating that cytochrome P450 is primarily responsible for the metabolic degradation of each isomer.

To determine the selectivity of each of the CYP3A4, CYP2136, and CYP2C19 isoforms of cytochrome P450, the substantially pure compound (II) and its (3E) diastereomer were each incubated at a concentration of 5 μM with each of the CYP3A4, CYP2C19, and CYP2136 isoforms. At five time points over the course of a 45 minute experiment, the compounds were analyzed by LC-MS/MS. The percentage of each compound remaining at each time point, along with the metabolic half-lives of each compound in the presence of each cytochrome P450 isoform, are indicated in Tables 10-12, below.

TABLE 10
Metabolism of substantially pure
(3Z) and (3E) isomers by CYP3A4 isoform

| | | | Compound Remaining | | | | |
|---|---|---|---|---|---|---|---|
| | t_{1/2} | | (% of compound present at t = 0 min) | | | | |
| Compound | (min) | n | 0 min | 5 min | 15 min | 30 min | 45 min |
| Compound | 73.7 | 5 | 100 | 86.5 | 75.9 | 67.3 | 63.9 |
| (II) | | | | | | | |
| (3E) | 281 | 5 | 100 | 107 | 88.8 | 92.5 | 92.2 |
| isomer | | | | | | | |
| Z/E ratio | 0.26^{a} | | | | | | |

^{a}Student’s t-test: p = 0.37

TABLE 11
Metabolism of substantially pure (3Z) and (3E)
isomers by CYP2D6 isoform

| | | | Compound Remaining | | | | |
|---|---|---|---|---|---|---|---|
| | t_{1/2} | | (% of compound present at t = 0 min) | | | | |
| Compound | (min) | n | 0 min | 5 min | 15 min | 30 min | 45 min |
| Compound | 14.5 | 5 | 100 | 80.3 | 50.5 | 22.5 | 12.2 |
| (II) | | | | | | | |
| (3E) | 5.62 | 4 | 100 | 49.3 | 14.1 | 2.43 | 0.845 |
| isomer | | | | | | | |
| Z/E ratio | 2.6^{b} | | | | | | |

^{b}Student’s t-test: p < 0.0001

TABLE 12
Metabolism of substantially pure
(3Z) and (3E) isomers by CYP2C19 isoform

| | | | Compound Remaining (% of | | | | |
|---|---|---|---|---|---|---|---|
| | t_{1/2} | | compound present at t = 0 min) | | | | |
| Compound | (min) | n | 0 min | 5 min | 15 min | 30 min | 45 min |
| Compound | 60.4 | 5 | 100 | 93.7 | 79.1 | 67.8 | 59.9 |
| (II) | | | | | | | |
| (3E) | 41.4 | 5 | 100 | 87.6 | 82.7 | 57.1 | 47.4 |
| isomer | | | | | | | |
| Z/E ratio | 1.5^{c} | | | | | | |

^{c}Student’s t-test: p = 0.016

The data shown in Tables 10-12 demonstrate that the substantially pure compound (II) is preferentially metabolized by the CYP3A4 isoform of cytochrome P450, while the substantially pure (3E) diastereomer is preferentially metabolized by the CYP2D6 and CYP2C19 isoforms of cytochrome P450. The selectivity exhibited by these cytochrome P450 isoforms provides a significant clinical benefit. Allelic variation in the CYP2D6 and CYP2D19 isoforms has been correlated with reduced drug metabolism in vivo in certain segments of the population (see, for example, Lynch et al., Am. Fam. Physician 76:391-396, 2007; the disclosure of which is incorporated herein by reference in its entirety). For example, according to Lynch, 7 percent of white persons and 2 to 7 percent of black persons are poor metabolizers of drugs dependent on CYP2D6, and one in five Asian persons is a poor metabolizer of drugs dependent on CYP2C19. In view of the discovery that the substantially pure compound (II) is preferentially metabolized by CYP3A4, this compound is expected to exhibit more uniform therapeutic and toxicity profiles than the substantially pure (3E) diastereomer.

### Example 4. Compound (II) Reduces Uterine Contractility and Increases Endometrial Blood Flow

The experiments described in this example were conducted as part of a randomized, double-blind clinical trial aimed at further elucidating the mechanism of action by which compound (II) enhances the likelihood of successful embryo implantation in patients undergoing embryo transfer procedures and reduces the probability of miscarriage. The clinical trial assessed the ability of compound (II) to decrease uterine contractions and increase endometrial perfusion, both of which improve uterine receptivity, thereby promoting embryo implantation, reducing the likelihood of miscarriage, and, ultimately, increasing the likelihood of achieving a pregnancy and live birth. The clinical trial further analyzed the effects of compound (II) on the expression of various genes in the endometrium.

### Clinical Trial Design

The randomized, double blind trial was conducted at a UK-based clinical pharmacology unit in 42 healthy female volunteers, aged between 18 and 37 years, who underwent a hormonal preparation identical to that used for infertile patients before frozen-thawed embryo transfer, prior to being administered a single oral dose of either 900 mg or 1,800 mg of compound (II) or matching placebo.

Particularly, subjects were pre-treated with estradiol valerate at a dosage of 2 mg administered 3 times per day (TID) for 16 days. Administration of estradiol valerate was followed by vaginal progesterone at a dosage of 200 mg administered TID. On the day corresponding to a day 5 embryo transfer, subjects received a single, oral administration of compound (II), in an amount of 900 mg or 1,800 mg, or matching placebo.

Pharmacodynamic assessments were performed at t=0 hours, 4 hours, 8 hours, and 24 hours after treatment with compound (II) or placebo. These assessments included measurements of uterine contractions by ultrasound and uterine perfusion by a 3D-power Doppler technique. After the last pharmacodynamic assessment at t=24 hours after treatment with compound (II) or placebo, an endometrial biopsy was collected to investigate potential effect of compound (II) on the expression of genes in the endometrium.

### Statistical Analyses

Mean and median changes in uterine contraction frequency and endometrial vascularity indices (particularly, endometrial flow index (FI), vascularity index (VI), and vascularity flow index (VFI)) were calculated. Exploratory non-parametric ANCOVA analyses were conducted to assess differences between each dose of compound (II) (900 mg or 1,800 mg) and placebo with respect to the number of uterine contractions per minute and the proportion of subjects with less than one uterine contraction per minute at t=4 hours, 8 hours, and 24 hours post-dose. The endometrial vascularity indices above were compared between each dose of compound (II) (900 mg or 1,800 mg) and placebo at t=4 hours, 8 hours, and 24 hours post-dose using the same methods. Differences in endometrial mRNA expression were identified and assessed for statistical significance.

### Effects of Compound (II) on Endometrial Receptivity

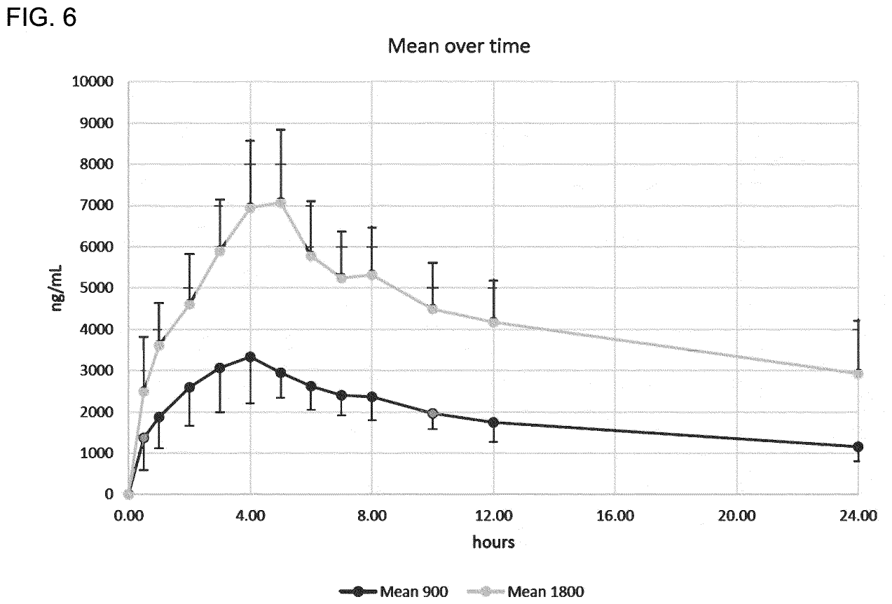

As shown in FIG. 6, compound (II) reaches a maximum concentration at about 4 hours following oral administration of either the 900 mg or the 1,800 mg dose.

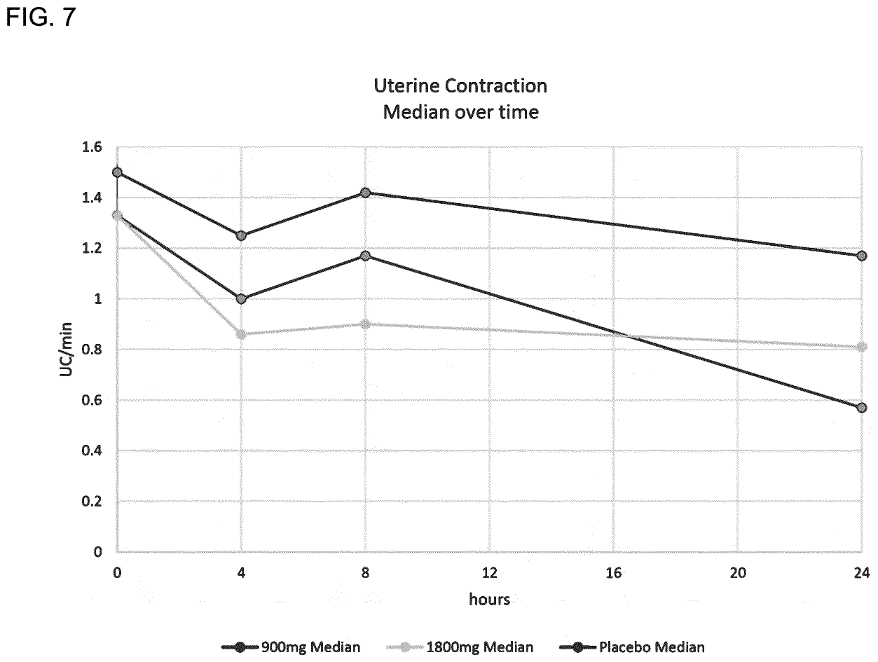

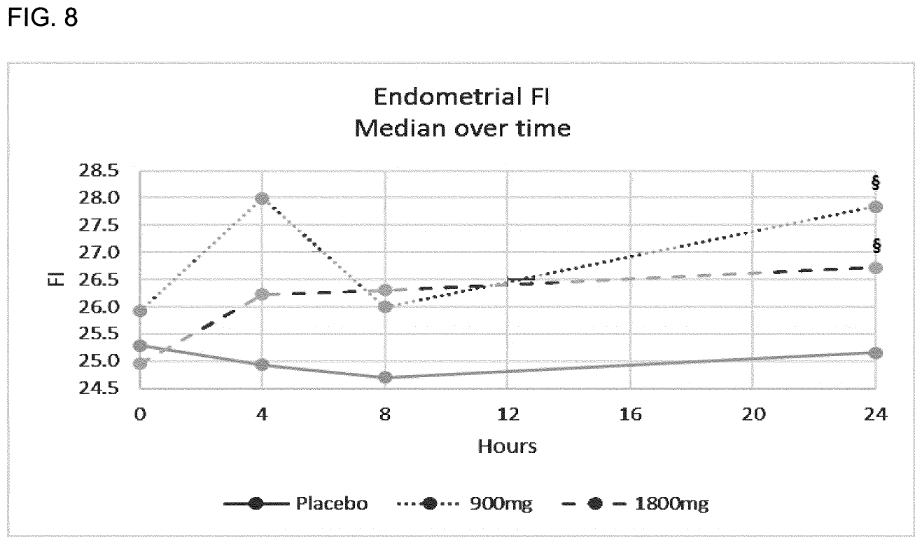

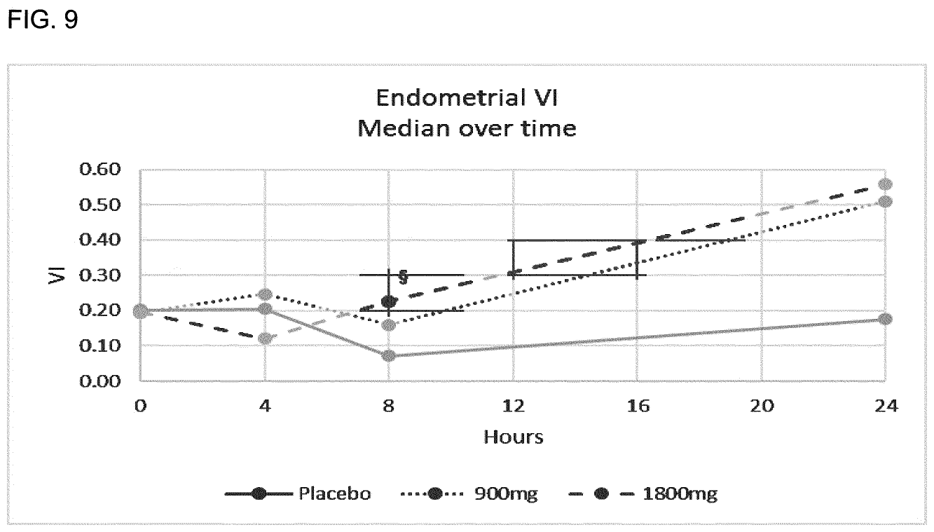

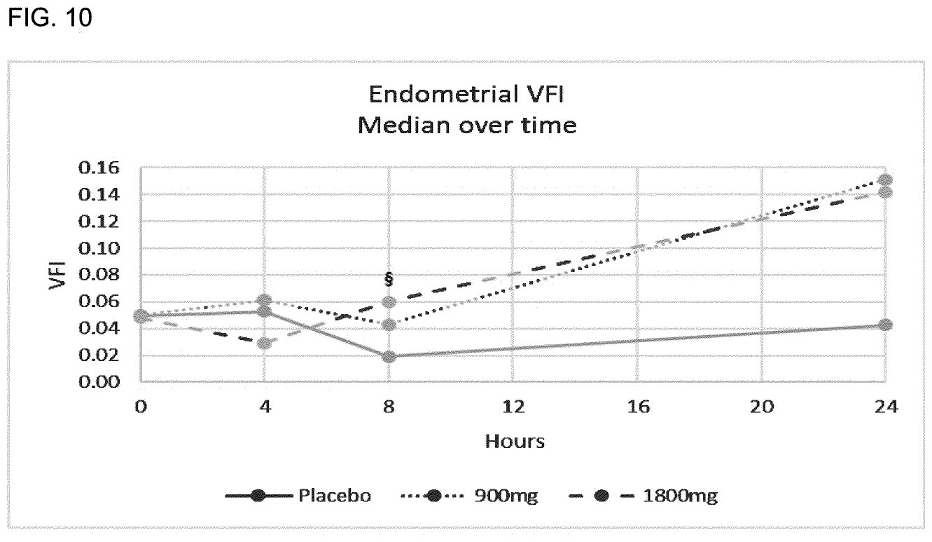

The results shown in FIGS. 7-10 confirm the ability of compound (II) to decrease uterine contractions (FIG. 7) and effectuate a marked and sustained increase in endometrial blood flow, as assessed by monitoring subjects' endometrial flow index (FI, FIG. 8), vascularity index (VI, FIG. 9), and vascularity flow index (VFI, FIG. 10). Taken together, these activities improve uterine receptivity, creating an environment in which the endometrium is likely to successfully receive a transferred embryo.

Additionally, as these data were collected in subject that underwent hormonal preparation mimicking that of patients undergoing frozen-thawed embryo transfer, these data particularly support the effectiveness of compound (II) in promoting successful implantation of embryos that have previously been cryopreserved and thawed.

Taken together, the results of these experiments not only demonstrate that compound (II) achieves a dose-dependent reduction in uterine contractility, but also that compound (II) engenders an increase in uterine blood flow. These effects combine to effectuate an environment in which the endometrium exhibits heightened receptivity, such that a patient undergoing an embryo transfer procedure and that has been administered compound (II) will have a higher likelihood of successful embryo implantation and a lower likelihood of miscarriage relative to a patient undergoing a similar procedure that has not been administered compound (II).

### Effects of Compound (II) on Endometrial Gene Expression

The experiments conducted in this trial additionally analyzed the effects of compound (II) on the expression of various genes in endometrial tissue. To do so, endometrial tissue samples were obtained from each subject both before and after administration of compound (II) or placebo. Using an RNA-Seq assay, each subject's expression of a variety of genes, including DPP4, CNTNAP3, CNTN4, CXCL12, TNXB, CTSE, OLFM4, KRT5, KRT6A, and IDO2, was then assessed to determine whether the expression of each gene increased, decreased, or underwent no significant change due to administration of the compound or placebo.

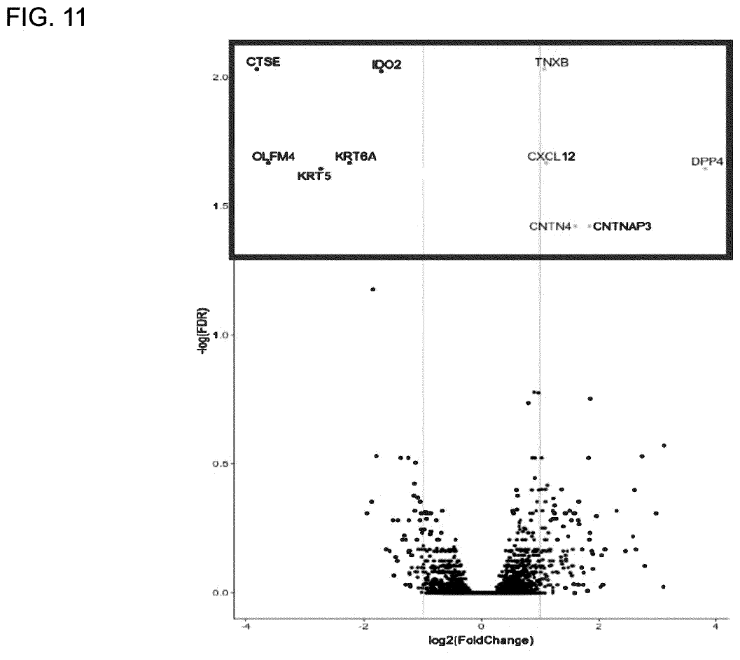

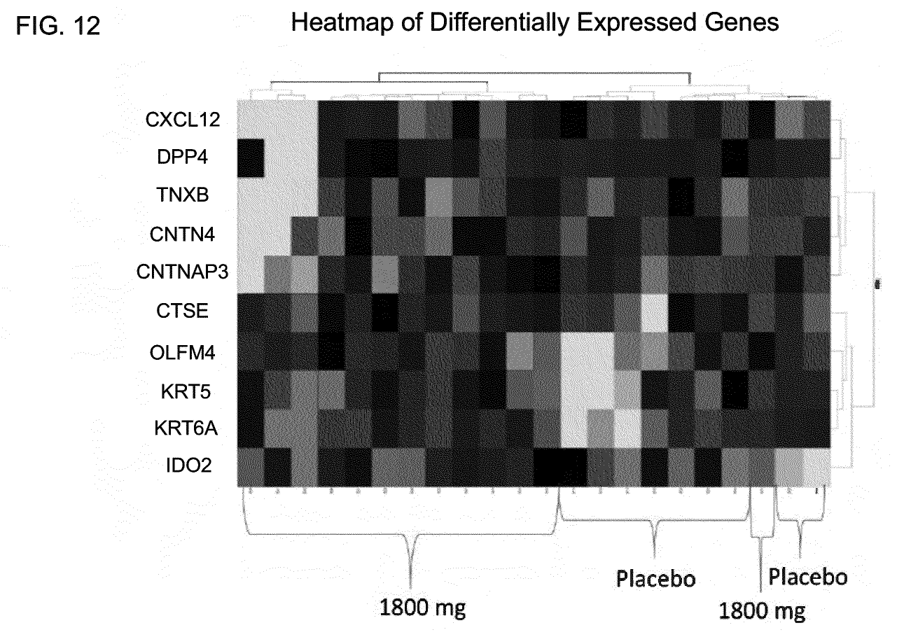

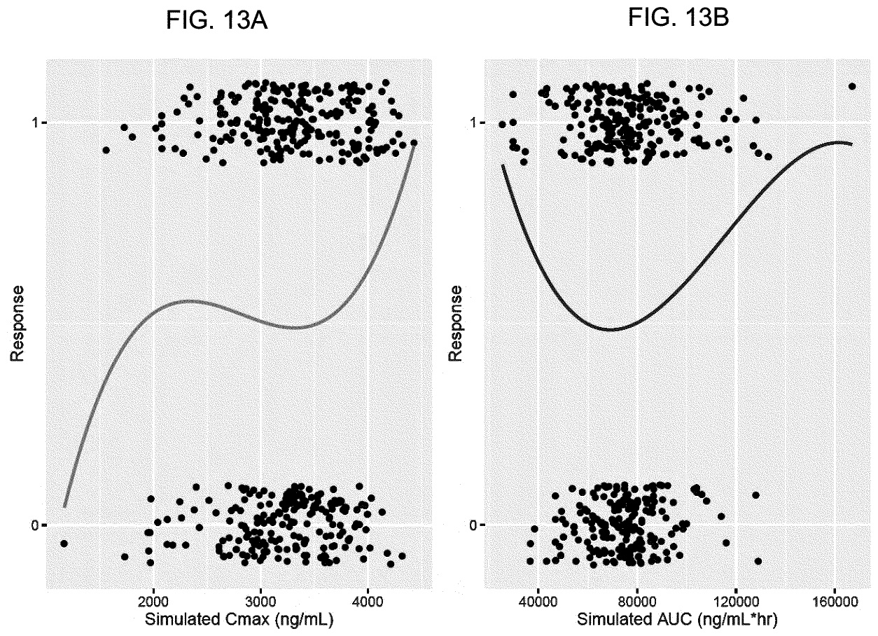

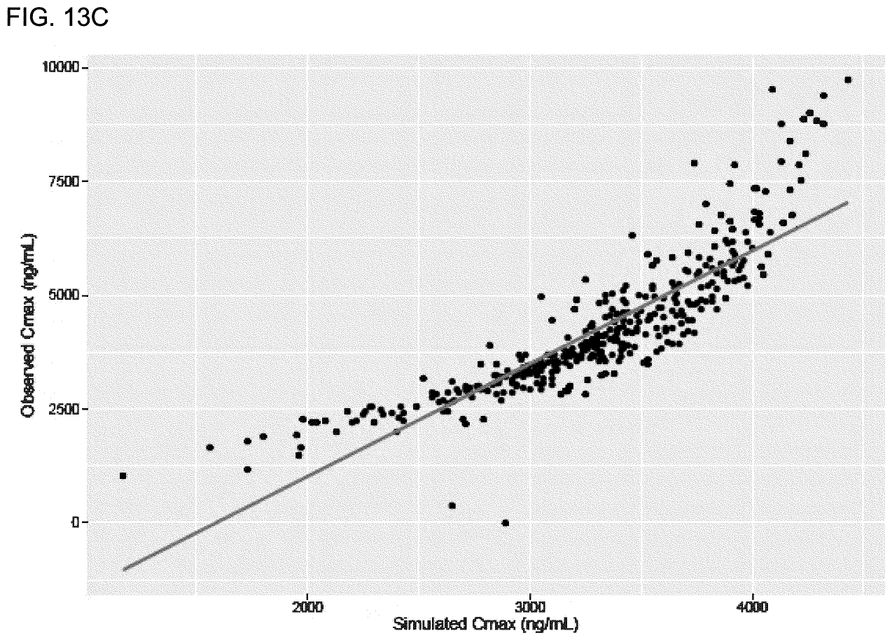

The effects of a single 1,800 mg dose of compound (II) on the expression of DPP4, CNTNAP3, CNTN4, CXCL12, TNXB, CTSE, OLFM4, KRT5, KRT6A, and IDO2 are shown graphically in FIG. 11 and in the form of a heatmap in FIG. 12. These data are additionally reported in detail in Table 13, below. As shown in FIG. 11, the majority of the genes tested in the RNA-Seq assay did not undergo a substantial change in endometrial expression following administration of compound (II). However, as shown in FIGS. 11 and 12, following administration of compound (II), expression of each of DPP4, CNTNAP3, CNTN4, CXCL12, TNXB in endometrial tissue increased significantly, and expression of each of CTSE, OLFM4, KRT5, KRT6A, and IDO2 in endometrial tissue decreased significantly. These observations are summarized in Table 13, below.

TABLE 13
Effects of compound (II) on endometrial
RNA transcript expression, as assessed by RNA-Seq

| | Log2 | Log (Counts | | False |
|---|---|---|---|---|
| Gene | (Fold Change) | per Million) | P value | Discovery Rate |
| CTSE | −3.816140885 | 2.566312321 | 1.09E−06 | 0.009312535 |
| DPP4 | 3.813404924 | 5.507977393 | 1.09E−05 | 0.022711225 |
| OLFM4 | −3.616062531 | 1.885484737 | 5.92E−06 | 0.021541929 |
| KRT5 | −2.728676989 | 3.252072528 | 1.18E−05 | 0.022711225 |
| KRT6A | −2.247460229 | 1.033476041 | 8.29E−06 | 0.021541929 |
| CNTNAP3 | 1.84141649 | −0.248619319 | 2.45E−05 | 0.037824449 |
| IDO2 | −1.70661053 | 4.470965333 | 1.85E−06 | 0.009510348 |
| CNTN4 | 1.596695597 | 3.645568029 | 2.22E−05 | 0.037824449 |
| CXCL12 | 1.097804359 | 5.5863452 | 8.38E−06 | 0.021541929 |
| TNXB | 1.067833642 | 7.143209489 | 1.21E−06 | 0.009312535 |

Taken together, these results demonstrate that compound (II) functions, at least in part, by augmenting endometrial expression of DPP4, CNTNAP3, CNTN4, CXCL12, and TNXB, while suppressing endometrial expression of CTSE, OLFM4, KRT5, KRT6A, and IDO2.

### CONCLUSION

The results of these experiments demonstrate that both the 900 mg and 1,800 mg doses of compound (II) engender measurable and durable effects on uterine contractions (FIG. 7). There was no significant difference in the number of uterine contractions per minute between the placebo and 900 mg dose groups (p>0.10 at all time points). However, at the 1,800 mg dosage regime, contraction-reducing effects were observed from the 4-hour time point onwards, with significance observed at the p<0.10 level at the 4-hour (p=0.0923) and 8-hour (p=0.0081) time points compared to placebo. A significant difference was observed between the placebo and the 900 mg dose groups with respect to the proportion of subjects showing less than one uterine contraction per minute at the 24-hour time point only (p=0.0437). At the 1,800 mg dose level, significance was seen at the 8-hour time point (p=0.0121).

Endometrial perfusion parameters showed marked and sustained increases in median values from baseline to 24 hours for both the 900 mg and 1,800 mg doses of compound (II). The most noticeable increases in VI for compound (II) compared to placebo occurred between the 8-hour and the 24-hour time points. The 1,800 mg dose group exhibited a particular substantial increase in VI at the 8-hour time point (p=0.0714). Endometrial FI also increased overtime, with significant increases at the 24-hour time point for both doses (p=0.0502 and p=0.0625 for the 900 mg and 1,800 mg groups, respectively). An approximately 3-fold increase in median endometrial VFI was observed for both doses between the pre-dose and 24-hour post-dose time points. This increase was significant for the 1,800 mg dose at the 8-hour time point (p=0.0754).

No differences in mRNA expression in endometrial biopsy tissue were observed after administration of 900 mg of compound (II) compared to placebo. In contrast, within 24 hours of administration of 1,800 mg of compound (II), 10 mRNAs were found to be significantly differentially expressed (adjusted p<0.05). Of these, 5 were upregulated and 5 were downregulated (FIGS. 11 and 12, Table 13). Particularly, OLFM4, DPP4, and CXCL12 were regulated in the same direction as Window-of-Implantation-associated genes. In addition, 3 genes (DPP4, CXCL12, and IDO2) that are implicated in endometrial receptivity were regulated in a direction supportive of successful embryo implantation.

In sum, the results of these experiments demonstrate the ability of compound (II) to improve endometrial receptivity, for example, by reducing uterine contractility, augmenting uterine blood flow, and modulating endometrial gene expression. Importantly, these results also indicate faster, broader, and stronger therapeutic effects achieved by doses greater than 900 mg of compound (II) (e.g., doses of from 1,500 mg to 2,700 mg, such as doses of from 1,800 mg to 2,000 mg (e.g., 1,800 mg), from 1,900 mg to 2,300 mg (e.g., 2,100 mg), and from 2,200 mg to 2,800 mg (e.g., 2,400 mg), among others described herein).

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of treating a subject undergoing embryo transfer therapy, the method comprising administering to the subject a therapeutically effective amount of a compound represented by formula (I) 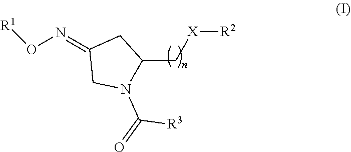
or a geometric isomer, enantiomer, diastereomer, racemate, or salt thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in an amount of from about 1,500 mg to about 2,700 mg per dose,
wherein the compound is administered to the subject prior to transfer of one or more embryos to the uterus of the subject, and optionally wherein the administering reduces the likelihood of embryo implantation failure and/or miscarriage.

2. A method of treating a subject undergoing embryo transfer therapy, the method comprising transferring one or more embryos to the uterus of the subject, wherein the subject has been previously administered a therapeutically effective amount of a compound represented by formula (I) 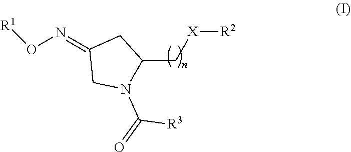
or a geometric isomer, enantiomer, diastereomer, racemate, or salt thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in an amount of from about 1,500 mg to about 2,700 mg per dose, and optionally
wherein administration of the compound reduces the likelihood of embryo implantation failure and/or miscarriage.

3. A method of treating a subject undergoing embryo transfer therapy, the method comprising:
a. administering to the subject a therapeutically effective amount of a compound represented by formula (I) 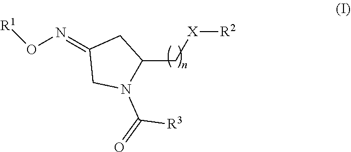
or a geometric isomer, enantiomer, diastereomer, racemate, or salt thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in an amount of from about 1,500 mg to about 2,700 mg per dose; and
b. transferring one or more embryos to the uterus of the subject following administration of the compound;
optionally wherein the administering reduces the likelihood of embryo implantation failure and/or miscarriage.

4. A method of treating a subject undergoing embryo transfer therapy, the method comprising administering to the subject a therapeutically effective amount of a compound represented by formula (I) 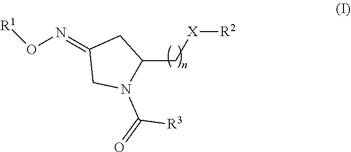
or a geometric isomer, enantiomer, diastereomer, racemate, or sat thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C3-Ce cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in one or more doses totaling from about 1,500 mg to about 2,700 mg,
wherein the compound is administered to the subject prior to transfer of one or more embryos to the uterus of the subject, and optionally wherein the administering reduces the likelihood of embryo implantation failure and/or miscarriage.

5. A method of treating a subject undergoing embryo transfer therapy, the method comprising transferring one or more embryos to the uterus of the subject, wherein the subject has been previously administered a therapeutically effective amount of a compound represented by formula (I) 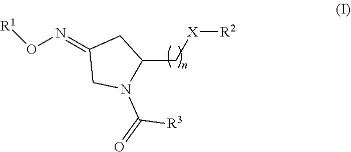
or a geometric isomer, enantiomer, diastereomer, racemate, or sat thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C3-Ce cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in one or more doses totaling from about 1,500 mg to about 2,700 mg, and optionally
wherein administration of the compound reduces the likelihood of embryo implantation failure and/or miscarriage.

6. A method of treating a subject undergoing embryo transfer therapy, the method comprising:
a. administering to the subject a therapeutically effective amount of a compound represented by formula (I) 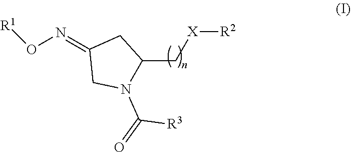
or a geometric isomer, enantiomer, diastereomer, racemate, or sat thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in one or more doses totaling from about 1,500 mg to about 2,700 mg; and
b. transferring one or more embryos to the uterus of the subject following administration of the compound;
optionally wherein the administering reduces the likelihood of embryo implantation failure and/or miscarriage.

7. The method of any one of claims 1-6, wherein the compound is administered to the subject from about 1 hour to about 24 hours prior to the transfer of the one or more embryos to the subject.

8. The method of claim 7, wherein the compound is administered to the subject from about 1 hour to about 8 hours prior to the transfer of the one or more embryos to the subject.

9. The method of claim 8, wherein the compound is administered to the subject from about 3 hours to about 5 hours prior to the transfer of the one or more embryos to the subject.

10. The method of claim 9, wherein the compound is administered to the subject about 4 hours prior to the transfer of the one or more embryos to the subject.

11. The method of any one of claims 1-10, wherein the compound is administered to the subject in a single dose.

12. The method of any one of claims 1-10, wherein the compound is administered to the subject in multiple doses.

13. The method of claim 12, wherein the compound is administered to the subject in from 1 to 20 doses per day prior to the transfer of the one or more embryos to the subject.

14. The method of claim 13, wherein the compound is administered to the subject in from 1 to 7 doses per day prior to the transfer of the one or more embryos to the subject.

15. The method of any one of claims 12-14, wherein the compound is administered to the subject once daily for from about 1 day to about 14 days prior to the transfer of the one or more embryos to the subject.

16. The method of claim 15, wherein the compound is administered to the subject once daily for from about 3 days to about 11 days prior to the transfer of the one or more embryos to the subject.

17. The method of claim 16, wherein the compound is administered to the subject once daily for 7 days prior to the transfer of the one or more embryos to the subject.

18. The method of any one of claims 12-17, wherein the compound is additionally administered to the subject concurrently with the transfer of the one or more embryos to the subject

19. The method of any one of claims 12-18, wherein the compound is additionally administered to the subject following the transfer of the one or more embryos to the subject.

20. The method of claim 19, wherein the compound is additionally administered to the subject from about 1 hour to about 24 hours following the transfer of the one or more embryos to the subject.

21. The method of claim 19 or 20, wherein the compound is additionally administered to the subject in from 1 to 20 doses per day following the transfer of the one or more embryos to the subject.

22. The method of claim 21, wherein the compound is additionally administered to the subject in from 1 to 7 doses per day following the transfer of the one or more embryos to the subject.

23. The method of any one of claims 19-21, wherein the compound is additionally administered to the subject once daily for from about 1 day to about 14 days following the transfer of the one or more embryos to the subject.

24. The method of claim 23, wherein the compound is additionally administered to the subject once daily for from about 3 days to about 11 days following the transfer of the one or more embryos to the subject.

25. The method of claim 24, wherein the compound is additionally administered to the subject once daily for 7 days following the transfer of the one or more embryos to the subject.

26. A method of treating a subject undergoing embryo transfer therapy, the method comprising administering to the subject a therapeutically effective amount of a compound represented by formula (I) 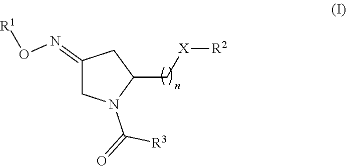
or a geometric isomer, enantiomer, diastereomer, racemate, or salt thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in an amount of from about 1,500 mg to about 2,700 mg per dose,
wherein the compound is administered concurrently with transfer of one or more embryos to the uterus of the subject, and optionally wherein the administering reduces the likelihood of embryo implantation failure and/or miscarriage.

27. A method of treating a subject undergoing embryo transfer therapy, the method comprising transferring one or more embryos to the uterus of the subject, wherein the subject is concurrently administered a therapeutically effective amount of a compound represented by formula (I) 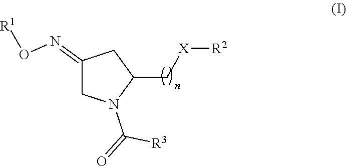
or a geometric isomer, enantiomer, diastereomer, racemate, or sat thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C3-Ce cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in an amount of from about 1,500 mg to about 2,700 mg per dose, and
optionally wherein administration of the compound reduces the likelihood of embryo implantation failure and/or miscarriage.

28. A method of treating a subject undergoing embryo transfer therapy, the method comprising:
a. administering to the subject a therapeutically effective amount of a compound represented by formula (I) 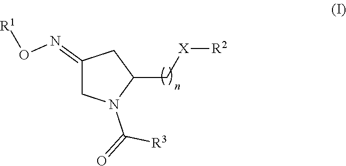
or a geometric isomer, enantiomer, diastereomer, racemate, or salt thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in an amount of from about 1,500 mg to about 2,700 mg per dose; and
b. transferring one or more embryos to the uterus of the subject concurrently with administration of the compound;
optionally wherein the administering reduces the likelihood of embryo implantation failure and/or miscarriage.

29. A method of treating a subject undergoing embryo transfer therapy, the method comprising administering to the subject a therapeutically effective amount of a compound represented by formula (I) 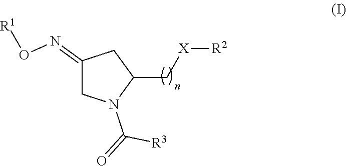
or a geometric isomer, enantiomer, diastereomer, racemate, or salt thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in one or more doses totaling from about 1,500 mg to about 2,700 mg,
wherein the compound is administered concurrently with transfer of one or more embryos to the uterus of the subject, and optionally wherein the administering reduces the likelihood of embryo implantation failure and/or miscarriage.

30. A method of treating a subject undergoing embryo transfer therapy, the method comprising transferring one or more embryos to the uterus of the subject, wherein the subject is concurrently administered a therapeutically effective amount of a compound represented by formula (I) 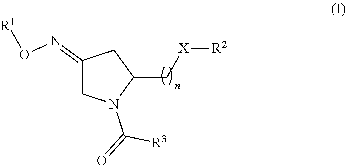
or a geometric isomer, enantiomer, diastereomer, racemate, or salt thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in one or more doses totaling from about 1,500 mg to about 2,700 mg, and
optionally wherein administration of the compound reduces the likelihood of embryo implantation failure and/or miscarriage.

31. A method of treating a subject undergoing embryo transfer therapy, the method comprising:
a. administering to the subject a therapeutically effective amount of a compound represented by formula (I) 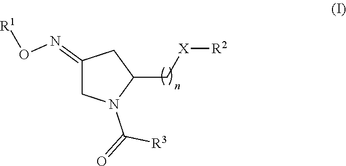
or a geometric isomer, enantiomer, diastereomer, racemate, or salt thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C3-Ce cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in one or more doses totaling from about 1,500 mg to about 2,700 mg; and
b. transferring one or more embryos to the uterus of the subject concurrently with administration of the compound;
optionally wherein the administering reduces the likelihood of embryo implantation failure and/or miscarriage.

32. The method of any one of claims 26-31, wherein the compound is administered to the subject in a single dose.

33. The method of any one of claims 26-31, wherein the compound is administered to the subject in multiple doses.

34. A method of treating a subject undergoing embryo transfer therapy, the method comprising administering to the subject a therapeutically effective amount of a compound represented by formula (I) 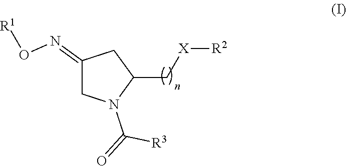
or a geometric isomer, enantiomer, diastereomer, racemate, or salt thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C3-Ce cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in an amount of from about 1,500 mg to about 2,700 mg per dose,
wherein the compound is administered to the subject following transfer of one or more embryos to the uterus of the subject, and optionally wherein the administering reduces the likelihood of embryo implantation failure and/or miscarriage.

35. A method of treating a subject undergoing embryo transfer therapy, the method comprising transferring one or more embryos to the uterus of the subject, wherein the subject is subsequently administered a therapeutically effective amount of a compound represented by formula (I) 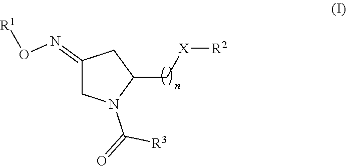
or a geometric isomer, enantiomer, diastereomer, racemate, or salt thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C3-Ce cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in an amount of from about 1,500 mg to about 2,700 mg per dose, and
wherein administration of the compound reduces the likelihood of embryo implantation failure and/or miscarriage.

36. A method of treating a subject undergoing embryo transfer therapy, the method comprising:
a. administering to the subject a therapeutically effective amount of a compound represented by formula (I) 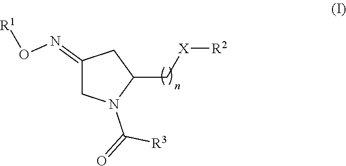
or a geometric isomer, enantiomer, diastereomer, racemate, or sat thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in an amount of from about 1,500 mg to about 2,700 mg per dose; and
b. transferring one or more embryos to the uterus of the subject prior to administration of the compound;
optionally wherein the administering reduces the likelihood of embryo implantation failure and/or miscarriage.

37. A method of treating a subject undergoing embryo transfer therapy, the method comprising administering to the subject a therapeutically effective amount of a compound represented by formula (I) 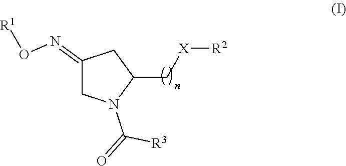
or a geometric isomer, enantiomer, diastereomer, racemate, or salt thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in one or more doses totaling from about 1,500 mg to about 2,700 mg,
wherein the compound is administered to the subject following transfer of one or more embryos to the uterus of the subject, and optionally wherein the administering reduces the likelihood of embryo implantation failure and/or miscarriage.

38. A method of treating a subject undergoing embryo transfer therapy, the method comprising transferring one or more embryos to the uterus of the subject, wherein the subject is subsequently administered a therapeutically effective amount of a compound represented by formula (I) 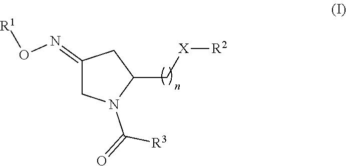
or a geometric isomer, enantiomer, diastereomer, racemate, or salt thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in one or more doses totaling from about 1,500 mg to about 2,700 mg, and
wherein administration of the compound reduces the likelihood of embryo implantation failure and/or miscarriage.

39. A method of treating a subject undergoing embryo transfer therapy, the method comprising:
a. administering to the subject a therapeutically effective amount of a compound represented by formula (I) 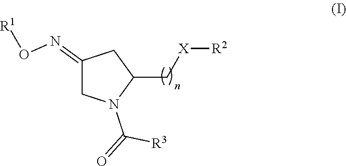
or a geometric isomer, enantiomer, diastereomer, racemate, or salt thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring,
wherein the compound is administered to the subject in one or more doses totaling from about 1,500 mg to about 2,700 mg; and
b. transferring one or more embryos to the uterus of the subject prior to administration of the compound;
optionally wherein the administering reduces the likelihood of embryo implantation failure and/or miscarriage.

40. The method of any one of claims 34-39, wherein the compound is administered to the subject from about 1 hour to about 24 hours following the transfer of the one or more embryos to the subject.

41. The method of any one of claims 34-40, wherein the compound is administered to the subject in a single dose.

42. The method of any one of claims 34-40, wherein the compound is administered to the subject in multiple doses.

43. The method of claim 42, wherein the compound is administered to the subject in from 1 to 20 doses per day following the transfer of the one or more embryos to the subject.

44. The method of claim 43, wherein the compound is administered to the subject in from 1 to 7 doses per day following the transfer of the one or more embryos to the subject.

45. The method of any one of claims 42-44, wherein the compound is administered to the subject once daily for from about 1 day to about 14 days following the transfer of the one or more embryos to the subject.

46. The method of claim 45, wherein the compound is administered to the subject once daily for from about 3 days to about 11 days following the transfer of the one or more embryos to the subject.

47. The method of claim 46, wherein the compound is administered to the subject once daily for 7 days following the transfer of the one or more embryos to the subject.

48. The method of any one of claims 42-47, wherein the compound is additionally administered to the subject concurrently with the transfer of the one or more embryos to the subject.

49. The method of any one of claims 42-48, wherein the compound is additionally administered to the subject prior to the transfer of the one or more embryos to the subject.

50. The method of claim 49, wherein the compound is additionally administered to the subject from about 1 hour to about 24 hours prior to the transfer of the one or more embryos to the subject.

51. The method of claim 50, wherein the compound is additionally administered to the subject from about 1 hour to about 8 hours prior to the transfer of the one or more embryos to the subject.

52. The method of claim 51, wherein the compound is additionally administered to the subject from about 3 hours to about 5 hours prior to the transfer of the one or more embryos to the subject.

53. The method of claim 52, wherein the compound is additionally administered to the subject about 4 hours prior to the transfer of the one or more embryos to the subject.

54. The method of any one of claims 49-53, wherein the compound is additionally administered to the subject in from 1 to 20 doses per day prior to the transfer of the one or more embryos to the subject.

55. The method of claim 54, wherein the compound is additionally administered to the subject in from 1 to 7 doses per day prior to the transfer of the one or more embryos to the subject.

56. The method of any one of claims 49-55, wherein the compound is additionally administered to the subject once daily for from about 1 day to about 14 days prior to the transfer of the one or more embryos to the subject.

57. The method of claim 56, wherein the compound is additionally administered to the subject once daily for from about 3 days to about 11 days prior to the transfer of the one or more embryos to the subject.

58. The method of claim 57, wherein the compound is additionally administered to the subject once daily for 7 days prior to the transfer of the one or more embryos to the subject.

59. The method of any one of claims 1-58, wherein administration of the compound reduces the likelihood of the subject having a miscarriage following the transfer of the one or more embryos.

60. The method of any one of claims 1-59, wherein the compound is administered to the subject in an amount sufficient to achieve a plasma concentration of the compound in the subject of from about 1 μM to about 20 μM.

61. The method of claim 60, wherein the plasma concentration is achieved within from about 1 hour to about 3 hours of administering the compound to the subject.

62. The method of any one of claims 1-51, wherein from 1 to 2 embryos are transferred to the subject.

63. The method of claim 62, wherein 1 embryo is transferred to the subject.

64. The method of claim 62, wherein 2 embryos are transferred to the subject.

65. The method of any one of claims 1-84, wherein the subject is a mammal and the one or more embryos are mammalian embryos.

66. The method of claim 65, wherein the mammal is a human and the one or more mammalian embryos are human embryos.

67. The method of any one of claims 1-66, wherein the one or more embryos are produced ex vivo by in vitro fertilization (IVF).

68. The method of claim 67, wherein the one or more embryos are produced ex vivo by IVF of one or more ova derived from the subject.

69. The method of any one of claims 1-66, wherein the one or more embryos are produced ex vivo by intracytoplasmic sperm injection (ICSI).

70. The method of claim 69, wherein the one or more embryos are produced ex vivo by ICSI into one or more ova derived from the subject.

71. The method of claim 68 or 70, wherein the one or more ova are derived from one or more oocytes isolated from the subject.

72. The method of claim 71, wherein the one or more oocytes are isolated from the subject from about 1 day to about 7 days prior to the transfer of the one or more embryos to the subject.

73. The method of claim 72, wherein the one or more oocytes are isolated from the subject about 2 days prior to the transfer of the one or more embryos to the subject.

74. The method of claim 72, wherein the one or more oocytes are isolated from the subject about 3 days prior to the transfer of the one or more embryos to the subject.

75. The method of claim 72, wherein the one or more oocytes are isolated from the subject about 4 days prior to the transfer of the one or more embryos to the subject.

76. The method of claim 72, wherein the one or more oocytes are isolated from the subject about 5 days prior to the transfer of the one or more embryos to the subject.

77. The method of any one of claims 71-76, wherein the one or more oocytes comprise from 1 to 4 mature oocytes.

78. The method of any one of claims 71-77, wherein a gonadotropin-releasing hormone (GnRH) antagonist is administered to the subject prior to isolation of the one or more oocytes from the subject.

79. The method of any one of claims 71-78, wherein human chorionic gonadotropin (hCG) is administered to the subject prior to isolation of the one or more oocytes from the subject.

80. The method of claim 79, wherein the hCG is administered to the subject by a single intravenous injection.

81. The method of any one of claims 71-80, wherein progesterone is administered to the subject following isolation of the one or more oocytes from the subject.

82. The method of claim 81, wherein the progesterone is administered intravaginally.

83. The method of claim 81 or 82, wherein from about 300 mg to about 600 mg of progesterone per dose is administered to the subject.

84. The method of any one of claims 81-83, wherein the progesterone is administered to the subject daily, preferably beginning within about 24 hours of isolation of the one or more oocytes from the subject and continuing for about 6 or more weeks following the transfer of the one or more embryos to the subject.

85. The method of claim 68 or 70, wherein the one or more ova are isolated directly from the subject.

86. The method of claim 85, wherein the one or more ova are isolated from the subject from about 1 day to about 7 days prior to the transfer of the one or more embryos to the subject.

87. The method of claim 86, wherein the one or more ova are isolated from the subject about 2 days prior to the transfer of the one or more embryos to the subject.

88. The method of claim 86, wherein the one or more ova are isolated from the subject about 3 days prior to the transfer of the one or more embryos to the subject.

89. The method of claim 86, wherein the one or more ova are isolated from the subject about 4 days prior to the transfer of the one or more embryos to the subject.

90. The method of claim 86, wherein the one or more ova are isolated from the subject about 5 days prior to the transfer of the one or more embryos to the subject.

91. The method of any one of claims 85-90, wherein a GnRH antagonist is administered to the subject prior to isolation of the one or more ova from the subject.

92. The method of any one of claims 85-91, wherein hCG is administered to the subject prior to isolation of the one or more ova from the subject.

93. The method of claim 92, wherein the hCG is administered to the subject by a single intravenous injection.

94. The method of any one of claims 85-93, wherein progesterone is administered to the subject following isolation of the one or more ova from the subject.

95. The method of claim 94, wherein the progesterone is administered intravaginally.

96. The method of claim 94 or 95, wherein from about 300 mg to about 600 mg of progesterone per dose is administered to the subject.

97. The method of any one of claims 94-96, wherein the progesterone is administered to the subject daily, preferably beginning within about 24 hours of isolation of the one or more ova from the subject and continuing for about 6 or more weeks following the transfer of the one or more embryos to the subject.

98. The method of any one of claims 71-84, wherein the one or more embryos are transferred to the subject during the same menstrual cycle as isolation of the one or more oocytes from the subject.

99. The method of any one of claims 85-97, wherein the one or more embryos are transferred to the subject during the same menstrual cycle as isolation of the one or more ova from the subject.

100. The method of any one of claims 1-99, wherein the one or more embryos are frozen and thawed prior to the transfer of the one or more embryos to the subject.

101. The method of any one of claims 1-100, wherein the one or more embryos each comprise from 6 to 8 blastomeres immediately prior to the transfer of the one or more embryos to the subject.

102. The method of claim 101, wherein the blastomeres are of approximately equal sizes as assessed by visual microscopy.

103. The method of any one of claims 1-102, wherein the compound is represented by formula (II)

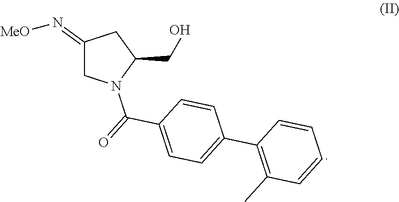 [SMILES: CO/N=C1/C[C@@H](CO)N(C(=O)c2ccc(-c3ccccc3C)cc2)C1]

104. The method of claim 103, wherein the compound is in a crystalline state.

105. The method of claim 104, wherein the compound exhibits characteristic X-ray powder diffraction peaks at about 7.05° 2θ, about 13.13° 2θ, and about 23.34° 2θ.

106. The method of any one of claims 1-105, wherein the compound is administered orally to the subject.

107. The method of any one of claims 1-106, wherein the compound is administered to the subject in the form of a tablet, capsule, gel cap, powder, liquid solution, or liquid suspension.

108. The method of claim 107, wherein the compound is administered to the subject in the form of a tablet.

109. The method of claim 108, wherein the tablet is a dispersible tablet.

110. The method of claim 109, wherein the dispersible tablet comprises:
a. about 1-20% by weight of calcium silicate;
b. about 0.1-20% by weight of PVP30K;
c. about 0.01-5% by weight of poloxamer 188;
d. about 0.5-20% by weight of sodium croscarmellose;
e. about 1-90% by weight of microcrystalline cellulose 112;
f. about 1-90% by weight of lactose monohydrate;
g. about 0.01-0.5% by weight of sodium saccharine; and
h. about 0.1-10% by weight of glycerol dibehenate.

111. The method of claim 110, wherein the dispersible tablet comprises:
a. about 5% by weight of calcium silicate;
b. about 1% by weight of PVP30K;
c. about 2% by weight of poloxamer 188;
d. about 5% by weight of sodium croscarmellose;
e. about 1.5% by weight of microcrystalline cellulose 112;
f. about 47.8% by weight of lactose monohydrate;
g. about 0.2% by weight of sodium saccharine; and
h. about 4% by weight of glycerol dibehenate.

112. The method of any one of claims 1-111, wherein the compound is administered to the subject in an amount of from about 1,600 mg to about 2,000 mg per dose, optionally wherein the compound is administered to the subject in a single dose of from about 1,600 mg to about 2,000 mg.

113. The method of claim 112, wherein the compound is administered to the subject in an amount of from about 1,650 mg to about 1,950 mg per dose, optionally wherein the compound is administered to the subject in a single dose of from about 1,650 mg to about 1,950 mg.

114. The method of claim 113, wherein the compound is administered to the subject in an amount of from about 1,700 mg to about 1,900 mg per dose, optionally wherein the compound is administered to the subject in a single dose of from about 1,700 mg to about 1,900 mg.

115. The method of claim 114, wherein the compound is administered to the subject in an amount of from about 1,750 mg to about 1,850 mg per dose, optionally wherein the compound is administered to the subject in a single dose of from about 1,750 mg to about 1,850 mg.

116. The method of claim 115, wherein the compound is administered to the subject in an amount of about 1,800 mg per dose, optionally wherein the compound is administered to the subject in a single dose of about 1,800 mg.

117. The method of any one of claims 1-116, wherein the compound is administered to the subject in one or more doses totaling from about 1,600 mg to about 2,000 mg.

118. The method of claim 117, wherein the compound is administered to the subject in one or more doses totaling from about 1,650 mg to about 1,950 mg.

119. The method of claim 118, wherein the compound is administered to the subject in one or more doses totaling from about 1,700 mg to about 1,900 mg.

120. The method of claim 119, wherein the compound is administered to the subject in one or more doses totaling from about 1,750 mg to about 1,850 mg.

121. The method of claim 120, wherein the compound is administered to the subject in one or more doses totaling about 1,800 mg.

122. The method of any one of claims 1-111, wherein the compound is administered to the subject in an amount of from about 1,900 mg to about 2,300 mg per dose, optionally wherein the compound is administered to the subject in a single dose of from about 1,900 mg to about 2,300 mg.

123. The method of claim 122, wherein the compound is administered to the subject in an amount of from about 1,950 mg to about 2,250 mg per dose, optionally wherein the compound is administered to the subject in a single dose of from about 1,950 mg to about 2,250 mg.

124. The method of claim 123, wherein the compound is administered to the subject in an amount of from about 2,000 mg to about 2,200 mg per dose, optionally wherein the compound is administered to the subject in a single dose of from about 2,000 mg to about 2,200 mg.

125. The method of claim 124, wherein the compound is administered to the subject in an amount of from about 2,050 mg to about 2,150 mg per dose, optionally wherein the compound is administered to the subject in a single dose of from about 2,050 mg to about 2,150 mg.

126. The method of claim 125, wherein the compound is administered to the subject in an amount of about 2,100 mg per dose, optionally wherein the compound is administered to the subject in a single dose of about 2,100 mg.

127. The method of any one of claims 1-111 and 122-126, wherein the compound is administered to the subject in one or more doses totaling from about 1,900 mg to about 2,300 mg.

128. The method of claim 127, wherein the compound is administered to the subject in one or more doses totaling from about 1,950 mg to about 2,250 mg.

129. The method of claim 128, wherein the compound is administered to the subject in one or more doses totaling from about 2,000 mg to about 2,200 mg.

130. The method of claim 129, wherein the compound is administered to the subject in one or more doses totaling from about 2,050 mg to about 2,150 mg.

131. The method of claim 130, wherein the compound is administered to the subject in one or more doses totaling about 2,100 mg.

132. The method of any one of claims 1-111, wherein the compound is administered to the subject in an amount of from about 2,200 mg to about 2,600 mg per dose, optionally wherein the compound is administered to the subject in a single dose of from about 2,200 mg to about 2,600 mg.

133. The method of claim 132, wherein the compound is administered to the subject in an amount of from about 2,250 mg to about 2,550 mg per dose, optionally wherein the compound is administered to the subject in a single dose of from about 2,250 mg to about 2,550 mg.

134. The method of claim 133, wherein the compound is administered to the subject in an amount of from about 2,300 mg to about 2,500 mg per dose, optionally wherein the compound is administered to the subject in a single dose of from about 2,300 mg to about 2,500 mg.

135. The method of claim 134, wherein the compound is administered to the subject in an amount of from about 2,350 mg to about 2,450 mg per dose, optionally wherein the compound is administered to the subject in a single dose of from about 2,350 mg to about 2,450 mg.

136. The method of claim 135, wherein the compound is administered to the subject in an amount of about 2,400 mg per dose, optionally wherein the compound is administered to the subject in a single dose of about 2,400 mg.

137. The method of any one of claims 1-111 and 132-136, wherein the compound is administered to the subject in one or more doses totaling from about 2,200 mg to about 2,600 mg.

138. The method of claim 137, wherein the compound is administered to the subject in one or more doses totaling from about 2,250 mg to about 2,550 mg.

139. The method of claim 138, wherein the compound is administered to the subject in one or more doses totaling from about 2,300 mg to about 2,500 mg.

140. The method of claim 139, wherein the compound is administered to the subject in one or more doses totaling from about 2,350 mg to about 2,450 mg.

141. The method of claim 140, wherein the compound is administered to the subject in one or more doses totaling about 2,400 mg.

142. The method of any one of claims 1-141, wherein the subject exhibits a reduction in the frequency of uterine contractions following administration of the compound to the subject.

143. The method of claim 142, wherein the reduction is from about 1% to about 20% relative to a measurement of the frequency of uterine contractions in the subject recorded prior to administration of the compound to the subject.

144. The method of any one of claims 1-143, wherein the subject has been determined to exhibit a serum progesterone (P4) concentration of less than 320 nM prior to the transfer of the one or more embryos to the subject, optionally wherein the subject has been determined to exhibit a serum P4 concentration of less than about 320 nM within 24 hours prior to the transfer of the one or more embryos to the subject.

145. The method of claim 144, wherein the subject has been determined to exhibit a serum P4 concentration of from 200 nM to 300 nM prior to the transfer of the one or more embryos to the subject, optionally wherein the subject has been determined to exhibit a serum P4 concentration of from about 200 nM to about 300 nM within 24 hours prior to the transfer of the one or more embryos to the subject.

146. The method of any one of claims 1-145, wherein the subject has been determined to exhibit a serum P4 concentration of less than 2.0 ng/ml (e.g., a serum P4 concentration of 1.54 ng/ml or less) prior to the transfer of the one or more embryos to the subject, optionally wherein the subject has been determined to exhibit a serum P4 concentration of less than 2.0 ng/ml from about 1 day to about 7 days prior to the transfer of the one or more embryos to the subject.

147. The method of claim 146, wherein the subject has been determined to exhibit a serum P4 concentration of less than 2.0 ng/ml (e.g., a serum P4 concentration of 1.54 ng/ml or less) about 2 days prior to the transfer of the one or more embryos to the subject.

148. The method of claim 146, wherein the subject has been determined to exhibit a serum P4 concentration of less than 2.0 ng/ml (e.g., a serum P4 concentration of 1.54 ng/ml or less) about 3 days prior to the transfer of the one or more embryos to the subject.

149. The method of claim 146, wherein the subject has been determined to exhibit a serum P4 concentration of less than 2.0 ng/ml (e.g., a serum P4 concentration of 1.54 ng/ml or less) about 4 days prior to the transfer of the one or more embryos to the subject.

150. The method of claim 146, wherein the subject has been determined to exhibit a serum P4 concentration of less than 2.0 ng/ml (e.g., a serum P4 concentration of 1.54 ng/ml or less) about 5 days prior to the transfer of the one or more embryos to the subject.

151. The method of any one of claims 148-150, wherein the subject has been determined to exhibit the serum P4 concentration on the day of isolation of one or more oocytes or ova from the subject.

152. The method of claim 151, wherein the subject has been determined to exhibit the serum P4 concentration within about 48 hours of administering hCG to the subject (e.g., so as to induce final follicular maturation).

153. The method of claim 146, wherein the subject has been determined to exhibit a serum P4 concentration of less than 1.5 ng/ml prior to the transfer of the one or more embryos to the subject, optionally wherein the subject has been determined to exhibit a serum P4 concentration of less than 1.5 ng/ml from about 1 day to about 7 days prior to the transfer of the one or more embryos to the subject.

154. The method of claim 153, wherein the subject has been determined to exhibit a serum P4 concentration of less than 1.5 ng/ml about 2 days prior to the transfer of the one or more embryos to the subject.

155. The method of claim 153, wherein the subject has been determined to exhibit a serum P4 concentration of less than 1.5 ng/ml about 3 days prior to the transfer of the one or more embryos to the subject.

156. The method of claim 153, wherein the subject has been determined to exhibit a serum P4 concentration of less than 1.5 ng/ml about 4 days prior to the transfer of the one or more embryos to the subject.

157. The method of claim 153, wherein the subject has been determined to exhibit a serum P4 concentration of less than 1.5 ng/ml about 5 days prior to the transfer of the one or more embryos to the subject.

158. The method of any one of claims 153-157 wherein the subject has been determined to exhibit the serum P4 concentration on the day of isolation of one or more oocytes or ova from the subject.

159. The method of claim 158, wherein the subject has been determined to exhibit the serum P4 concentration within about 48 hours of administering hCG to the subject.

160. The method of any one of claims 1-159, wherein the subject exhibits an increase in endometrial prostaglandin F2α (PGF2α) expression following administration of the compound to the subject.

161. The method of any one of claims 1-160, wherein the subject exhibits a reduction in PGF2α signaling following administration of the compound to the subject.

162. The method of any one of claims 1-161, wherein the subject exhibits an increase in endometrial prostaglandin E2 (PGE2) expression following administration of the compound to the subject.

163. The method of any one of claims 1-162, wherein the subject sustains pregnancy for at least about 14 days following the transfer of the one or more embryos to the subject.

164. The method of claim 163, wherein the subject sustains pregnancy for at least about 6 weeks following the transfer of the one or more embryos to the subject.

165. The method of claim 164, wherein the subject sustains pregnancy for at least about 10 weeks following retrieval of one or more oocytes or ova from the subject.

166. The method of any one of claims 163-165, wherein pregnancy is assessed by a blood pregnancy test.

167. The method of claim 166, wherein the blood pregnancy test comprises detecting hCG in a blood sample isolated from the subject.

168. The method of claim 164 or 165, wherein pregnancy is assessed by detecting intrauterine embryo heartbeat.

169. The method of any one of claims 1-168, wherein the subject sustains pregnancy and exhibits a live birth following administration of the compound to the subject.

170. The method of claim 169, wherein the subject exhibits the live birth at a gestational age of at least about 24 weeks.

171. A kit comprising a package insert and a compound represented by formula (I) 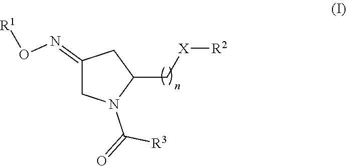
or a geometric isomer, enantiomer, diastereomer, racemate, or salt thereof, wherein
n is an integer from 1 to 3;
R¹ is selected from the group consisting of hydrogen and C₁-C₆ alkyl;
R² is selected from the group consisting of hydrogen, C₁-C₆ alkyl, C₁-C₆ alkyl aryl, heteroaryl, C₁-C₆ alkyl heteroaryl, C₂-C₆ alkenyl, C₂-C₆ alkenyl aryl, C₁-C₆ alkenyl heteroaryl, C₂-C₆ alkynyl, C₂-C₆ alkynyl aryl, C₂-C₆ alkynyl heteroaryl, C₁-C₆ cycloalkyl, heterocycloalkyl, C₁-C₆ alkyl cycloalkyl, C₁-C₆ alkyl heterocycloalkyl, C₁-C₆ alkyl carboxy, acyl, C₁-C₆ alkyl acyl, C₁-C₆ alkyl acyloxy, C₁-C₆ alkyl alkoxy, alkoxycarbonyl, C₁-C₆ alkyl alkoxycarbonyl, aminocarbonyl, C₁-C₆ alkyl aminocarbonyl, C₁-C₆ alkyl acylamino, C₁-C₆ alkyl ureido, amino, C₁-C₆ alkyl amino, sulfonyloxy, C₁-C₆ alkyl sulfonyloxy, sulfonyl, C₁-C₆ alkyl sulfonyl, sulfinyl, C₁-C₆ alkyl sulfinyl, C₁-C₆ alkyl sulfanyl, and C₁-C₆ alkyl sulfonylamino;
R³ is selected from the group consisting of aryl and heteroaryl;
X is selected from the group consisting of oxygen and NR⁴; and
R⁴ is selected from the group consisting of hydrogen, C₁-C₆ alkyl, C₁-C₆ alkyl aryl, C₁-C₆ alkyl heteroaryl, aryl, and heteroaryl, wherein R² and R⁴, together with the nitrogen to which they are bound, can form a 5-8 membered saturated or unsaturated heterocycloalkyl ring;
wherein the package insert instructs a user of the kit to perform the method of any one of claims 1-170.

172. The kit of claim 171, wherein the compound is represented by formula (II)

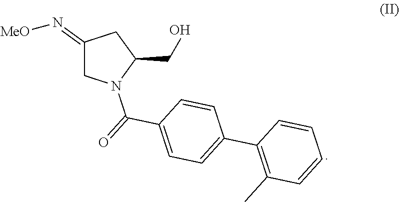 [SMILES: CO/N=C1/C[C@@H](CO)N(C(=O)c2ccc(-c3ccccc3C)cc2)C1]

173. The kit of claim 171 or 172, wherein the compound is formulated for oral administration to the subject.

174. The kit of claim 173, wherein the compound is formulated as a tablet, capsule, gel cap, powder, liquid solution, or liquid suspension.

175. The kit of claim 174, wherein the compound is formulated as a tablet.

176. The kit of claim 175, wherein the tablet is a dispersible tablet.

177. The kit of any one of claims 171-176, wherein the compound is formulated in a unit dosage form comprising about 50 mg of the compound.

178. The kit of any one of claims 171-176, wherein the compound is formulated in a unit dosage form comprising about 200 mg of the compound.

179. The kit of any one of claims 171-178, wherein the kit comprises from about 1,600 mg to about 2,000 mg of the compound.

180. The kit of claim 179, wherein the kit comprises from about 1,650 mg to about 1,950 mg of the compound.

181. The kit of claim 180, wherein the kit comprises from about 1,700 mg to about 1,900 mg of the compound.

182. The kit of claim 181, wherein the kit comprises from about 1,750 mg to about 1,850 mg of the compound.

183. The kit of claim 182, wherein the kit comprises about 1,800 mg of the compound.

184. The kit of any one of claims 171-178, wherein the kit comprises from about 1,900 mg to about 2,300 mg of the compound.

185. The kit of claim 184, wherein the kit comprises from about 1,950 mg to about 2,250 mg of the compound.

186. The kit of claim 185, wherein the kit comprises from about 2,000 mg to about 2,200 mg of the compound.

187. The kit of claim 186, wherein the kit comprises from about 2,050 mg to about 2,150 mg of the compound.

188. The kit of claim 187, wherein the kit comprises about 2,100 mg of the compound.

189. The kit of any one of claims 171-178, wherein the kit comprises from about 2,200 mg to about 2,600 mg of the compound.

190. The kit of claim 189, wherein the kit comprises from about 2,250 mg to about 2,550 mg of the compound.

191. The kit of claim 190, wherein the kit comprises from about 2,300 mg to about 2,500 mg of the compound.

192. The kit of claim 191, wherein the kit comprises from about 2,350 mg to about 2,450 mg of the compound.

193. The kit of claim 192, wherein the kit comprises about 2,400 mg of the compound.
